(19) United States
(12) Patent Application Publication
(10) Pub. No.: US 2026/0152757 A1
(43) Pub. Date: Jun. 4, 2026

(54) METHODS AND COMPOSITIONS FOR PPO HERBICIDE TOLERANCE

(71) Applicant: Monsanto Technology LLC, St. Louis, MO (US)

(72) Inventors: Udo Bickers, Köln (DE); Jens Frackenpohl, Frankfurt (DE); Ines Heinemann, Hofheim (DE); Hendrik Helmke, Liederbach (DE); Anna Reingruber, Heppenheim (DE)

(21) Appl. No.: 19/268,483

(22) Filed: Jul. 14, 2025

Related U.S. Application Data
(60) Provisional application No. 63/673,291, filed on Jul. 19, 2024.

(51) Int. Cl. C12N 15/82; A01N 43/54; A01N 43/58; A01P 13/02; C12N 9/02

(52) U.S. Cl. CPC C12N 15/8274; A01N 43/54; A01N 43/58; A01P 13/02; C12N 9/001; C12Y 103/03004; C12N 2830/34

(57) ABSTRACT

The present disclosure relates to the use of substituted uracil pyridines or agrochemically acceptable salts thereof, for controlling or preventing weed growth in plant growth areas of transgenic crop plants that are tolerant to PPO inhibiting herbicides. The disclosure also provides herbicide tolerant transgenic plants, seeds, cells, and plant parts containing the recombinant DNA molecules, as well as methods of using the same.

### CROSS-REFERENCE TO RELATED APPLICATIONS

This application claims priority to U.S. Provisional Application Ser. No. 63/673,291, filed Jul. 19, 2024, the entire disclosure of which is incorporated herein by reference.

### FIELD OF THE INVENTION

The present disclosure relates to the fields of agriculture, plant biotechnology, and molecular biology. More specifically, the disclosure relates to the use of substituted uracil pyridines or agrochemically acceptable salts thereof, for controlling or preventing weed growth in plant growth areas of transgenic crop plants that are tolerant to protoporphyrinogen oxidase (PPO) inhibiting herbicides.

### INCORPORATION OF SEQUENCE LISTING

A computer readable form of a sequence listing is filed with this application by electronic submission and is incorporated into this application by reference in its entirety. The sequence listing is contained in the file named MONS598US_ST26.xml, which is 686,321 bytes in size (measured in operating system MS Windows®) and was created on Jul. 1, 2025.

### BACKGROUND OF THE INVENTION

Chemical herbicides are often used to control the growth and spread of weeds or other plants that are unwanted in a particular environment. These chemicals are active at one or more target sites within a plant where they interrupt normal plant functions. Herbicides vary in their modes of action, in their effects on weeds and crop plants, and how they are used. While herbicides are very effective in controlling growth of undesirable vegetation, their use may also cause incidental damage to desired plants located in the same vicinity, such as crop plants. In order to minimize crop damage, extensive research has been directed toward the development of herbicide tolerant plants, especially through use of transgenic traits. Examples of transgenic herbicide tolerance traits include glyphosate tolerance, glufosinate tolerance, and dicamba tolerance. With the increase of weed species resistant to the commonly used herbicides, especially glyphosate, growers have turned to use of herbicides having different modes of action and thus new herbicide tolerance traits are needed in the field. Herbicides of particular interest include herbicides that inhibit protoporphyrinogen oxidase (PPO, EC 1.3.3.4), referred to as PPO inhibitor herbicides. PPO inhibitor herbicides provide control of a spectrum of herbicide-resistant weeds, thus making a trait conferring tolerance to these herbicides particularly useful in a cropping system combined with one or more other herbicide-tolerance trait(s).

### SUMMARY OF THE INVENTION

Provided herein is a method for controlling or preventing weed growth in a plant growth area, wherein the method comprises the steps of:
(a) providing in said plant growth area a plant or a seed that when grown produces said plant, wherein the plant comprises a recombinant DNA molecule comprising a DNA sequence encoding a heterologous HemG protein, wherein said protein confers tolerance in said plant to an herbicidally active compound of the general formula (I) or an agrochemically acceptable salt thereof

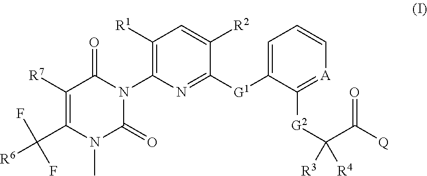

in which
A represents N (nitrogen) or a group C—R⁵,
G¹ and G² independently of each other represent O (oxygen) or S (sulfur),
R¹ represents fluorine, hydrogen, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents chlorine, fluorine, bromine, iodine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O—(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl, or
R³ and R⁴ together with the carbon atom to which they are bonded form a fully saturated or partly saturated 3- to 10-membered carbocyclic ring optionally having further substitution,
R⁵ represents hydrogen, halogen, (C₁-C₈)-alkyl, (C₁-C₈)-haloalkyl, or OR¹³,
R⁶ represents fluorine, hydrogen, chlorine, bromine, trifluoromethyl, difluoromethyl, methoxy, ethoxy, prop-1-yloxy, but-1-yloxy or methyl,
R⁷ represents hydrogen or methyl,
Q represents hydroxy or a group Q-1, Q-2

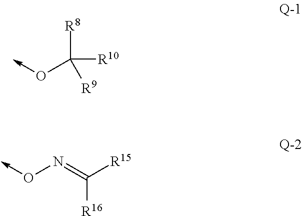

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₁C₈)-haloalkyl, (C₃C₈)-cycloalkyl, (C₃C₈)-cycloalkyl-(C₁C₈)-alkyl, (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O—(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₈)-cycloalkyl, aryl, heteroaryl, or heterocyclyl;
and (b) applying to said area an amount of said compound effective to control or prevent weed growth in the area.

In certain embodiments, the compound of the general formula (I) or an agrochemically acceptable salt thereof is further characterized in that
A represents N (nitrogen) or a group C—R⁵,
G¹ and G² independently of each other represent O (oxygen) or S (sulfur),
R¹ represents hydrogen, fluorine,
R² represents chlorine, bromine, iodine, nitro or cyano,
R³ and R⁴ independently of each other represent hydrogen, methyl, or ethyl, or
R³ and R⁴ together with the carbon atom to which they are bonded form a fully saturated or partly saturated 3- to 6-membered carbocyclic ring optionally having further substitution,
R⁵ represents hydrogen, chlorine, or methoxy,
R⁶ represents fluorine, methyl,
R⁷ represents hydrogen, methyl,
and
Q represents one of the following moieties Q-1 to Q-500:

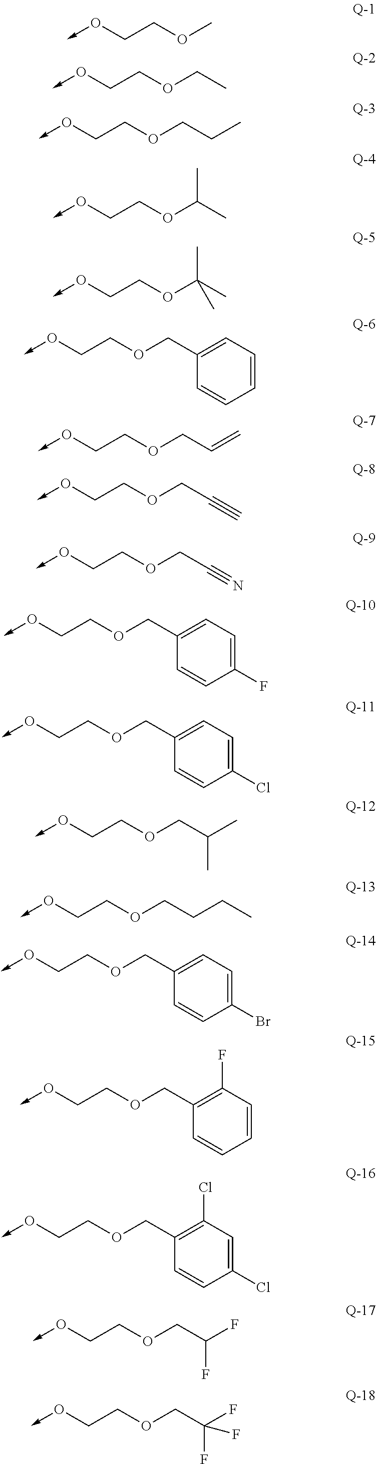

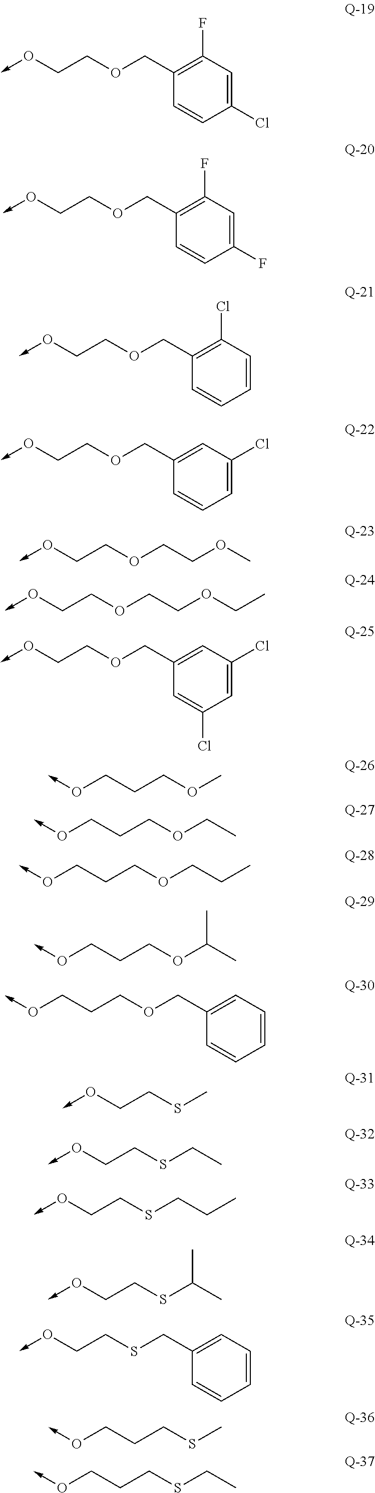

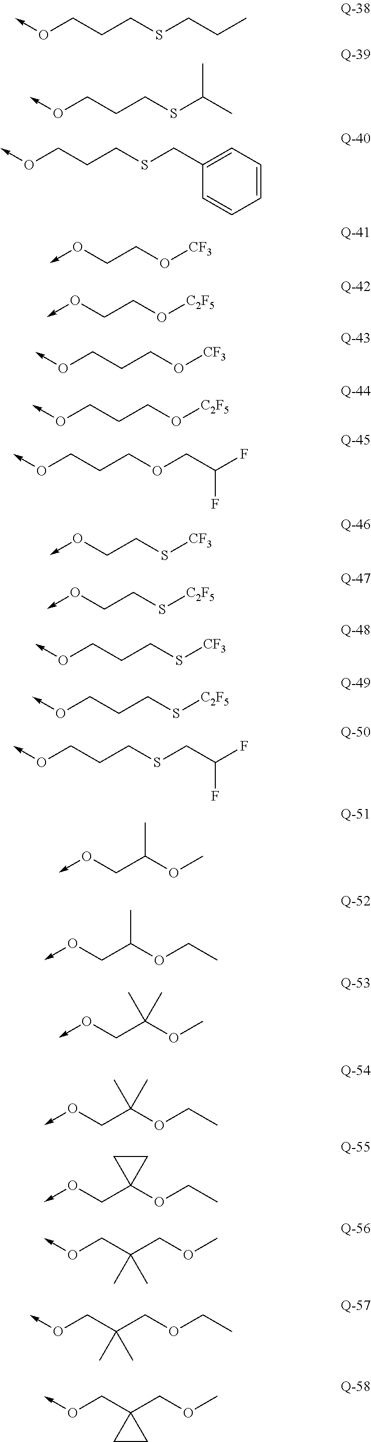

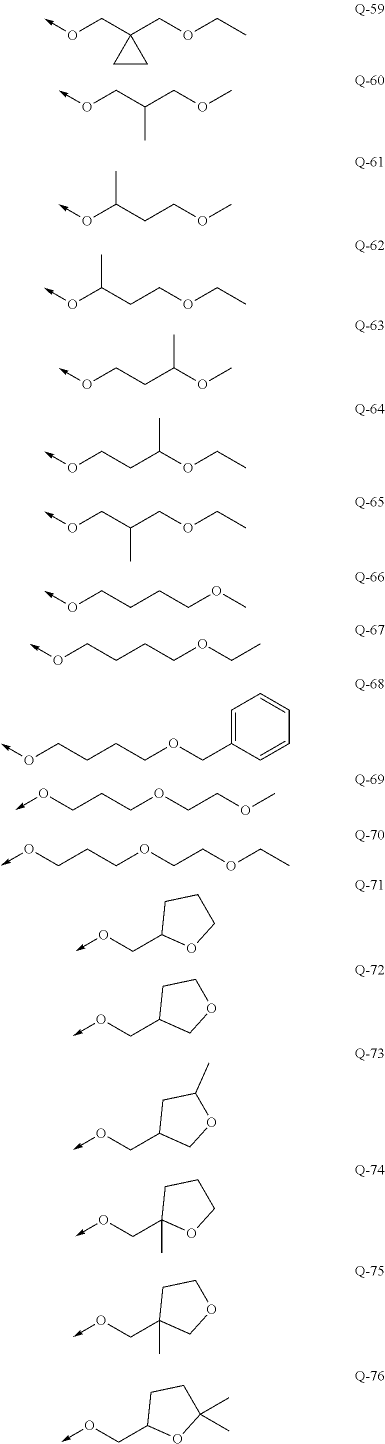

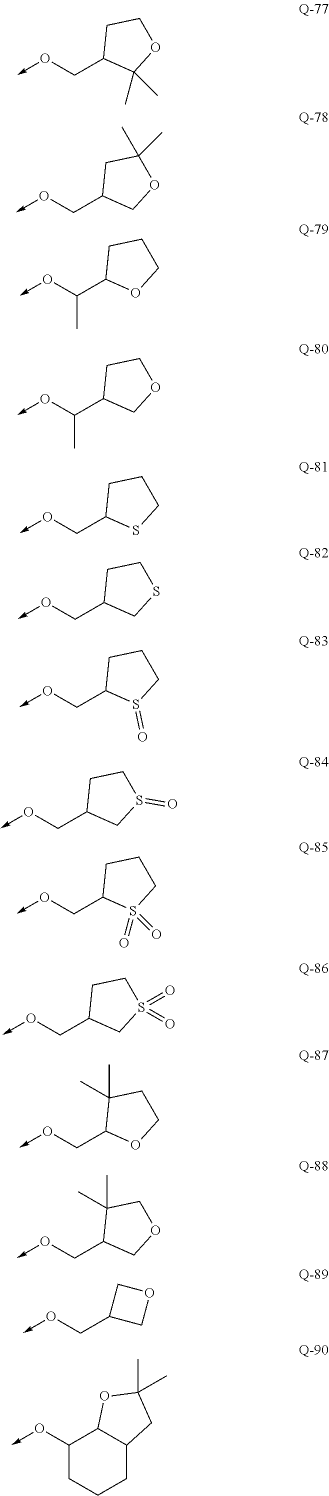

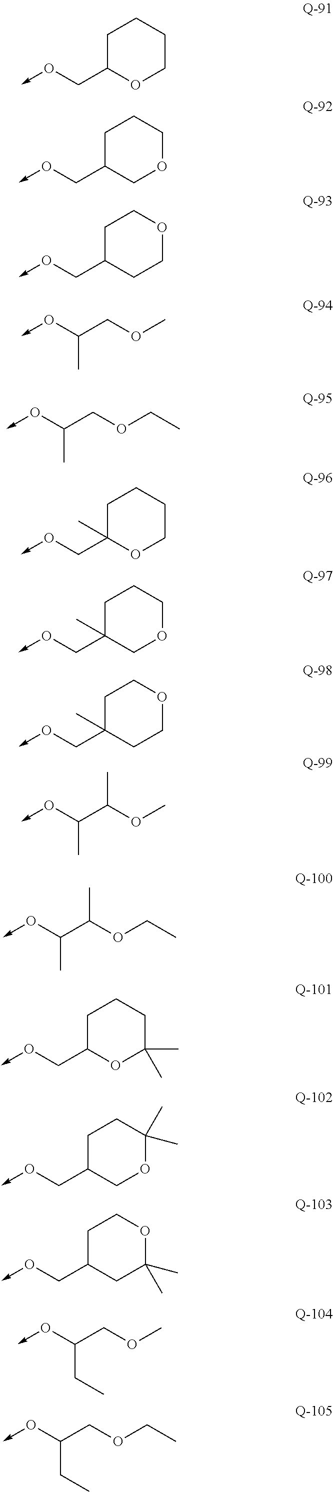

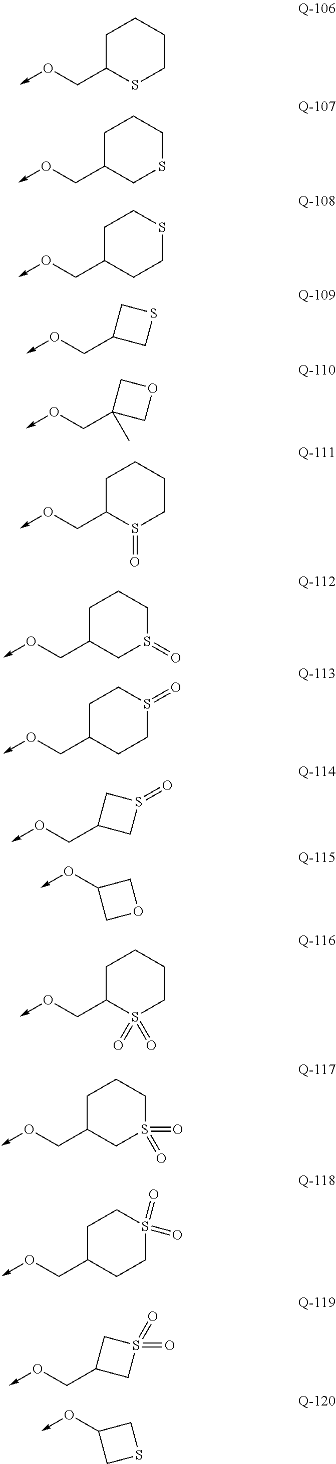

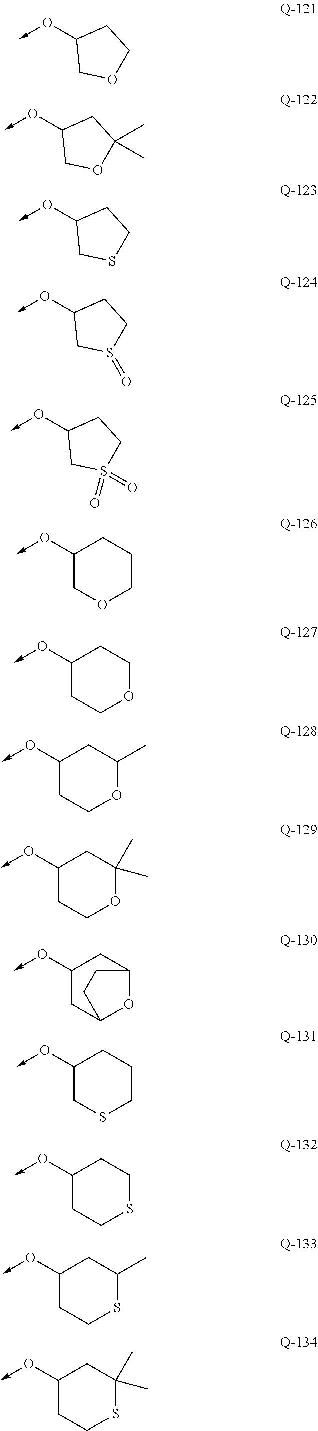

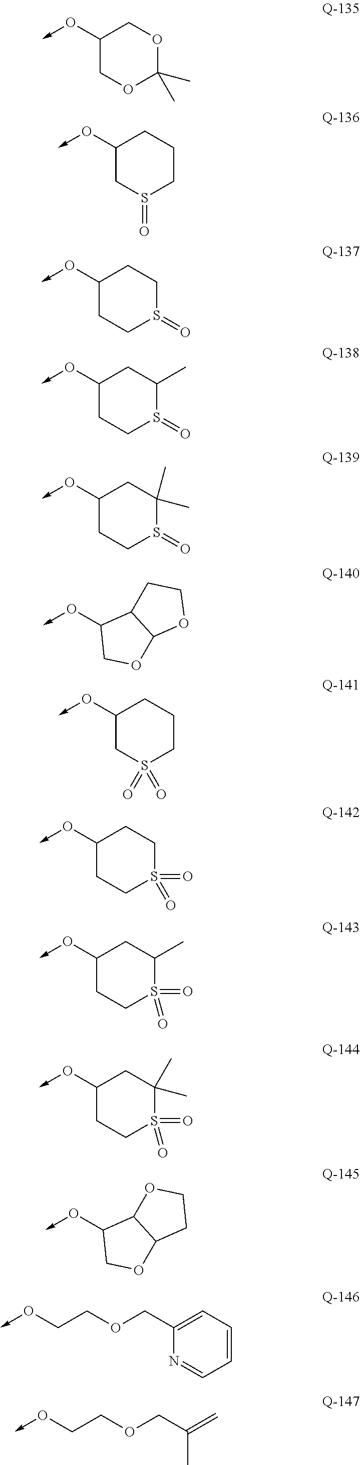

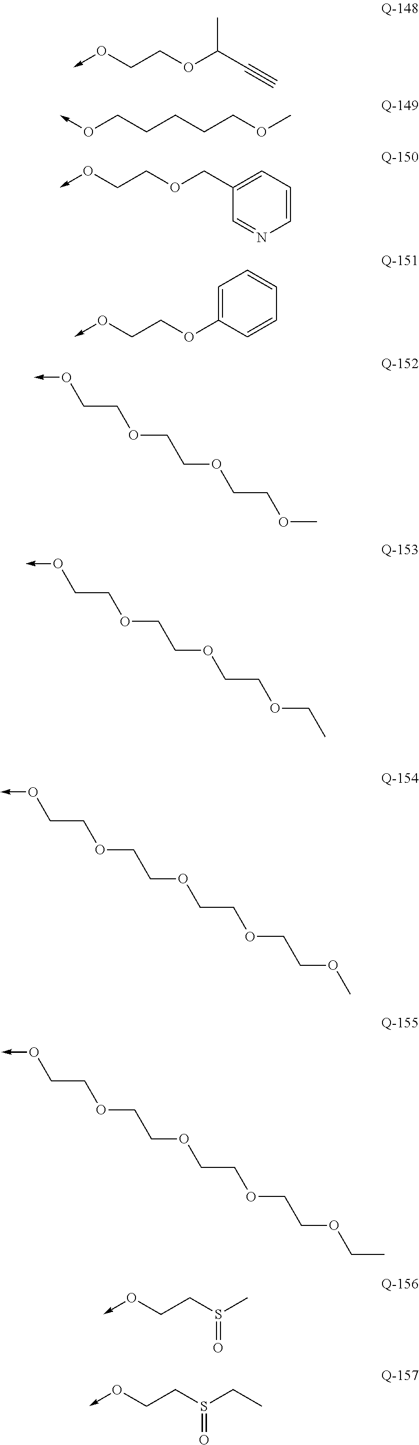

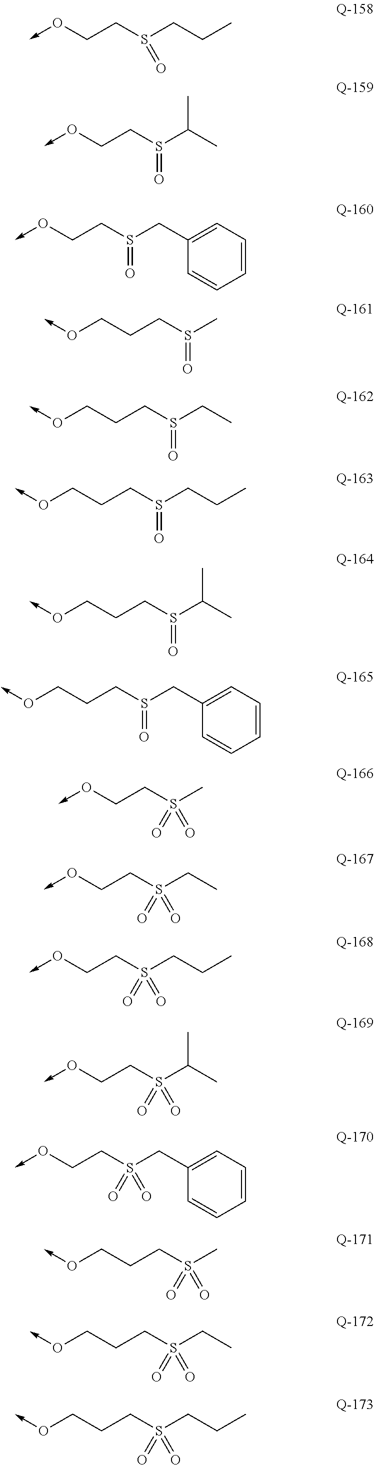

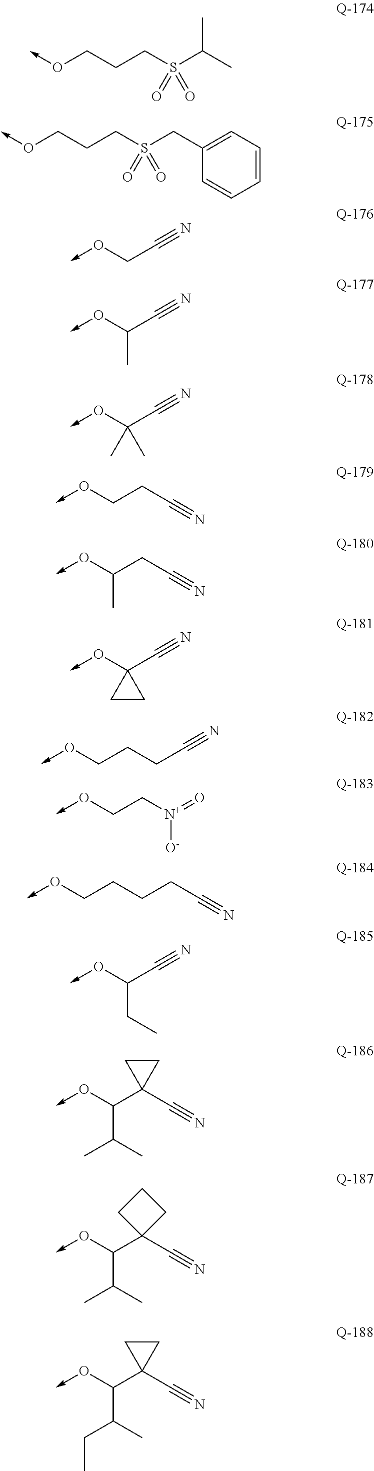

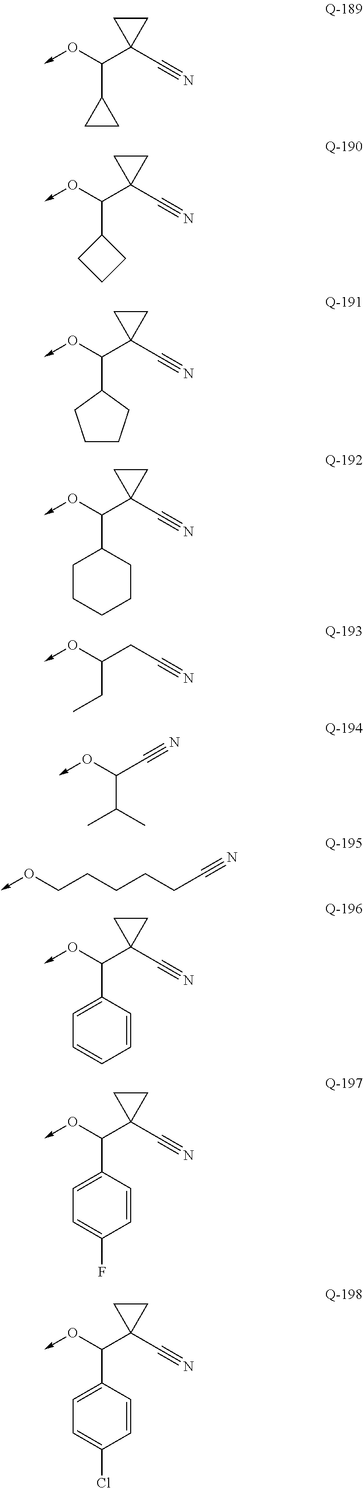

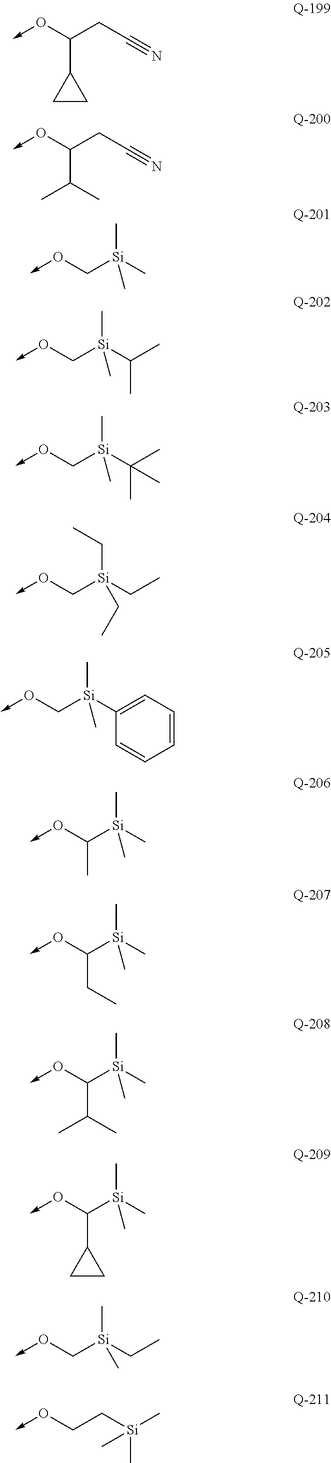

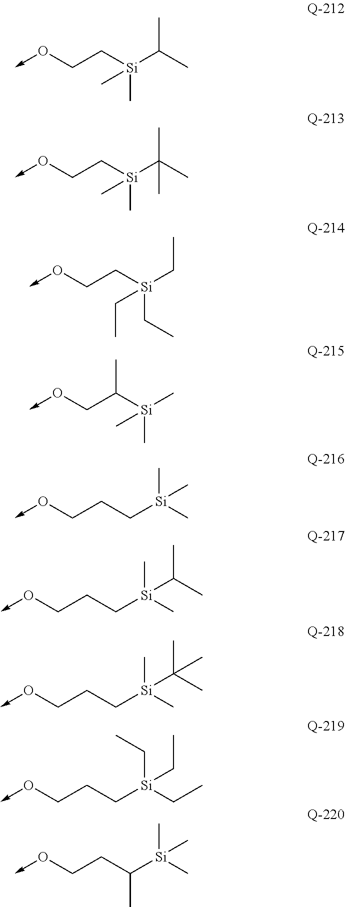

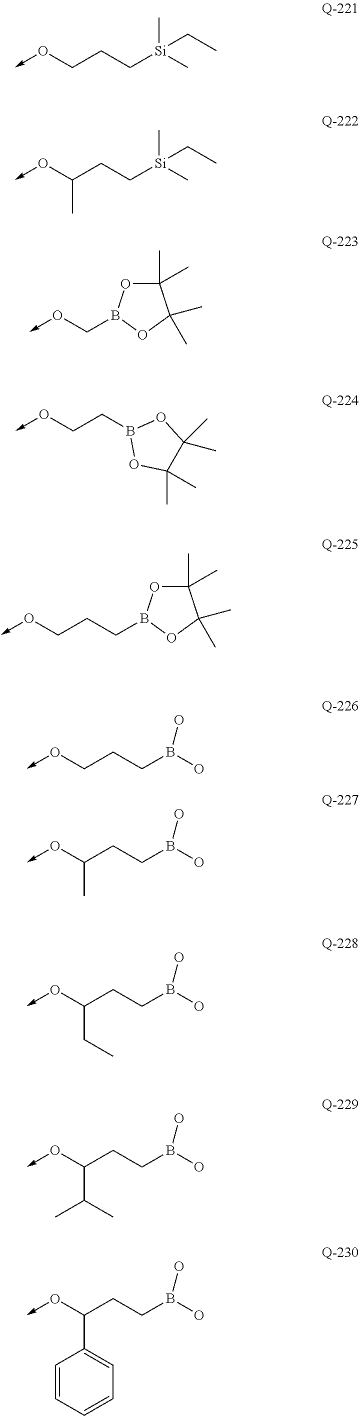

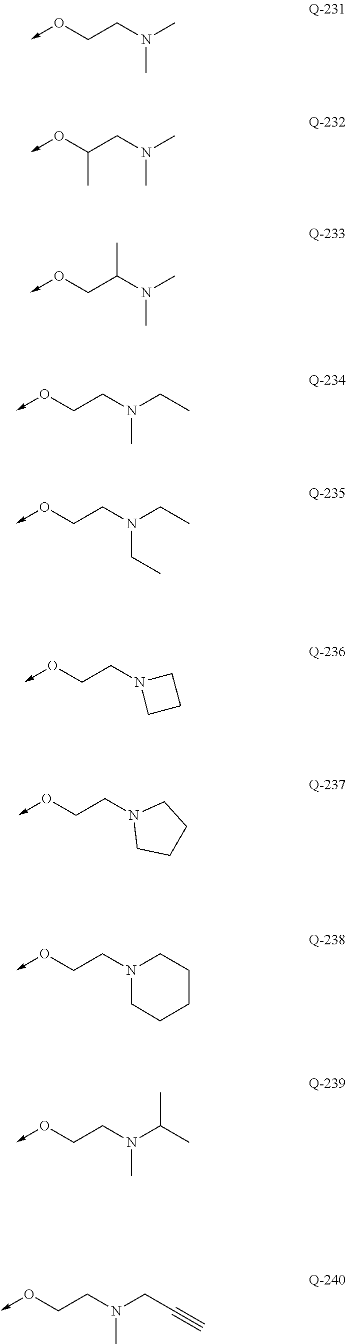

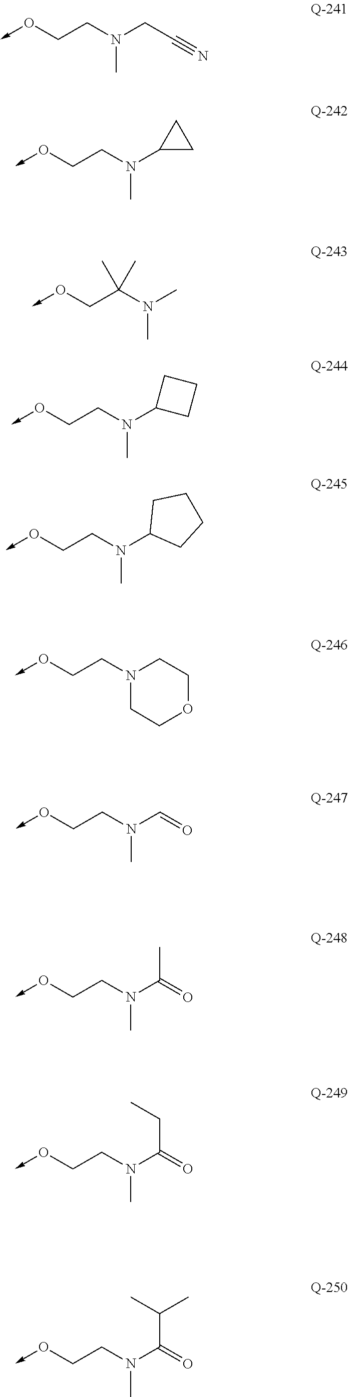

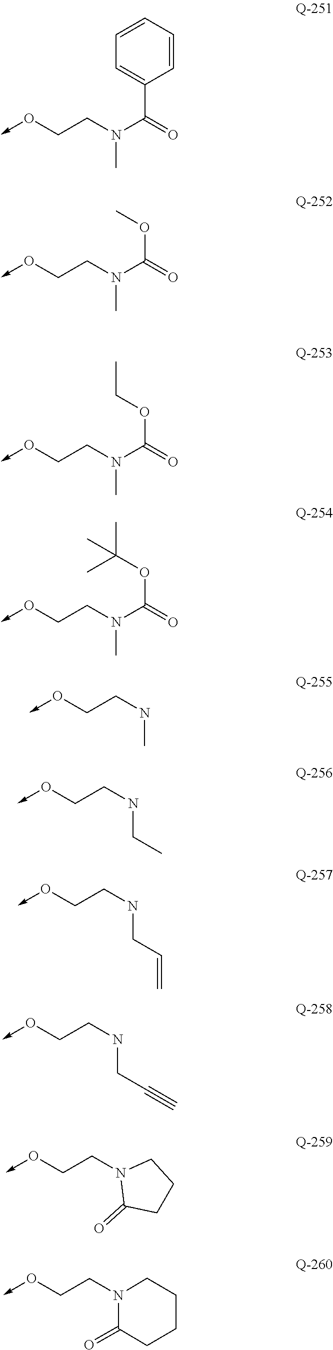

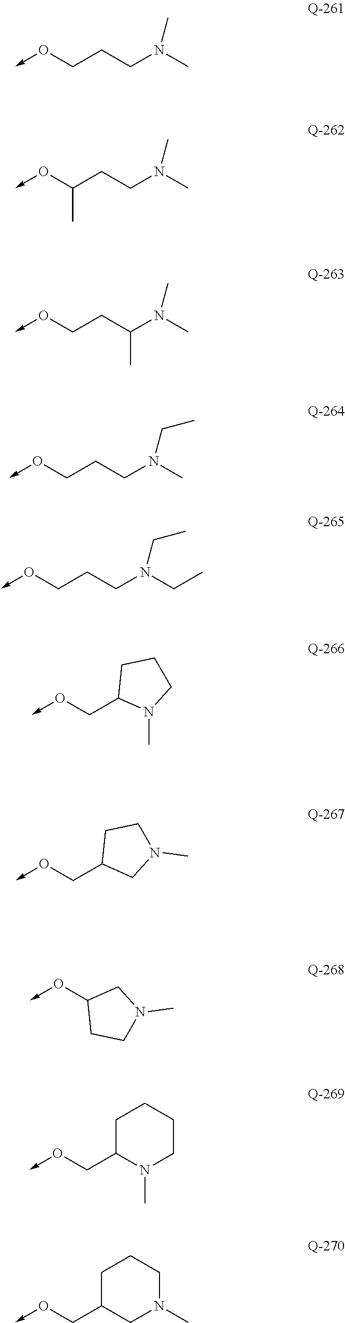

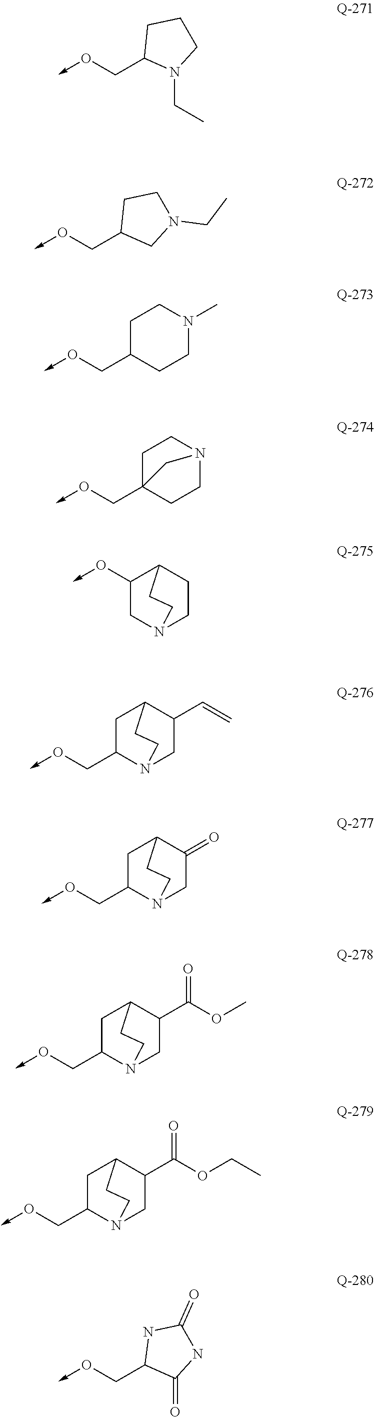

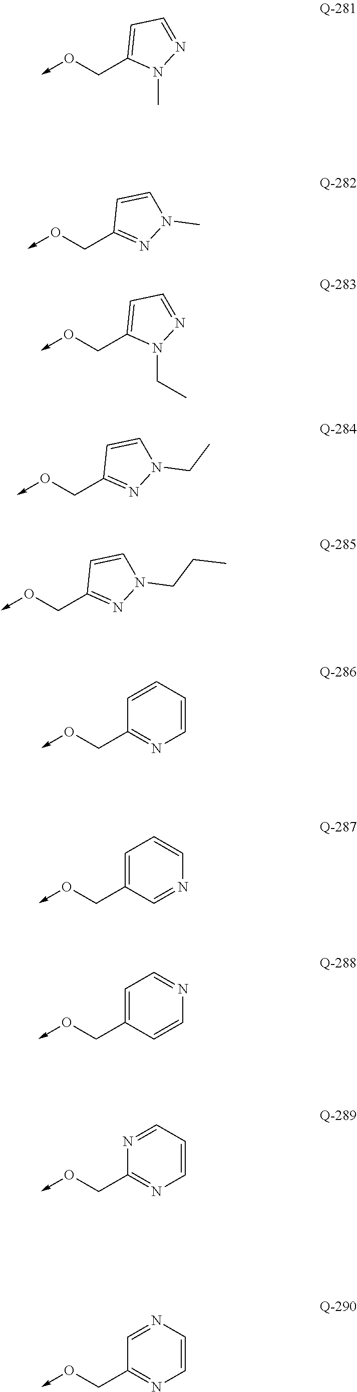

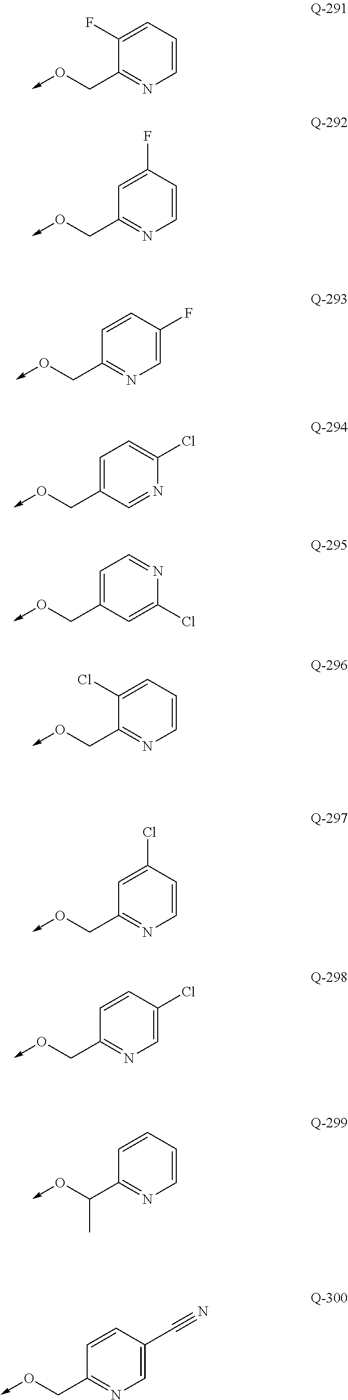

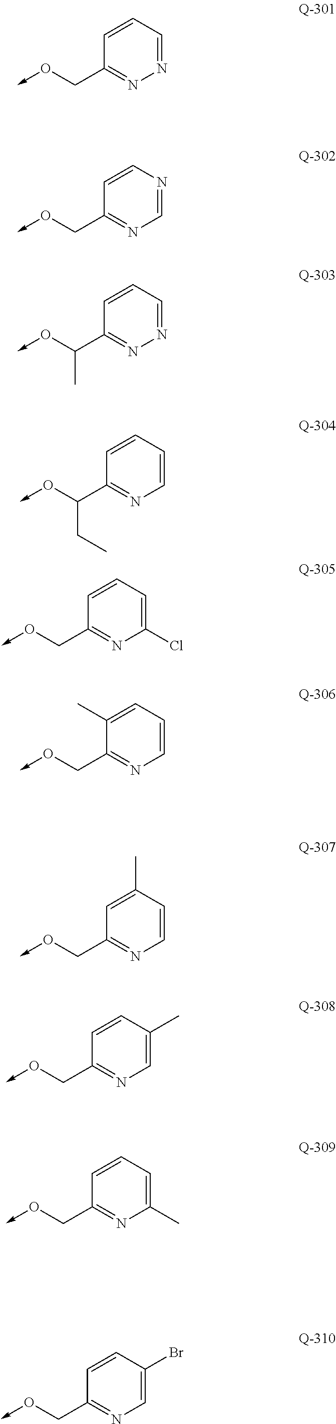

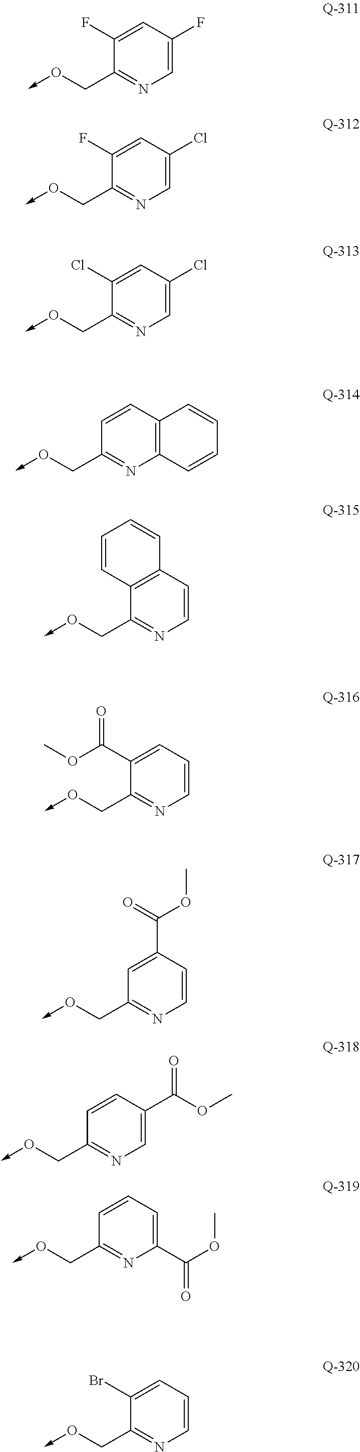

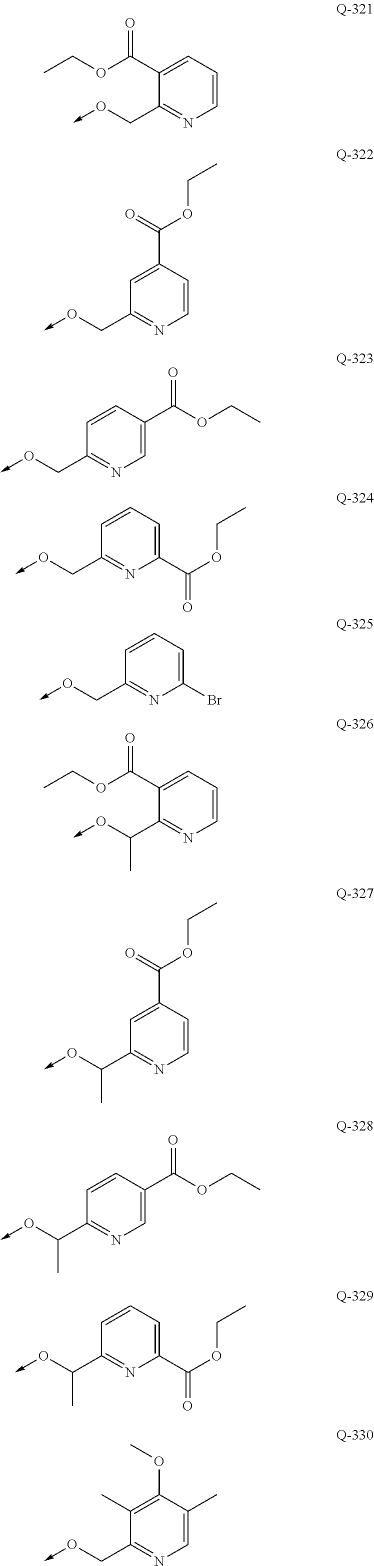

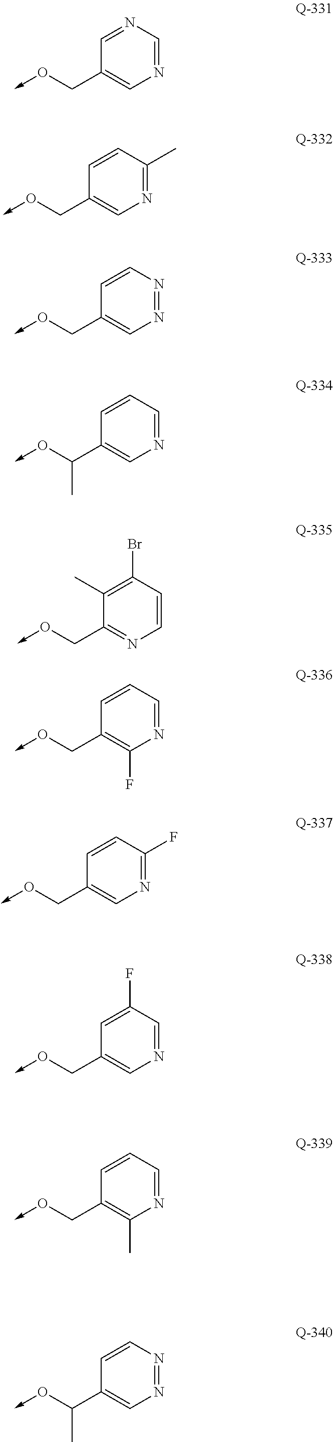

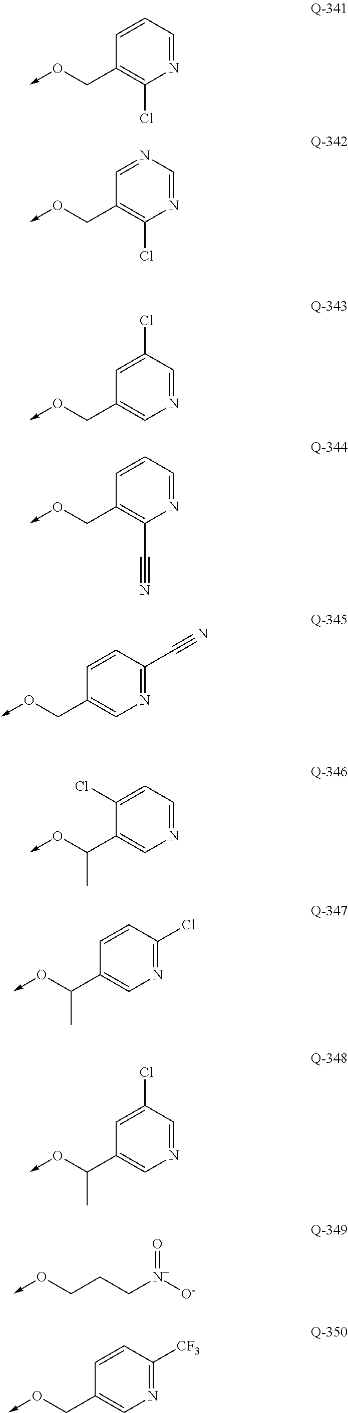

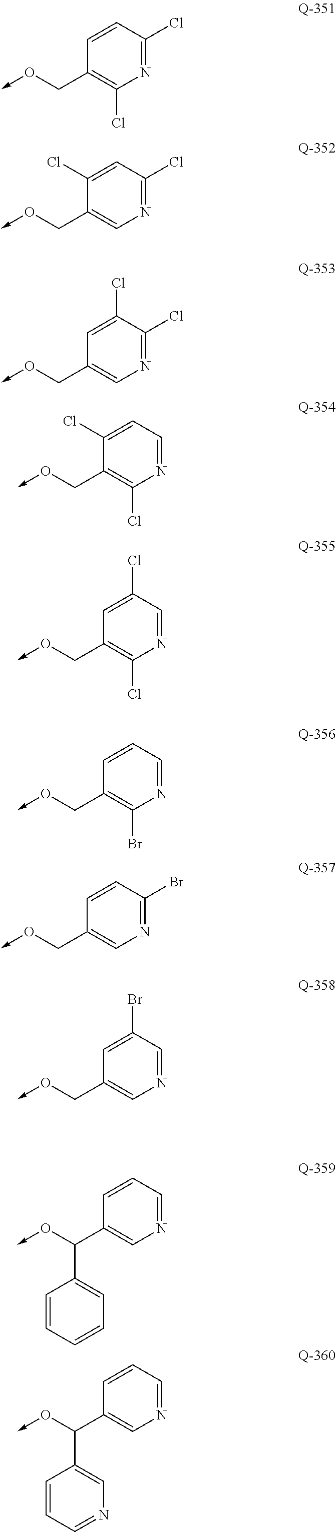

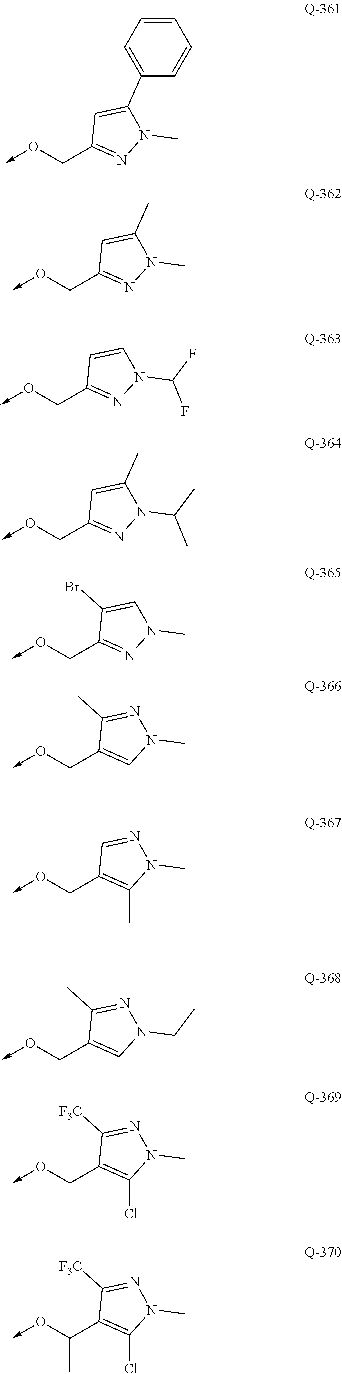

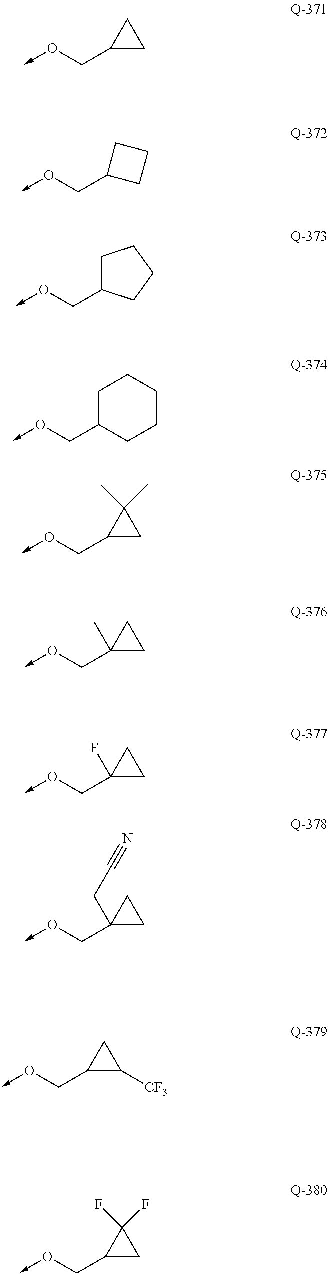

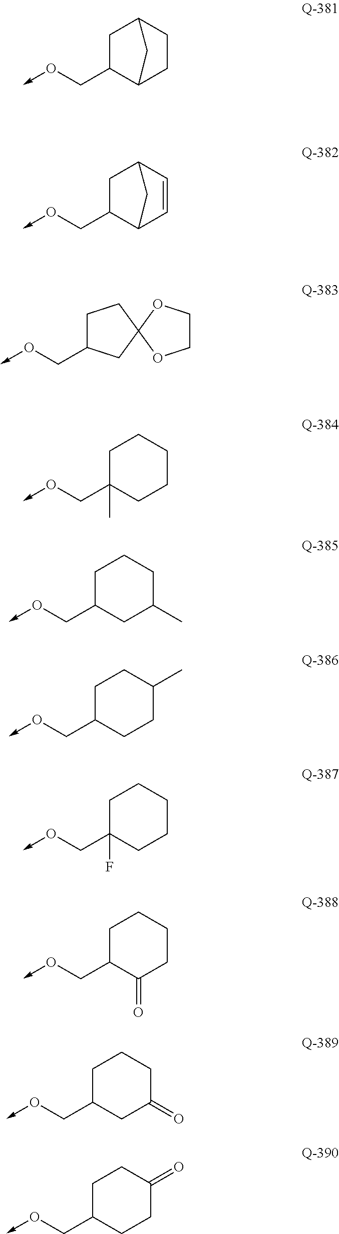

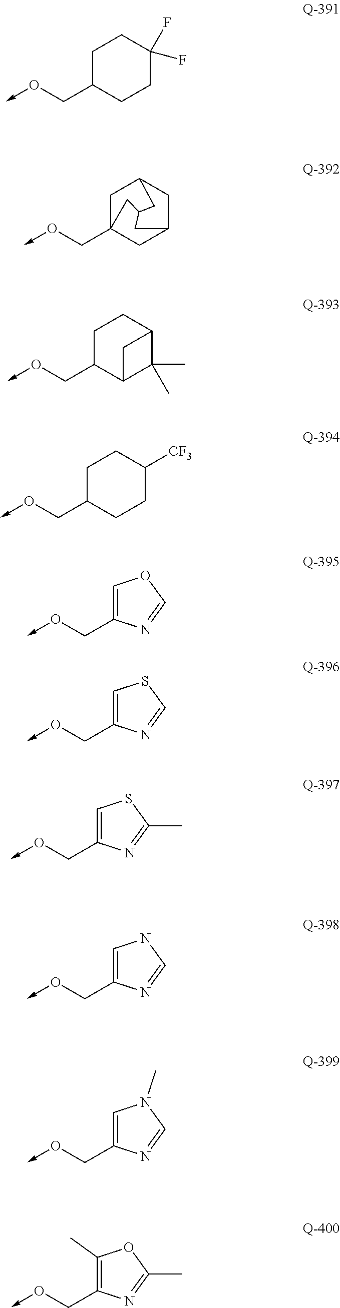

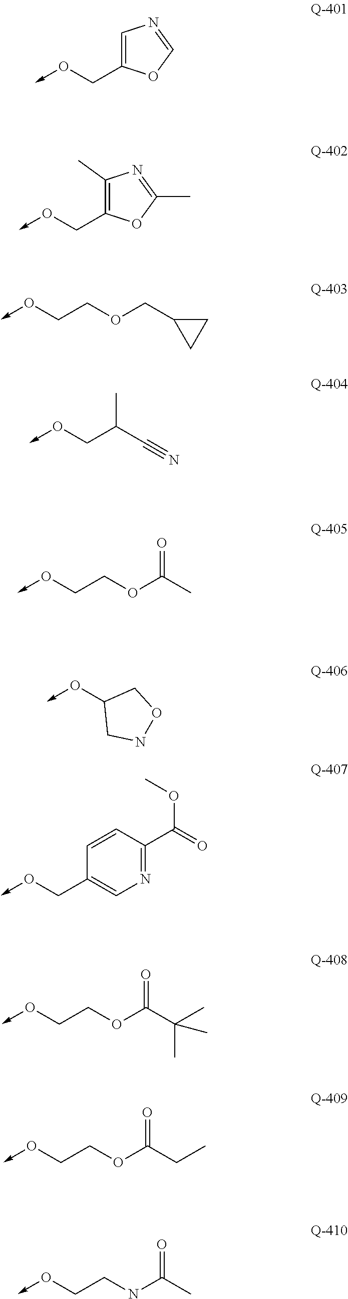

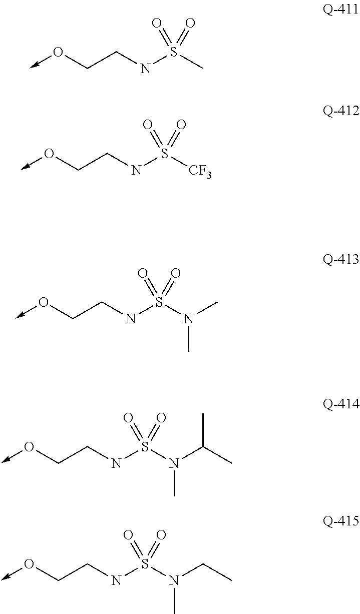

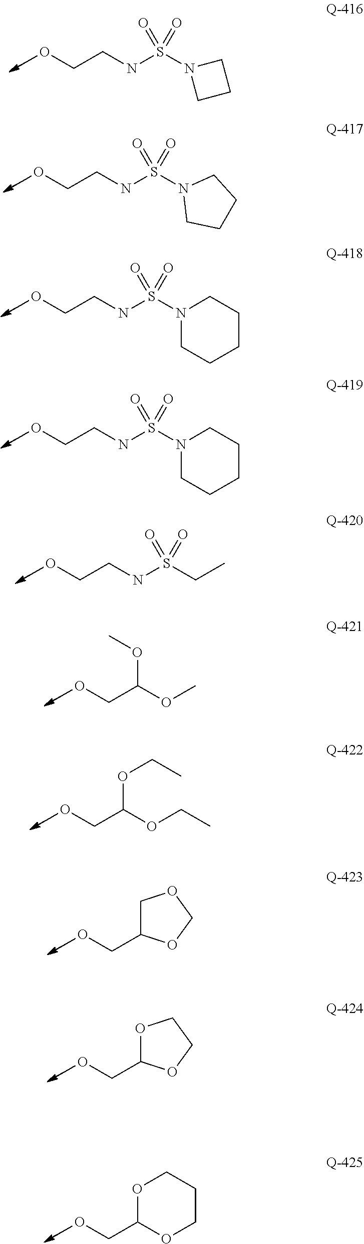

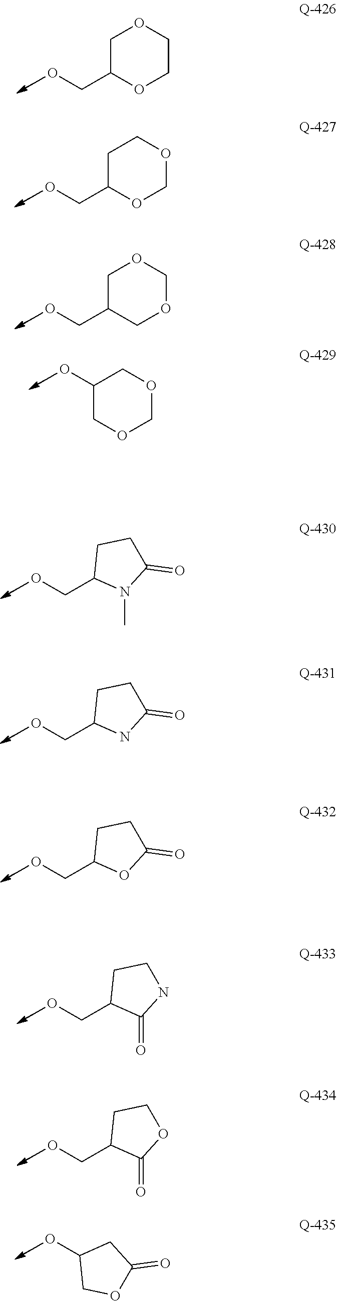

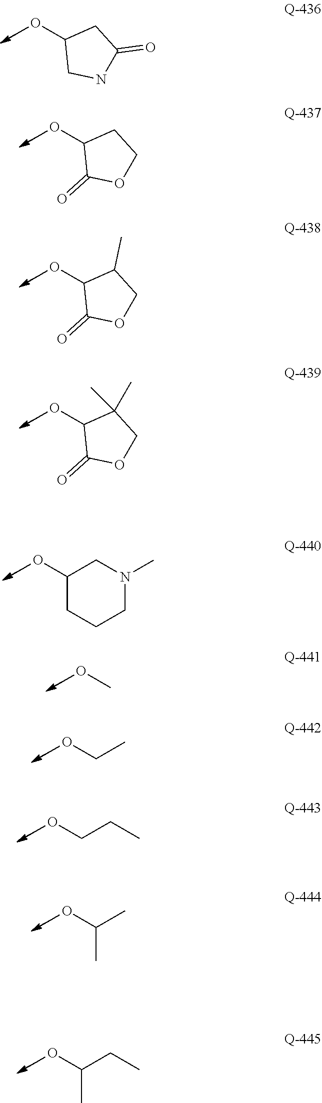

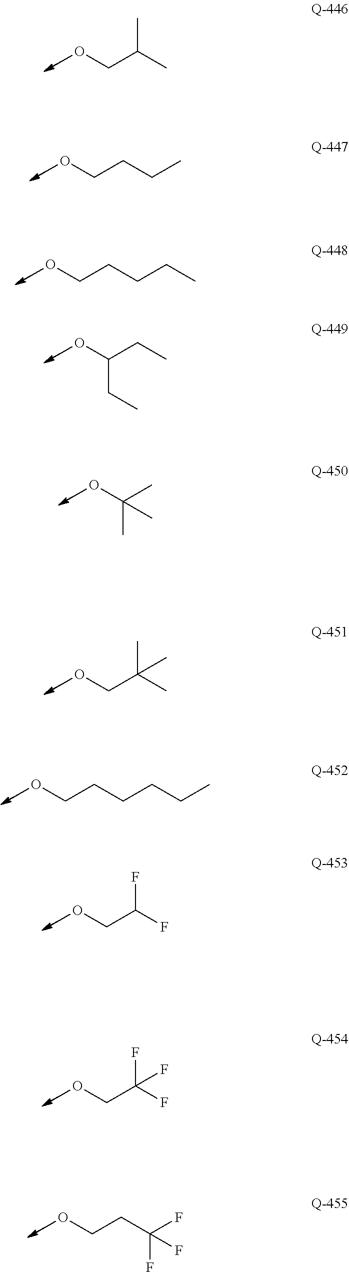

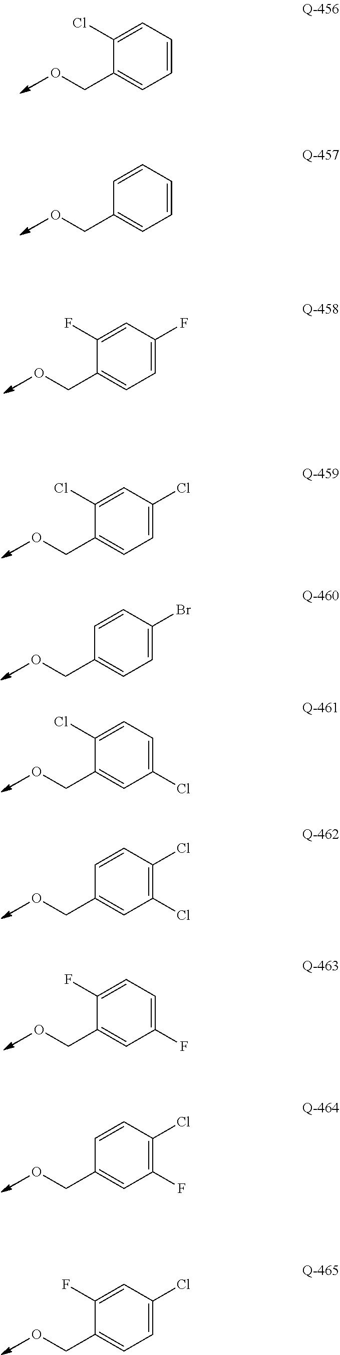

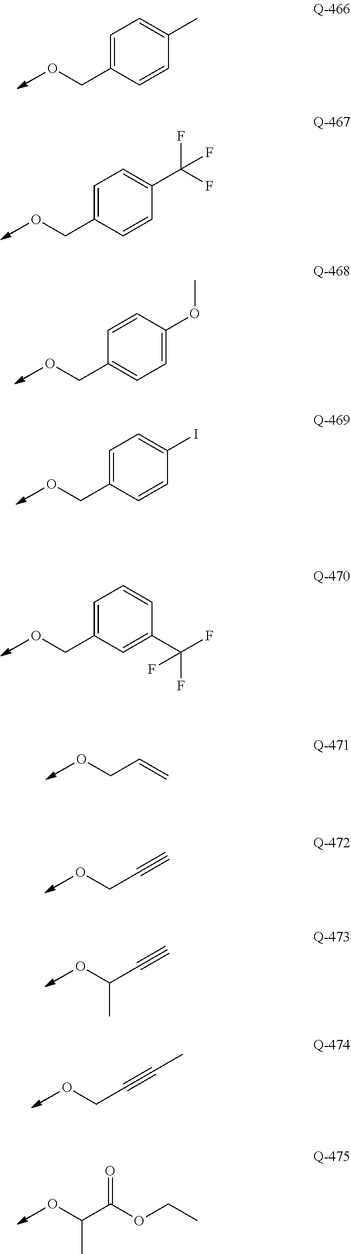

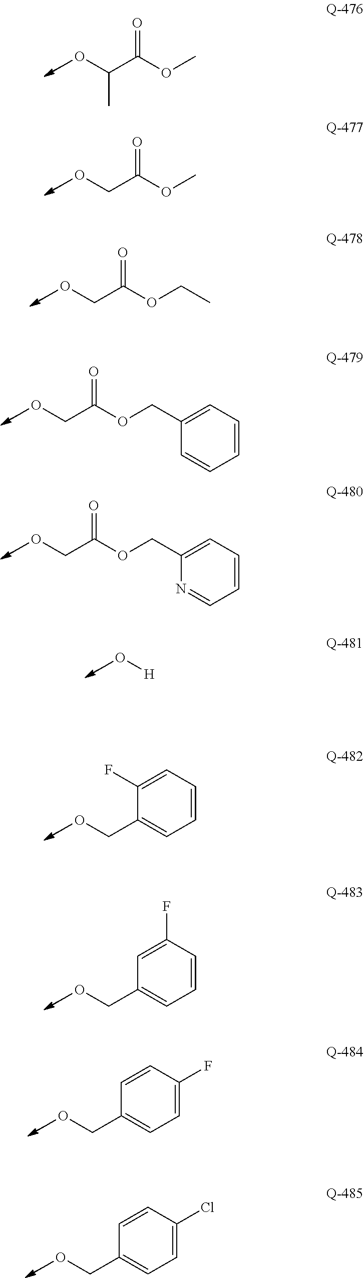

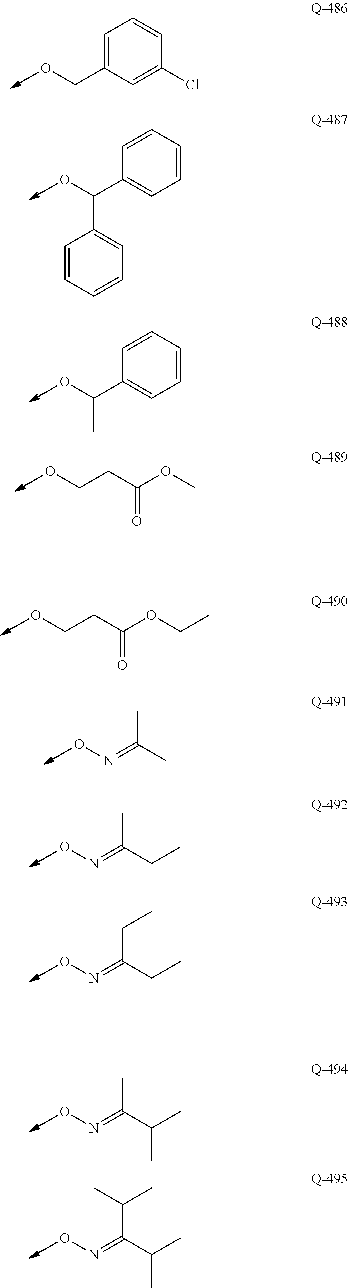

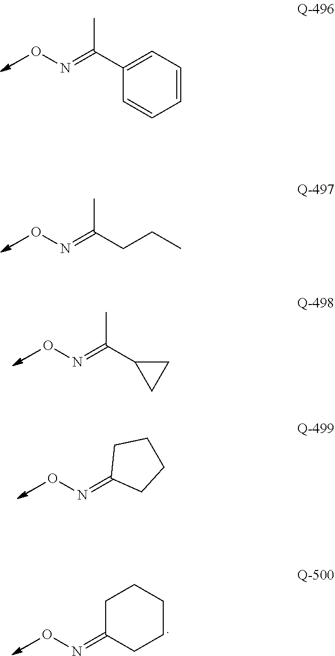

In preferred embodiments, the compound of the general formula (I) or an agrochemically acceptable salt thereof, corresponds to one of the compounds denoted (a)-(y) below, with their corresponding IUPAC names and structures.

| Compound | IUPAC name | Structure |
|---|---|---|
| (a) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 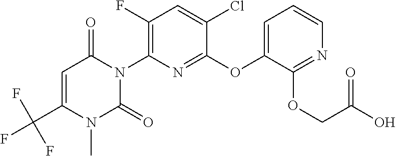 [SMILES: Cn1c(C(F)(F)F)cc(=O)n(-c2nc(Oc3cccnc3OCC(=O)O)c(Cl)cc2F)c1=O] |
| (b) | ethyl {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetate | 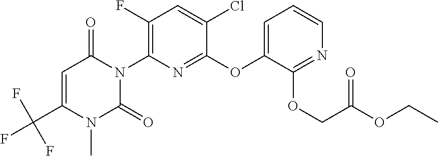 [SMILES: CCOC(=O)COc1ncccc1Oc1nc(-n2c(=O)cc(C(F)(F)F)n(C)c2=O)c(F)cc1Cl] |
| (c) | methyl {[3-({3-chloro-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 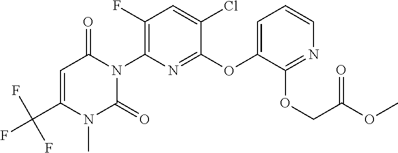 [SMILES: COC(=O)COc1ncccc1Oc1nc(-n2c(=O)cc(C(F)(F)F)n(C)c2=O)c(F)cc1Cl] |
| (d) | pyridazin-3-ylmethyl{[3-({3-chloro-5-fluoro- 6-[3-methyl-2,6-dioxo-4-(trifluoromethyl)- 3,6-dihydropyrimidin-1(2H)-yl]pyridin-2- yl}oxy)pyridin-2-yl]oxy}acetate | 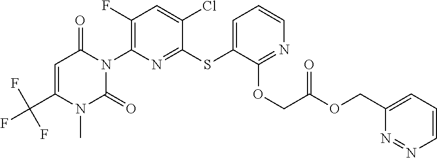 [SMILES: Cn1c(C(F)(F)F)cc(=O)n(-c2nc(Sc3cccnc3OCC(=O)OCc3cccnn3)c(Cl)cc2F)c1=O] |
| (e) | pyridin-2-ylmethyl{[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 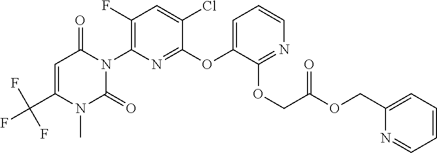 [SMILES: Cn1c(C(F)(F)F)cc(=O)n(-c2nc(Oc3cccnc3OCC(=O)OCc3ccccn3)c(Cl)cc2F)c1=O] |
| (f) | pyridin-4-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 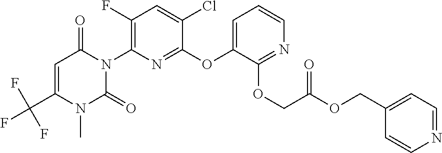 [SMILES: Cn1c(C(F)(F)F)cc(=O)n(-c2nc(Oc3cccnc3OCC(=O)OCc3ccncc3)c(Cl)cc2F)c1=O] |
| (g) | (4-trifluoromethylpyridin-3-yl)methyl{ [3-({3- chloro-5-fluoro-6-[3-methyl-2,6-dioxo-4- (trifluoromethyl)-3,6-dihydropyrimidin- 1(2H)-yl]pyridin-2-yl}oxy)pyridin-2- yl]oxy}acetate | 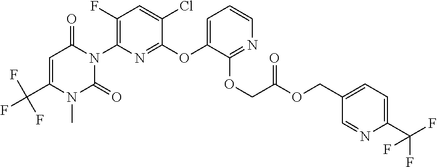 [SMILES: Cn1c(C(F)(F)F)cc(=O)n(-c2nc(Oc3cccnc3OCC(=O)OCc3ccc(C(F)(F)F)nc3)c(Cl)cc2F)c1=O] |
| (h) | ethyl [2-({3-chloro-5-fluoro-6-[3-methyl-2,6- dioxo-4-(trifluoromethyl)-3,6- dihydropyrimidin-1(2H)-yl]pyridin-2- yl}oxy)phenoxy]acetate | 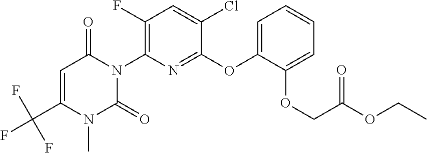 [SMILES: CCOC(=O)COc1ccccc1Oc1nc(-n2c(=O)cc(C(F)(F)F)n(C)c2=O)c(F)cc1Cl] |
| (i) | methyl[2-({3-chloro-5-fluoro-6-[3-methyl- 2,6-dioxo-4-(trifluoromethyl)-3,6- dihydropyrimidin-1(2H)-yl]pyridin-2- yl}oxy)phenoxy]acetate | 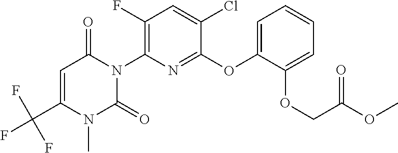 [SMILES: COC(=O)COc1ccccc1Oc1nc(-n2c(=O)cc(C(F)(F)F)n(C)c2=O)c(F)cc1Cl] |
| (j) | [2-({3-chloro-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 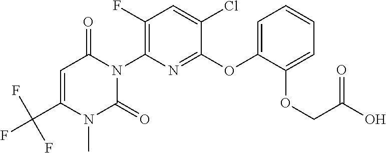 [SMILES: Cn1c(C(F)(F)F)cc(=O)n(-c2nc(Oc3ccccc3OCC(=O)O)c(Cl)cc2F)c1=O] |
| (k) | ethyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 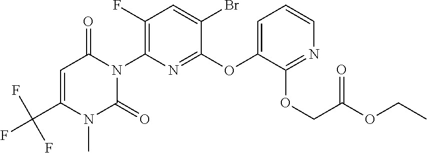 [SMILES: CCOC(=O)COc1ncccc1Oc1nc(-n2c(=O)cc(C(F)(F)F)n(C)c2=O)c(F)cc1Br] |
| (l) | methyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 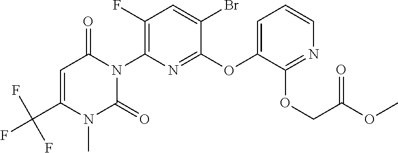 [SMILES: COC(=O)COc1ncccc1Oc1nc(-n2c(=O)cc(C(F)(F)F)n(C)c2=O)c(F)cc1Br] |
| (m) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 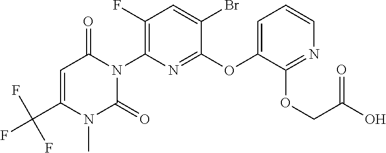 [SMILES: Cn1c(C(F)(F)F)cc(=O)n(-c2nc(Oc3cccnc3OCC(=O)O)c(Br)cc2F)c1=O] |
| (n) | ethyl [2-({3-bromo-5-fluoro-6-[3-methyl-2,6- dioxo-4-(trifluoromethyl)-3,6- dihydropyrimidin-1(2H)-yl]pyridin-2- yl}oxy)phenoxy]acetate | 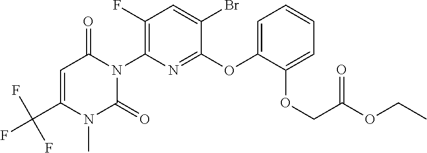 [SMILES: CCOC(=O)COc1ccccc1Oc1nc(-n2c(=O)cc(C(F)(F)F)n(C)c2=O)c(F)cc1Br] |
| (o) | methyl [2-({3-bromo-5-fluoro-6-[3-methyl- 2,6-dioxo-4-(trifluoromethyl)-3,6- dihydropyrimidin-1(2H)-yl]pyridin-2- yl}oxy)phenoxy]acetate | 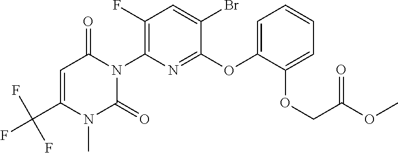 [SMILES: COC(=O)COc1ccccc1Oc1nc(-n2c(=O)cc(C(F)(F)F)n(C)c2=O)c(F)cc1Br] |
| (p) | [2-({3-bromo-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 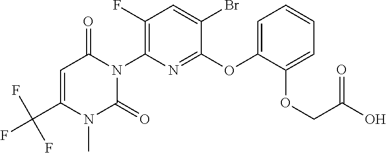 [SMILES: Cn1c(C(F)(F)F)cc(=O)n(-c2nc(Oc3ccccc3OCC(=O)O)c(Br)cc2F)c1=O] |
| (q) | ethyl {[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetate | 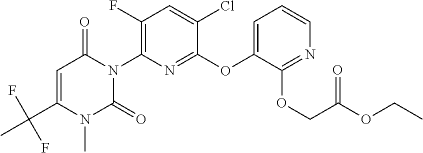 [SMILES: CCOC(=O)COc1ncccc1Oc1nc(-n2c(=O)cc(C(C)(F)F)n(C)c2=O)c(F)cc1Cl] |
| (r) | methyl {[3-({3-chloro-5-fluoro-6-[3-methyl- 2,6-dioxo-4-(1,1-difluoroethyl)-3,6- dihydropyrimidin-1(2H)-yl]pyridin-2- yl}oxy)pyridin-2-yl]oxy}acetate | 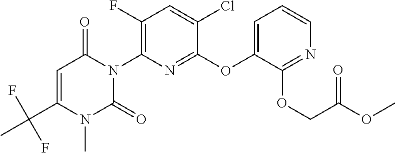 [SMILES: COC(=O)COc1ncccc1Oc1nc(-n2c(=O)cc(C(C)(F)F)n(C)c2=O)c(F)cc1Cl] |
| (s) | {[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetic acid | 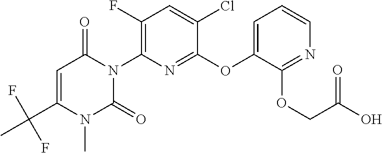 [SMILES: Cn1c(C(C)(F)F)cc(=O)n(-c2nc(Oc3cccnc3OCC(=O)O)c(Cl)cc2F)c1=O] |
| (t) | ethyl [2-({3-chloro-5-fluoro-6-[3-methyl-2,6- dioxo-4-(1,1-difluoroethyl)-3,6- dihydropyrimidin-1(2H)-yl]pyridin-2- yl}oxy)phenoxy]acetate | 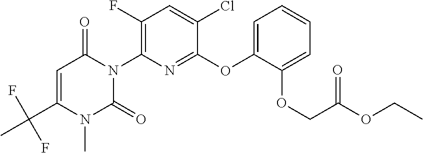 [SMILES: CCOC(=O)COc1ccccc1Oc1nc(-n2c(=O)cc(C(C)(F)F)n(C)c2=O)c(F)cc1Cl] |
| (u) | methyl [2-({3-chloro-5-fluoro-6-[3-methyl- 2,6-dioxo-4-(1,1-difluoroethyl)-3,6- dihydropyrimidin-1(2H)-yl]pyridin-2- yl}oxy)phenoxy]acetate | 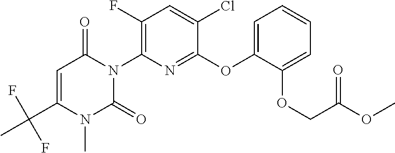 [SMILES: COC(=O)COc1ccccc1Oc1nc(-n2c(=O)cc(C(C)(F)F)n(C)c2=O)c(F)cc1Cl] |
| (v) | [2-({3-chloro-5-fluoro-6-[3-methyl-2,6-dioxo- 4-(1,1-difluoroethyl)-3,6-dihydropyrimidin- 1(2H)-yl]pyridin-2-yl}oxy)phenoxy]acetic acid | 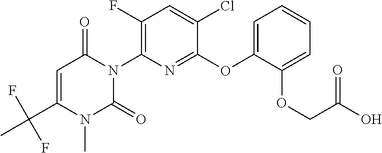 [SMILES: Cn1c(C(C)(F)F)cc(=O)n(-c2nc(Oc3ccccc3OCC(=O)O)c(Cl)cc2F)c1=O] |
| (x) | ethyl{[3-({3-chloro-5-fluoro-6-[3-methyl-2,6- dioxo-4-(trifluoromethyl)-3,6- dihydropyrimidin-1(2H)-yl]pyridin-2- yl}sulfanyl)pyridin-2-yl]oxy}acetate | 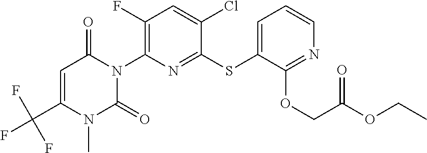 [SMILES: CCOC(=O)COc1ncccc1Sc1nc(-n2c(=O)cc(C(F)(F)F)n(C)c2=O)c(F)cc1Cl] |
| (y) | methyl{[3-({3-chloro-5-fluoro-6-[3-methyl- 2,6-dioxo-4-(trifluoromethyl)-3,6- dihydropyrimidin-1(2H)-yl]pyridin-2- yl}sulfanyl)pyridin-2-yl]oxy}acetate | 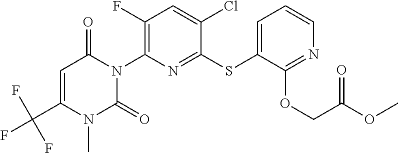 [SMILES: COC(=O)COc1ncccc1Sc1nc(-n2c(=O)cc(C(F)(F)F)n(C)c2=O)c(F)cc1Cl] |

In more preferred embodiments, the compound of the general formula (I) or an agrochemically acceptable salt thereof, corresponds to one of the compounds denoted (a)-(c), (h)-(j), and (x)-(y) below, with their corresponding IUPAC names: (a) ethyl {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetate; (b) methyl {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetate; (c) {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetic acid; (h) ethyl [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetate; (i) methyl [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetate; (j) [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetic acid; (x) ethyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate; (y) methyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate.

Compounds of formula (I), herbicidal compositions comprising same, and processes for their synthesis have been described in WO2017/202768, WO2024/110228, and/or WO 2019/101533.

In some embodiments of the methods provided herein, the heterologous HemG protein has herbicide-insensitive protoporphyrinogen oxidase activity. In some embodiments, the heterologous HemG protein has at least 85% sequence identity to a polypeptide sequence selected from the group consisting of SEQ ID NOs:1-20 and 65-193. In some embodiments, the DNA sequence encoding the heterologous HemG protein is selected from the group consisting of SEQ ID NOs:22-64 and 194-322. In further embodiments, the heterologous HemG protein comprises an amino acid sequence selected from the group consisting of SEQ ID NOs:1-20 and 65-193. In some embodiments, the DNA sequence encoding a heterologous HemG protein is operably linked to a DNA sequence encoding a chloroplast transit peptide (CTP). In some embodiments, the CTP comprises an amino acid sequence with at least 97% sequence identity to a sequence selected from the group consisting of SEQ ID NOs:323-328, 340, and 342-407. In further embodiments, the DNA sequence encoding the CTP comprises at least 97% identity to a sequence selected from the group consisting of SEQ ID NOs:329-339, 341, and 408-483. In additional embodiments, said recombinant DNA molecule further comprises a heterologous promoter operably linked to the DNA sequence encoding said HemG protein. In some embodiments, the plant comprising the recombinant DNA molecule is a monocotyledonous plant. In some embodiments, the plant comprising the recombinant DNA molecule is a dicotyledonous plant.

In some embodiments of the methods provided herein, the herbicidally active compound is applied to the area at a rate of about 0.02 g a.i./ha to about 750 g a.i./ha, about 0.05 g a.i./ha to about 400 g a.i./ha, or about 0.25 g a.i./ha to about 300 g a.i./ha. In some embodiments, the method is further defined as comprising applying said compound to said area at least twice. In some embodiments, the herbicidally active compound is applied in an amount that does not damage said plant comprising the recombinant DNA molecule. In some embodiments, said applying of the compound is carried out pre-emergence. In some embodiments, said applying of the compound is carried out post-emergence. In some embodiments, said applying of the compound comprises contacting said plant with the compound. In further embodiments, said applying of the compound comprises an over the top application of said compound. In additional embodiments, said applying of the compound results in an increase in the growth or yield of said plant relative to a plant of the same genotype cultivated in a growth area in which said compound has not been applied.

In some embodiments of the methods provided herein, the method further comprises applying to said area an effective amount of at least a second herbicide. In further embodiments, the second herbicide is selected from the group consisting of an ACCase inhibitor, an ALS inhibitor, an EPSPS inhibitor, a synthetic auxin, a photosynthesis inhibitor, a glutamine synthesis inhibitor, a HPPD inhibitor, a PPO inhibitor, and a long-chain fatty acid inhibitor. In even further embodiments, the ACCase inhibitor is an aryloxyphenoxy propionate or a cyclohexanedione; the ALS inhibitor is a sulfonylurea, imidazolinone, triazoloyrimidine, or triazolinone; the EPSPS inhibitor is glyphosate; the synthetic auxin is a phenoxy herbicide, a benzoic acid, a carboxylic acid, or a semicarbazone; the photosynthesis inhibitor is a triazine, a triazinone, a nitrile, a benzothiadiazole, or a urea; the glutamine synthesis inhibitor is glufosinate; the HPPD inhibitor is an isoxazole, a pyrazolone, or a triketone; the PPO inhibitor is a diphenylether, a N-phenylphthalimide, an aryl triazinone, or a pyrimidinedione; or the long-chain fatty acid inhibitor is a chloroacetamide, an oxyacetamide, or a pyrazole.

### BRIEF DESCRIPTION OF THE SEQUENCES

SEQ ID NO:1 is the amino acid sequence of the HemG PPO H_N90.
SEQ ID NO:2 is the amino acid sequence of the HemG PPO H_N20.
SEQ ID NO:3 is the amino acid sequence of the HemG PPO H_N60.
SEQ ID NO:4 is the amino acid sequence of H_N10, which is the E. coli wild-type HemG protoporphyrinogen oxidase (NCBI GenBank Accession No. WP_021498199).
SEQ ID NO:5 is the amino acid sequence of the HemG PPO H_N30.
SEQ ID NO:6 is the amino acid sequence of the HemG PPO H_N40.
SEQ ID NO:7 is the amino acid sequence of the HemG PPO H_N50.
SEQ ID NO:8 is the amino acid sequence of the HemG PPO H_N70.
SEQ ID NO:9 is the amino acid sequence of the HemG PPO H_N100.
SEQ ID NO:10 is the amino acid sequence of the HemG PPO H_N110.
SEQ ID NOs:11-17 are amino acid sequences lacking the start methionine and corresponding to SEQ ID NOs:1, 2, 4, 5, 6, 7, and 9, respectively.
SEQ ID NOs:18-19 are amino acid sequences of two variants of SEQ ID NO:11.
SEQ ID NO:20 is the amino acid sequence of a variant of SEQ ID NO:17.
SEQ ID NO:21 is the amino acid sequence of the wild-type PPO from Amaranthus tuberculatus (waterhemp).
SEQ ID NOs:22-31 are nucleotide sequences encoding SEQ ID NOs:1-10, respectively, codon optimized for E. coli expression.
SEQ ID NOs:32-41 are nucleotide sequences encoding SEQ ID NOs:1-10, respectively, codon optimized for dicot expression.
SEQ ID NOs:42-48 are nucleotide sequences encoding SEQ ID NOs:11-17, respectively, codon optimized for dicot expression.
SEQ ID NOs:49-50 are recombinant nucleotide sequences encoding SEQ ID NO:11.
SEQ ID NO:51 is a recombinant nucleotide sequence encoding SEQ ID NO:12.
SEQ ID NOs:52-54 are recombinant nucleotide sequences encoding SEQ ID NOs:18-20.
SEQ ID NOs:55-64 are nucleotide sequences encoding SEQ ID NOs:1-10, respectively, codon optimized for monocot expression.
SEQ ID NOs:65-77 are amino acid sequences of HemG PPOs from different species with variations in the long chain insert loop.
SEQ ID NOs:78-193 are amino acid sequences of recombinant HemG PPO variants, each incorporating a mutation to the long chain insert loop.
SEQ ID NOs:194-206 are nucleotide sequences encoding the HemG PPOs of SEQ ID NOs:65-77.
SEQ ID NOs:207-322 are nucleotide sequences encoding the recombinant HemG PPO variants of SEQ ID NOs:78-193.
SEQ ID NO:323 is the amino acid sequence of the Arabidopsis thaliana albino and pale green (APG6) chloroplast transit peptide (CTP).
SEQ ID NO:324 is the amino acid sequence of an amino-terminal optimized variant of the APG6 CTP.
SEQ ID NO:325 is the amino acid sequence of the Arabidopsis thaliana 90 kDa heat shock protein (CR88) CTP.
SEQ ID NO:326 is the amino acid sequence of the petunia ShkG-EPSPS CTP.
SEQ ID NO:327 is the amino acid sequence of the pea rbcS-3C CTP.
SEQ ID NO:328 is the amino acid sequence of the rice Waxy CTP.
SEQ ID NOs:329-333 are the nucleotide sequences encoding APG6 CTP of SEQ ID NO:323, optimized for plant expression.
SEQ ID NO:334 is the nucleotide sequence encoding APG6 CTP of SEQ ID NO:324.
SEQ ID NOs:335-336 are nucleotide sequences encoding AtCR88 CTP optimized for dicot and monocot expression, respectively.
SEQ ID NOs:337-339 are nucleotide sequences encoding SEQ ID NOs:326-328.
SEQ ID NO:340 is the amino acid sequence of the cotton 12G088600TP CTP.
SEQ ID NO:341 is the nucleotide sequence encoding the cotton 12G088600TP CTP, optimized for dicot expression.
SEQ ID NOs:342-407 are amino acid sequences of transit peptides from different species.
SEQ ID NOs:408-483 are nucleotide sequences encoding SEQ ID NOs:342-407.

### DETAILED DESCRIPTION

The following descriptions and definitions are provided to better define the invention and to guide those of ordinary skill in the art in the practice of the invention. Unless otherwise noted, terms are to be understood according to conventional usage by those of ordinary skill in the relevant art.

The present disclosure provides for the use of substituted uracil pyridines or agrochemically acceptable salts thereof, in controlling or preventing weed growth in plant growth areas of transgenic crop plants that express herbicide insensitive PPOs (protoporphyrinogen oxidases), and that are therefore tolerant to PPO inhibiting herbicides. PPO is an essential enzyme in plants that catalyzes the dehydrogenation of protoporphyrinogen IX to form protoporphyrin IX, which is the precursor to heme and chlorophyll. PPO inhibition in plant cells causes accumulation of intermediate tetrapyrroles and the formation of reactive oxygen species, resulting in membrane disruption and ultimately cell death. There are several herbicide families that are classified as PPO inhibitors, such as diphenyl ethers, aryl triazolinones, pyrimidinediones, and N-phenylphthalimides.

It has been found that compounds of the structure class of substituted uracil pyridines have herbicidal activity (see WO2017202768A1) and can be used for controlling monocotyledonous and dicotyledonous weeds. These compounds are effective against a broad spectrum of harmful plants when applied both preemergence and postemergence, with the possibility of non-selective use for control of unwanted plant growth or selective use in plant crops. The present application shows that these specific substituted uracil pyridines or salts thereof can be applied to transgenic crop plants that comprise one or more genes conferring tolerance to PPO inhibitor herbicides. The herbicide tolerance trait described herein provides tolerance to one of more of the herbicidally active compounds of the general formula (I) or agrochemically acceptable salts thereof.

### I. Herbicides and Herbicidally Active Compounds

Provided herein are uses for specific substituted uracil pyridines of the general formula (I):

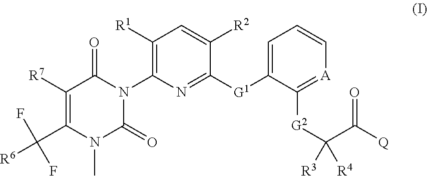

in which
A represents N (nitrogen) or a group C—R⁵,
G¹ and G² independently of each other represent O (oxygen) or S (sulfur),
R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, iodine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O—(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl, or
R³ and R⁴ together with the carbon atom to which they are bonded form a fully saturated or partly saturated 3- to 10-membered carbocyclic ring optionally having further substitution,
R⁵ represents hydrogen, halogen, (C₁-C₈)-alkyl, (C₁-C₈)-haloalkyl, or OR¹³,
R⁶ represents hydrogen, fluorine, chlorine, bromine, trifluoromethyl, difluoromethyl, methoxy, ethoxy, prop-1-yloxy, but-1-yloxy or methyl,
R⁷ represents hydrogen or methyl,
Q represents hydroxy or a group Q-1, Q-2

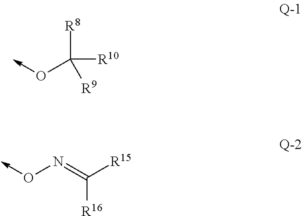

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₁C₈)-haloalkyl, (C₃C₈)-cycloalkyl, (C₃C₈)-cycloalkyl-(C₁C₈)-alkyl, (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O—(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR¹³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₈)-cycloalkyl, aryl, heteroaryl, or heterocyclyl;
and (b) applying to said area an amount of said compound effective to control or prevent weed growth in the area.

In certain embodiments, the compound of the general formula (I) or an agrochemically acceptable salt thereof is further characterized in that
A represents N (nitrogen) or a group C—R⁵,
G¹ and G² independently of each other represent O (oxygen) or S (sulfur),
R¹ represents hydrogen, fluorine,
R² represents chlorine, bromine, iodine, nitro or cyano,
R³ and R⁴ independently of each other represent hydrogen, methyl, or ethyl, or
R³ and R⁴ together with the carbon atom to which they are bonded form a fully saturated or partly saturated 3- to 6-membered carbocyclic ring optionally having further substitution,
R⁵ represents hydrogen, chlorine, or methoxy,
R⁶ represents fluorine, methyl,
R⁷ represents hydrogen, methyl,
and
Q represents one of the following moieties Q-1 to Q-500:

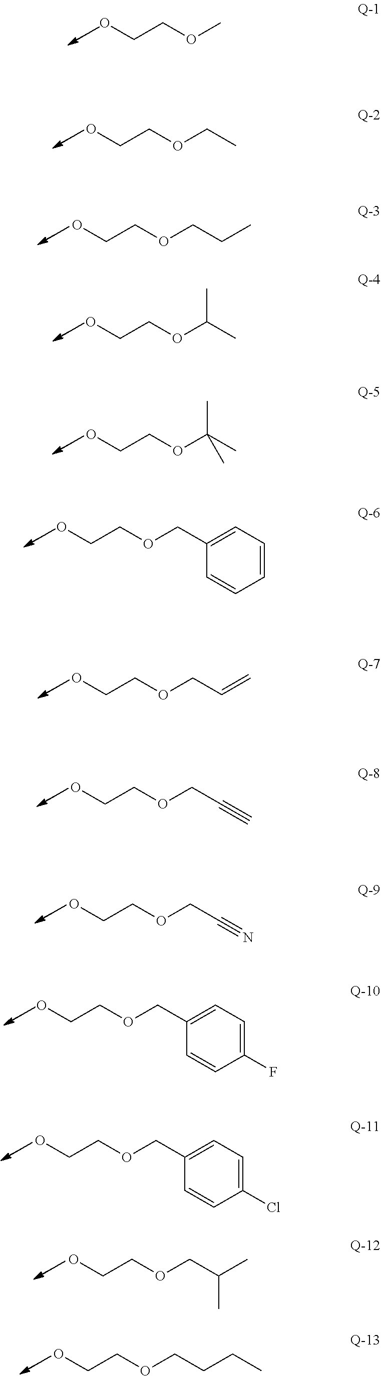

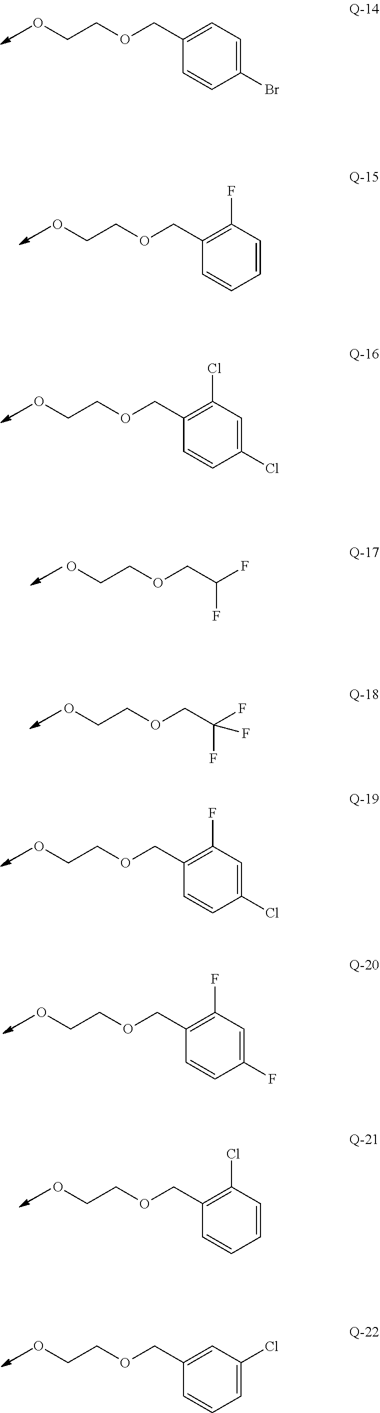

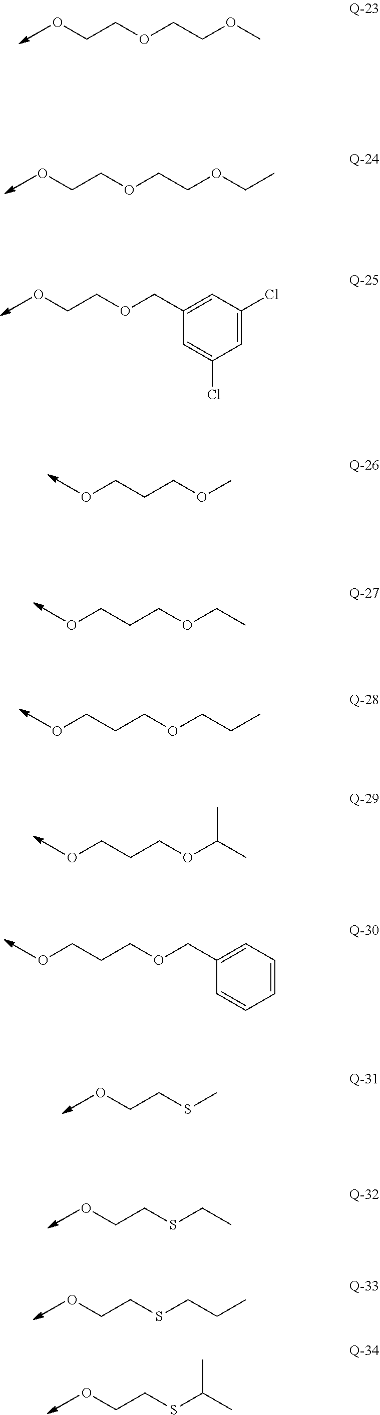

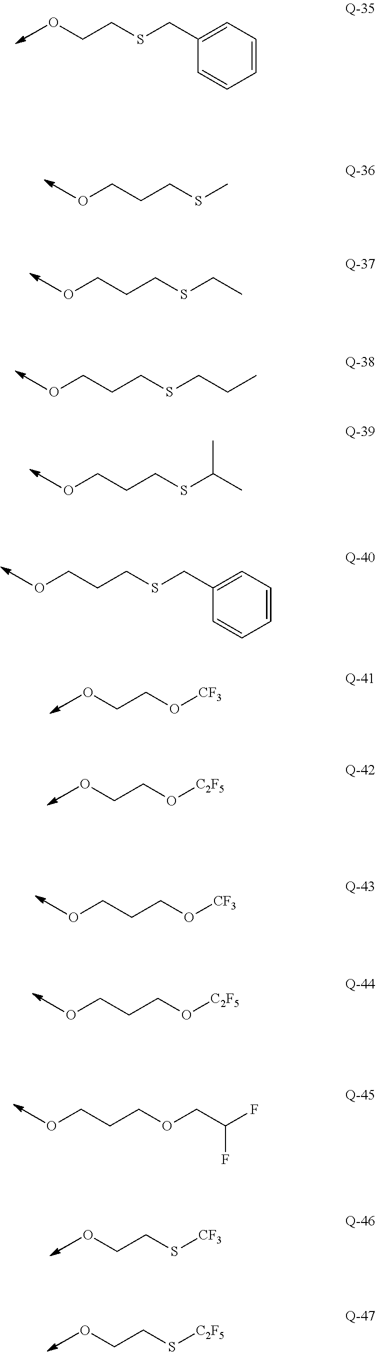

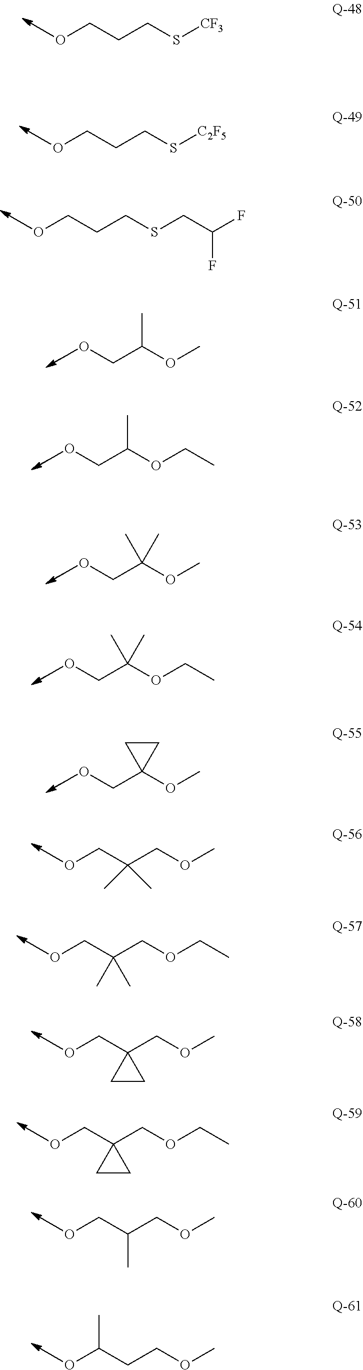

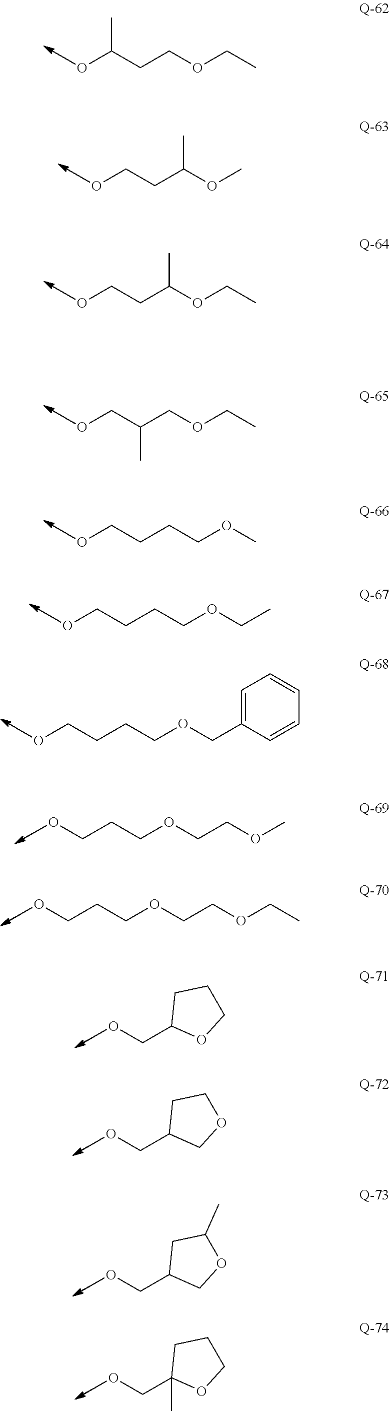

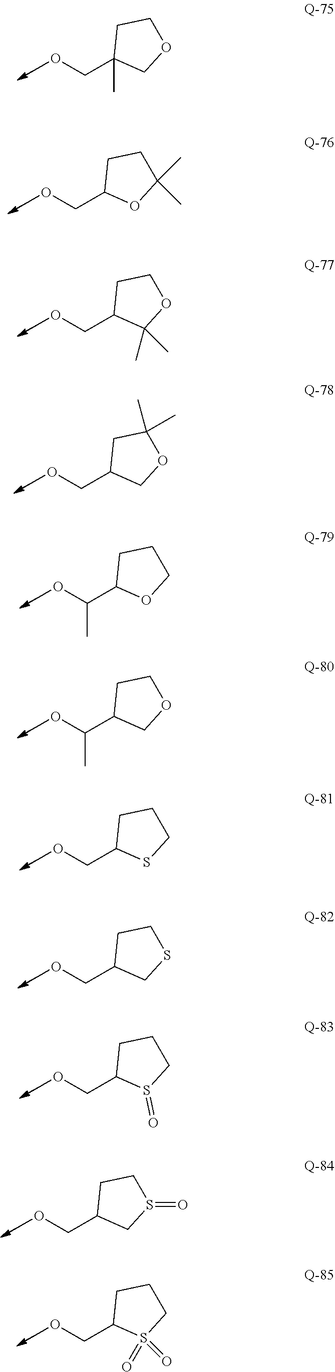

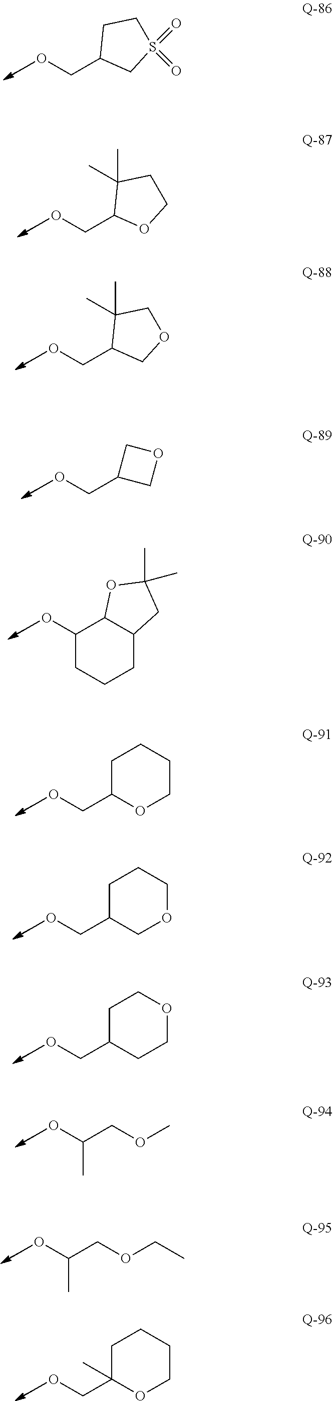

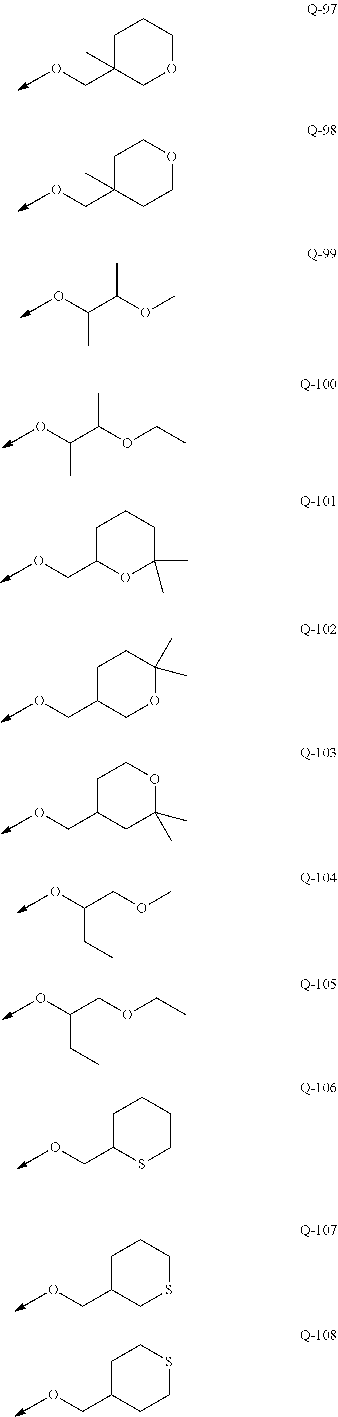

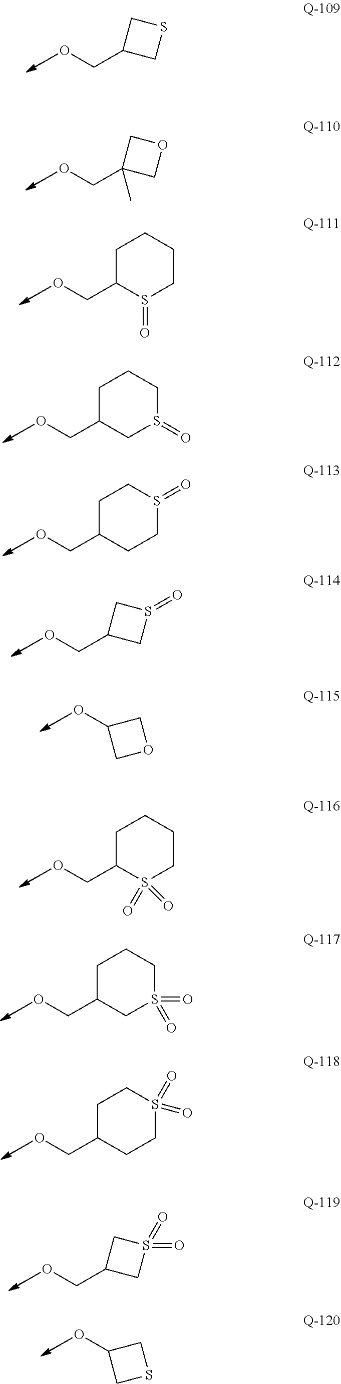

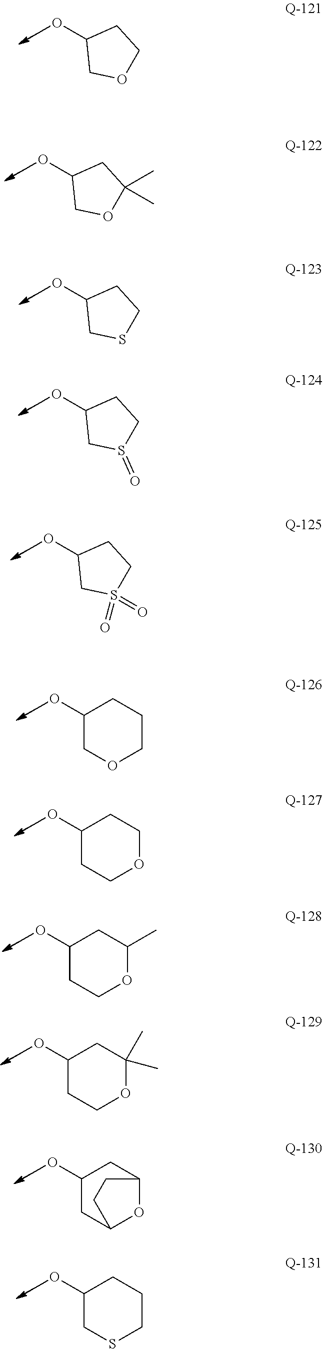

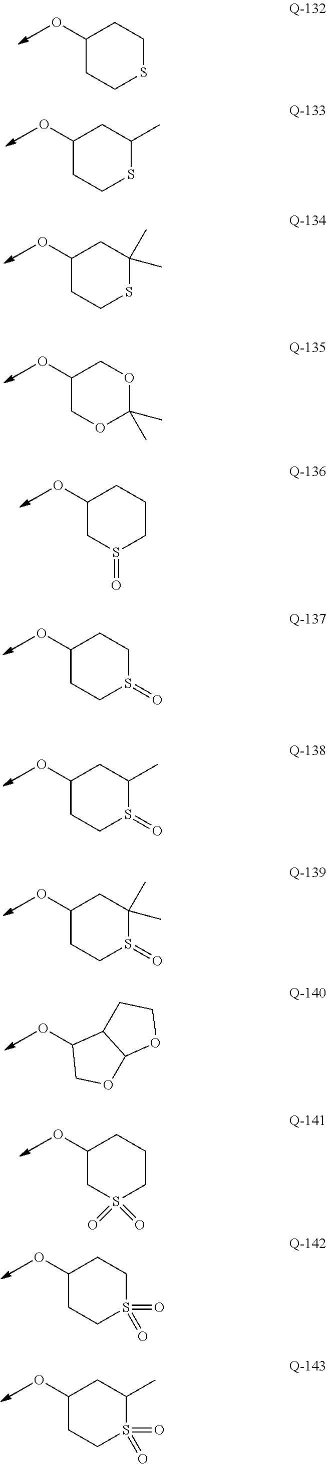

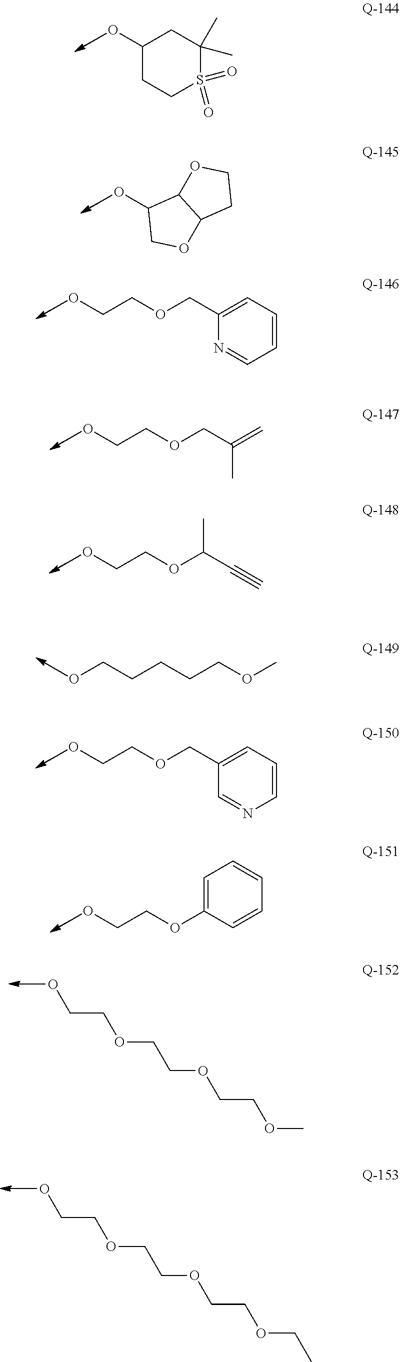

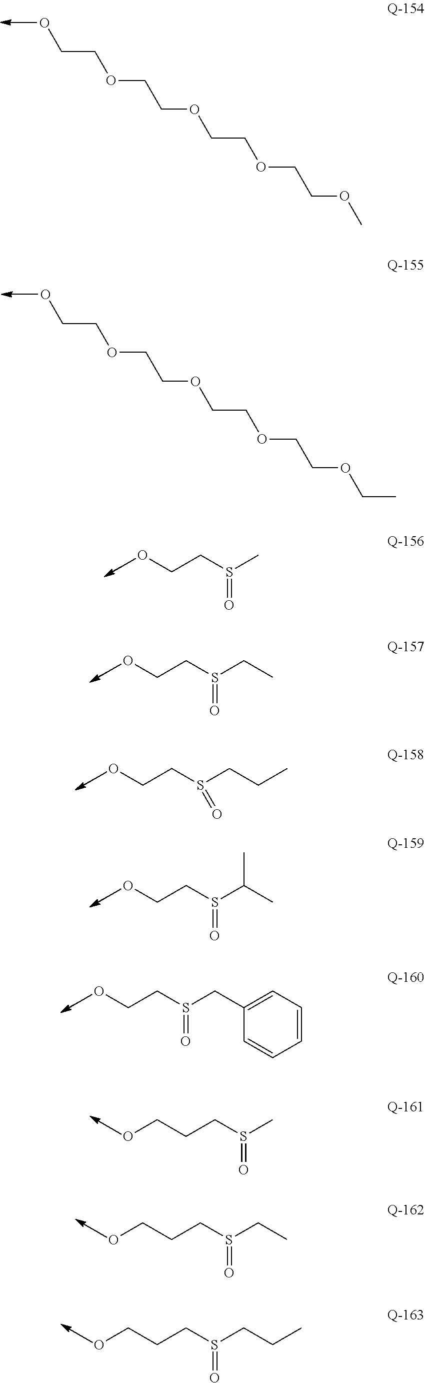

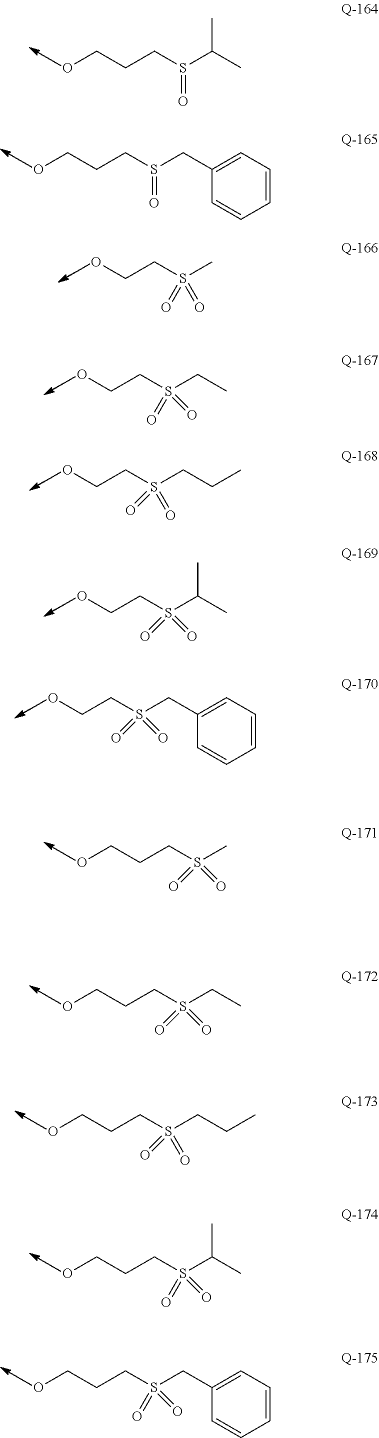

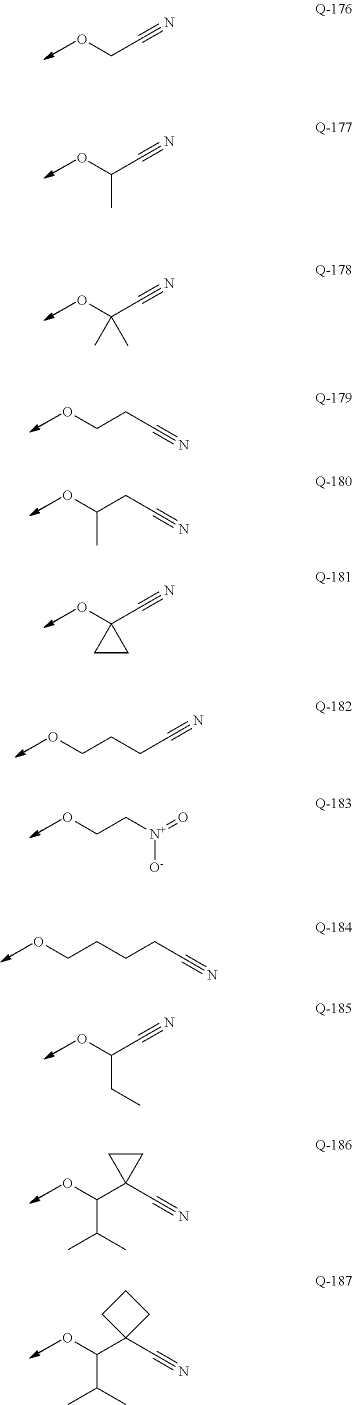

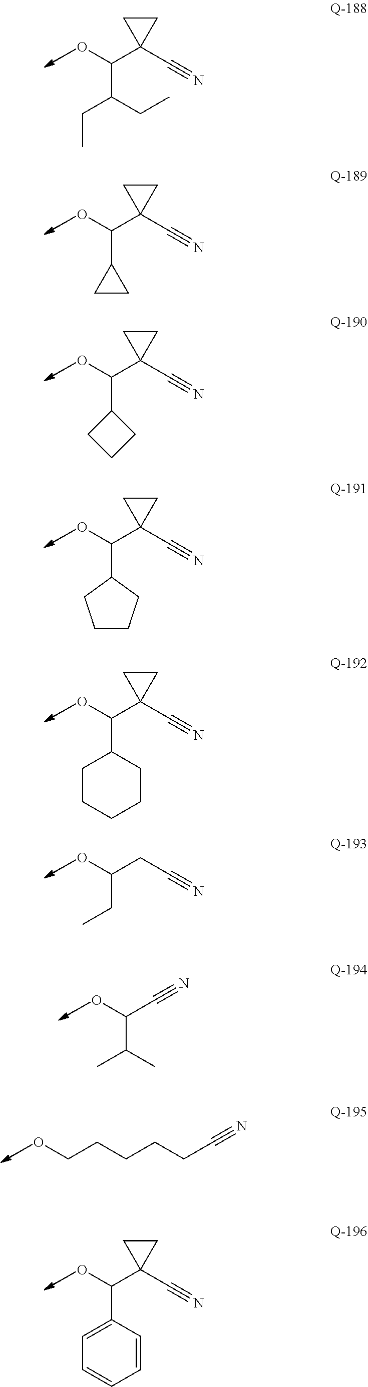

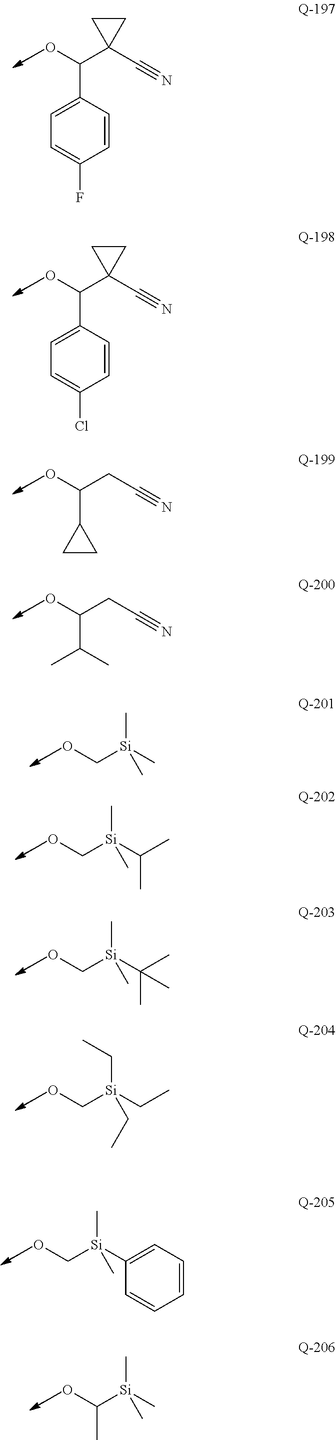

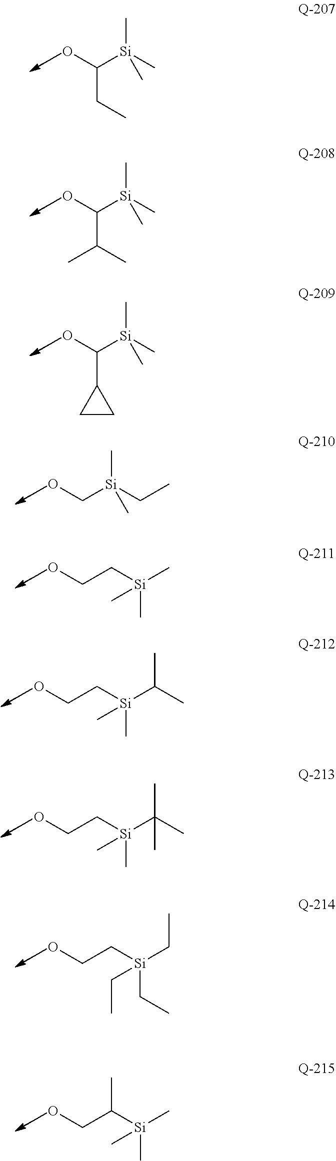

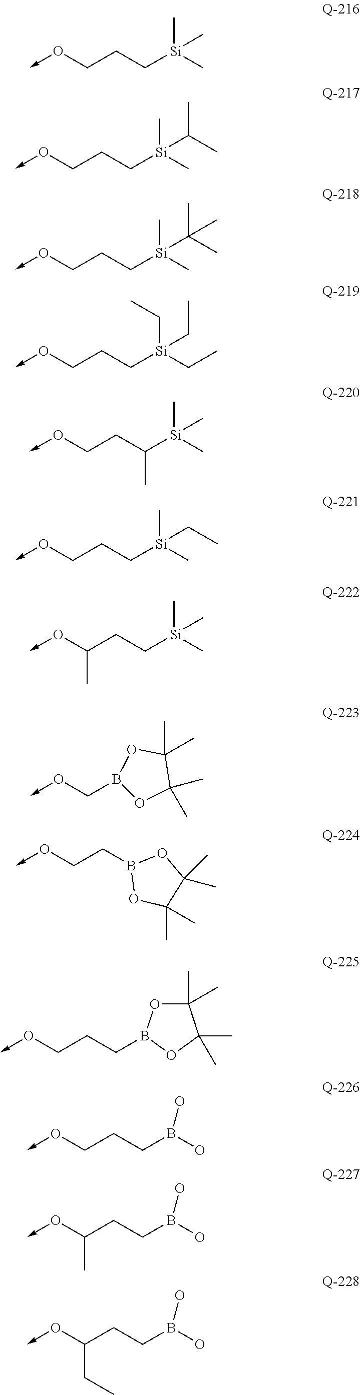

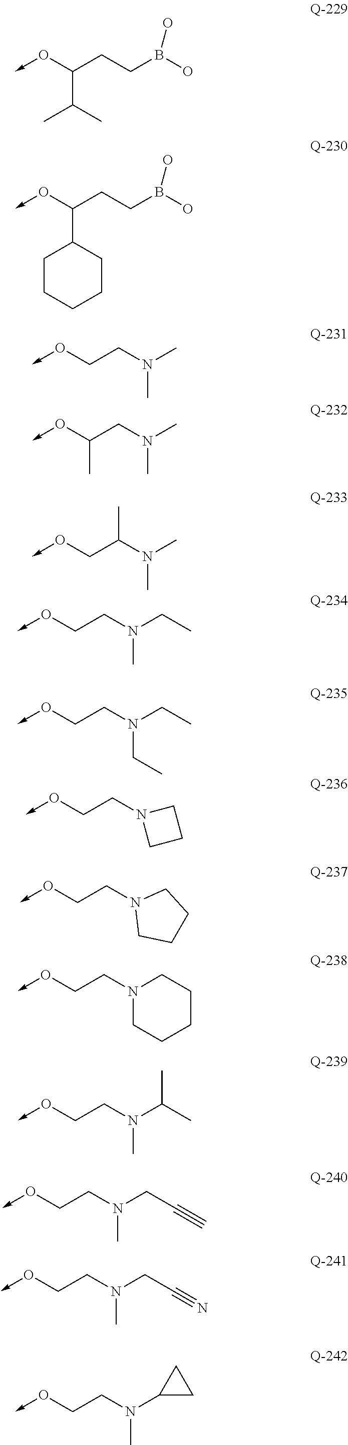

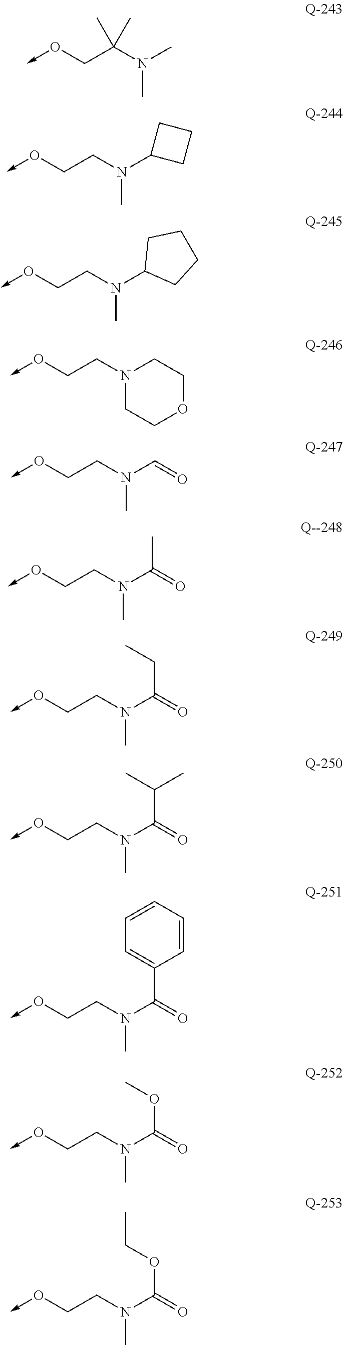

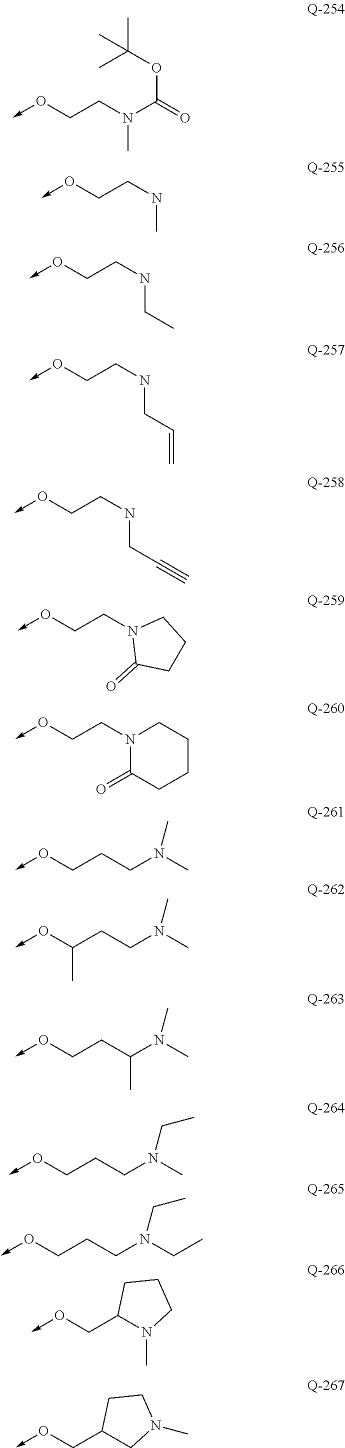

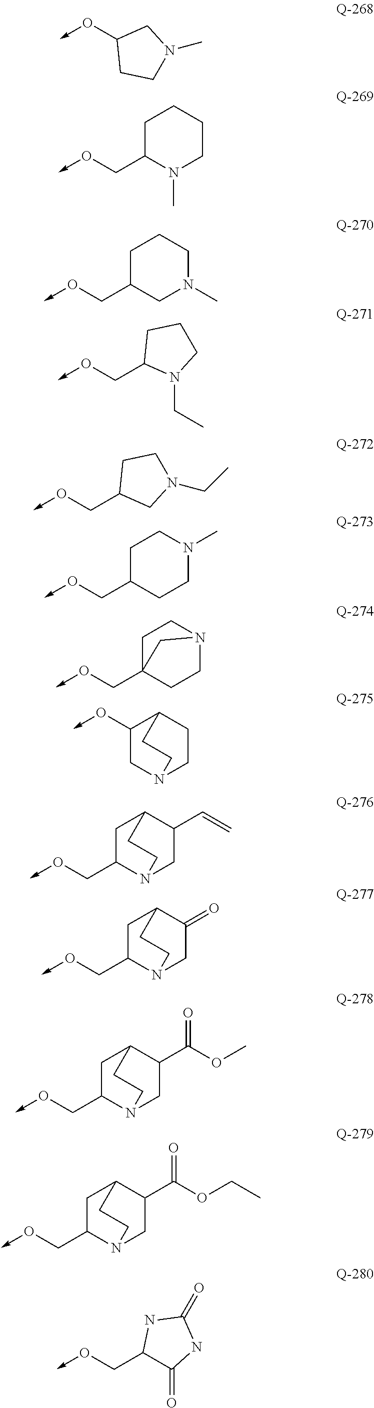

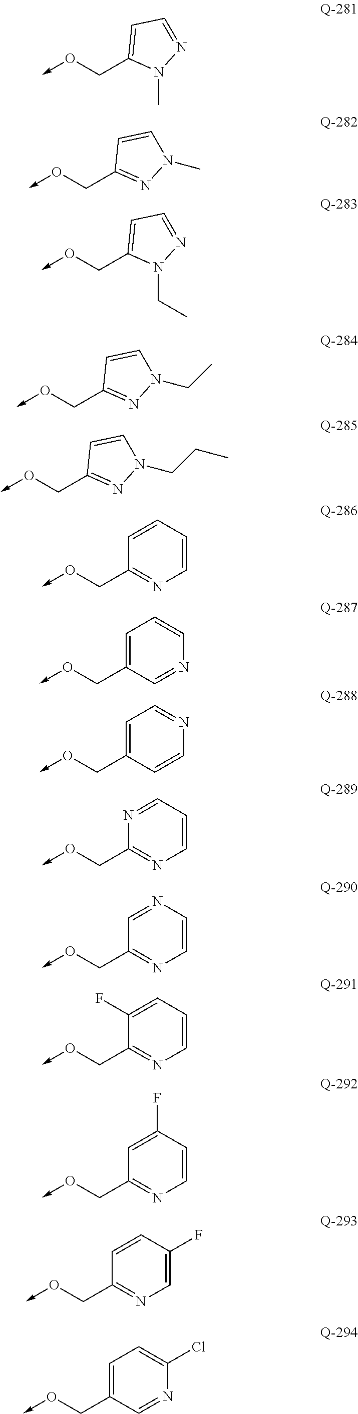

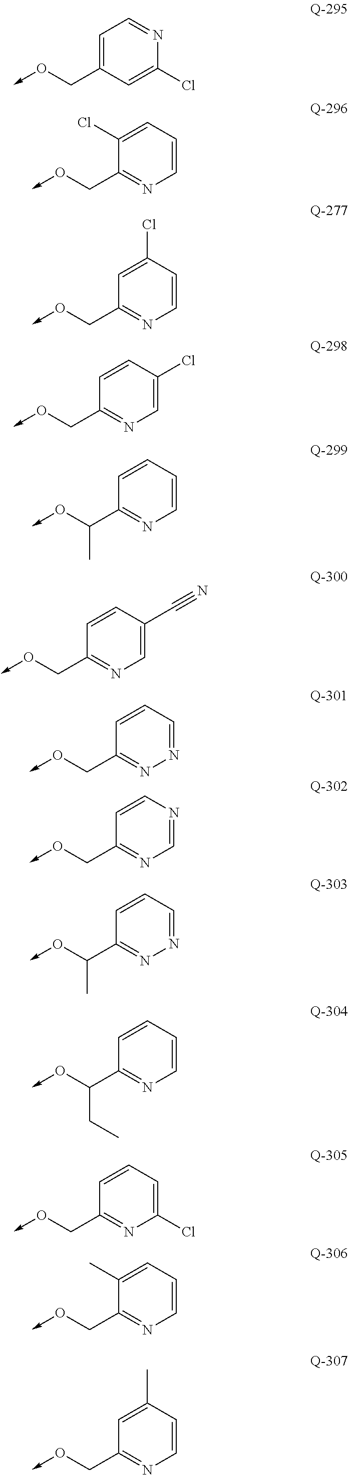

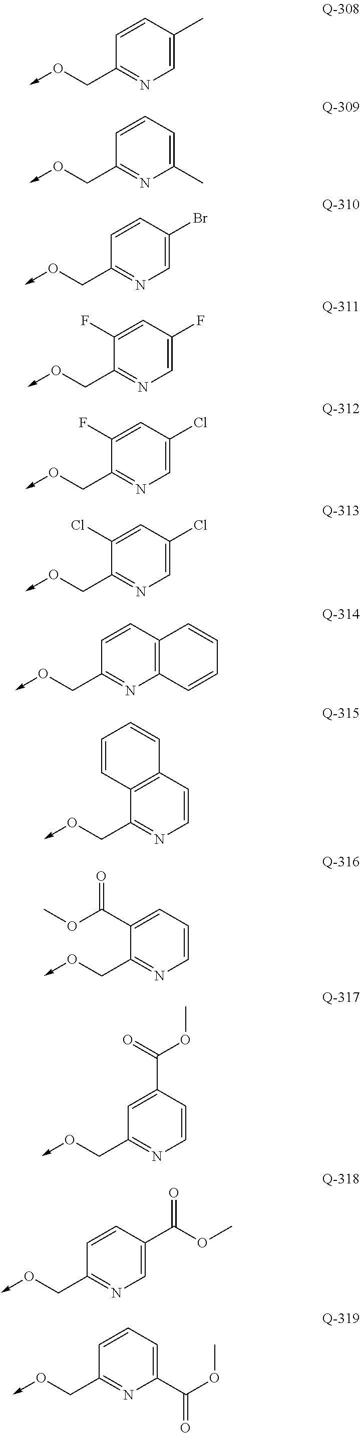

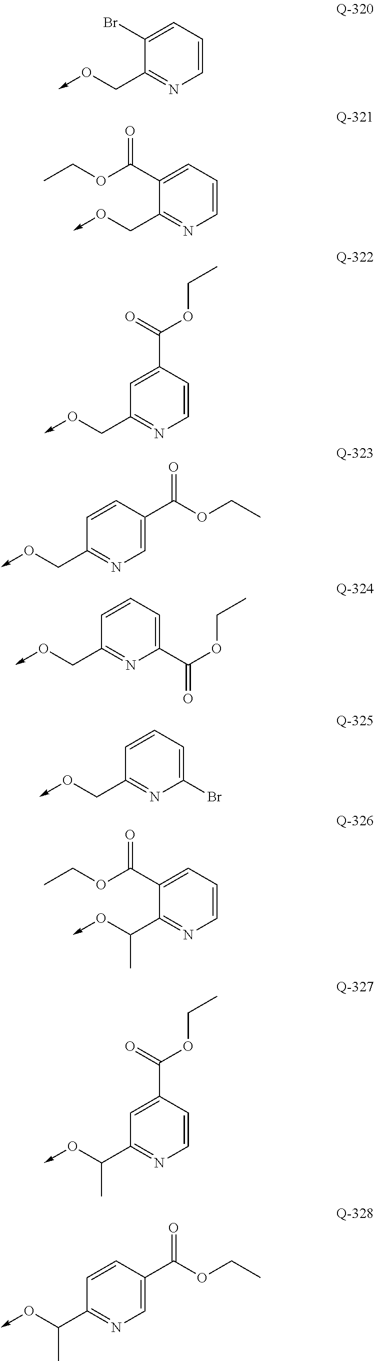

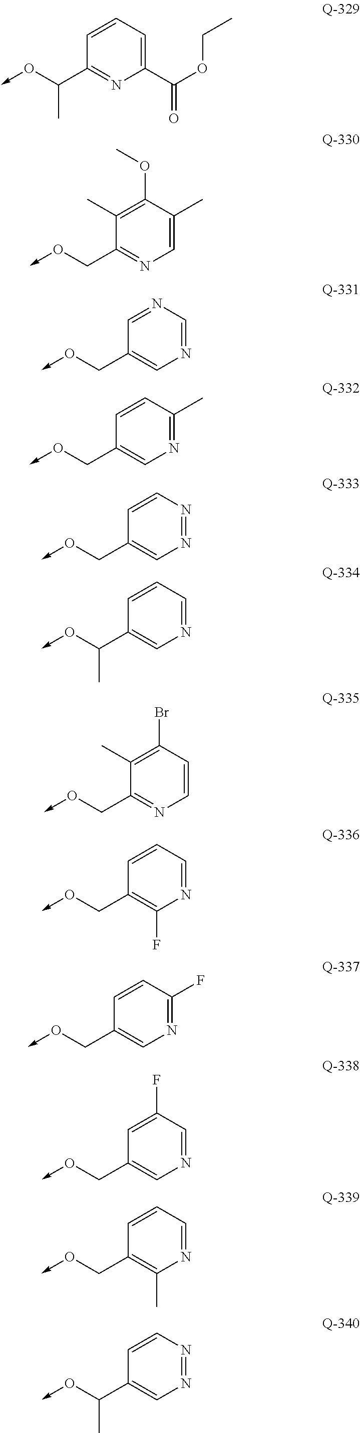

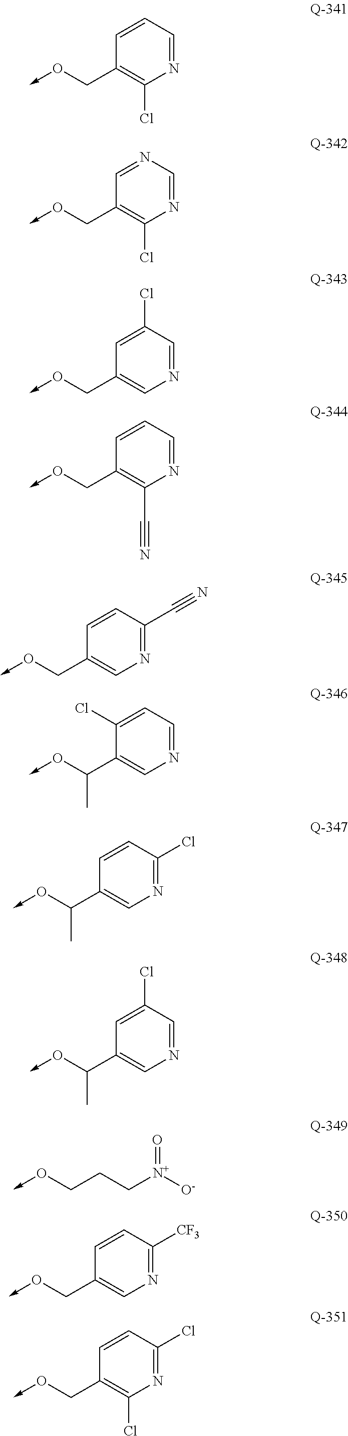

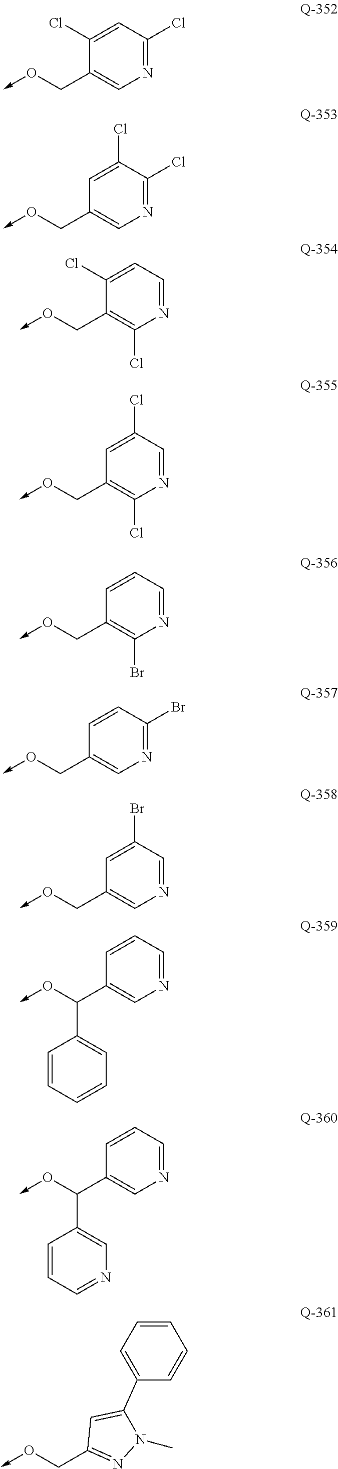

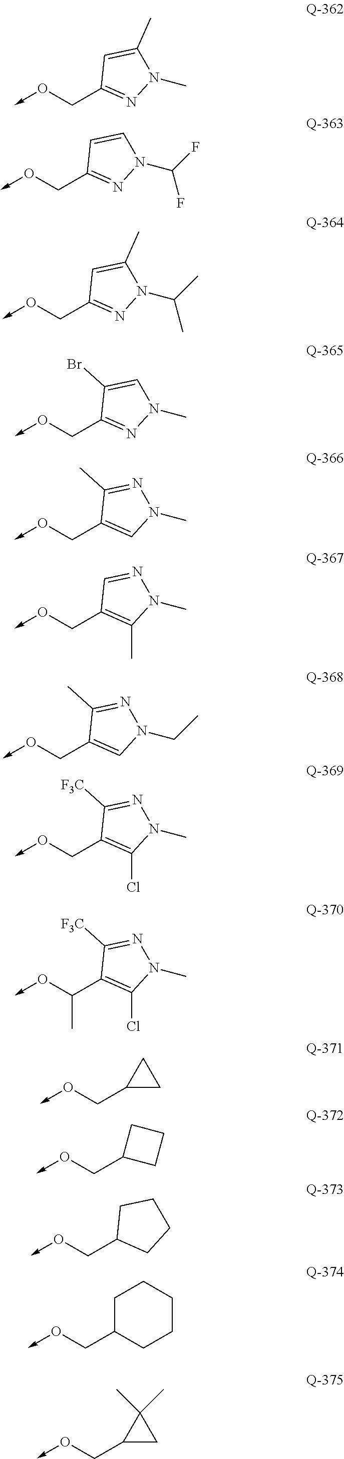

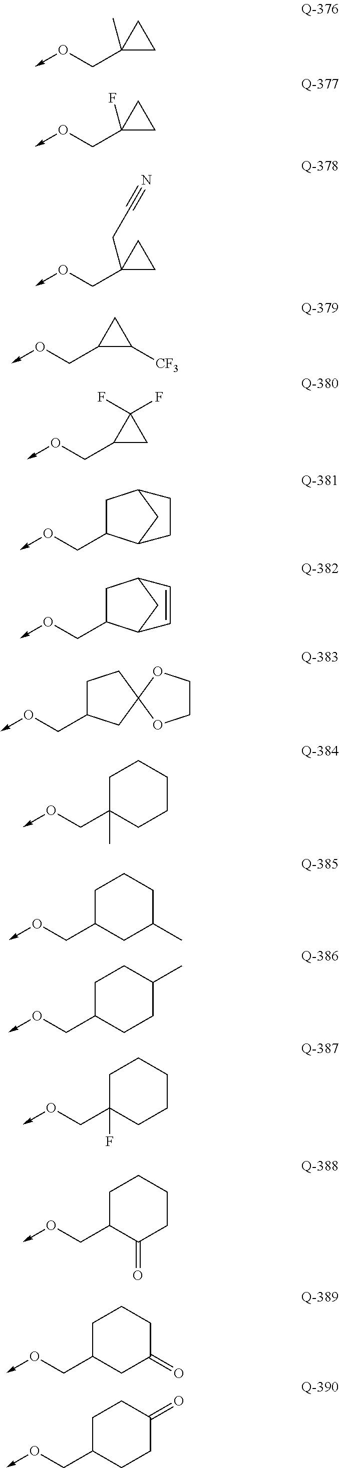

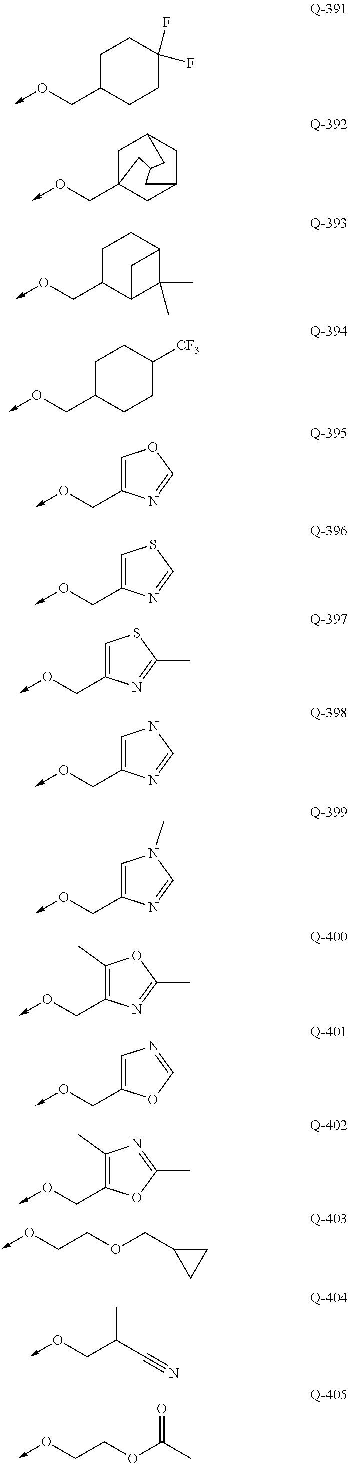

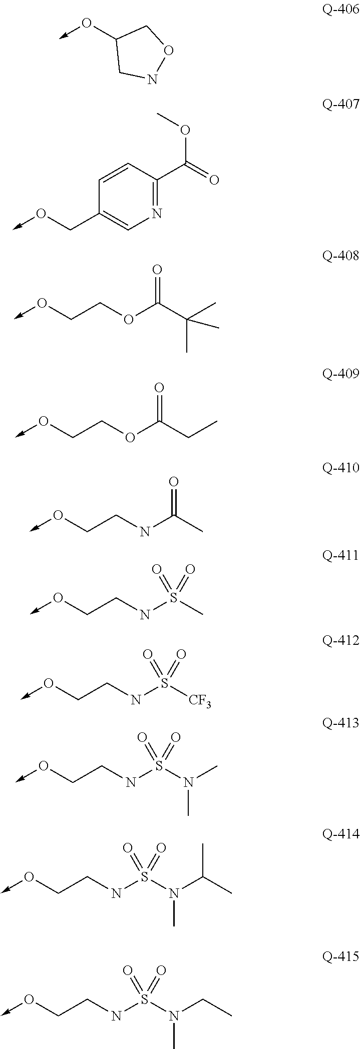

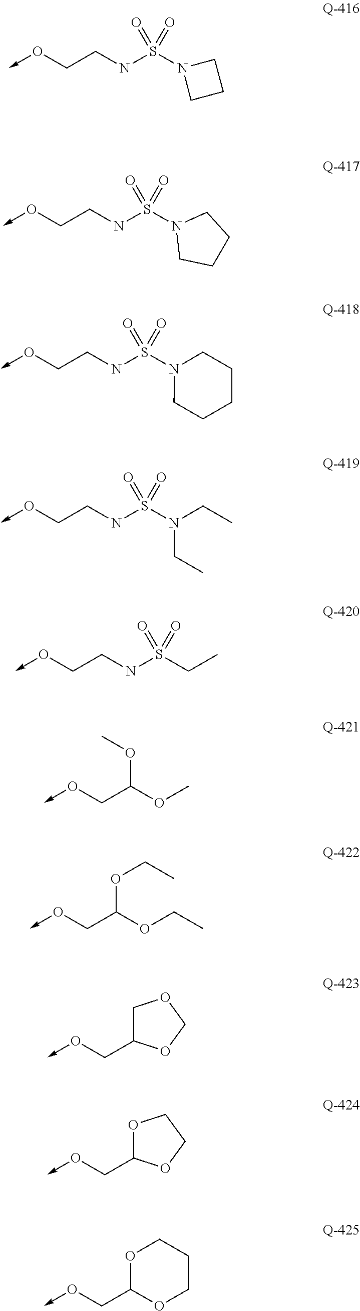

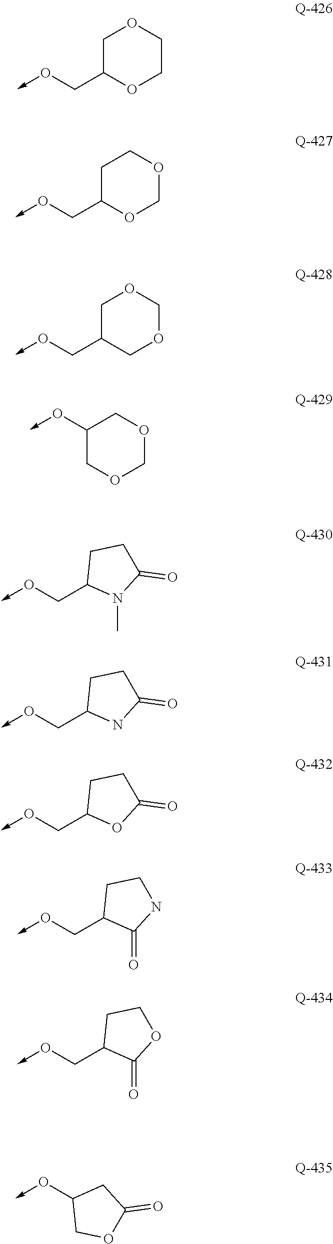

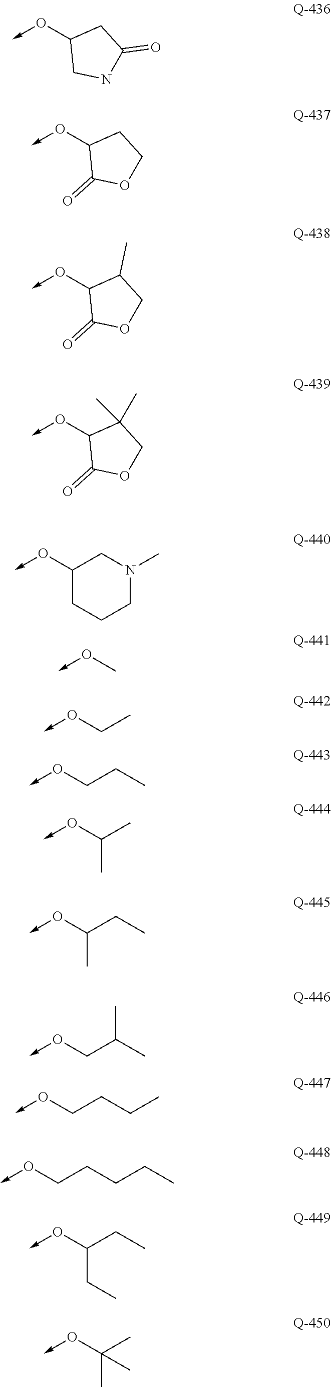

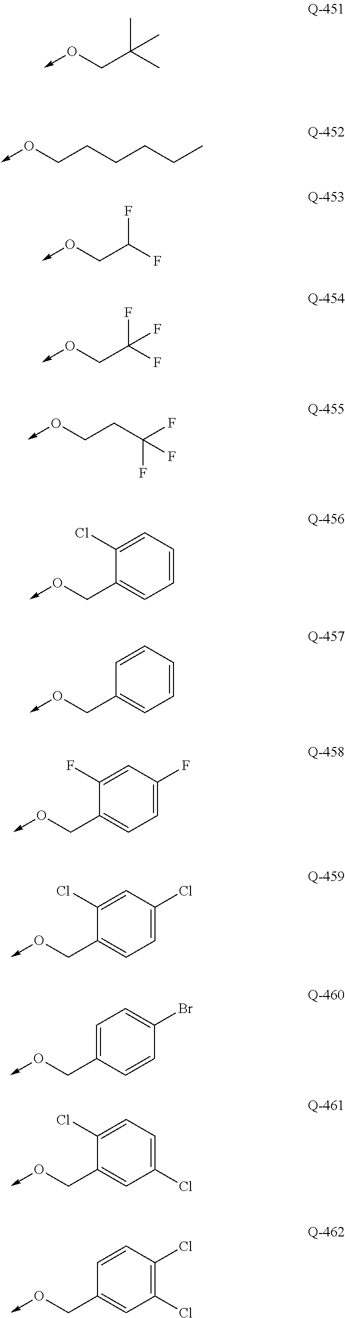

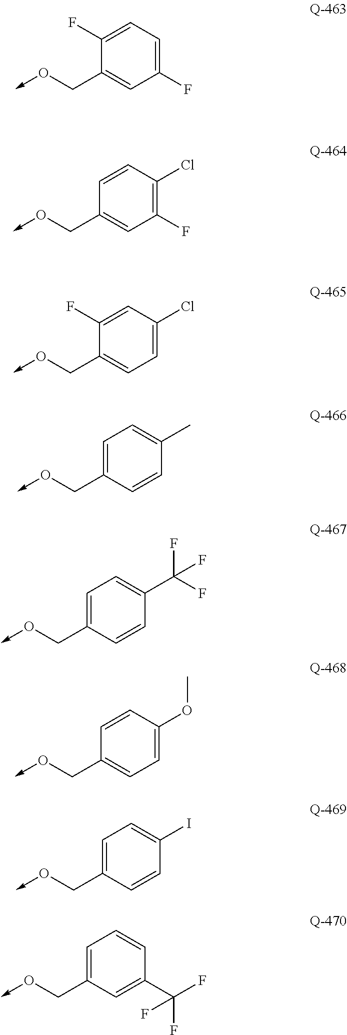

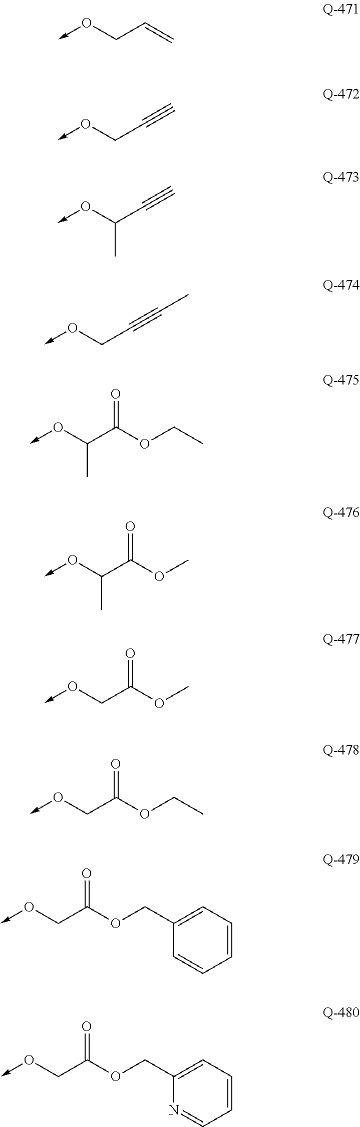

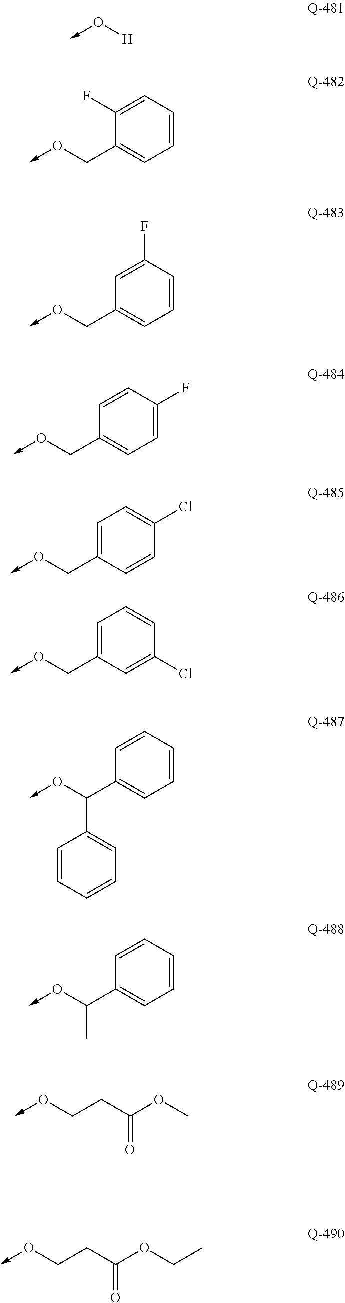

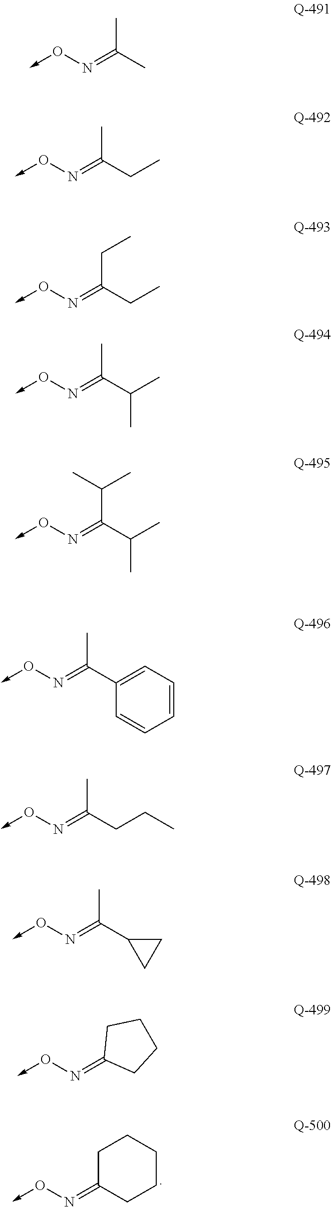

In preferred embodiments, the compound of the general formula (I) or an agrochemically acceptable salt thereof, corresponds to one of the compounds denoted (a)-(y) below, with their corresponding IUPAC names and structures.

| Compound | IUPAC name | Structure |
|---|---|---|
| (a) | ethyl {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetate | 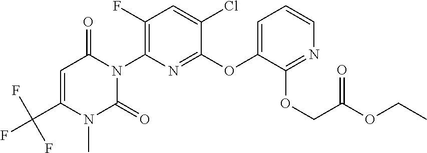 [SMILES: CCOC(=O)COc1ncccc1Oc1nc(-n2c(=O)cc(C(F)(F)F)n(C)c2=O)c(F)cc1Cl] |
| (b) | methyl {[3-({3-chloro-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 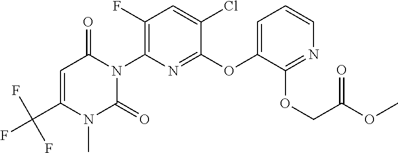 [SMILES: COC(=O)COc1ncccc1Oc1nc(-n2c(=O)cc(C(F)(F)F)n(C)c2=O)c(F)cc1Cl] |
| (c) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 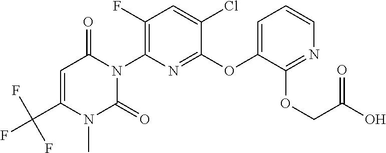 [SMILES: Cn1c(C(F)(F)F)cc(=O)n(-c2nc(Oc3cccnc3OCC(=O)O)c(Cl)cc2F)c1=O] |
| (d) | pyridazin-3-ylmethyl {[3-({3-chloro-5-fluoro- 6-[3-methyl-2,6-dioxo-4-(trifluoromethyl)- 3,6-dihydropyrimidin-1(2H)-yl]pyridin-2- yl}oxy)pyridin-2-yl]oxy}acetate | 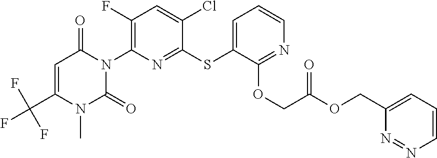 [SMILES: Cn1c(C(F)(F)F)cc(=O)n(-c2nc(Sc3cccnc3OCC(=O)OCc3cccnn3)c(Cl)cc2F)c1=O] |
| (e) | pyridin-2-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 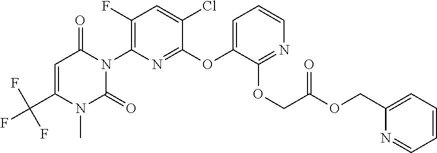 [SMILES: Cn1c(C(F)(F)F)cc(=O)n(-c2nc(Oc3cccnc3OCC(=O)OCc3ccccn3)c(Cl)cc2F)c1=O] |
| (f) | pyridin-4-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 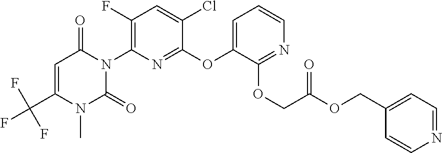 [SMILES: Cn1c(C(F)(F)F)cc(=O)n(-c2nc(Oc3cccnc3OCC(=O)OCc3ccncc3)c(Cl)cc2F)c1=O] |
| (g) | (4-trifluoromethylpyridin-3-yl)methyl{ [3-({3- chloro-5-fluoro-6-[3-methyl-2,6-dioxo-4- (trifluoromethyl)-3,6-dihydropyrimidin- 1(2H)-yl]pyridin-2-yl}oxy)pyridin-2- yl]oxy}acetate | 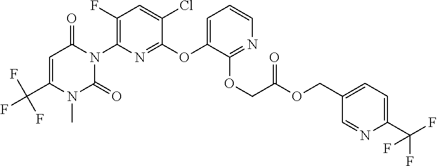 [SMILES: Cn1c(C(F)(F)F)cc(=O)n(-c2nc(Oc3cccnc3OCC(=O)OCc3ccc(C(F)(F)F)nc3)c(Cl)cc2F)c1=O] |
| (h) | ethyl [2-({3-chloro-5-fluoro-6-[3-methyl-2,6- dioxo-4-(trifluoromethyl)-3,6- dihydropyrimidin-1(2H)-yl]pyridin-2- yl}oxy)phenoxy]acetate | 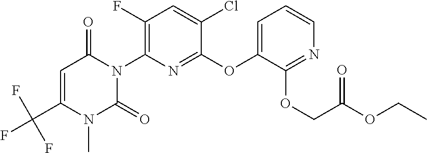 [SMILES: CCOC(=O)COc1ncccc1Oc1nc(-n2c(=O)cc(C(F)(F)F)n(C)c2=O)c(F)cc1Cl] |
| (i) | methyl [2-({3-chloro-5-fluoro-6-[3-methyl- 2,6-dioxo-4-(trifluoromethyl)-3,6- dihydropyrimidin-1(2H)-yl]pyridin-2- yl}oxy)phenoxy]acetate | 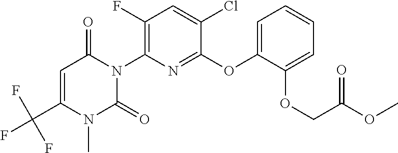 [SMILES: COC(=O)COc1ccccc1Oc1nc(-n2c(=O)cc(C(F)(F)F)n(C)c2=O)c(F)cc1Cl] |
| (j) | [2-({3-chloro-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 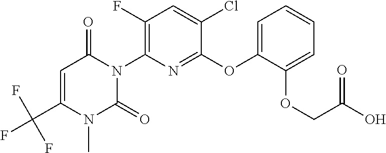 [SMILES: Cn1c(C(F)(F)F)cc(=O)n(-c2nc(Oc3ccccc3OCC(=O)O)c(Cl)cc2F)c1=O] |
| (k) | ethyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 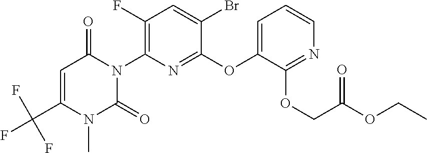 [SMILES: CCOC(=O)COc1ncccc1Oc1nc(-n2c(=O)cc(C(F)(F)F)n(C)c2=O)c(F)cc1Br] |
| (l) | methyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 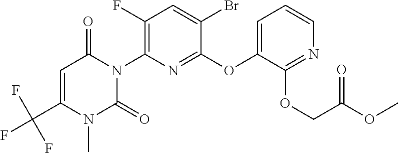 [SMILES: COC(=O)COc1ncccc1Oc1nc(-n2c(=O)cc(C(F)(F)F)n(C)c2=O)c(F)cc1Br] |
| (m) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 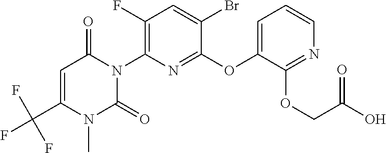 [SMILES: Cn1c(C(F)(F)F)cc(=O)n(-c2nc(Oc3cccnc3OCC(=O)O)c(Br)cc2F)c1=O] |
| (n) | ethyl [2-({3-bromo-5-fluoro-6-[3-methyl-2,6- dioxo-4-(trifluoromethyl)-3,6- dihydropyrimidin-1(2H)-yl]pyridin-2- yl}oxy)phenoxy]acetate | 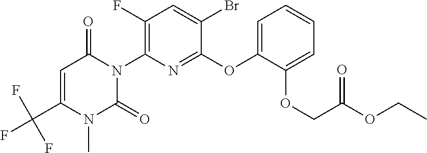 [SMILES: CCOC(=O)COc1ccccc1Oc1nc(-n2c(=O)cc(C(F)(F)F)n(C)c2=O)c(F)cc1Br] |
| (o) | methyl [2-({3-bromo-5-fluoro-6-[3-methyl- 2,6-dioxo-4-(trifluoromethyl)-3,6- dihydropyrimidin-1(2H)-yl]pyridin-2- yl}oxy)phenoxy]acetate | 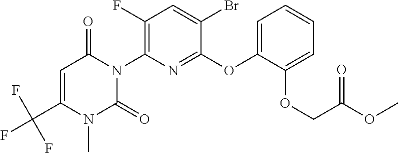 [SMILES: COC(=O)COc1ccccc1Oc1nc(-n2c(=O)cc(C(F)(F)F)n(C)c2=O)c(F)cc1Br] |
| (p) | [2-({3-bromo-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 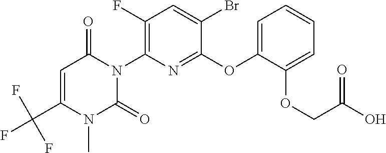 [SMILES: Cn1c(C(F)(F)F)cc(=O)n(-c2nc(Oc3ccccc3OCC(=O)O)c(Br)cc2F)c1=O] |
| (q) | ethyl{[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetate | 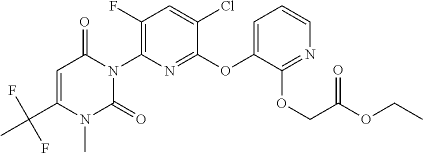 [SMILES: CCOC(=O)COc1ncccc1Oc1nc(-n2c(=O)cc(C(C)(F)F)n(C)c2=O)c(F)cc1Cl] |
| (r) | methyl {[3-({3-chloro-5-fluoro-6-[3-methyl- 2,6-dioxo-4-(1,1-difluoroethyl)-3,6- dihydropyrimidin-1(2H)-yl]pyridin-2- yl}oxy)pyridin-2-yl]oxy}acetate | 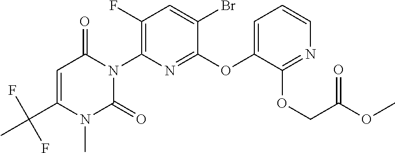 [SMILES: COC(=O)COc1ncccc1Oc1nc(-n2c(=O)cc(C(C)(F)F)n(C)c2=O)c(F)cc1Br] |
| (s) | {[3-({3-chloro-5-fluoro-6-[3-methyl-2,6- dioxo-4-(1,1-difluoroethyl)-3,6- dihydropyrimidin-1(2H)-yl]pyridin-2- yl} oxy)pyridin-2-yl]oxy}acetic acid | 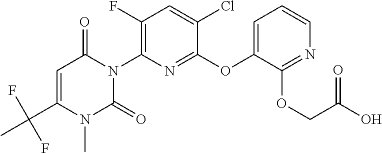 [SMILES: Cn1c(C(C)(F)F)cc(=O)n(-c2nc(Oc3cccnc3OCC(=O)O)c(Cl)cc2F)c1=O] |
| (t) | ethyl [2-({3-chloro-5-fluoro-6-[3-methyl-2,6- dioxo-4-(1,1-difluoroethyl)-3,6- dihydropyrimidin-1(2H)-yl]pyridin-2- yl}oxy)phenoxy]acetate | 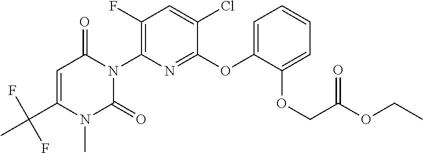 [SMILES: CCOC(=O)COc1ccccc1Oc1nc(-n2c(=O)cc(C(C)(F)F)n(C)c2=O)c(F)cc1Cl] |
| (u) | methyl [2-({3-chloro-5-fluoro-6-[3-methyl- 2,6-dioxo-4-(1,1-difluoroethyl)-3,6- dihydropyrimidin-1(2H)-yl]pyridin-2- yl}oxy)phenoxy]acetate | 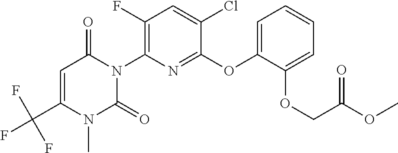 [SMILES: COC(=O)COc1ccccc1Oc1nc(-n2c(=O)cc(C(F)(F)F)n(C)c2=O)c(F)cc1Cl] |
| (v) | [2-({3-chloro-5-fluoro-6-[3-methyl-2,6-dioxo- 4-(1,1-difluoroethyl)-3,6-dihydropyrimidin- 1(2H)-yl]pyridin-2-yl}oxy)phenoxy]acetic acid | 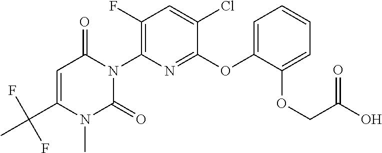 [SMILES: Cn1c(C(C)(F)F)cc(=O)n(-c2nc(Oc3ccccc3OCC(=O)O)c(Cl)cc2F)c1=O] |
| (x) | ethyl {[3-({3-chloro-5-fluoro-6-[3-methyl-2,6- dioxo-4-(trifluoromethyl)-3,6- dihydropyrimidin-1(2H)-yl]pyridin-2- yl}sulfanyl)pyridin-2-yl]oxy}acetate | 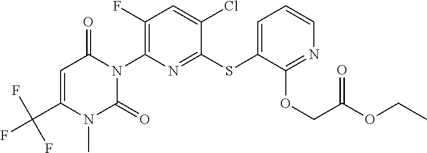 [SMILES: CCOC(=O)COc1ncccc1Sc1nc(-n2c(=O)cc(C(F)(F)F)n(C)c2=O)c(F)cc1Cl] |
| (y) | methyl {[3-({3-chloro-5-fluoro-6-[3-methyl- 2,6-dioxo-4-(trifluoromethyl)-3,6- dihydropyrimidin-1(2H)-yl]pyridin-2- yl}sulfanyl)pyridin-2-yl]oxy}acetate | 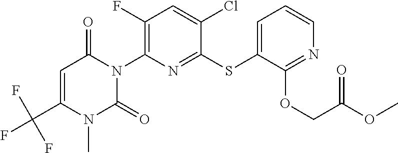 [SMILES: COC(=O)COc1ncccc1Sc1nc(-n2c(=O)cc(C(F)(F)F)n(C)c2=O)c(F)cc1Cl] |

In more preferred embodiments, the compound of the general formula (I) or an agrochemically acceptable salt thereof, corresponds to one of the compounds denoted (a)-(c), (h)-(j), and (n)-(o) below, with their corresponding IUPAC names: (a) ethyl {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetate; (b) methyl {[3-({3-chloro-5-fluoro-6-[3-methyl-2,6-dioxo-4-(trifluoromethyl)-3,6-dihydropyrimidin-1(2H)-yl]pyridin-2-yl} oxy)pyridin-2-yl]oxy}acetate; (c) {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetic acid; (h) ethyl [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetate; (i) methyl [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetate; (j) [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetic acid; (x) ethyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate; (y) methyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate.

The definitions of radicals listed above in general terms or within areas of preference apply to the end products of the general formula (I) and as well as to the starting materials or intermediates required for preparation in each case. These radical definitions can be combined with one another as desired, i.e. including combinations between the given preferred ranges. Primarily for reasons of higher herbicidal activity, better selectivity and/or better producibility, compounds of the general formula (I) described herein or their salts and their use according to the present disclosure are of particular interest in which individual radicals have one of the preferred meanings already specified or specified below, or in particular those in which one or more of the preferred meanings already specified or specified below occur in combination. With regard to the compounds according to the present disclosure, the terms used above and further below will be elucidated. These are familiar to the person skilled in the art and especially have the definitions elucidated hereinafter.

Unless defined differently, names of chemical groups are generally to be understood such that attachment to the skeleton or the remainder of the molecule is via the structural element mentioned last, i.e. for example in the case of (C₂-C₈)-alkenyloxy via the oxygen atom and in the case of (C₁-C₈)-alkoxy-(C₁-C₄)-alkyl or (C₁-C₈)-alkoxycarbonyl-(C₁-C₈)-alkyl, in each case via the carbon atom of the alkyl group. In the case of specified groups, such as OR¹³, NR¹¹R¹², S(O)ₘR¹⁴, C(═O)R¹³, C(═O)OR¹³, or C(═O)NR¹¹R¹² the attachment to the skeleton is via the structural element mentioned first, i.e. for example in the case of OR¹³ via the oxygen atom and in the case of C(═O)OR¹³ via the carbonyl atom.

As used herein and when described alone or as part of a chemical group, “alkylsulfonyl” refers to straight-chain or branched alkylsulfonyl, preferably having 1 to 6 or 1 to 8 carbon atoms. of such alkylsulfonyls include, but are but not limited to, (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

As used herein and when described alone or as part of a chemical group, “alkylthio” refers to a straight-chain or branched S-alkyl, preferably having 1 to 6 or 1 to 8 carbon atoms, such as (C₁-C₁₀)-, (C₁-C₆)- or (C₁-C₄)-alkylthio. Examples of such alkylthios include, but are but not limited to, (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio, and 1-ethyl-2-methylpropylthio.

As used herein and unless defined differently elsewhere, “alkylsulfinyl (alkyl-S(═O)—),” refers to alkyl radicals that are attached to the skeleton via —S(═O)—, such as (C₁-C₁₀)-, (C₁-C₆)-, or (C₁-C₄)-alkylsulfinyl. Examples of such alkylsulfinyls include, but are but not limited to, (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl, and 1-ethyl-2-methylpropylsulfinyl.

As used herein, “alkoxy” refers to an alkyl radical bonded via an oxygen atom. Examples of such alkoxys include, but are but not limited to, (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.

As used herein, “haloalkoxy” is, for example, OCF₃, OCHF₂, OCH₂F, OCF₂CF₃, OCH₂CF₃, and OCH₂CH₂Cl; this applies correspondingly to haloalkenyloxy, haloalkynyloxy, and other halogen-substituted radicals. The term “C₁-C₆-haloalkoxy” as mentioned herein by way of example thus refers to a C₁-C₆-alkoxy group, as defined above, in which one or more hydrogen atoms are replaced with halogen atoms that may be the same or different. Examples of C₁-C₆-haloalkoxy groups include, but are not limited to, chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, and 1,1,1-trifluoroprop-2-oxy.

Likewise, “alkenyloxy” refers to an alkenyl radical attached via an oxygen atom, and alkynyloxy refers to an alkynyl radical attached via an oxygen atom, such as (C₂-C₁₀)-, (C₂-C₆)-, or (C₂-C₄)-alkenyloxy, and (C₃-C₁₀)-, (C₃-C₆)-, or (C₃-C₄)-alkynyloxy.

The term “C₂-C₆-alkenyloxy” mentioned here by way of example refers to a formula (C₂-C₆-alkenyl)-O—, in which the term “C₂-C₆-alkenyl” group is which the as defined herein. Examples of C₂-C₆-alkenyloxy include but are not limited to ethenyloxy (or “vinyloxy”), prop-2-en-1-yloxy (or “allyloxy”), prop-1-en-1-yloxy, prop-1-en-2-yloxy (or “isopropenyloxy”), but-3-enyloxy, but-2-enyloxy, but-1-enyloxy, 2-methylprop-2-enyloxy, 1-methylprop-2-enyloxy, 2-methylprop-1-enyloxy, and 1-methylprop-1-enyloxy.

As used herein, “cycloalkoxy” refers to a cycloalkyl radical attached via an oxygen atom and cycloalkenyloxy refers to a cycloalkenyl radical attached via an oxygen atom.

As used herein, “alkylcarbonyl” (alkyl-C(═O)—), unless defined differently elsewhere, represents alkyl radicals attached to the skeleton via —C(═O)—, such as (C₁-C₁₀)-, (C₁-C₆)-, or (C₁-C₄)-alkylcarbonyl. Here, the number of the carbon atoms refers to the alkyl radical in the alkylcarbonyl group.

Analogously, unless defined differently elsewhere, “alkenylcarbonyl” and “alkynylcarbonyl” represent alkenyl and alkynyl radicals, respectively, attached to the skeleton via —C(═O)—, such as (C₂-C₁₀)-, (C₂-C₆)-, or (C₂-C₄)-alkenylcarbonyl and (C₂-C₁₀)-, (C₂-C₆)-, or (C₂-C₄)-alkynylcarbonyl. Here, the number of the carbon atoms refers to the alkenyl or alkynyl radical in the alkenyl or alkynyl group.

“Alkoxycarbonyl (alkyl-O—C(═O)—),” unless defined differently elsewhere refers to alkyl radicals attached to the skeleton via —O—C(═O)—, such as (C₁-C₁₀)-, (C₁-C₆)-, or (C₁-C₄)-alkoxycarbonyl. Here, the number of the carbon atoms refers to the alkyl radical in the alkoxycarbonyl group.

Analogously, “alkenyloxycarbonyl” and “alkynyloxycarbonyl,” unless defined differently elsewhere, respectively represent alkenyl and alkynyl radicals attached to the skeleton via —O—C(═O)—, such as (C₂-C₁₀)-, (C₂-C₆)-, or (C₂-C₄)-alkenyloxycarbonyl and (C₃-C₁₀)-, (C₃-C₆)-, or (C₃-C₄)-alkynyloxycarbonyl. Here, the number of the carbon atoms refers to the alkenyl or alkynyl radical in the alkenyloxycarbonyl or alkynyloxycarbonyl group.

The term “aryl” denotes an optionally substituted mono-, bi-, or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl.” Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, dialkylamino-alkoxy, tris-[alkyl]silyl, di-[alkyl]arylsilyl, di-[alkyl]alkylsilyl, tris-[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, dialkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, di-alkylaminocarbonyl, heteroarylalkoxy, and arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partially saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, such as, for example, 1-oxa-5-aza-spiro[2.3]hexyl. Unless defined otherwise, the heterocyclic ring preferably contains 3 to 9 ring atoms, in particular 3 to 6 ring atoms, and one or more, preferably 1 to 4, in particular 1, 2 or 3 heteroatoms in the heterocyclic ring, preferably from the group N, O, and S, where, however, two oxygen atoms must not be directly adjacent to one another, for example having one heteroatom from the group consisting of N, O, and S 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or - 3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or -3- or -4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group of N, O and S, for example 1- or 2- or 3- or 4-pyrazolidinyl; 4,5-dihydro-3H-pyrazol-3- or -4- or -5-yl; 4,5-dihydro-1H-pyrazol-1- or -3- or -4- or -5-yl; 2,3-dihydro-1H-pyrazol-1- or -2- or -3- or -4- or -5-yl; 1- or -2- or -3- or -4-imidazolidinyl; 2,3-dihydro-1H-imidazol-1- or -2- or -3- or -4-yl; 2,5-dihydro-1H-imidazol-1- or -2- or -4- or -5-yl; 4,5-dihydro-1H-imidazol-1- or -2- or -4- or -5-yl; hexahydropyridazin-1- or -2- or -3- or -4-yl; 1,2,3,4-tetrahydropyridazin-1- or -2- or -3- or -4- or -5- or -6-yl; 1,2,3,6-tetrahydropyridazin-1- or -2- or -3- or -4- or -5- or -6-yl; 1,4,5,6-tetrahydropyridazin-1- or -3- or -4- or -5- or -6-yl; 3,4,5,6-tetrahydropyridazin-3- or -4- or -5-yl; 4,5-dihydropyridazin-3- or -4-yl; 3,4-dihydropyridazin-3- or -4- or -5- or -6-yl; 3,6-dihydropyridazin-3- or -4-yl; 1,6-dihydropyridazin-1- or -3- or -4- or -5- or -6-yl; hexahydropyrimidin-1- or -2- or -3- or -4-yl; 1,4,5,6-tetrahydropyrimidin-1- or -2- or -4- or -5- or -6-yl; 1,2,5,6-tetrahydropyrimidin-1- or -2- or -4- or -5- or -6-yl; 1,2,3,4-tetrahydropyrimidin-1- or -2- or -3- or -4- or -5- or -6-yl; 1,6-dihydropyrimidin-1- or -2- or -4- or -5- or -6-yl; 1,2-dihydropyrimidin-1- or -2- or -4- or -5- or -6-yl; 2,5-dihydropyrimidin-2- or -4- or -5-yl; 4,5-dihydropyrimidin-4- or -5- or -6-yl; 1,4-dihydropyrimidin-1- or -2- or -4- or -5- or -6-yl; 1- or -2- or -3-piperazinyl; 1,2,3,6-tetrahydropyrazin-1- or -2- or -3- or -5- or -6-yl; 1,2,3,4-tetrahydropyrazin-1- or -2- or -3- or -4- or -5- or -6-yl; 1,2-dihydropyrazin-1- or -2- or -3- or -5- or -6-yl; 1,4-dihydropyrazin-1- or -2- or -3-yl; 2,3-dihydropyrazin-2- or -3- or -5- or -6-yl; 2,5-dihydropyrazin-2- or -3-yl; 1,3-dioxolan-2- or -4- or -5-yl; 1,3-dioxol-2- or -4-yl; 1,3-dioxan-2- or -4- or -5-yl; 4H-1,3-dioxin-2- or -4- or -5- or -6-yl; 1,4-dioxan-2- or -3- or -5- or -6-yl; 2,3-dihydro-1,4-dioxin-2- or -3- or -5- or -6-yl; 1,4-dioxin-2- or -3-yl; 1,2-dithiolan-3- or -4-yl; 3H-1,2-dithiol-3- or -4- or -5-yl; 1,3-dithiolan-2- or -4-yl; 1,3-dithiol-2- or -4-yl; 1,2-dithian-3- or -4-yl; 3,4-dihydro-1,2-dithiin-3- or -4- or -5- or -6-yl; 3,6-dihydro-1,2-dithiin-3- or -4-yl; 1,2-dithiin-3- or -4-yl; 1,3-dithian-2- or -4- or -5-yl; 4H-1,3-dithiin-2- or -4- or -5- or -6-yl; isoxazolidin-2- or -3- or -4- or -5-yl; 2,3-dihydroisoxazol-2- or -3- or -4- or -5-yl; 2,5-dihydroisoxazol-2- or -3- or -4- or -5-yl; 4,5-dihydroisoxazol-3- or -4- or -5-yl; 1,3-oxazolidin-2- or -3- or -4- or -5-yl; 2,3-dihydro-1,3-oxazol-2- or -3- or -4- or -5-yl; 2,5-dihydro-1,3-oxazol-2- or -4- or -5-yl; 4,5-dihydro-1,3-oxazol-2- or -4- or -5-yl; 1,2-oxazinan-2- or -3- or -4- or -5- or -6-yl; 3,4-dihydro-2H-1,2-oxazin-2- or -3- or -4- or -5- or -6-yl; 3,6-dihydro-2H-1,2-oxazin-2- or -3- or -4- or -5- or -6-yl; 5,6-dihydro-2H-1,2-oxazin-2- or -3- or -4- or -5- or -6-yl; 5,6-dihydro-4H-1,2-oxazin-3- or -4- or -5- or -6-yl; 2H-1,2-oxazin-2- or -3- or -4- or -5- or -6-yl; 6H-1,2-oxazin-3- or -4- or -5- or -6-yl; 4H-1,2-oxazin-3- or -4- or -5- or -6-yl; 1,3-oxazinan-2- or -3- or -4- or -5- or -6-yl; 3,4-dihydro-2H-1,3-oxazin-2- or -3- or -4- or -5- or -6-yl; 3,6-dihydro-2H-1,3-oxazin-2- or -3- or -4- or -5- or -6-yl; 5,6-dihydro-2H-1,3-oxazin-2- or -4- or -5- or -6-yl; 5,6-dihydro-4H-1,3-oxazin-2- or -4- or -5- or -6-yl; 2H-1,3-oxazin-2- or -4- or -5- or -6-yl; 6H-1,3-oxazin-2- or -4- or -5- or -6-yl; 4H-1,3-oxazin-2- or -4- or -5- or -6-yl; morpholin-2- or -3- or -4-yl; 3,4-dihydro-2H-1,4-oxazin-2- or -3- or -4- or -5- or -6-yl; 3,6-dihydro-2H-1,4-oxazin-2- or -3- or -5- or -6-yl; 2H-1,4-oxazin-2- or -3- or -5- or -6-yl; 4H-1,4-oxazin-2- or -3-yl; 1,2-oxazepan-2- or -3- or -4- or -5- or -6- or -7-yl; 2,3,4,5-tetrahydro-1,2-oxazepin-2- or -3- or -4- or -5- or -6- or -7-yl; 2,3,4,7-tetrahydro-1,2-oxazepin-2- or -3- or -4- or -5- or -6- or -7-yl; 2,3,6,7-tetrahydro-1,2-oxazepin-2- or -3- or -4- or -5- or -6- or -7-yl; 2,5,6,7-tetrahydro-1,2-oxazepin-2- or -3- or -4- or -5- or -6- or -7-yl; 4,5,6,7-tetrahydro-1,2-oxazepin-3- or -4- or -5- or -6- or -7-yl; 2,3-dihydro-1,2-oxazepin-2- or -3- or -4- or -5- or -6- or -7-yl; 2,5-dihydro-1,2-oxazepin-2- or -3- or -4- or -5- or -6- or -7-yl; 2,7-dihydro-1,2-oxazepin-2- or -3- or -4- or -5- or -6- or -7-yl; 4,5-dihydro-1,2-oxazepin-3- or -4- or -5- or -6- or -7-yl; 4,7-dihydro-1,2-oxazepin-3- or -4- or -5- or -6- or -7-yl; 6,7-dihydro-1,2-oxazepin-3- or -4- or -5- or -6- or -7-yl; 1,2-oxazepin-3- or -4- or -5- or -6- or -7-yl; 1,3-oxazepan-2- or -3- or -4- or -5- or -6- or -7-yl; 2,3,4,5-tetrahydro-1,3-oxazepin-2- or -3- or -4- or -5- or -6- or -7-yl; 2,3,4,7-tetrahydro-1,3-oxazepin-2- or -3- or -4- or -5- or -6- or -7-yl; 2,3,6,7-tetrahydro-1,3-oxazepin-2- or -3- or -4- or -5- or -6- or -7-yl; 2,5,6,7-tetrahydro-1,3-oxazepin-2- or -4- or -5- or -6- or -7-yl; 4,5,6,7-tetrahydro-1,3-oxazepin-2- or -4- or -5- or -6- or -7-yl; 2,3-dihydro-1,3-oxazepin-2- or -3- or -4- or -5- or -6- or -7-yl; 2,5-dihydro-1,3-oxazepin-2- or -4- or -5- or -6- or -7-yl; 2,7-dihydro-1,3-oxazepin-2- or -4- or -5- or -6- or -7-yl; 4,5-dihydro-1,3-oxazepin-2- or -4- or -5- or -6- or -7-yl; 4,7-dihydro-1,3-oxazepin-2- or -4- or -5- or -6- or -7-yl; 6,7-dihydro-1,3-oxazepin-2- or -4- or -5- or -6- or -7-yl; 1,3-oxazepin-2- or -4- or -5- or -6- or -7-yl; 1,4-oxazepan-2- or -3- or -5- or -6- or -7-yl; 2,3,4,5-tetrahydro-1,4-oxazepin-2- or -3- or -4- or -5- or -6- or -7-yl; 2,3,4,7-tetrahydro-1,4-oxazepin-2- or -3- or -4- or -5- or -6- or -7-yl; 2,3,6,7-tetrahydro-1,4-oxazepin-2- or -3- or -5- or -6- or -7-yl; 2,5,6,7-tetrahydro-1,4-oxazepin-2- or -3- or -5- or -6- or -7-yl; 4,5,6,7-tetrahydro-1,4-oxazepin-2- or -3- or -4- or -5- or -6- or -7-yl; 2,3-dihydro-1,4-oxazepin-2- or -3- or -5- or -6- or -7-yl; 2,5-dihydro-1,4-oxazepin-2- or -3- or -5- or -6- or -7-yl; 2,7-dihydro-1,4-oxazepin-2- or -3- or -5- or -6- or -7-yl; 4,5-dihydro-1,4-oxazepin-2- or -3- or -4- or -5- or -6- or -7-yl; 4,7-dihydro-1,4-oxazepin-2- or -3- or -4- or -5- or -6- or -7-yl; 6,7-dihydro-1,4-oxazepin-2- or -3- or -5- or -6- or -7-yl; 1,4-oxazepin-2- or -3- or -5- or -6- or -7-yl; isothiazolidin-2- or -3- or -4- or -5-yl; 2,3-dihydroisothiazol-2- or -3- or -4- or -5-yl; 2,5-dihydroisothiazol-2- or -3- or -4- or -5-yl; 4,5-dihydroisothiazol-3- or -4- or -5-yl; 1,3-thiazolidin-2- or -3- or -4- or -5-yl; 2,3-dihydro-1,3-thiazol-2- or -3- or -4- or -5-yl; 2,5-dihydro-1,3-thiazol-2- or -4- or -5-yl; 4,5-dihydro-1,3-thiazol-2- or -4- or -5-yl; 1,3-thiazinan-2- or -3- or -4- or -5- or -6-yl; 3,4-dihydro-2H-1,3-thiazin-2- or -3- or -4- or -5- or -6-yl; 3,6-dihydro-2H-1,3-thiazin-2- or -3- or -4- or -5- or -6-yl; 5,6-dihydro-2H-1,3-thiazin-2- or -4- or -5- or -6-yl; 5,6-dihydro-4H-1,3-thiazin-2- or -4- or -5- or -6-yl; 2H-1,3-thiazin-2- or -4- or -5- or -6-yl; 6H-1,3-thiazin-2- or -4- or -5- or -6-yl; 4H-1,3-thiazin-2- or -4- or -5- or -6-yl. Further examples of “heterocyclyl” are a partially or fully hydrogenated heterocyclic radical having 3 heteroatoms from the group of N, O and S, for example 1,4,2-dioxazolidin-2- or -3- or -5-yl; 1,4,2-dioxazol-3- or -5-yl; 1,4,2-dioxazinan-2- or -3- or -5- or -6-yl; 5,6-dihydro-1,4,2-dioxazin-3- or -5- or -6-yl; 1,4,2-dioxazin-3- or -5- or -6-yl; 1,4,2-dioxazepan-2- or -3- or -5- or -6- or -7-yl; 6,7-dihydro-5H-1,4,2-dioxazepin-3- or -5- or -6- or -7-yl; 2,3-dihydro-7H-1,4,2-dioxazepin-2- or -3- or -5- or -6- or -7-yl; 2,3-dihydro-5H-1,4,2-dioxazepin-2- or -3- or -5- or -6- or -7-yl; 5H-1,4,2-dioxazepin-3- or -5- or -6- or -7-yl; 7H-1,4,2-dioxazepin-3- or -5- or -6- or -7-yl. Structural examples of heterocycles that are optionally substituted further are also listed below:

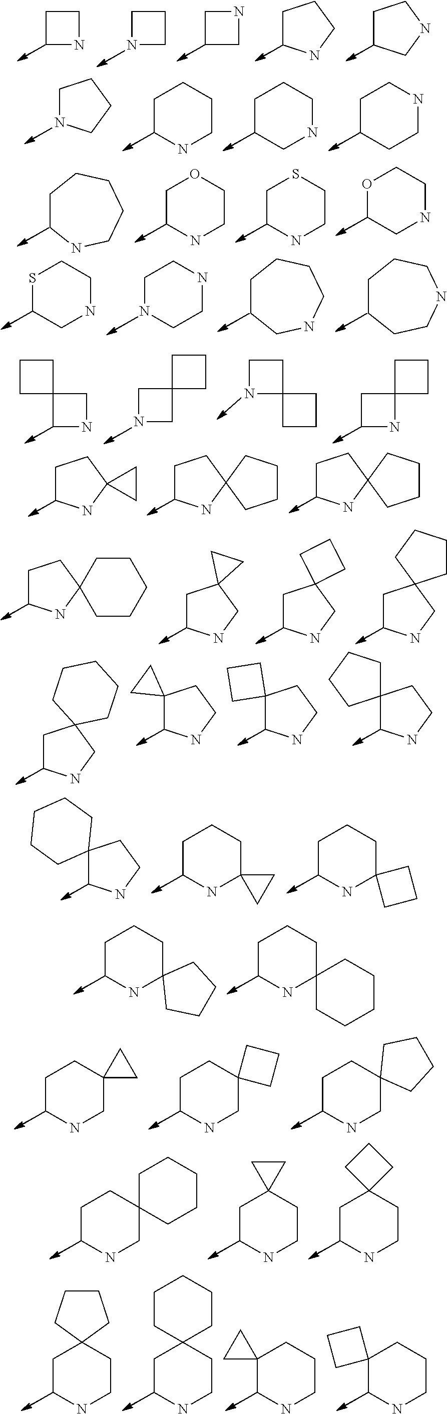

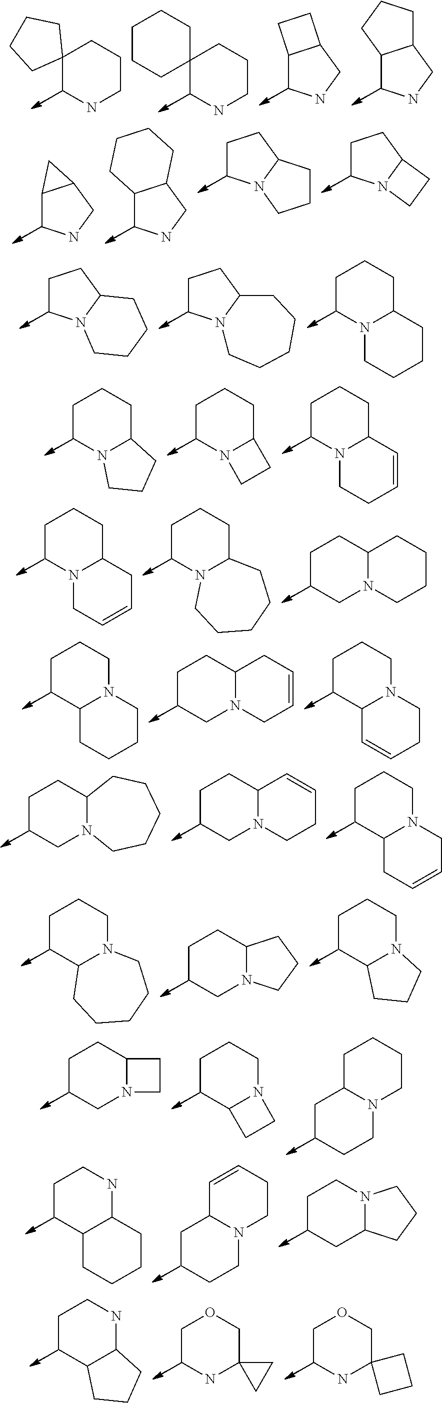

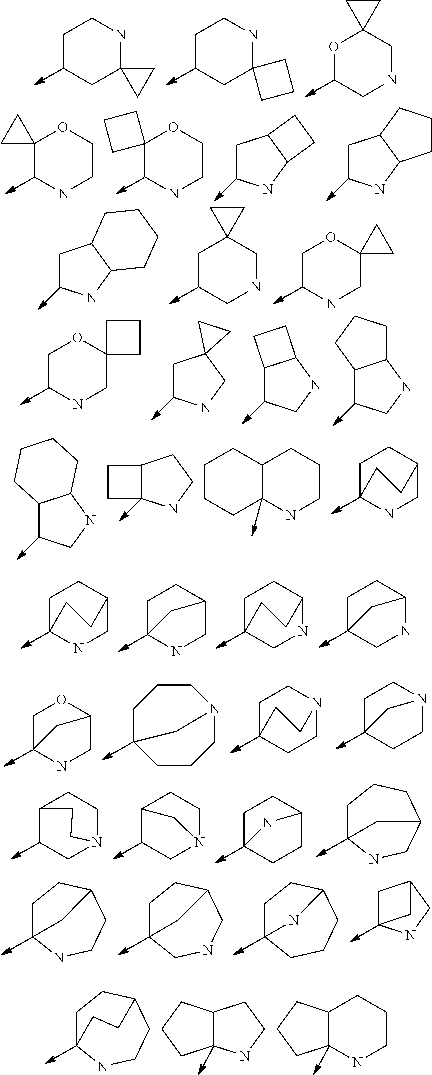

The heterocycles listed above are preferably substituted by, for example, hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, dialkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, dialkylaminocarbonyl, cycloalkylaminocarbonyl, hydroxycarbonylalkylaminocarbonyl, alkoxycarbonylalkylaminocarbonyl, and arylalkoxycarbonylalkylaminocarbonyl.

When a base structure is substituted “by one or more substituents” from a list of radicals (=group) or a generically defined group of radicals, this in each case includes simultaneous substitution by a plurality of identical and/or structurally different radicals. In the case of a partially or fully saturated nitrogen heterocycle, this may be joined to the remainder of the molecule either via carbon or via the nitrogen. Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)— and —S(O)— groups, both enantiomers in each case are included.

As used herein, “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably O, S, or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups described herein may also be substituted by one or more identical or different radicals. If two adjacent carbon atoms are part of a further aromatic ring, the systems are fused heteroaromatic systems, such as benzofused or polyannealed heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

As used herein, “alkyl” refers to a straight-chain or branched open-chain, saturated hydrocarbon radical that is optionally mono- or polysubstituted, and in the latter case is referred to as “substituted alkyl.” Preferred substituents are halogen atoms, alkoxy, haloalkoxy, cyano, alkylthio, haloalkylthio, amino, or nitro groups, particular preference being given to methoxy, methyl, fluoroalkyl, cyano, nitro, fluorine, chlorine, bromine, or iodine.

The prefix “di” includes the combination of equal or different alkyl radicals, e.g. dimethyl or methyl(ethyl) or ethyl(methyl).

As used herein, “haloalkyl,” “-alkenyl,” and “-alkynyl” denote an alkyl, alkenyl, and alkynyl, respectively, that is partially or fully substituted by identical or different halogen atoms, for example monohaloalkyl, such as CH₂CH₂Cl, CH₂CH₂Br, CHClCH₃, CH₂Cl, and CH₂F; perhaloalkyl or perfluoroalkyl, such as CCl₃, CClF₂, CFCl₂, CF₂CClF₂, and CF₂CClFCF₃; polyhaloalkyl, such as CH₂CHFCl, CF₂CClFH, CF₂CBrFH, and CH₂CF₃.

The term “C₂-C₆-haloalkenyl” as mentioned herein by way of example refers to a C₂-C₆-alkenyl group as defined above in which one or more hydrogen atoms are replaced with one or more halogen atoms that may be the same or different. Typically, C₂-C₆-haloalkenyl comprises up to 9 halogen atoms that can be the same or different.

The term “C₂-C₆-haloalkynyl” as mentioned herein by way of example refers to a C₂-C₆-alkynyl group as defined above in which one or more hydrogen atoms are replaced with one or more halogen atoms that may be the same or different. Typically, C₂-C₆-haloalkynyl comprises up to 9 halogen atoms that can be the same or different.

As used herein, “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl, or tert-butyl radicals. General alkyl radicals with a larger specified range of carbon atoms, e.g. “(C₁-C₆)-alkyl,” also encompass straight-chain or branched alkyl radicals with a greater number of carbon atoms, i.e. alkyl radicals having 5 and 6 carbon atoms. Unless stated specifically, preference is given to the lower carbon skeletons, for example, having from 1 to 6 carbon atoms, or having from 2 to 6 carbon atoms in the case of unsaturated groups, in the case of the hydrocarbyl radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls; hexyls, such as n-hexyl, i-hexyl, and 1,3-dimethylbutyl; heptyls, such as n-heptyl, 1-methylhexyl, and 1,4-dimethylpentyl.

Alkenyl and alkynyl radicals are defined as the possible unsaturated radicals corresponding to the alkyl radicals, where at least one double bond or triple bond is present. Preference is given to radicals having one double bond or triple bond.

As used herein, the term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl.

As used herein, the term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl.

As used herein, the term “cycloalkyl” denotes a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, dialkylamino, alkoxycarbonyl, hydroxycarbonyl, arylalkoxycarbonyl, aminocarbonyl, alkylaminocarbonyl, and cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl, and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms. In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, and spiro[3.3]hept-2-yl.

“Cycloalkenyl” denotes a carbocyclic, nonaromatic, partially unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

As used herein, “haloalkylthio”—on its own or as constituent part of a chemical group—represents straight-chain or branched S-haloalkyl, preferably having 1 to 8, or having 1 to 6 carbon atoms, such as (C₁-C₈)-, (C₁-C₆)-, or (C₁-C₄)-haloalkylthio, for example (but not limited thereto) trifluoromethylthio, pentafluoroethylthio, difluoromethyl, 2,2-difluoroeth-1-ylthio, 2,2,2-difluoroeth-1-ylthio, and 3,3,3-prop-1-ylthio.

“Halocycloalkyl” and “halocycloalkenyl” denote cycloalkyl and cycloalkenyl, respectively, which are partially or fully substituted by identical or different halogen atoms, such as F, Cl, and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, and 3,3-difluorocyclobutyl.

As used herein, “trialkylsilyl” used alone or as constituent part of a chemical group represents a straight-chain or branched Si-alkyl, preferably having 1 to 8, or having 1 to 6 carbon atoms, such as tri[(C₁-C₈)-, (C₁-C₆)- or (C₁-C₄)-alkyl]silyl, for example (but not limited thereto) trimethylsilyl, triethylsilyl, tri(n-propyl)silyl, tri(isopropyl)silyl, tri(n-butyl)silyl, tri(1-methylprop-1-yl)silyl, tri(2-methylprop-1-yl)silyl, tri(1,1-dimethyleth-1-yl)silyl, and tri(2,2-dimethyleth-1-yl)silyl.

As used herein, “oxo” refers to an oxygen atom that is bonded to a carbon atom or sulfur atom by a double bond.

As used herein, “methylidene” refers to a CH₂ group connected to a carbon atom by a double bond.

As used herein, “halomethylidene” refers to a CX₂ group connected to a carbon atom by a double bond, where X is halogen.

If the compounds can form, through a hydrogen shift, tautomers whose structure is not formally covered by the general formula (I), these tautomers are nevertheless covered by the definition of the inventive compounds of the general formula (I), unless a particular tautomer is under consideration. For example, many carbonyl compounds may be present both in the keto form and in the enol form, both forms being encompassed by the definition of the compound of the general formula (I).

According to the nature of the substituents defined above, the compounds of the formula (I) have acidic properties and can form salts, and if appropriate also internal salts or adducts with inorganic or organic bases or with metal ions. If the compounds of the formula (I) bear hydroxyl, carboxyl or other groups which induce acidic properties, these compounds can be reacted with bases to give salts. Suitable bases are, for example, hydroxides, carbonates, hydrogen carbonates of the alkali metals and alkaline earth metals, especially those of sodium, potassium, magnesium, and calcium, and also ammonia, primary, secondary, and tertiary amines having (C₁-C₄)-alkyl groups, mono-, di-, and trialkanolamines of (C₁-C₄)-alkanols, choline and chlorocholine, and organic amines, such as trialkylamines, morpholine, piperidine, or pyridine. These salts are compounds in which the acidic hydrogen is replaced by an agriculturally suitable cation, for example metal salts, especially alkali metal salts or alkaline earth metal salts, especially sodium and potassium salts, or else ammonium salts, salts with organic amines or quaternary ammonium salts, for example with cations of the formula [NRR′R″R′″]⁺ in which R to R′″ are each independently an organic radical, especially alkyl, aryl, aralkyl, or alkylaryl. Also suitable are alkyl sulfonium and alkyl sulfoxonium salts, such as (C₁-C₄)-trialkyl sulfonium and (C₁-C₄)-trialkyl sulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-Toluenesulfonic acid, onto a basic group, for example amino, alkylamino, dialkylamino, piperidino, morpholino, or pyridino. In this case, these salts comprise the conjugate base of the acid as the anion.

Suitable substituents present in deprotonated form, for example sulfonic acids or carboxylic acids, may form internal salts with groups which for their part can be protonated, such as amino groups.

If a group is polysubstituted by radicals, this means that this group is substituted by one or more identical or different radicals from those mentioned.

The present disclosure also relates to all stereoisomers and mixtures thereof which are encompassed by the formula (I) but not defined specifically. For the sake of simplicity, however, reference will always be made hereinafter to compounds of the formula (I), even though this means not only the pure compounds but also, if appropriate, mixtures with different proportions of isomeric compounds.

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The present disclosure thus also relates to all stereoisomers which are embraced by the general formula (I) but are not shown in their specific stereometric form, and to mixtures thereof.

As used herein, a “herbicide” is any molecule that is used to control, prevent, or interfere with the growth of one or more plants. Illustrative herbicides include acetyl-CoA carboxylase (ACCase) inhibitors (for example, aryloxyphenoxy propionates and cyclohexanediones); acetolactate synthase (ALS) inhibitors (for example, sulfonylureas, imidazolinones, triazolopyrimidines, and triazolinones); 5-enolpyruvylshikimate-3-phosphate synthase (EPSPS) inhibitors (for example, glyphosate), synthetic auxins (for example, phenoxys, benzoic acids, carboxylic acids, and semicarbazones), photosynthesis (photosystem II) inhibitors (for example, triazines, triazinones, nitriles, benzothiadiazoles, and ureas), glutamine synthetase (GS) inhibitors (for example, glufosinate and bialaphos), 4-hydroxyphenylpyruvate dioxygenase (HPPD) inhibitors (for example, isoxazoles, pyrazolones, and triketones), protoporphyrinogen oxidase (PPO) inhibitors (for example, diphenylethers, N-phenylphthalimide, aryl triazinones, and pyrimidinediones), very long-chain fatty acid inhibitors (for example, chloroacetamides, oxyacetamides, and pyrazoles), cellulose biosynthesis inhibitors (for example, indaziflam), photosystem I inhibitors (for example, paraquat), microtubule assembly inhibitors (for example, pendimethalin), and phytoene desaturase (PDS) inhibitors (for example, norflurazone), among others.

Specific PPO inhibiting herbicides are known in the art and commercially available. Examples of PPO inhibiting herbicides include, but are not limited to, diphenylethers (such as acifluorfen, its salts and esters, bifenox, its salts and esters, ethoxyfen, its salts and esters, fluoronitrofen, furyloxyfen, halosafen, chlomethoxyfen, fluoroglycofen, its salts and esters, lactofen, its salts and esters, oxyfluorfen, and fomesafen, its salts and esters); thiadiazoles (such as fluthiacet-methyl and thidiazimin); pyrimidinediones or phenyluracils (such as benzfendizone, butafenacil, epyrifenacil (ethyl [3-2-chloro-4-fluoro-5-(1-methyl-6-trifluoromethyl-2,4-dioxo-1,2,3,4-tetrahydropyrimidin-3-yl)phenoxy]-2-pyridyloxy]acetate; CAS Registry Number 353292-31-6 and also referred to herein as S-3100), flupropacil, saflufenacil, and tiafenacil); phenylpyrazoles (such as fluazolate, pyraflufen, and pyraflufen-ethyl); oxadiazoles (such as oxadiargyl and oxadiazon); triazolinones (such as azafenidin, bencarbazone, carfentrazone, its salts and esters, and sulfentrazone); oxazolidinediones (such as pentoxazone); N-phenylphthalimides (such as cinidon-ethyl, flumiclorac, flumiclorac-pentyl, and flumioxazin); benzoxazinone derivatives (such as 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)); flufenpyr and flufenpyr-ethyl; pyraclonil; and profluazol.

As used herein, “herbicide-tolerant” or “herbicide-tolerance” means the ability to be wholly or partially unaffected by the presence or application of one or more herbicide(s), for example to resist the toxic effects of an herbicide when applied. A cell or organism is “herbicide-tolerant” if it is able to maintain at least some normal growth or phenotype in the presence of one or more herbicide(s). A trait is an herbicide-tolerance trait if its presence can confer improved tolerance to an herbicide upon a cell, plant, or seed as compared to the wild-type or control cell, plant, or seed. Crops comprising a herbicide-tolerance trait can continue to grow and are minimally affected by the presence of the herbicide. A target enzyme is “herbicide-tolerant” if it exhibits improved enzyme activity relative to a wild-type or control enzyme in the presence of the herbicide. Herbicide-tolerance may be complete or partial insensitivity to a particular herbicide, and may be expressed as a percent (%) tolerance or insensitivity to a particular herbicide.

Contemplated plants which might be produced with an herbicide tolerance trait of the present disclosure could include, for instance, any plant susceptible to a PPO inhibitor herbicide, including crop plants such as soybean (Glycine max), maize (Zea mays), cotton (Gossypium sp.), Brassica plants, alfalfa, barley, beans, beet, broccoli, cabbage, carrot, canola, cauliflower, celery, Chinese cabbage, cucumber, eggplant, leek, lettuce, melon, oat, onion, pea, pepper, peanut, potato, pumpkin, radish, rice, sweet corn, sorghum, spinach, squash, sugar beet, sugar cane, sunflower, tomato, watermelon, and wheat, among others.

Herbicides may be applied to a plant growth area comprising the plants and seeds provided by the disclosure as a method for controlling weeds. Plants and seeds provided by the disclosure comprise an herbicide tolerance trait and as such are tolerant to the application of one or more PPO inhibiting herbicides. The herbicide application may be the recommended commercial rate (1×) or any fraction or multiple thereof, such as twice the recommended commercial rate (2×). Herbicide rates may be expressed as acid equivalent per pound per acre (lb ae/acre) or acid equivalent per gram per hectare (g ae/ha) or as pounds active ingredient per acre (lb ai/acre) or grams active ingredient per hectare (g ai/ha), depending on the herbicide and the formulation. The herbicide application comprises at least one PPO inhibiting herbicide. The plant growth area may or may not comprise weed plants at the time of herbicide application. A herbicidally-effective dose of PPO inhibiting herbicide(s) for use in an area for controlling weeds may consist of a range from about 0.1× to about 30× label rate(s) over a growing season. One (1) hectare is equivalent to 2.47105 acres and one (1) pound is equivalent to 453.592 grams. Herbicide rates can be converted between English and metric as: (lb ai/ac) multiplied by 1.12=(kg ai/ha) and (kg ai/ha) multiplied by 0.89=(lb ai/ac).

The desired application rate of the compounds of the general formula (I) and/or their salts is generally impacted to a certain extent by external conditions such as temperature, humidity, etc. The application rate may therefore vary within wide limits, and can be determined empirically by one of skill in the art in view of the present disclosure. For the application as a herbicide for controlling weeds or other undesirable plants, the total amount of compounds of the general formula (I) and their salts is often desirably in the range from about 0.02 g a.i./ha to about 750 g a.i./ha, about 0.05 g a.i./ha to about 400 g a.i./ha, or about 0.25 g a.i./ha to about 300 g a.i./ha, but preferably from about 0.02 g a.i./ha to about 250 g a.i./ha, especially from about 0.05 g a.i./ha to about 150 g a.i./ha, and most preferably from about 0.25 g a.i./ha to about 120 g a.i./ha. This applies both to the pre-emergence or the post-emergence application.

Herbicide applications may be sequentially or tank mixed with one, two, or a combination of several PPO inhibiting herbicides or any other compatible herbicide. Multiple applications of one herbicide or of two or more herbicides, in combination or alone, may be used over a growing season to areas comprising transgenic plants of the disclosure for the control of a broad spectrum of dicot weeds, monocot weeds, or both, for example, two applications (such as a pre-planting application and a post-emergence application or a pre-emergence application and a post-emergence application) or three applications (such as a pre-planting application, a pre-emergence application, and a post-emergence application or a pre-emergence application and two post-emergence applications).

Substituted pyridyl uracils to be used according to the disclosure and its salts, have excellent herbicidal efficacy against a broad spectrum of economically important monocotyledonous and dicotyledonous annual harmful plants. The present disclosure therefore provides methods for controlling weeds, in areas of transgenic crop plants being tolerant to PPO inhibitor herbicides wherein the plants comprises a recombinant DNA molecule comprising a DNA sequence encoding a heterologous HemG protein, wherein the protein confers tolerance to such herbicides, comprising the application of the compounds of the general formula (I) and/or salts as defined above, to the plants (for example harmful plants such as monocotyledonous or dicotyledonous weeds or undesired crop plants), to the seed (for example grains, seeds or vegetative propagules such as tubers or shoot parts with buds) or to the area on which the plants grow (for example the area under cultivation), including any possible combinations thereof. Specific examples may be mentioned of some representatives of the monocotyledonous and dicotyledonous weed flora which can be controlled by the compounds and methods as described herein, without the enumeration being restricted to certain species. Among the monocotyledonous weed species, for example, Aegilops, Agropyron, Agrostis, Alopecurus, Apera, Avena, Brachicaria, Bromus, Cynodon, Dactyloctenium, Digitaria, Echinochloa, Eleocharis, Eleusine, Eragrostis, Eriochloa, Festuca, Fimbristylis, Imperata, Ischaemum, Heteranthera, Imperata, Leptochloa, Lolium, Monochoria, Panicum, Paspalum, Phalaris, Phleum, Poa, Rottboellia, Sagittaria, Scirpus, Setaria, Sorghum, Sphenoclea, and Cyperus species are covered by the annual group. In the case of dicotyledonous weed species, the spectrum of action extends to species such as, for example, Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erodium, Erysimum, Euphorbia, Galeopsis, Galinsoga, Galium, Geranium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, and Xanthium.

Although the compounds described herein display outstanding herbicidal activity against monocotyledonous and dicotyledonous weeds, many economically important crop plants, depending on the structure of the respective active ingredients and the application rate thereof, are damaged only insignificantly, if at all. Economically important crops here are, for example, dicotyledonous crops from the genera of Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, and Vicia, or monocotyledonous crops from the genera of Allium, Ananas, Asparagus, Avena, Hordeum, Oryza, Panicum, Saccharum, Secale, Sorghum, Triticale, Triticum, and Zea.

The compounds of the general formula (I) and/or salts thereof can be formulated in various ways according to which biological and/or physicochemical parameters are required. Examples of general formulation options are: wettable powders (WP), water-soluble powders (SP), emulsifiable concentrates (EC), water-soluble concentrates, aqueous solutions (SL), emulsions (EW), such as oil-in-water and water-in-oil emulsions, sprayable solutions or emulsions, dispersions based on oil or water, oil dispersions (OD), suspoemulsions (SE), suspension concentrates (SC), oil-miscible solutions, capsule suspensions (CS), dusting products (DP), dressings, granules for soil application or scattering, granules (GR) in the form of microgranules, spray granules, absorption and adsorption granules, water-dispersible granules (WG), water-soluble granules (SG), ULV formulations, microcapsules, or waxes.

The individual formulation types are known in principle and are described in, for example, Winnacker-Küchler, “Chemische Technologie,” Vol. 7, 4^{th} ed., Carl Hanser Verlag, Munich, 1986; Van Valkenburg, “Pesticide Formulations,” Marcel Dekker Inc., New York, NY, 1973; and Masters, “Spray Drying Handbook,” 3^{rd} ed., George Goodwin Ltd. London, 1979. The necessary formulation auxiliaries such as inert materials, surfactants, solvents and further additives are likewise known and are described in, for example, Watkins, “Handbook of Insecticide Dust Diluents and Carriers,” 2^{nd} ed., Dorland Books, Caldwell, NJ, 1955; Van Olphen, “An Introduction to Clay Colloid Chemistry,” 2^{nd} ed., J. Wiley & Sons, New York, NY, 1974; Marsden, “Solvents Guide,” 2^{nd} ed., Interscience Publishers Inc., New York, NY, 1963; McCutcheon's “Detergents and Emulsifiers Annual,” MC Publishing Corp., Ridgewood, NJ, 1998; Sisley and Wood, “Encyclopedia of Surface-Active Agents,” Chemical Publishing Company, New York, NY, 1964; Schönfeldt, “Grenzflächenaktive Äthylenoxid-Addukte,” Wissenschaftliche Verlagsgesellschaft mbH, Stuttgart, 1976; and Winnacker-Küchler, “Chemische Technologie,” Vol. 7, 4^{th} ed., Carl Hanser Verlag, Munich, 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzene sulfonates, sodium lignosulfonate, sodium 2,2′-dinaphthylmethane-6,6′-disulfonate, sodium dibutylnaphthalenesulfonate, or sodium oleoyl methyltaurate. To produce the wettable powders, the active herbicidal ingredients are finely ground, for example, in customary apparatuses such as hammer mills, blower mills, and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkyl aryl sulfonate salts, such as calcium dodecylbenzenesulfonate, or nonionic emulsifiers such as fatty acid polyglycol esters, alkyl aryl polyglycol ethers, fatty alcohol polyglycol ethers, propylene oxide-ethylene oxide condensation products, alkyl polyethers, sorbitan esters, for example sorbitan fatty acid esters, or for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Active compounds that can be employed in combination with the compounds of the general formula (I) described herein in compositions described herein (for example in mixed formulations or in the tank mix) are, for example, known active compounds which are based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, or protoporphyrinogen oxidase, as are described in, for example, “Glossary of Common Names and Abbreviations of Herbicides,” Weed Research 26:441-445, 1986 or MacBean, “The Pesticide Manual,” 19^{th} ed., British Crop Protection Council, Alton, U K, 2021, and the literature cited therein. Known herbicides or plant growth regulators which can be combined with the compounds of the described herein are, for example, the following, where said active compounds are designated either with their “common name” in accordance with the International Organization for Standardization (ISO) or with the chemical name or with the code number. They always encompass all the use forms, for example acids, salts, esters and also all isomeric forms such as stereoisomers and optical isomers, even if they are not mentioned explicitly.

Examples of such herbicidal mixing partners include one or more of the following: Acetochlor, Acifluorfen, Acifluorfen-methyl, Acifluorfen-Sodium, Aclonifen, Alachlor, Allidochlor, Alloxydim, Alloxydim-Sodium, Ametryn, Amicarbazone, Amidochlor, Amidosulfuron, 4-Amino-3-chloro-6-(4-chloro-2-fluoro-3-methylphenyl)-5-fluoropyridin-2-carboxylic acid, Aminocyclopyrachlor, Aminocyclopyrachlor-potassium, Aminocyclopyrachlor-methyl, Aminopyralid, Aminopyralid-dimethylammonium, Aminopyralid-tripromine, Amitrol, Ammoniumsulfamate, Anilofos, Asulam, Asulam-potassium, Asulam-sodium, Atrazin, Azafenidin, Azimsulfuron, Beflubutamide, (S)-(−)-Beflubutamide, Beflubutamide-M, Benazolin, Benazolin-ethyl, Benazolin-dimethylammonium, Benazolin-Potassium, Benfluralin, Benfuresate, Bensulfuron, Bensulfuron-methyl, Bensulid, Bentazone, Bentazone-Sodium, Benzobicyclon, Benzofenap, Bicyclopyrone, Bifenox, Bilanafos, Bilanafos-Sodium, Bipyrazone, Bispyribac, Bispyribac-Natium, Bixlozone, Bromacil, Bromacil-lithium, Bromacil-Sodium, Bromobutid, Bromofenoxim, Bromoxynil, Bromoxynilbutyrate, Bromoxynil-Potassium, Bromoxynil-heptanoate and Bromoxynil-octanoate, Busoxinone, Butachlor, Butafenacil, Butamifos, Butenachlor, Butralin, Butroxydim, Butylate, Cafenstrole, Cambendichlor, Carbetamide, Carfentrazone, Carfentrazone-Ethyl, Chloramben, Chloramben-ammonium, Chloramben-diolamin, Chlroamben-methyl, Chloramben-methylammonium, Chloramben-Sodium, Chlorbromuron, Chlorfenac, Chlorfenac-ammonium, Chlorfenac-Sodium, Chlorfenprop, Chlorfenprop-methyl, Chlorflurenol, Chlorflurenol-methyl, Chloridazon, Chlorimuron, Chlorimuron-ethyl, Chlorophthalim, Chlorotolurone, Chlorsulfuron, Chlorthal, Chlorthal-dimethyl, Chlorthal-monomethyl, Cinflubrolin, (1S,2R,4R)-2-[(2-Bromo-6-fluorophenyl)methoxy]-1-methyl-4-(1-methylethyl)-7-oxabicyclo[2.2.1]heptan, (1R,2S,4S)-2-[(2-Bromo-6-fluorophenyl)methoxy]-1-methyl-4-(1-methylethyl)-7-oxabicyclo[2.2.1]heptane, Cinidon, Cinidon-ethyl, Cinmethylin, exo-(+)-Cinmethylin, i.e. (1R,2S,4S)-4-isopropyl-1-methyl-2-[(2-methylbenzyl)oxy]-7-oxabicyclo[2.2.1]heptan, exo-(−)-Cinmethylin, i.e. (1R,2S,4S)-4-isopropyl-1-methyl-2-[(2-methylbenzyl)oxy]-7-oxabicyclo[2.2.1]heptan, Cinosulfuron, Clacyfos, Clethodim, Clodinafop, Clodinafop-ethyl, Clodinafop-propargyl, Clomazone, Clomeprop, Clopyralid, Clopyralid-methyl, Clopyralid-olamin, Clopyralid-Potassium, Clopyralid-tripomin, Cloransulam, Cloransulam-methyl, Cumylurone, Cyanamide, Cyanazine, Cycloate, Cyclopyranil, Cyclopyrimorat, Cyclosulfamuron, Cycloxydim, Cyhalofop, Cyhalofop-butyl, Cyprazin, 2,4-D (including the Ammonium, Butotyl, Butyl, Choline, Diethylammonium, Dimethylammonium, Diolamin, Doboxyl, Dodecylammonium, Etexyl, Ethyl, 2-Ethylhexyl, Heptylammonium, Isobutyl, Isooctyl, Isopropyl, Isopropylammonium, Lithium, Meptyl, Methyl, Potassium, Sodium, Tetradecylammonium, Triethylammonium, Triisopropanolammonium, Tripromin and Trolamin salts thereof), 2,4-DB, 2,4-DB-butyl, 2,4-DB-Dimethylammonium, 2,4-DB-isooctyl, 2,4-DB-Potassium and 2,4-DB-Sodium, Daimurone (Dymron), Dalapon, Dalapon-Calcium, Dalapon-Magnesium, Dalapon-Natium, Dazomet, Dazomet-Sodium, n-Decanol, 7-Deoxy-D-sedoheptulose, Desmedipham, Detosyl-pyrazolat (DTP), Dicamba and its salts (e.g. Dicamba-biproamine, Dicamba-N,N-Bis(3-aminopropyl)methylamine, Dicamba-butotyl, Dicamba-choline, Dicamba-Diglycolamine, Dicamba-Dimethylammonium, Dicamba-Diethanolaminemmonium, Dicamba-Diethylammonium, Dicamba-isopropylammonium, Dicamba-methyl, Dicamba-monoethanolamine, Dicamba-olamine, Dicamba-Potassium, Dicamba-Sodium, Dicamba-Triethanolamin), Dichlobenil, 2-(2,4-Dichlorobenzyl)-4,4-dimethyl-1,2-oxazolidin-3-one, 2-(2,5-Dichlorobenzyl)-4,4-dimethyl-1,2-oxazolidin-3-one, Dichlorprop, Dichlorprop-butotyl, Dichlorprop-Dimethylammonium, Dichhlorprop-etexyl, Dichlorprop-ethylammonium, Dichlorprop-isoctyl, Dichlorprop-methyl, Dichlorprop-Potassium, Dichlorprop-Sodium, Dichlorprop-P, Dichlorprop-P-Dimethylammonium, Dichlorprop-P-etexyl, Dichlorprop-P-Potassium, Dichlorprop-Sodium, Diclofop, Diclofop-methyl, Diclofop-P, Diclofop-P-methyl, Diclosulam, Difenzoquat, Difenzoquat-metilsulfate, Diflufenican, Diflufenzopyr, Diflufenzopyr-Sodium, Dimefuron, Dimepiperate, Dimesulfazet, Dimethachlor, Dimethametryn, Dimethenamid, Dimethenamid-P, Dimetrasulfuron, Dinitramine, Dinoterb, Dinoterb-Acetate, Diphenamid, Diquat, Diquat-Dibromide, Diquat-Dichloride, Dithiopyr, Diuron, DNOC, DNOC-Ammonium, DNOC-Potassium, DNOC-Sodium, Endothal, Endothal-Diammonium, Endothal-Dipotassium, Endothal-Disodium, Epyrifenacil, EPTC, Esprocarb, Ethalfluralin, Ethametsulfuron, Ethamet-sulfuron-Methyl, Ethiozin, Ethofumesate, Ethoxyfen, Ethoxyfen-Ethyl, Ethoxysulfuron, Etobenzanid, F-5231, d.h. N-[2-Chloro-4-fluoro-5-[4-(3-fluoropropyl)-4,5-dihydro-5-oxo-1H-tetrazol-1-yl]-phenyl]-ethansulfonamide, F-7967, i.e. 3-[7-Chloro-5-fluoro-2-(trifluoromethyl)-1H-benzimidazol-4-yl]-1-methyl-6-(trifluoromethyl)pyrimidin-2,4(1H,3H)-dione, Fenoxaprop, Fenoxaprop-P, Fenoxaprop-Ethyl, Fenoxaprop-P-Ethyl, Fenoxasulfone, Fenpyrazone, Fenquinotrione, Fentrazamide, Feproxydim, Flamprop, Flamprop-Isoproyl, Flamprop-Methyl, Flamprop-M-Isopropyl, Flamprop-M-Methyl, Flazasulfuron, Florasulam, Florpyrauxifen, Florpyrauxifen-benzyl, Fluazifop, Fluazifop-Butyl, Fluazifop-Methyl, Fluazifop-P, Fluazifop-P-Butyl, Flucarbazone, Flucarbazone-Sodium, Flucetosulfuron, Fluchloralin, Fluchloraminopyr, Fluchloraminopyr-tefuryl, Flufenacet, Flufenpyr, Flufenpyr-Ethyl, Flumetsulam, Flumiclorac, Flumiclorac-Pentyl, Flumioxazin, Fluometuron, Flurenol, Flurenol-Butyl, -Dimethylammonium and -Methyl, Fluoroglycofen, Fluoroglycofen-Ethyl, Flupropanat, Flupropanat-Sodium, Flupyrsulfuron, Flupyrsulfuron-Methyl, Flupyrsulfuron-Methyl-Sodium, Fluridon, Flurochloridon, Fluroxypyr, Fluroxypyr-Butometyl, Fluroxypyr-Meptyl, Flurtamon, Fluthiacet, Fluthiacet-Methyl, Fomesafen, Fomesafen-Sodium, Foramsulfuron, Foramsulfuron-Sodium, Fosamine, Fosamine-Ammonium, Glufosinate, Glufosinate-Ammonium, Glufosinate-Sodium, L-Glufosinate-Ammonium, L-Glufosinate-Sodium, Glufosinate-P-Sodium, Glufosinate-P-Ammonium, Glyphosate, Glyphosate-Ammonium, Glyphosate-Isopropylammonium, Glyphosate-Diammonium, Glyphosate-Dimethylammonium, Glyphosate-Potassium, Glyphosate-Sodium, Glyphosate-Sesquisodium and Glyphosate-Trimesium, H-9201, i.e. O-(2,4-Dimethyl-6-nitrophenyl)-O-ethyl-isopropylphosphoramidothioate, Halauxifen, Halauxifen-methyl, Halosafen, Halosulfuron, Halosulfuron-Methyl, Haloxyfop, Haloxyfop-P, Haloxyfop-Ethoxyethyl, Haloxyfop-P-Ethoxyethyl, Haloxyfop-Methyl, Haloxyfop-P-Methyl, Haloxifop-Sodium, Hexazinon, HNPC-A8169, i.e. Prop-2-yn-1-yl (2S)-2-{3-[(5-tert-butylpyridin-2-yl)oxy]phenoxy}propanoate, HW-02, i.e. 1-(Dimethoxyphosphoryl)-ethyl-(2,4-dichlorophenoxy)acetate, Hydantocidine, Icafolin, Icafolin-Methyl, Imazamethabenz, Imazamethabenz-Methyl, Imazamox, Imazamox-Ammonium, Imazapic, Imazapic-Ammonium, Imazapyr, Imazapyr-Isopropylammonium, Imazaquin, Imazaquin-Ammonium, Imazaquin-Methyl, Imazethapyr, Imazethapyr-Ammonium, Imazosulfuron, Indanofan, Indaziflam, Indolauxipyr, Iodosulfuron, Iodosulfuron-Methyl, Iodosulfuron-Methyl-Sodium, Ioxynil, Ioxynil-Lithium, -Octanoate, -Potassium and Sodium, Ipfencarbazone, Iptriazopyrid, i.e. 3-[(Isopropylsulfonyl)methyl]-N-(5-methyl-1,3,4-oxadiazol-2-yl)-5-(trifluoromethyl)[1,2,4]triazolo-[4,3-a]pyridin-8-carboxamide, Isoproturon, Isouron, Isoxaben, Isoxaflutole, Karbutilate, KUH-043, i.e. 3-({[5-(Difluoromethyl)-1-methyl-3-(trifluoromethyl)-1H-pyrazol-4-yl]methyl}sulfonyl)-5,5-dimethyl-4,5-dihydro-1,2-oxazole, Ketospiradox, Ketospiradox-Potassium, Lactofen, Lenacil, Linuron, MCPA, MCPA-Butotyl, -Butyl, -Dimethyl-ammonium, -Diolamin, -2-Ethylhexyl, -Ethyl, -Isobutyl, Isoctyl, -Isopropyl, -Isopropylammonium, -Methyl, Olamin, -Potassium, -Sodium and -Trolamin, MCPB, MCPB-Methyl, -Ethyl and -Sodium, Mecoprop, Mecoprop-Butotyl, Mecoprop-dimethylammonium, Mecoprop-Diolamin, Mecoprop-Etexyl, Mecoprop-Ethadyl, Mecoprop-Isoctyl, Mecoprop-Methyl, Mecoprop-Potassium, Mecoprop-Sodium, and Mecoprop-Trolamin, Mecoprop-P, Mecoprop-P-Butotyl, -Dimethylammonium, -2-Ethylhexyl and -Potassium, Mefenacet, Mefluidid, Mefluidid-Diolamine, Mefluidid-Potassium, Mesosulfuron, Mesosulfuron-Methyl, Mesosulfuron-Sodium, Mesotrion, Methabenzthiazuron, Metam, Metamifop, Metamitron, Metazachlor, Metazo-sulfuron, Methabenzthiazuron, Methiopyrsulfuron, Methiozoline, Methyl isothiocyanate, Meto-bromuron, Metolachlor, S-Metolachlor, Metosulam, Metoxuron, Metproxybicyclone, Metribuzine, Metsulfuron, Metsulfuron-Methyl, Molinat, Monolinuron, Monosulfuron, Monosulfuron-Methyl, MT-5950, i.e. N-[3-Chloro-4-(1-methylethyl)-phenyl]-2-methylpentanamide, NGGC-011, Napropamid, NC-310, i.e. 4-(2,4-Dichlorobenzoyl)-1-methyl-5-benzyloxypyrazole, Neburone, Nicosulfuron, Nonanoic acid (Pelargonic acid), Norflurazone, Orbencarb, Orthosulfamuron, Oryzalin, Oxadiargyl, Oxadiazone, Oxasulfuron, Oxaziclomefone, Oxyfluorfen, Paraquat, Paraquat-dichloride, Paraquat-Dimethylsulfate, Pebulat, Pendimethaline, Penoxsulam, Pentachlorphenol, Pentoxazone, Pethoxamid, Petroleum oil, Phenmedipham, Phenmedipham-Ethyl, Picloram, Picloram-dimethylammonium, Picloram-Etexyl, Picloram-Isoctyl, Picloram-Methyl, Picloram-Olamin, Picloram-Potassium, Picloram-Triethylammonium, Picloram-Tripromin, Picloram-Trolamin, Picolinafen, Pinoxaden, Piperophos, Pretilachlor, Primisulfuron, Primisulfuron-Methyl, Prodiamine, Profoxydim, Prometon, Prometryn, Propachlor, Propanil, Propaquizafop, Propazine, Propham, Propisochlor, Propoxycarbazone, Propoxycarbazone-Sodium, Propyrisulfuron, Propyzamid, Prosulfocarb, Prosulfuron, Pyraclonil, Pyraflufen, Pyraflufen-Ethyl, Pyraquinate, Pyrasulfotol, Pyrazolynat (Pyrazolat), Pyrazosulfuron, Pyrazosulfuron-Ethyl, Pyrazoxyfen, Pyribambenz, Pyribambenz-Isopropyl, Pyribambenz-Propyl, Pyribenzoxim, Pyributicarb, Pyridafol, Pyridat, Pyriftalid, Pyriminobac, Pyriminobac-Methyl, Pyrimisulfan, Pyrithiobac, Pyrithiobac-Sodium, Pyroxasulfon, Pyroxsulam, Quinclorac, Quinclorac-Dimethylammonium, Quinclorac-Methyl, Quinmerac, Quinoclamin, Quizalofop, Quizalofop-Ethyl, Quizalofop-P, Quizalofop-P-Ethyl, Quizalofop-P-Tefuryl, QYM201, i.e. 1-{2-Chloro-3-[(3-cyclopropyl-5-hydroxy-1-methyl-1H-pyrazol-4-yl)carbonyl]-6-(trifluoromethyl)phenyl}piperidin-2-on, Rimisoxafen, Rimsulfuron, Saflufenacil, Sethoxydim, Siduron, Simazine, Simetryn, SL-261, Sulcotrione, Sulfentrazone, Sulfometuron, Sulfometuron-Methyl, Sulfosulfuron, SYP-249, i.e. 1-Ethoxy-3-methyl-1-oxobut-3-en-2-yl-5-[2-chloro-4-(trifluoromethyl)phenoxy]-2-nitrobenzoate, SYP-300, i.e. 1-[7-Fluoro-3-oxo-4-(prop-2-yn-1-yl)-3,4-dihydro-2H-1,4-benzoxazin-6-yl]-3-propyl-2-thioxoimidazolidin-4,5-dione, 2,3,6-TBA, TCA (Trichloroacetic acid) and its salts, z.B. TCA-ammonium, TCA-Calcium, TCA-Ethyl, TCA-Magnesium, TCA-Sodium, Tebuthiuron, Tefuryltrione, Tembotrione, Tepraloxydim, Terbacil, Terbucarb, Terbumeton, Terbuthylazine, Terbutryn, Tetflupyrolimet, Thaxtomin, Thenylchlor, Thiazopyr, Thiencarbazone, Thiencarbazone-Methyl, Thifensulfuron, Thifensulfuron-Methyl, Thiobencarb, Tiafenacil, Tolpyralat, Topramezone, Tralkoxydim, Triafamon, Tri-allate, Triasulfuron, Triaziflam, Tribenuron, Tribenuron-Methyl, Triclopyr, Triclopyr-Butotyl, Triclopyr-Choline, Triclopyr-Ethyl, Triclopyr-Triethylammonium, Trietazine, Trifloxysulfuron, Trifloxysulfuron-Sodium, Trifludimoxazin, Trifluralin, Triflusulfuron, Triflusulfuron-Methyl, Tritosulfuron, Harnstoffsulfat, Vernolat, XDE-848, ZJ-0862, i.e. 3,4-Dichloro-N-{2-[(4,6-dimethoxypyrimidin-2-yl)oxy]benzyl}aniline, Methyl 3-(2-Chloro-4-fluoro-5-(3-methyl-2,6-dioxo-4-trifluoromethyl-3,6-dihydropyrimidin-1(2H)-yl)phenyl)-5-methyl-4,5-dihydroisoxazol-5-carboxylate, Ethyl 3-(2-Chloro-4-fluoro-5-(3-methyl-2,6-dioxo-4-trifluoromethyl-3,6-dihydropyrimidin-1(2H)-yl)phenyl)-5-methyl-4,5-dihydroisoxazol-5-carboxylate, 3-(2-Chloro-4-fluoro-5-(3-methyl-2,6-dioxo-4-trifluoromethyl-3,6-dihydropyrimidin-1(2H)-yl)phenyl)-5-methyl-4,5-dihydroisoxazol-5-carboxylic acid, Ethyl-1[(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 3-Chloro-2-[3-(difluoromethyl)isoxazolyl-5-yl]phenyl-5-chloropyrimidin-2-ylether, 2-(3,4-Dimethoxyphenyl)-4-[(2-hydroxy-6-oxocyclohex-1-en-1-yl)carbonyl]-6-methylpyridazin-3(2H)-one, 2-({2-[(2-Methoxyethoxy)methyl]-6-methylpyridin-3-yl}carbonyl)cyclohexane-1,3-dione, (5-Hydroxy-1-methyl-1H-pyrazol-4-yl)(3,3,4-trimethyl-1,1-dioxido-2,3-dihydro-1-benzothiophen-5-yl)methanone, 1-Methyl-4-[(3,3,4-trimethyl-1,1-dioxido-2,3-dihydro-1-benzothiophen-5-yl)carbonyl]-1H-pyrazol-5-yl propan-1-sulfonate, 4-{2-Chloro-3-[(3,5-dimethyl-1H-pyrazol-1-yl)methyl]-4-(methylsulfonyl)benzoyl}-1-methyl-1H-pyrazol-5-yl-1,3-dimethyl-1H-pyrazol-4-carboxylate; Cyanomethyl-4-amino-3-chloro-5-fluoro-6-(7-fluoro-1H-indol-6-yl)pyridin-2-carboxylate, Prop-2-yn-1-yl 4-amino-3-chloro-5-fluoro-6-(7-fluoro-1H-indol-6-yl)pyridin-2-carboxylate, Methyl-4-amino-3-chloro-5-fluoro-6-(7-fluoro-1H-indol-6-yl)pyridin-2-carboxylate, Benzyl-4-amino-3-chloro-5-fluoro-6-(7-fluoro-1H-indol-6-yl)pyridin-2-carboxylate, Ethyl-4-amino-3-chloro-5-fluoro-6-(7-fluoro-1H-indol-6-yl)pyridin-2-carboxylate, Methyl-4-amino-3-chloro-5-fluoro-6-(7-fluoro-1-isobutyryl-1H-indol-6-yl)pyridin-2-carboxylate, Methyl 6-(1-acetyl-7-fluoro-1H-indol-6-yl)-4-amino-3-chloro-5-fluoropyridin-2-carboxylate, Methyl-4-amino-3-chloro-6-[1-(2,2-dimethylpropanoyl)-7-fluoro-1H-indol-6-yl]-5-fluoropyridin-2-carboxylate, Methyl-4-amino-3-chloro-5-fluoro-6-[7-fluoro-1-(methoxyacetyl)-1H-indol-6-yl]pyridin-2-carboxylate, Potassium 4-amino-3-chloro-5-fluoro-6-(7-fluoro-1H-indol-6-yl)pyridin-2-carboxylate, Sodium-4-amino-3-chloro-5-fluoro-6-(7-fluoro-1H-indol-6-yl)pyridin-2-carboxylate, Butyl-4-amino-3-chloro-5-fluoro-6-(7-fluoro-1H-indol-6-yl)pyridin-2-carboxylate, 4-Hydroxy-1-methyl-3-[4-(trifluoromethyl)pyridin-2-yl]imidazolidin-2-one, 3-(5-tert-butyl-1,2-oxazol-3-yl)-4-hydroxy-1-methylimidazolidin-2-one, 3-[5-Chloro-4-(trifluoromethyl)pyridin-2-yl]-4-hydroxy-1-methylimidazolidin-2-one, 4-Hydroxy-1-methoxy-5-methyl-3-[4-(trifluoromethyl)pyridin-2-yl]imidazolidin-2-one, 6-[(2-Hydroxy-6-oxocyclohex-1-en-1-yl)carbonyl]-1,5-dimethyl-3-(2-methylphenyl)quinazolin-2,4(1H,3H)-dione, 3-(2,6-Dimethylphenyl)-6-[(2-hydroxy-6-oxocyclohex-1-en-1-yl)carbonyl]-1-methylquinazolin-2,4(1H,3H)-dione, 2-[2-chloro-4-(methylsulfonyl)-3-(morpholin-4-ylmethyl)benzoyl]-3-hydroxycyclohex-2-en-1-one, 1-(2-carboxyethyl)-4-(pyrimidin-2-yl)pyridazin-1-ium salt (combined with suitable anions such as chloride, acetate or trifluoroacetate), 1-(2-Carboxyethyl)-4-(pyridazin-3-yl)pyridazin-1-ium salt (combined with suitable anions such as chloride, acetate or trifluoroacetate), 4-(Pyrimidin-2-yl)-1-(2-sulfoethyl)pyridazin-1-ium salt (combined with suitable anions such as chloride, acetate or trifluoroacetate), 4-(Pyridazin-3-yl)-1-(2-sulfoethyl)pyridazin-1-ium salt (combined with suitable anions such as chloride, acetate or trifluoroacetate), 1-(2-Carboxyethyl)-4-(1,3-thiazol-2-yl)pyridazin-1-ium salt (combined with suitable anions such as chloride, acetate or trifluoroacetate), 1-(2-Carboxyethyl)-4-(1,3,4-thiadiazol-2-yl)pyridazin-1-ium salt (combined with suitable anions such as chloride, acetate or trifluoroacetate), Methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methyliden)amino]oxy}propanoate, Methyl (2S)-2-{[(E)-({2-chloro-4-fluoro-5-[3-methyl-2,6-dioxo-4-(trifluoromethyl)-3,6-dihydropyrimidin-1(2H)-yl]phenyl}methyliden)amino]oxy}propanoate, Methyl (2R/S)-2-{[(E)-({2-chloro-4-fluoro-5-[3-methyl-2,6-dioxo-4-(trifluoromethyl)-3,6-dihydropyrimidin-1(2H)-yl]phenyl}methyliden)amino]oxy}propanoate, (E)-2-(Trifluoromethyl)benzaldehyde-O-{2,6-bis[(4,6-dimethoxypyrimidin-2-yl)oxy]benzoyl}oxim, 2-Fluoro-N-(5-methyl-1,3,4-oxadiazol-2-yl)-3-[(R)-propylsulfinyl]-4-(trifluoromethyl)benzamide, (2R)-2-[(4-Amino-3,5-dichloro-6-fluoro-2-pyridyl)oxy]propane carboxylic acid, 2-Ethoxy-2-oxoethyl-1-{2-chloro-4-fluoro-5-[3-methyl-2,6-dioxo-4-(trifluoromethyl)-3,6-dihydropyrimidin-1(2H)-yl]phenoxy}cyclopropancarboxylate, 2-Methoxy-2-oxoethyl-1-{2-chloro-4-fluoro-5-[3-methyl-2,6-dioxo-4-(trifluoromethyl)-3,6-dihydropyrimidin-1(2H)-yl]phenoxy}cyclopropancarboxylate, {[(1-{2-Chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, 2-(2-Bromo-4-chlorobenzyl)-4,4-dimethyl-1,2-oxazolidin-3-one, Methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazol-6a-carboxylate, Ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazol-6a-carboxylate, Methyl-3-{2-chloro-4-fluoro-5-[3-methyl-2,6-dioxo-4-(trifluoromethyl)-3,6-dihydropyrimidin-1(2H)-yl]phenyl}-6-methyl-3a,4,5,6-tetrahydro-6aH-cyclopenta[d][1,2]oxazol-6a-carboxylate, 3-{2-Chloro-4-fluoro-5-[3-methyl-2,6-dioxo-4-(trifluoromethyl)-3,6-dihydropyrimidin-1(2H)-yl]phenyl}-6-methyl-3a,4,5,6-tetrahydro-6aH-cyclopenta[d][1,2]oxazol-6a-carboxylic acid, 3-{2-Chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazol-6a-carboxylic acid.

Examples of plant growth regulators as possible mixing partners are: Abscisic acid and related analogs [e.g. (2Z,4E)-5-[6-Ethynyl-1-hydroxy-2,6-dimethyl-4-oxocyclohex-2-en-1-yl]-3-methylpenta-2,4-dienoic acid, methyl-(2Z,4E)-5-[6-ethynyl-1-hydroxy-2,6-dimethyl-4-oxocyclohex-2-en-1-yl]-3-methylpenta-2,4-dienoate, (2Z,4E)-3-ethyl-5-(1-hydroxy-2,6,6-trimethyl-4-oxocyclohex-2-en-1-yl)penta-2,4-dienoic acid, (2E,4E)-5-(1-hydroxy-2,6,6-trimethyl-4-oxocyclohex-2-en-1-yl)-3-(trifluoromethyl)penta-2,4-dienoic acid, methyl (2E,4E)-5-(1-hydroxy-2,6,6-trimethyl-4-oxocyclohex-2-en-1-yl)-3-(trifluoromethyl)penta-2,4-dienoate, (2Z,4E)-5-(2-hydroxy-1,3-dimethyl-5-oxobicyclo[4.1.0]hept-3-en-2-yl)-3-methylpenta-2,4-dienoic acid], acibenzolar, acibenzolar-S-methyl, S-adenosylhomocysteine, allantoin, 2-Aminoethoxyvinylglycine (AVG), aminooxyacetic acid and related esters [e.g. (Isopropylidene)-aminooxyacetic acid-2-(methoxy)-2-oxoethylester, (Isopropylidene)-aminooxyacetic acid-2-(hexyloxy)-2-oxoethylester, (Cyclohexylidene)-aminooxyacetic acid-2-(isopropyloxy)-2-oxoethylester], 1-aminocycloprop-1-ylcarboxylic acid, N-Methyl-1-aminocyclopropyl-1-carboxylic acid, 1-aminocyclopropyl-1-carboxamide, 1-aminocycloprop-1-yl carboxylic acid and derivatives thereof, e.g. disclosed in DE3335514, EP30287, DE2906507 or U.S. Pat. No. 5,123,951, 1-aminocyclopropyl-1-hydroxamic acid, 5-aminolevulinic acid, ancymidol, 6-benzylaminopurine, bikinin, brassinolide, brassinolide-ethyl, L-canaline, catechin and catechines (e.g. (2S,3R)-2-(3,4-Dihydroxyphenyl)-3,4-dihydro-2H-chromen-3,5,7-triol), chitooligosaccharides (CO; COs differ from LCOs in that they lack the pendant fatty acid chain that is characteristic of LCOs. COs, sometimes referred to as N-acetylchitooligosaccharides, are also composed of GlcNAc residues but have side chain decorations that make them different from chitin molecules [(C₈H₁₃NO₅)ₙ, CAS No. 1398-61-4] and chitosan molecules [(C₅H₁₁NO₄)ₙ, CAS No. 9012-76-4]), chitinous compounds, chlormequat chloride, cloprop, cyclanilide, 3-(Cycloprop-1-enyl)propionic acid, 1-[2-(4-cyano-3,5-dicyclopropylphenyl)acetamido]cyclohexanecarboxylic acid, 1-[2-(4-cyano-3-cyclopropylphenyl)acetamido]cyclohexanecarboxylic acid, daminozide, dazomet, dazomet-sodium, n-decanol, dikegulac, dikegulac-sodium, endothal, endothal-dipotassium, -disodium, and mono(N,N-dimethylalkylammonium), ethephon, flumetralin, flurenol, flurenol-butyl, flurenol-methyl, flurprimidol, forchlorfenuron, gibberellic acid, inabenfide, indol-3-acetic acid (IAA), 4-indol-3-ylbutyric acid, isoprothiolane, probenazole, jasmonic acid, Jasmonic acid or derivatives thereof (e.g. jasmonic acid methyl ester, jasmonic acid ethyl ester), lipo-chitooligosaccharides (LCO, sometimes referred to as symbiotic nodulation (Nod) signals (or Nod factors) or as Myc factors, consist of an oligosaccharide backbone of β-1,4-linked N-acetyl-D-glucosamine (“GlcNAc”) residues with an N-linked fatty acyl chain condensed at the non-reducing end. As understood in the art, LCOs differ in the number of GlcNAc residues in the backbone, in the length and degree of saturation of the fatty acyl chain and in the substitutions of reducing and non-reducing sugar residues), linoleic acid or derivatives thereof, linolenic acid or derivatives thereof, maleic hydrazide, mepiquat chloride, mepiquat pentaborate, 1-methylcyclopropene, 3-methylcyclopropene, 1-ethylcyclopropene, 1-n-propylcyclopropene, 1-cyclopropenylmethanol, methoxyvinylglycin (MVG), 3′-methyl abscisic acid, 1-(4-methylphenyl)-N-(2-oxo-1-propyl-1,2,3,4-tetrahydroquinolin-6-yl)methanesulfonamide and related substituted tetrahydroquinolin-6-yl)methanesulfonamides, (3E,3αR,8βS)-3-({[(2R)-4-Methyl-5-oxo-2,5-dihydrofuran-2-yl]oxy}methylen)-3,3α,4,8β-tetrahydro-2H-indeno[1,2-b]furan-2-one and related lactones as outlined in EP2248421, 2-(1-naphthyl)acetamide, 1-naphthylacetic acid, 2-naphthyloxyacetic acid, nitrophenolate-mixture, 4-Oxo-4 [(2-phenylethyl)amino]butyric acid, paclobutrazol, 4-phenylbutyric acid and its related salts (e.g. sodium-4-phenylbutanoate, potassium-4-phenylbutanoate), phenylalanine, N-phenylphthalamic acid, prohexadione, prohexadione-calcium, putrescine, prohydrojasmon, rhizobitoxin, salicylic acid, salicylic acid methyl ester, sarcosine, sodium cycloprop-1-en-1-yl acetate, sodium cycloprop-2-en-1-yl acetate, sodium-3-(cycloprop-2-en-1-yl)propanoate, sodium-3-(cycloprop-1-en-1-yl) propanoate, sidefungin, spermidine, spermine, strigolactone, tecnazene, thidiazuron, triacontanol, trinexapac, trinexapac-ethyl, tryptophan, tsitodef, uniconazole, uniconazole-P, 2-fluoro-N-(3-methoxyphenyl)-9H-purin-6-amine, 2-chloro-N-(3-methoxyphenyl)-9H-purin-6-amine.

Active compounds which can be employed in combination with the compounds of the general formula (I) according to the present disclosure in compositions according to the present disclosure (for example in mixed formulations or in the tank mix) are, for example, the following safeners:
S1) Compounds from the group of heterocyclic carboxylic Acid Derivatives (Formula S1)

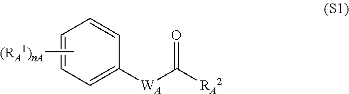

wherein symbols and indices are defined as follows:
n_{A} is an integer value in the range of 0 to 5, preferably 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic moiety selected from the group of partially unsaturated or aromatic five-membered heterocycles carrying 1 to 3 hetero ring atoms selected from the group of nitrogen (N) und oxygen (O), and carrying at least one N-atom and not more than one O-atom in the ring, preferably a five-membered heterocyclic moiety selected from the group (W_{A}¹) to (W_{A}⁴),

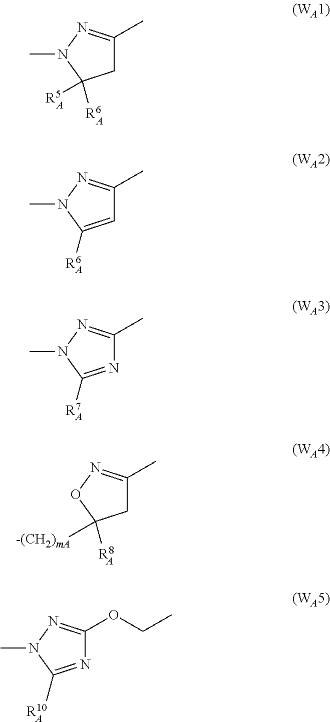

m_{A} is 0 or 1;
R_{A}² is OR_{A}³, SR_{A}³ or NR_{A}³R_{A}⁴ or a saturated or unsaturated 3- to 7-membered heterocycle containing at least one N-atom and up to 3 heteroatoms, preferably combined with other heteroatoms from the group of O (oxygen) and S (sulfur), and which is linked to the carbonyl group in (S1) via a nitrogen atom, and which is unsubstituted or substituted by moieties selected from the group of (C₁-C₄)-alkyl, (C₁-C₄)-alkoxy or possibly substituted phenyl, preferably OR_{A}³, NHR_{A}⁴ or N(CH₃)₂, particularly OR_{A}³
R_{A}³ is hydrogen or an unsubstituted or substituted aliphatic hydrocarbon moiety, preferably containing 1 to 18 C-atoms;
R_{A}⁴ is hydrogen, (C₁-C₆)-alkyl, (C₁-C₆)-alkoxy or substituted or unsubstituted phenyl;
R_{A}⁵ is hydrogen, (C₁-C₈)-alkyl, (C₁-C₈)-haloalkyl, (C₁-C₄)-alkoxy-(C₁-C₈)-alkyl, cyano or COOR_{A}⁹, wherein R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tris-(C₁-C₄)-alkylsilyl;
R_{A}⁶, R_{A}⁷, R_{A}⁸ are independently hydrogen, (C₁-C₈)-alkyl, (C₁-C₈)-haloalkyl, (C₃-C₁₂)-cycloalkyl or substituted or unsubstituted phenyl;
R_{A}¹⁰ is hydrogen, (C₃-C₁₂)-cycloalkyl, substituted or unsubstituted phenyl, or substituted or unsubstituted heteroaryl;
S1^{a}) Compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds, as described in WO-A-91/07874;
S1^{b}) Derivatives of dichlorophenylpyrazolecarboxylic acid (Sib), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 and EP-A-269806;
S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;
S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897.
S1^{f}) compounds of the triazolyloxy acetic acid type (S1^{f}), preferably compounds such as methyl-{[1,5-bis(4-chloro-2-fluorophenyl)-1H-1,2,4-triazol-3-yl]oxy}acetate (S1-14) or {[1,5-Bis(4-chloro-2-fluorophenyl)-1H-1,2,4-triazol-3-yl]oxy}acetic acid (S1-15) or methyl-{[5-(4-chloro-2-fluorophenyl)-1-(2,4-difluorophenyl)-1H-1,2,4-triazol-3-yl]oxy}acetate (S1-16) or {[5-(4-chloro-2-fluorphenyl)-1-(2,4-difluorophenyl)-1H-1,2,4-triazol-3-yl]oxy}acetic acid (S1-17) or methyl-{[1-(4-chloro-2-fluorophenyl)-5-(2,4-difluorophenyl)-1H-1,2,4-triazol-3-yl]oxy}acetate (S1-18) or {[1-(4-chloro-2-fluorophenyl)-5-(2,4-difluorophenyl)-1H-1,2,4-triazol-3-yl]oxy}acetic acid (S1-19), as described in patent application WO2021105101.
S2) Compounds from the group of the 8-quinolinoxy derivatives (S2):
S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;
S2^{b}) Compounds of the (5-chloro-8-quinolinoxy)malonic acid type (S2b), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active ingredients of the dichloroacetamide type (S3), which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “diclonon” (dicyclonon) or “BAS145138” or “LAB145138” (S3-9)
   ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acyl sulfonamides (S4):
S4^{a})N-acylsulfonamides of the formula (S4^{a}) and salts thereof, as described in WO-A-97/45016,

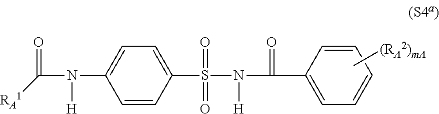

in which
R_{A}¹ is (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² is halogen, (C₁-C₄)-alkyl, (C₁-C₄)-alkoxy, CF₃;
m_{A} is 1 or 2;
v_{A} is 0, 1, 2 or 3;
S4b) Compounds of the 4-(benzoylsulfamoyl)benzamide type of the formula (S4b) and salts thereof, as described in WO-A-99/16744,

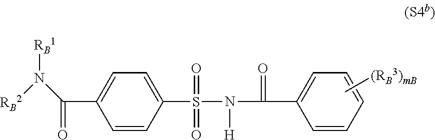

in which
R_{B}¹, R_{B}² are independently hydrogen, (C₁-C₆)-alkyl, (C₃-C₆)-cycloalkyl, (C₃-C₆)-alkenyl, (C₃-C₆)-alkynyl;
R_{B}³ is halogen, (C₁-C₄)-alkyl, (C₁-C₄)-haloalkyl or (C₁-C₄)-alkoxy; and
m_{B} is 1 or 2,
e.g. those in which
R_{B}¹=cyclopropyl, R_{B}²=hydrogen, and (R_{B}³)=2-OMe (“cyprosulfamide,” S4-1),
R_{B}¹=cyclopropyl, R_{B}²=hydrogen, and (R_{B}³)=5-Cl-2-OMe (S4-2),
R_{B}¹=ethyl, R_{B}²=hydrogen, and (R_{B}³)=2-OMe (S4-3),
R_{B}¹=isopropyl, R_{B}²=hydrogen, and (R_{B}³)=5-Cl-2-OMe (S4-4) and
R_{B}=isopropyl, R_{B}²=hydrogen, and (R_{B}³)=2-OMe (S4-5);
S4^{c}) Compounds from the class of the benzoylsulfamoylphenylureas of the formula (S4^{c}), as described in EP-A-365484,

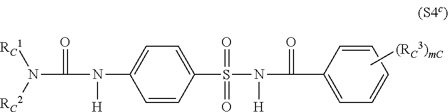

in which
R_{C}¹, R_{C}² are independently hydrogen, (C₁-C₈)-alkyl, (C₃-C₈)-cycloalkyl, (C₃-C₆)-alkenyl, (C₃-C₆)-alkynyl,
R_{C}³ is halogen, (C₁-C₄)-alkyl, (C₁-C₄)-alkoxy, CF₃ and
m_{C} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

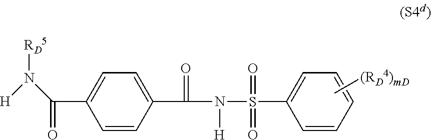

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active ingredients from the class of the hydroxyaromatics and the aromatic-aliphatic carboxylic acid derivatives (S5), for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

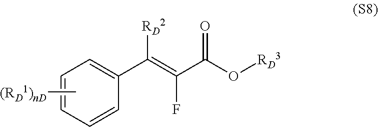

in which the symbols and indices are defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,
R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof,
n_{D} is an integer from 0 to 2.
S9) Active ingredients from the class of the 3-(5-tetrazolylcarbonyl)-2-quinolones (S9), for example
   1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S10^{b})
   as described in WO-A-2007/023719 and WO-A-2007/023764

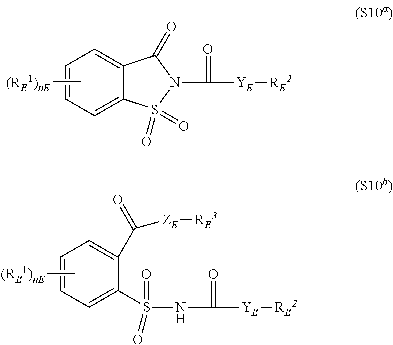

in which
R_{E}¹ is halogen, (C₁-C₄)-alkyl, methoxy, nitro, cyano, CF₃, OCF₃,
Y_{E}, Z_{E} are independently O or S,
n_{E} is an integer from 0 to 4,
R_{E}² is (C₁-C₁₆)-alkyl, (C₂-C₆)-alkenyl, (C₃-C₆)-cycloalkyl, aryl; benzyl, halobenzyl,
R_{E}³ is hydrogen or (C₁-C₆)-alkyl.
S11) Active ingredients of the oxyimino compounds type (S11), which are known as seed-dressing agents, for example
   “oxabetrinil” ((Z)-1,3-dioxolan-2-ylmethoxyimino(phenyl)acetonitrile) (S11-1), which is known as a seed-dressing safener for millet/sorghum against metolachlor damage, “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for maize against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8)
   (4-carboxy-3,4-dihydro-2H-1-benzopyran-4-acetic acid) (S13-4) from American Cyanamid, which is known as a safener for maize against damage by imidazolinones, “MG 191” (CAS Reg. No. 96420-72-3) (2-dichloromethyl-2-methyl-1,3-dioxolane) (S13-5) from Nitrokemia, which is known as a safener for maize,
   “MG 838” (CAS Reg. No. 133993-74-5)
   (2-propenyl 1-oxa-4-azaspiro[4.5]decane-4-carbodithioate) (S13-6) from Nitrokemia
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active ingredients which, in addition to herbicidal action against harmful plants, also have safener action on crop plants such as rice, for example
   “dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against damage by the herbicide imazosulfuron,
   “cumyluron” or “JC-940” (3-(2-chlorophenylmethyl)-1-(1-methyl-1-phenylethyl)urea, see JP-A-60087254), which is known as a safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

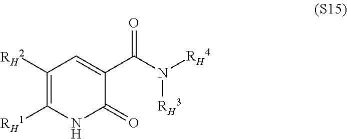

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or
   (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring, and
   where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O, and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active ingredients which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Safeners that may be used in combination with the herbicidal compounds described herein include, but are not limited to, cloquintocet-mexyl, cyprosulfamide, fenchlorazole ethyl ester, isoxadifen-ethyl, mefenpyr-diethyl, fenclorim, cumyluron, S4-1, and S4-5. Preferred safeners include cloquintocet-mexyl, cyprosulfamide, isoxadifen-ethyl, and mefenpyr-diethyl.

The herbicide combinations described herein may comprise further components, for example plant growth regulators or compounds that prevent or eliminate unwanted species. Such compounds include, but are not limited to herbicides, fungicides, insecticides, acaricides, nematicides, miticides, and related substances.

Examples of plant growth regulators that may be used include, but are not limited to, acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4 [(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, and uniconazole-P.

Active compounds that may be used in combination with the compounds of the general formula (I) described herein (in, for example, mixed formulations or a tank mix) are, for example, fungicidally active compounds. The preferred fungicidally active compounds comprise at least one standard commercial active ingredient, and include, but are not limited to:
1) Ergosterol biosynthesis inhibitors, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazole, (1.016) prochloraz, (1.017) propiconazole, (1.018) prothioconazole, (1.019) pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) mefentrifluconazole, (1.082) ipfentrifluconazole.
2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)-N-cyclopropyl-3-(difluoromethyl)-5-fluoro-1-methyl-1H-pyrazole-4-carboxamide, (2.038)N-(5-chloro-2-isopropylbenzyl)-N-cyclopropyl-3-(difluoromethyl)-5-fluoro-1-methyl-1H-pyrazole-4-carboxamide, (2.039)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041)N-[1-(2,4-dichlorophenyl)-1-methoxypropan-2-yl]-3-(difluoromethyl)-1-methyl-1H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-1H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide.
3) Respiratory chain inhibitors acting on complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[l-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate.
4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.
5) Compounds with multisite activity, for example (5.001) Bordeaux mixture, (5.002) captafol, (5.003) captan, (5.004) chlorthalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) zinc metiram, (5.017) copper oxine, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.
6) Compounds capable of triggering host defense, for example (6.001) acibenzolar-S-methyl, (6.002) isotianil, (6.003) probenazole, (6.004) tiadinil.
7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.
8) ATP production inhibitors, for example (8.001) silthiofam.
9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Lipid and membrane synthesis inhibitors, for example (10.001) propamocarb, (10.002) propamocarb hydrochloride, (10.003) tolclofos-methyl.
11) Melanin biosynthesis inhibitors, for example (11.001) tricyclazole, (11.002) 2,2,2-trifluoroethyl-{3-methyl-1-[(4-methylbenzoyl)amino]butan-2-yl}carbamate.
12) Nucleic acid synthesis inhibitors, for example (12.001) benalaxyl, (12.002) benalaxyl-M (kiralaxyl), (12.003) metalaxyl, (12.004) metalaxyl-M (mefenoxam).
13) Signal transduction inhibitors, for example (13.001) fludioxonil, (13.002) iprodione, (13.003) procymidone, (13.004) proquinazid, (13.005) quinoxyfen, (13.006) vinclozolin.
14) Compounds that can act as decouplers, for example (14.001) fluazinam, (14.002) meptyldinocap.
15) Further compounds, for example (15.001) abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenon, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphonic acid and salts thereof, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts thereof, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′-phenyl-N′-(prop-2-yn-1-yl)thiophene 2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1)sulfonyl]-3,4-dihydropyrimidin-2(1H)-one.

Preferred fungicides are selected from the group consisting of benalaxyl, bitertanol, bromuconazole, captafol, carbendazim, carpropamid, cyazofamid, cyproconazole, diethofencarb, edifenphos, fenpropimorph, fentin acetate, fluquinconazole, fosetyl, fluoroimide, folpet, iminoctadine, iprodione, iprovalicarb, kasugamycin, maneb, nabam, pencycuron, prochloraz, propamocarb, propineb, prothioconazole, pyrimethanil, spiroxamine, quintozene, tebuconazole, tolylfluanid, triadimefon, triadimenol, trifloxystrobin, and zineb.

Active compounds which can be employed in combination with the compounds of the general formula (I) according to the present disclosure in compositions described herein (for example in mixed formulations or in the tank mix) are, for example, insecticidal, acaricidal, nematicidal, miticidal and related active ingredients are, for example (the compounds are, if possible, referred to by their common names):
(1) Acetylcholinesterase (AChE) inhibitors, preferably carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazemate, trimethacarb, XMC, and xylylcarb; or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon, and vamidothion.
(2) GABA-gated chloride channel blockers, preferably cyclodiene-organochlorines selected from chlordane and endosulfan, or phenylpyrazoles (fiproles) selected from ethiprole and fipronil.
(3) Sodium channel modulators, preferably pyrethroids selected from acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.
(4) Nicotinic acetylcholine receptor (nAChR) competitive modulators, preferably neonicotinoids selected from acetamiprid, clothianidin, dinotefuran, imidacloprid, nitenpyram, thiacloprid, and thiamethoxam, or nicotine, or sulfoximines such as sulfoxaflor, or butenolides such as flupyradifurone.
(5) Nicotinic acetylcholine receptor (nAChR) allosteric modulators, preferably spinosyns selected from spinetoram and spinosad.
(6) Glutamate-gated chloride channel (GluCl) allosteric modulators, preferably avermectins/milbemycins selected from abamectin, emamectin benzoate, lepimectin, and milbemectin.
(7) Juvenile hormone mimics, preferably juvenile hormone analogues selected from hydroprene, kinoprene and methoprene, or fenoxycarb or pyriproxyfen.
(8) Miscellaneous non-specific (multi-site) inhibitors, preferably alkyl halides selected from methyl bromide and other alkyl halides; or chloropicrin or sulfuryl fluoride or borax or tartar emetic or methyl isocyanate generators selected from diazomet and metam.
(9) Chordotonal organ TRPV channel modulators selected from pymetrozine and pyrifluquinazon.
(10) Mite growth inhibitors selected from clofentezine, hexythiazox, diflovidazin, and etoxazole.
(11) Microbial disruptors of insect midgut membranes selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins selected from Cry1Ab, Cry1Ac, Cry1Fa, Cry1A.105, Cry2Ab, VIP3A, mCry3A, Cry3Ab, Cry3Bb, and Cry34Ab1/35Ab1.
(12) Inhibitors of mitochondrial ATP synthase, preferably ATP disruptors selected from diafenthiuron, or organotin compounds selected from azocyclotin, cyhexatin and fenbutatin oxide, or propargite or tetradifon.
(13) Uncouplers of oxidative phosphorylation via disruption of the proton gradient selected from chlorfenapyr, DNOC, and sulfluramid.
(14) Nicotinic acetylcholine receptor channel blockers selected from bensultap, cartap hydrochloride, thiocyclam, and thiosultap-sodium.
(15) Inhibitors of chitin biosynthesis, type 0, selected from bistrifluron, chlorfluazuron, diflubenzuron, flucycloxuron, flufenoxuron, hexaflumuron, lufenuron, novaluron, noviflumuron, teflubenzuron, and triflumuron.
(16) Inhibitors of chitin biosynthesis, type 1, such as buprofezin.
(17) Molting disruptors (especially in the case of Diptera) such as cyromazine.
(18) Ecdysone receptor agonists selected from chromafenozide, halofenozide, methoxyfenozide, and tebufenozide.
(19) Octopamine receptor agonists such as amitraz.
(20) Mitochondrial complex III electron transport inhibitors selected from hydramethylnon, acequinocyl, and fluacrypyrim.
(21) Mitochondrial complex I electron transport inhibitors, preferably METI acaricides selected from fenazaquin, fenpyroximate, pyrimidifen, pyridaben, tebufenpyrad and tolfenpyrad, or rotenone (Derris).
(22) Voltage-dependent sodium channel blockers selected from indoxacarb and metaflumizone.
(23) Inhibitors of acetyl-CoA carboxylase, preferably tetronic and tetramic acid derivatives selected from spirodiclofen, spiromesifen, and spirotetramat.
(24) Mitochondrial complex IV electron transport inhibitors, preferably phosphines selected from aluminum phosphide, calcium phosphide, phosphine, and zinc phosphide, or cyanides selected from calcium cyanide, potassium cyanide, and sodium cyanide.
(25) Mitochondrial complex II electron transport inhibitors, preferably beta-keto nitrile derivatives selected from cyenopyrafen and cyflumetofen, or carboxanilides such as pyflubumide.
(26) Ryanodine receptor modulators, preferably diamides selected from chlorantraniliprole, cyantraniliprole, and flubendiamide.
(27) Chordotonal organ modulators (with undefined target structure) such as flonicamid.
(28) Further active ingredients selected from acynonapyr, afidopyropen, afoxolaner, azadirachtin, benclothiaz, benzoximate, benzpyrimoxan, bifenazate, broflanilide, bromopropylate, chinomethionat, chloroprallethrin, cryolite, cyclaniliprole, cycloxaprid, cyhalodiamide, dicloromezotiaz, dicofol, epsilon metofluthrin, epsilon momfluthrin, flometoquin, fluazaindolizine, fluensulfone, flufenerim, flufenoxystrobin, flufiprole, fluhexafon, fluopyram, flupyrimin, fluralaner, fluxametamide, fufenozide, guadipyr, heptafluthrin, imidaclothiz, iprodione, kappa bifenthrin, kappa tefluthrin, lotilaner, meperfluthrin, oxazosulfyl, paichongding, pyridalyl, pyrifluquinazon, pyriminostrobin, spirobudiclofen, spiropidion, tetramethylfluthrin, tetraniliprole, tetrachlorantraniliprole, tigolaner, tioxazafen, thiofluoximate, triflumezopyrim, and iodomethane; additionally preparations based on Bacillus firmus (I-1582, BioNeem, Votivo), and the following compounds: 1-{2-fluoro-4-methyl-5-[(2,2,2-trifluoroethyl)sulfinyl]phenyl}-3-(trifluoromethyl)-1H-1,2,4-triazole-5-amine (known from WO 2006/043635) (CAS 885026-50-6), {1′-[(2E)-3-(4-chlorophenyl)prop-2-en-1-yl]-5-fluorospiro[indole-3,4′-piperidine]-1(2H)-yl}(2-chloropyridin-4-yl)methanone (known from WO 2003/106457) (CAS 637360-23-7), 2-chloro-N-[2-{1-[(2E)-3-(4-chlorophenyl)prop-2-en-1-yl]piperidin-4-yl}-4-(trifluoromethyl) phenyl]isonicotinamide (known from WO 2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS-1440516-42-6), 4-(but-2-yn-1-yloxy)-6-(3,5-dimethylpiperidin-1-yl)-5-fluoropyrimidine (known from WO 2004/099160) (CAS 792914-58-0), PF1364 (known from JP2010/018586) (CAS Reg. No. 1204776-60-2), (3E)-3-[1-[(6-chloro-3-pyridyl)methyl]-2-pyridylidene]-1,1,1-trifluoropropan-2-one (known from WO 2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO 2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN 103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]propanamide, (+)-N-[3-chloro-1-(3-pyridinyl)-1H-pyrazol-4-yl]-N-ethyl-3-[(3,3,3-trifluoropropyl)sulfinyl]propanamide and (−)-N-[3-chloro-1-(3-pyridinyl)-1H-pyrazol-4-yl]-N-ethyl-3-[(3,3,3-trifluoropropyl)sulfinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); cyclopropanecarboxylic acid 3-(2,2-dichloroethenyl)-2,2-dimethyl-4-(1H-benzimidazol-2-yl)phenyl ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]indeno [1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]pyrimidine (known from WO 2013/115391 A1) (CAS 1449021-97-9), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), ethyl 3-(4-chloro-2,6-dimethylphenyl)-8-methoxy-1-methyl-2-oxo-1,8-diazaspiro[4.5]dec-3-en-4-ylcarboxylate (known from WO 2010/066780 A1, WO 2011151146 A1) (CAS 1229023-00-0), 4-[(5S)-5-(3,5-dichloro-4-fluorophenyl)-4,5-dihydro-5-(trifluoromethyl)-3-isoxazolyl]-N-[(4R)-2-ethyl-3-oxo-4-isoxazolidinyl]-2-methylbenzamide (known from WO 2011/067272, WO2013/050302) (CAS 1309959-62-3).

Insecticides that can preferably be used together with the herbicides are, for example, as follows: acetamiprid, acrinathrin, aldicarb, amitraz, acinphos-methyl, cyfluthrin, carbaryl, cypermethrin, deltamethrin, endosulfan, ethoprophos, fenamiphos, fenthion, fipronil, imidacloprid, methamidophos, methiocarb, niclosamide, oxydemeton-methyl, prothiophos, silafluofen, thiacloprid, thiodicarb, tralomethrin, triazophos, trichlorfon, triflumuron, terbufos, fonofos, phorate, chlorpyriphos, carbofuran, and tefluthrin.

### II. Recombinant DNA Molecules

The disclosure relates, in certain embodiments, to recombinant DNA molecules that encode herbicide-insensitive protoporphyrinogen oxidases (PPOs) and the proteins encoded thereby. As used herein, the term “engineered” refers to a non-natural DNA, protein, cell, or organism that would not normally be found in nature and was created by human intervention. An “engineered protein,” “engineered enzyme,” or “engineered PPO,” refers to a protein, enzyme, or PPO whose amino acid sequence was conceived of and created in the laboratory using one or more of the techniques of biotechnology, protein design, or protein engineering, such as molecular biology, protein biochemistry, bacterial transformation, plant transformation, site-directed mutagenesis, directed evolution using random mutagenesis, genome editing, gene editing, gene cloning, DNA ligation, DNA synthesis, protein synthesis, and DNA shuffling. For example, an engineered protein may have one or more deletions, insertions, or substitutions relative to the coding sequence of the wild-type protein and each deletion, insertion, or substitution may consist of one or more amino acids. Genetic engineering can be used to create a DNA molecule encoding an engineered protein, such as an engineered PPO that is herbicide tolerant and comprises at least a first amino acid substitution relative to a wild-type PPO protein as described herein.

In one embodiment, proteins provided herein have herbicide-tolerant protoporphyrinogen oxidase activity. As used herein, “herbicide-tolerant protoporphyrinogen oxidase” means the ability of a protoporphyrinogen oxidase to maintain at least some of its protoporphyrinogen oxidase activity in the presence of one or more PPO inhibiting herbicide(s). The term “protoporphyrinogen oxidase activity” means the ability to catalyze the six-electron oxidation (removal of electrons) of protoporphyrinogen IX to form protoporphyrin IX, that is, to catalyze the dehydrogenation of protoporphyrinogen to form protoporphyrin. Enzymatic activity of a protoporphyrinogen oxidase can be measured by any means known in the art, for example, by an enzymatic assay in which the production of the product of protoporphyrinogen oxidase or the consumption of the substrate of protoporphyrinogen oxidase in the presence of one or more PPO inhibiting herbicide(s) is measured via fluorescence, high performance liquid chromatography (HPLC), or mass spectrometry (MS). Another example of an assay for measuring enzymatic activity of a protoporphyrinogen oxidase is a bacterial assay, such as the assays described herein, whereby a recombinant protoporphyrinogen oxidase is expressed in a bacterial cell otherwise lacking PPO activity and the ability of the recombinant protoporphyrinogen oxidase to complement this knockout phenotype is measured. As used herein, a “hemG knockout strain” means an organism or cell of an organism, such as E. coli, that lacks HemG activity to the extent that it is unable to grow on heme-free growth medium, or such that its growth is detectably impaired in the absence of heme relative to an otherwise isogenic strain comprising a functional HemG. A hemG knockout strain of, for instance, E. coli may be prepared in view of knowledge in the art, for instance in view of the E. coli HemG PPO sequence (Ecogene Accession No. EG11485; Sasarman et al., “Nucleotide sequence of the hemG gene involved in the protoporphyrinogen oxidase activity of E. coli K12” Can. J. Microbiol. 39:1155-1161, 1993).

As used herein, the term “recombinant” refers to a non-naturally occurring DNA, protein, cell, seed, or organism that is the result of genetic engineering and was created by human intervention. A “recombinant DNA molecule” is a DNA molecule comprising a DNA sequence that does not naturally occur and as such is the result of human intervention, such as a DNA molecule comprising at least two DNA molecules heterologous to each other. An example of a recombinant DNA molecule is a DNA molecule provided herein encoding an herbicide-tolerant protoporphyrinogen oxidase operably linked to a heterologous promoter. A “recombinant protein” is a protein comprising an amino acid sequence that does not naturally occur and as such is the result of human intervention, such as an engineered protein. A recombinant cell, seed, or organism is a cell, seed, or organism comprising transgenic or heterologous DNA or protein, for example a transgenic plant cell, seed, or plant comprising a DNA construct or engineered protein described herein.

As used herein, “wild-type” means a naturally occurring. A “wild-type DNA molecule,” “wild-type protein” is a naturally occurring version of a DNA molecule or protein, that is, a version of a DNA molecule or protein pre-existing in nature. A wild-type version of a DNA molecule or protein may be useful for comparison with a recombinant or engineered DNA molecule or protein. An example of a wild-type protein useful for comparison with the engineered proteins provided by the present disclosure is the PPO enzyme from E. cloacae (H_N90) provided as SEQ ID NO:1.

A “wild-type plant” is a naturally occurring plant. Such wild-type plants may also be useful for comparison with a plant comprising a recombinant or engineered DNA molecule or protein. An example of a wild-type plant useful for comparison with plants comprising a recombinant or engineered DNA molecule or protein may be a plant of the same type as the plant comprising the engineered DNA molecule or protein, such as a protein conferring an herbicide tolerance trait, and as such is genetically distinct from the plant comprising the herbicide tolerance trait.

In certain embodiments, wild-type plants may also be used or referred to as “control plants.” As used herein, “control” means an experimental control designed for comparison purposes. For example, a control plant in a transgenic plant analysis is a plant of the same type as the experimental plant (that is, the plant to be tested) but does not contain the transgenic insert, recombinant DNA molecule, or DNA construct of the experimental plant. Examples of control plants useful for comparison with transgenic plants include: for maize plants, non-transgenic LH244 maize (U.S. Pat. No. 6,252,148) or non-transgenic 01DKD2 maize (U.S. Pat. No. 7,166,779); for comparison with soybean plants, non-transgenic A3555 soybean (ATCC deposit number PTA-10207); for comparison with cotton plants, non-transgenic DP393 (U.S. Pat. No. 6,930,228, PVP 200400266); for comparison with canola or Brassica napus plants, non-transgenic Brassica napus variety 65037 Restorer line (Canada Plant Breeders' Rights Application 06-5517); for comparison with wheat plants, non-transgenic wheat variety Samson germplasm (PVP 1994).

As used herein, the term “DNA” or “DNA molecule” refers to a double-stranded DNA molecule of genomic or synthetic origin (that is, a polymer of deoxyribonucleotide bases or a polynucleotide molecule) read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, the term “protein-coding DNA molecule” refers to a DNA molecule comprising a DNA sequence that encodes a protein. As used herein, the term “protein” refers to a chain of amino acids linked by peptide (amide) bonds and includes both polypeptide chains that are folded or arranged in a biologically functional way and polypeptide chains that are not. As used herein, a “protein-coding sequence” means a DNA sequence that encodes a protein. As used herein, a “sequence” means a sequential arrangement of nucleotides or amino acids. A “DNA sequence” may refer to a sequence of nucleotides or to the DNA molecule comprising of a sequence of nucleotides; a “protein sequence” may refer to a sequence of amino acids or to the protein comprising a sequence of amino acids. The boundaries of a protein-coding sequence are usually determined by a translation start codon at the 5′-terminus and a translation stop codon at the 3′-terminus.

As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

Any number of methods well known to those skilled in the art can be used to isolate and manipulate a DNA molecule, or fragment thereof, as disclosed herein. For example, polymerase chain reaction (PCR) technology can be used to amplify a particular starting DNA molecule or to produce variants of the original molecule. DNA molecules, or fragment thereof, can also be obtained by other techniques, such as by directly synthesizing the fragment by chemical means, as is commonly practiced by using an automated oligonucleotide synthesizer.

Because of the degeneracy of the genetic code, a variety of different DNA sequences can encode proteins, such as the altered or engineered proteins disclosed herein. It is well within the capability of one of skill in the art to create alternative DNA sequences encoding the same, or essentially the same, altered or engineered proteins as described herein. These variant or alternative DNA sequences are within the scope of the embodiments described herein. As used herein, references to “essentially the same” sequence refers to sequences which encode amino acid substitutions, deletions, additions, or insertions that do not materially alter the functional activity of the protein encoded by the DNA molecule of the embodiments described herein. Allelic variants of the nucleotide sequences encoding a wild-type or engineered protein are also encompassed within the scope of the embodiments described herein. Substitution of amino acids other than those specifically exemplified or naturally present in a wild-type or engineered PPO enzyme are also contemplated within the scope of the embodiments described herein, so long as the PPO enzyme having the substitution still retains substantially the same functional activity described herein.

Recombinant DNA molecules of the present disclosure may be synthesized and modified by methods known in the art, either completely or in part, where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present disclosure includes recombinant DNA molecules and engineered proteins having at least 50% sequence identity, at least 60% sequence identity, at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, and at least 99% sequence identity to any of the recombinant DNA molecule or amino acid sequences provided herein, and having herbicide-tolerant protoporphyrinogen oxidase activity. As used herein, the term “percent sequence identity” or “% sequence identity” refers to the percentage of identical nucleotides or amino acids in a linear polynucleotide or amino acid sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar Inc., 1228 S. Park St., Madison, WI 53715), and MUSCLE (version 3.6) (Edgar, “MUSCLE: multiple sequence alignment with high accuracy and high throughput,” Nucleic Acids Research 32(5):1792-7, 2004) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

### III. Expression Constructs

As used herein, a “DNA construct” is a recombinant DNA molecule comprising two or more heterologous DNA sequences. DNA constructs are useful for transgene expression and may be comprised in vectors and plasmids. DNA constructs may be used in vectors for transformation (that is, the introduction of heterologous DNA into a host cell) to produce recombinant bacteria or transgenic plants and cells (and as such may also be contained in the plastid DNA or genomic DNA of a transgenic plant, seed, cell, or plant part). As used herein, a “vector” means any recombinant DNA molecule that may be used for bacterial or plant transformation. DNA molecules provided by the present disclosure can, for example, be inserted into a vector as part of a DNA construct having the DNA molecule operably linked to a heterologous gene expression element that functions in a plant to affect expression of the engineered protein encoded by the DNA molecule. Methods for making and using DNA constructs and vectors are well known in the art and described in detail in, for example, handbooks and laboratory manuals including Green and Sambrook, “Molecular Cloning: A Laboratory Manual” Vol. 1, 4^{th} ed., Cold Spring Harbor Laboratory Press, Cold Spring Harbor, NY, 2012. The components for a DNA construct, or a vector comprising a DNA construct, include one or more gene expression elements operably linked to a transcribable nucleic acid sequence, such as the following: a promoter for the expression of an operably linked DNA, an operably linked protein-coding DNA molecule, and an operably linked 3′ untranslated region (UTR). Gene expression elements useful in practicing the present disclosure include, but are not limited to, one or more of the following type of elements: promoter, 5′ UTR, enhancer, leader, cis-acting element, intron, transit sequence, 3′ UTR, and one or more selectable marker transgenes.

The term “transgene” refers to a DNA molecule artificially incorporated into the genome of an organism as a result of human intervention, such as by plant transformation methods. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. Transgenic plants of the present disclosure comprise the recombinant DNA molecules and proteins described herein.

As used herein, the term “heterologous” refers to the relationship between two or more things not normally associated in nature, for instance that are derived from different sources or not normally found in nature together in any other manner. For example, a DNA molecule or protein may be heterologous with respect to another DNA molecule, protein, cell, plant, seed, or organism if not normally found in nature together or in the same context. In certain embodiments, a first DNA molecule is heterologous to a second DNA molecule if the two DNA molecules are not normally found in nature together in the same context. For instance, a protein-coding recombinant DNA molecule is heterologous with respect to an operably linked promoter if such a combination is not normally found in nature. Similarly, a protein is heterologous with respect to a second operably linked protein, such as a transit peptide, if such combination is not normally found in nature. In another embodiment, a recombinant DNA molecule encoding a PPO enzyme is heterologous with respect to an operably linked promoter that is functional in a plant cell if such combination is not normally found in nature. A recombinant DNA molecule also may be heterologous with respect to a cell, seed, or organism into which it is inserted when it would not naturally occur in that cell, seed, or organism.

A “heterologous protein” is a protein present in a plant, seed, cell, tissue, or organism in which it does not naturally occur or operably linked to a protein with which it is not naturally linked. Examples of heterologous proteins are the PPO enzymes described herein that is expressed in any plant, seed, cell, tissue, or organism. Another example is a protein operably linked to a second protein, such as a transit peptide or herbicide-tolerant protein, with which it is not naturally linked, or a protein introduced into a plant cell in which it does not naturally occur using the techniques of genetic engineering.

As used herein, “operably linked” means two or more DNA molecules or two or more proteins linked in manner so that one may affect the function of the other. Operably linked DNA molecules or operably linked proteins may be part of a single contiguous molecule and may or may not be adjacent. For example, a promoter is operably linked with a protein-coding DNA molecule in a DNA construct where the two DNA molecules are so arranged that the promoter may affect the expression of the transgene.

The DNA constructs described herein may include a promoter operably linked to a protein-coding DNA molecule provided herein, whereby the promoter drives expression of the protein. Useful promoters include those that function in a cell for expression of an operably linked DNA molecule, such as a bacterial or plant promoter. Plant promoters are varied and well known in the art and include, for instance, those that are inducible, viral, synthetic, constitutive, temporally regulated, spatially regulated, or spatio-temporally regulated.

In one embodiment, a DNA construct provided herein includes a DNA sequence encoding a transit sequence that is operably linked to a heterologous DNA sequence encoding a PPO enzyme, whereby the transit sequence facilitates localizing the protein molecule within the cell. Transit sequences are known in the art as signal sequences, targeting peptides, targeting sequences, localization sequences, and transit peptides. An example of a transit sequence is a chloroplast transit peptide (CTP), a mitochondrial transit sequence (MTS), or a dual chloroplast and mitochondrial transit peptide. By facilitating protein localization within the cell, the transit sequence may increase the accumulation of recombinant protein, protect the protein from proteolytic degradation, or enhance the level of herbicide tolerance, and thereby reduce levels of injury in the cell, seed, or organism after herbicide application. CTPs and other targeting molecules that may be used in connection with the present disclosure are well known in the art.

As used herein, “transgene expression,” “expressing a transgene,” “protein expression,” and “expressing a protein,” mean the production of a protein through the process of transcribing a DNA molecule into messenger RNA (mRNA) and translating the mRNA into polypeptide chains, which are ultimately folded into proteins. A protein-coding DNA molecule may be operably linked to a heterologous promoter in a DNA construct for use in expressing the protein in a cell transformed with the recombinant DNA molecule.

### IV. Transgenic Plants

In one aspect, cells, tissues, plants, and seeds that comprising the recombinant DNA molecules or proteins are provided herein. These cells, tissues, plants, and seeds comprising the recombinant DNA molecules or proteins exhibit tolerance to one or more PPO inhibiting herbicide(s).

In the commercial production of crops, it is desirable to eliminate under reliable pesticidal management unwanted plants (i.e., “weeds”) from a field of crop plants. An ideal treatment would be one which could be applied to an entire field but which would eliminate only the unwanted plants while leaving the crop plants unaffected. One such treatment system would involve the use of crop plants which are tolerant to an herbicide so that when the herbicide is sprayed on a field of herbicide-tolerant crop plants, the crop plants would continue to thrive while non-herbicide-tolerant weeds are killed or severely damaged. Ideally, such treatment systems would take advantage of varying herbicide properties so that weed control could provide the best possible combination of flexibility and economy. For example, individual herbicides have different longevities in the field, and some herbicides persist and are effective for a relatively long time after they are applied to a field while other herbicides are quickly broken down into other and/or non-active compounds. An ideal treatment system would allow the use of different herbicides so that growers could tailor the choice of herbicides for a particular situation.

While a number of herbicide-tolerant crop plants are presently commercially available, one issue that has arisen for many commercial herbicides and herbicide/crop combinations is that individual herbicides typically have incomplete spectrum of activity against common weed species. For most individual herbicides which have been in use for some time, populations of herbicide resistant weed species and biotypes have become more prevalent (see, e.g., Tranel and Wright, Weed Science 50:700-712, 2002; Owen and Zelaya, Pest Manag. Sci. 61:301-311, 2005). Transgenic plants which are resistant to more than one herbicide have been described (see, e.g., WO 2005/012515). However, improvements in every aspect of crop production, weed control options, extension of residual weed control, and improvement in crop yield are continuously in demand.

One method of producing such cells, tissues, plants, and seeds is through plant transformation. Suitable methods for transformation of host plant cells for use with the current disclosure include any method by which DNA can be introduced into a cell (for example, where a recombinant DNA construct is stably integrated into a plant chromosome) and are well known in the art. Two effective, and widely utilized, methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. A cell with a recombinant DNA molecule or protein of the present disclosure may be selected for the presence of the recombinant DNA molecule or protein, for instance through its encoded enzymatic activity, before or after regenerating such a cell into a plant.

Another method of producing the cells, plants, and seeds of the present disclosure is through genome modification using site-specific integration or genome editing. Targeted modification of plant genomes through the use of genome editing methods can be used to create improved plant lines through modification of plant genomic DNA. As used herein “site-directed integration” refers to genome editing methods the enable targeted insertion of one or more nucleic acids of interest into a plant genome. Suitable methods for altering a wild-type DNA sequence or a preexisting transgenic sequence or for inserting DNA into a plant genome at a pre-determined chromosomal site include any method known in the art. Exemplary methods include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system). Several embodiments relate to methods of genome editing by using single-stranded oligonucleotides to introduce precise base pair modifications in a plant genome, as described by Sauer et al., Plant Physiology 170(4):1917-1928, 2016. Methods of genome editing to modify, delete, or insert nucleic acid sequences into genomic DNA are known in the art.

In certain embodiments, the present disclosure provides modification or replacement of an existing coding sequence, such as a PPO coding sequence or another existing transgenic insert, within a plant genome with a sequence encoding a protein, such as a PPO coding sequence of the present disclosure, or an expression cassette comprising such a protein. Several embodiments relate to the use of a known genome editing methods, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system).

Several embodiments may therefore relate to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing or an expression cassette comprising nucleic acid sequence encoding a PPO protein as described herein (in cis) or on a separate molecule or vector (in trans). Several methods for site-directed integration are known in the art involving different sequence-specific nucleases (or complexes of proteins or guide RNA or both) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ).

As used herein, the term “double-strand break inducing agent” refers to any agent that can induce a double-strand break (DSB) in a DNA molecule. In some embodiments, the double-strand break inducing agent is a site-specific genome modification enzyme.

As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases and any combination thereof. In some embodiments, the site-specific genome modification enzyme is a sequence-specific nuclease.

In one aspect, the endonuclease is selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Cas12a (also known as Cpf1) Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof).

In some embodiments, the site-specific genome modification enzyme is a recombinase. Non-limiting examples of recombinases include a tyrosine recombinase attached to a DNA recognition motif and is selected from the group consisting of a Cre recombinase, a Gin recombinase, a Flp recombinase, and a Tnp1 recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Any of the DNA of interest provided herein can be integrated into a target site of a chromosome sequence by introducing the DNA of interest and the provided site-specific genome modification enzymes. Any method provided herein can utilize any site-specific genome modification enzyme provided herein.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant).

The transgenic plants, progeny, seeds, plant cells, and plant parts described herein may also contain one or more additional traits. Additional traits may be introduced by crossing a plant containing a transgene comprising the recombinant DNA molecules provided herein with another plant containing one or more additional trait(s). As used herein, “crossing” means breeding two individual plants to produce a progeny plant. Two plants may thus be crossed to produce progeny that contain the desirable traits from each parent. As used herein “progeny” means the offspring of any generation of a parent plant, and transgenic progeny comprise a DNA construct provided herein and inherited from at least one parent plant.

Additional trait(s) also may be introduced by co-transforming a DNA construct for that additional transgenic trait(s) with a DNA construct comprising the recombinant DNA molecules provided herein (for example, with all the DNA constructs present as part of the same vector used for plant transformation) or by inserting the additional trait(s) into a transgenic plant comprising a DNA construct provided by the herein or vice versa (for example, by using any of the methods of plant transformation or genome editing on a transgenic plant or plant cell). Such additional traits include, but are not limited to, increased insect resistance, increased water use efficiency, increased yield performance, increased drought resistance, increased seed quality, improved nutritional quality, hybrid seed production, and herbicide tolerance, in which the trait is measured with respect to a wild-type plant. Illustrative additional herbicide-tolerance traits may include transgenic or non-transgenic tolerance to one or more herbicides such as ACCase inhibitors (for example aryloxyphenoxy propionates and cyclohexanediones), ALS inhibitors (for example sulfonylureas, imidazolinones, triazolopyrimidines, and triazolinones) EPSPS inhibitors (for example glyphosate), synthetic auxins (for example phenoxys, benzoic acids, carboxylic acids, semicarbazones), photosynthesis inhibitors (for example triazines, triazinones, nitriles, benzothiadiazoles, and ureas), glutamine synthesis inhibitors (for example glufosinate), HPPD inhibitors (for example isoxazoles, pyrazolones, and triketones), PPO inhibitors (for example diphenylethers, N-phenylphthalimide, aryl triazinones, and pyrimidinediones), and long-chain fatty acid inhibitors (for example chloroacetamindes, oxyacetamides, and pyrazoles), among others. Examples of herbicide-tolerance proteins useful for producing additional herbicide-tolerance traits are well known in the art and include, but are not limited to, glyphosate-tolerant 5-enolypyruvyl shikimate 3-phosphate synthases (e.g., CP4 EPSPS, 2mEPSPS), glyphosate oxidoreductases (GOX), glyphosate N-acetyltransferases (GAT), herbicide-tolerant acetolactate synthases (ALS)/acetohydroxyacid synthases (AHAS), herbicide-tolerant 4-hydroxyphenylpyruvate dioxygenases (HPPD), dicamba monooxygenases (DMO), phosphinothricin acetyl transferases (PAT), herbicide-tolerant glutamine synthetases (GS), 2,4-dichlorophenoxyproprionate dioxygenases (TfdA), R-2,4-dichlorophenoxypropionate dioxygenases (RdpA), S-2,4-dichlorophenoxypropionate dioxygenases (SdpA), herbicide-tolerant protoporphyrinogen oxidases (PPO), and cytochrome P450 monooxygenases. Exemplary insect resistance traits may include resistance to one or more insect members within one or more of the orders of Lepidoptera, Coleoptera, Hemiptera, Thysanoptera, Diptera, Hymenoptera, and Orthoptera, among others. Such additional traits are well known to one of skill in the art; for example, and a list of such transgenic traits is provided by the United States Department of Agriculture's (USDA) Animal and Plant Health Inspection Service (APHIS).

Transgenic plants and progeny that are tolerant to PPO inhibiting herbicides may be used with any breeding methods that are known in the art. In plant lines comprising two or more traits, the traits may be independently segregating, linked, or a combination of both in plant lines comprising three or more transgenic traits. Backcrossing to a parental plant and out-crossing with a non-transgenic plant are also contemplated, as is vegetative propagation. Descriptions of breeding methods that are commonly used for different traits and crops are well known to those of skill in the art. To confirm the presence of the transgene(s) in a particular plant or seed, a variety of assays may be performed. Such assays include, for example, molecular biology assays, such as Southern and Northern blotting, PCR, and DNA sequencing; biochemical assays, such as detecting the presence of a protein product, for example, by immunological means (ELISAs and western blots) or by enzymatic function; plant part assays, such as leaf or root assays; and also, by analyzing the phenotype of the whole plant.

Introgression of a transgenic trait into a plant genotype is achieved as the result of the process of backcross conversion. A plant genotype into which a transgenic trait has been introgressed may be referred to as a backcross converted genotype, line, inbred, or hybrid. Similarly, a plant genotype lacking the desired transgenic trait may be referred to as an unconverted genotype, line, inbred, or hybrid.

As used herein, the term “comprising” means “including but not limited to.”

Having described the invention in detail, it will be apparent that modifications, variations, and equivalent embodiments are possible without departing the scope of the invention defined in the appended claims. Furthermore, it should be appreciated that the examples in the present disclosure are provided as non-limiting examples.

### EXAMPLES

### Example 1: HemG Protoporphyrinogen Oxidases and Transit Peptides

Novel microbial HemG protoporphyrinogen oxidases that are tolerant to PPO inhibitor herbicides were previously identified from microbial sequence databases using bioinformatic methods and a herbicide bacterial screening system and are provided as SEQ ID NOs:1-20 and recombinant variants of these microbial HemG protoporphyrinogen oxidases are provided as SEQ ID NOs:65-193. DNA sequences encoding microbial HemG protoporphyrinogen oxidases and their variants, along with DNA sequences that are optimized for expression in a monocot or dicot can optionally be synthesized and are provided as SEQ ID NOs:22-64 and 194-322.

At the 5′ end of the DNA sequence encoding a protoporphyrinogen oxidase, a codon for a methionine, commonly known as a start codon, may be present. Alternatively, this codon (and optionally a few amino-terminal amino acids, for example two to seven), can be eliminated to facilitate operable linkage of a transit peptide sequence to the 5′ end of the coding sequence. Novel transit peptides were previously identified by using bioinformatic methods and tools, such as hidden Markov models (HMM), the Pfam database, and basic local alignment search tool (BLAST), to identify thousands of EST and genomic sequences predicted to encode proteins known to be localized to the chloroplast and mitochondria in plant cells and are provided herein as SEQ ID NOs:323-328, 340, and 342-407, along with their corresponding nucleotide sequences, provided herein as SEQ ID NOs:329-339, 341, and 408-483.

### Example 2: Herbicide Tolerance Evaluation of Transgenic Plants Expressing HemG PPOs

Protoporphyrinogen oxidases operably linked to transit peptides are tested in transgenic soybean, corn, and cotton plants for tolerance to PPO inhibiting herbicides and for weed control in the field.

Plant transformation vectors are constructed for expressing a chloroplast transit peptide operably linked to the PPO H_N90 (SEQ ID NO:1) in transgenic soybean, inbred corn, hybrid corn, and cotton plants, and introduced into seed-derived explants of soybean, inbred and hybrid corn, and cotton, respectively, through Agrobacterium tumefaciens-mediated transformation using standard methods known in the art. The regenerated R₀ plants are analyzed to select for events with a single copy insertion for advancement to R₁ nursery for R₁ seed production.

Seeds of the regenerated transgenic plants described above are sown in 12-cm tall plastic pots containing standard soil (14.7% sand, 19.9% clay, 65.4% silt, and 1.8% organic matter). Transgenic cotton and corn seed are sown at a density of one seed per pot, whereas soybean is sown at a density of three seeds per pot. Plants are grown in a greenhouse with 60% relative humidity in a light cycle of 13 hour day and 11 hour night. The temperature is kept at 23° C. during the day and 12° C. at night.

To confirm herbicide tolerance of transgenic soybean, inbred corn, hybrid corn, and cotton plants, experimental herbicidal formulations comprising one of compounds (a) to (y) are applied to plants at the 2-4 leaf stage (corresponding to BBCH 12-14). Each herbicide treatment is applied at one of two rates as follows: 100 or 200 g ai/ha. Methylated rapeseed oil (Mero) is used as an adjuvant and added to the spray mix for each herbicide at 0.5% v/v. Each treatment consists of four replicates (pots).

Plants are visually assessed for herbicide injury at the following time points: 7 days, 14 days, 21 days, and 28 days after application. Unsprayed transgenic plants are used for phenotypic comparison with unsprayed wild-type plants. Injury rating is determined as the percentage of leaf area of a plant exhibiting damage such as necrosis (brown or dead tissue), chlorosis (yellow tissue or yellow spotting), and malformation (misshapen leaves or plant structures, epinasty or twisting of stem, cupping of leaves) caused by herbicide application and is measured on a scale of 0-100, with zero being no injury and 100 being complete crop death. No injuries are expected for compounds (a) to (y).

1. A method for controlling or preventing weed growth in a plant growth area, wherein the method comprises the steps of:
(a) providing in said plant growth area a plant or a seed that when grown produces said plant, wherein the plant comprises a recombinant DNA molecule comprising a DNA sequence encoding a heterologous HemG protein, wherein said protein confers tolerance in said plant to an herbicidally active compound of the general formula (I) or an agrochemically acceptable salt thereof 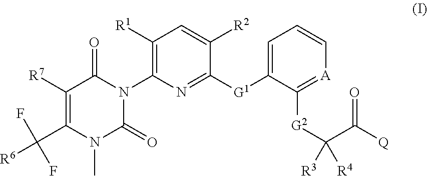
in which
A represents N (nitrogen) or a group C—R⁵,
G¹ and G² independently of each other represent O (oxygen) or S (sulfur),
R¹ represents fluorine, hydrogen, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents chlorine, fluorine, bromine, iodine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O—(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl, or
R³ and R⁴ together with the carbon atom to which they are bonded form a fully saturated or partly saturated 3- to 10-membered carbocyclic ring optionally having further substitution,
R⁵ represents hydrogen, halogen, (C₁-C₈)-alkyl, (C₁-C₈)-haloalkyl, or OR¹³,
R⁶ represents fluorine, hydrogen, chlorine, bromine, trifluoromethyl, difluoromethyl, methoxy, ethoxy, prop-1-yloxy, but-1-yloxy or methyl,
R⁷ represents hydrogen or methyl,
Q represents hydroxy or a group Q-1, Q-2 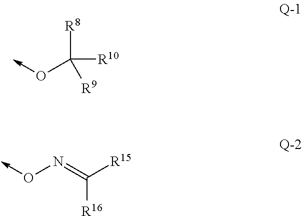
R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₁C₈)-haloalkyl, (C₃C₈)-cycloalkyl, (C₃C₈)-cycloalkyl-(C₁C₈)-alkyl, (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O—(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR¹³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₈)-cycloalkyl, aryl, heteroaryl, or heterocyclyl; and
(b) applying to said area an amount of said compound effective to control or prevent weed growth in the area.

2. The method of claim 1, wherein said compound of the general formula (I) is characterized as follows:
A represents N (nitrogen) or a group C—R⁵,
G¹ and G² independently of each other represent O (oxygen) or S (sulfur),
R¹ represents hydrogen, fluorine,
R² represents chlorine, bromine, iodine, nitro or cyano,
R³ and R⁴ independently of each other represent hydrogen, methyl, or ethyl, or
R³ and R⁴ together with the carbon atom to which they are bonded form a fully saturated or partly saturated 3- to 6-membered carbocyclic ring optionally having further substitution,
R⁵ represents hydrogen, chlorine, or methoxy,
R⁶ represents fluorine, methyl,
R⁷ represents hydrogen, methyl,
and
Q represents one of the following moieties Q-1 to Q-500:

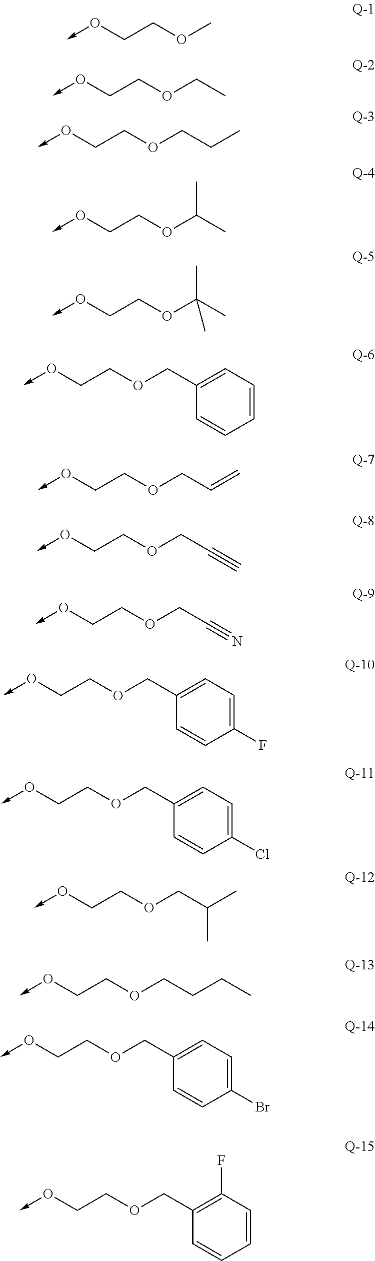

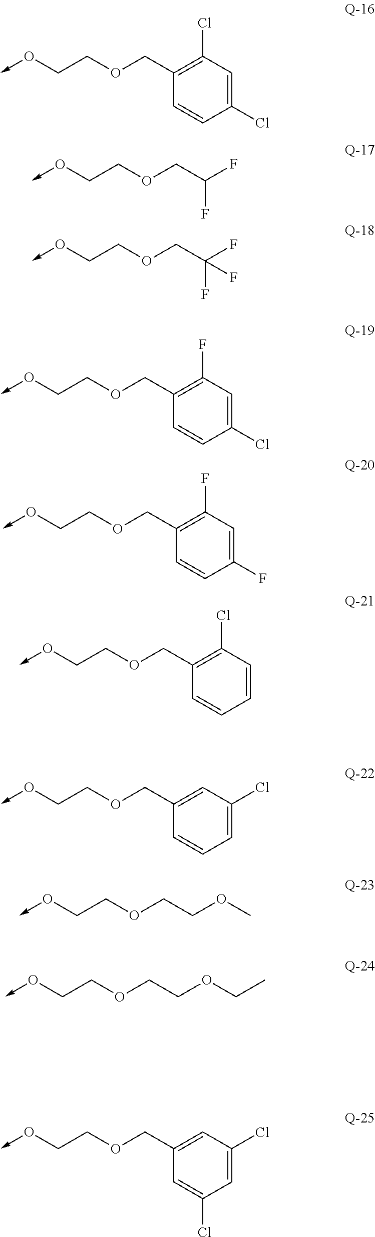

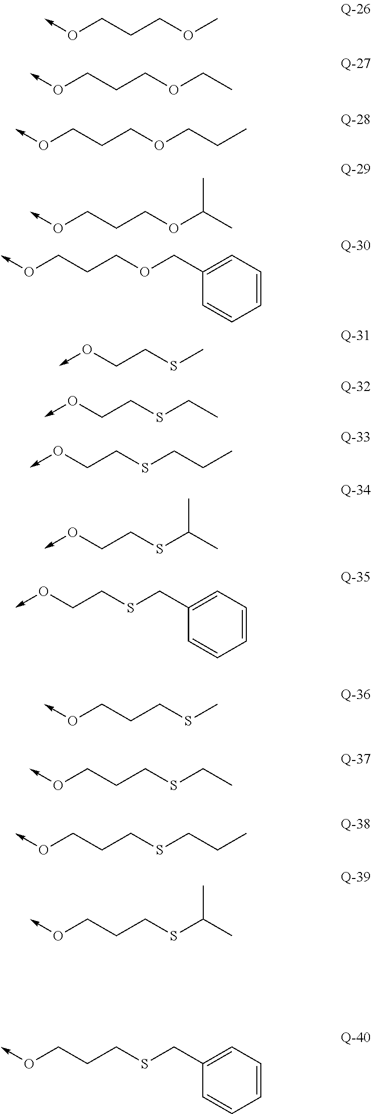

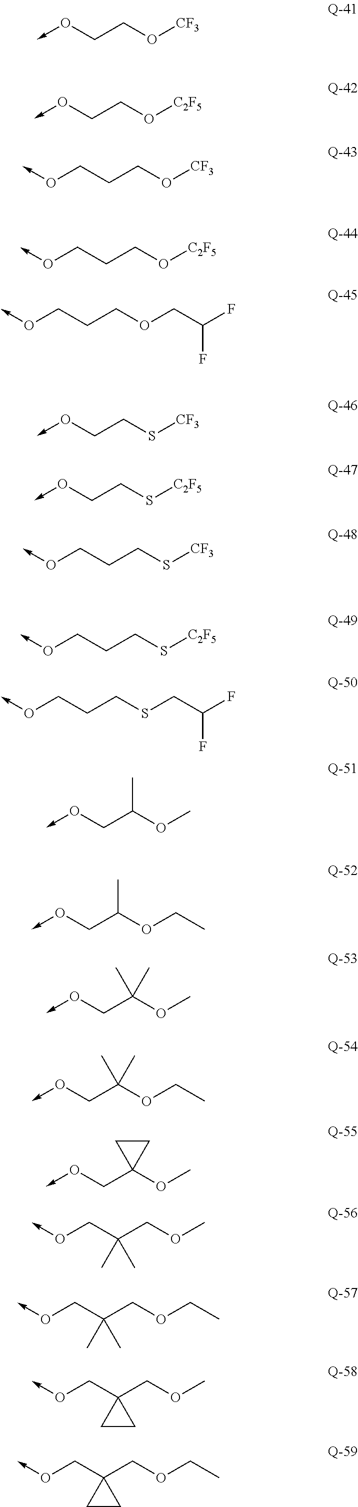

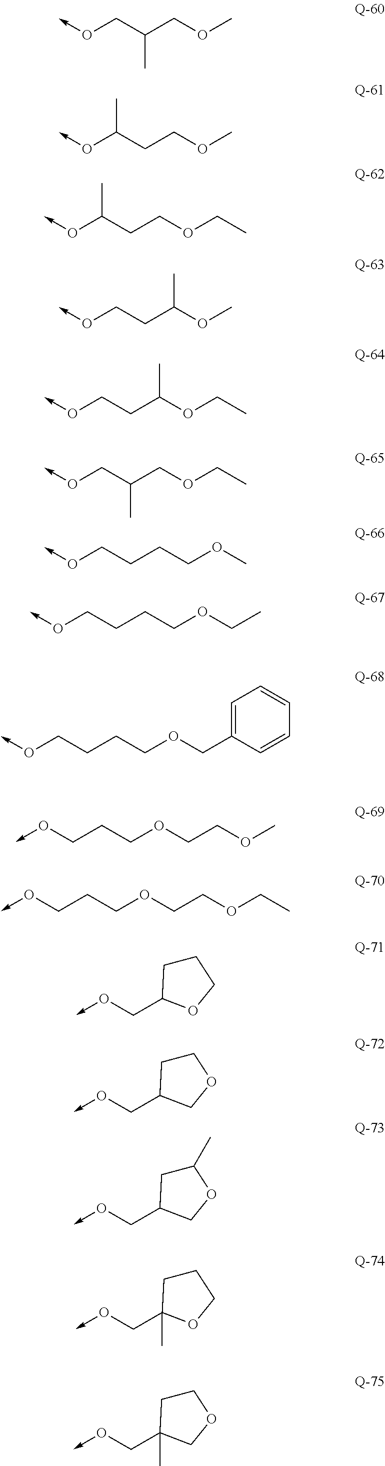

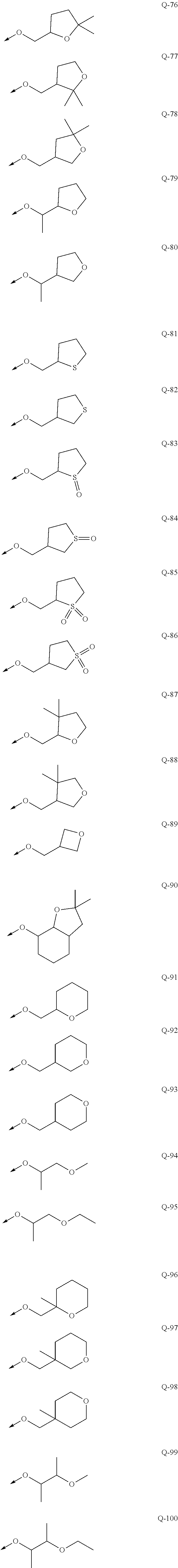

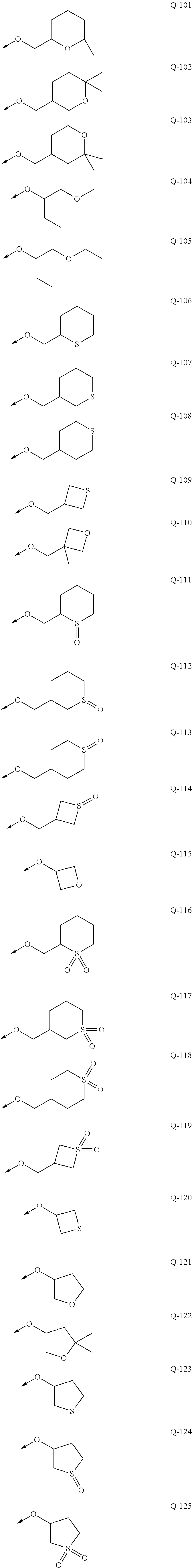

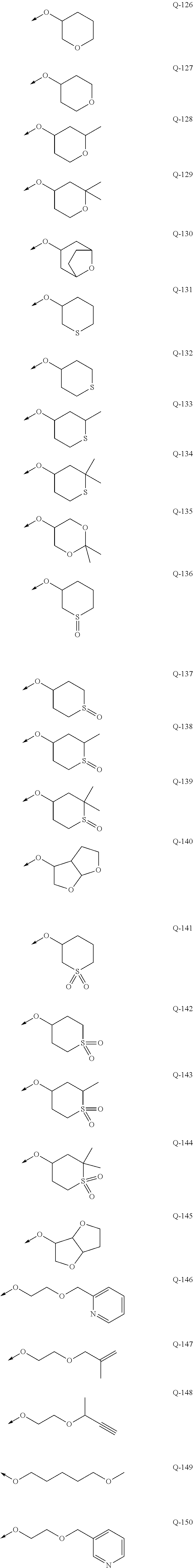

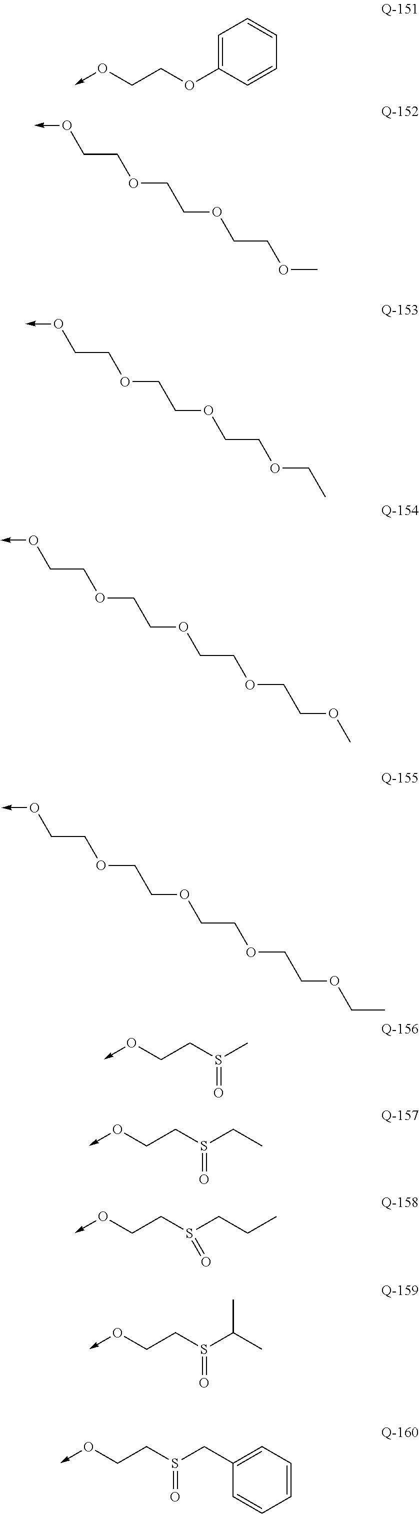

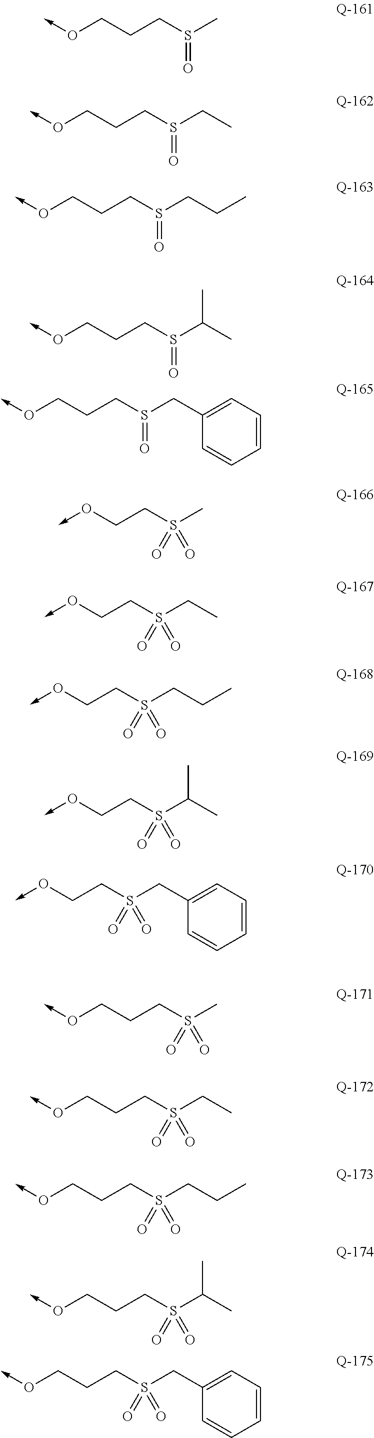

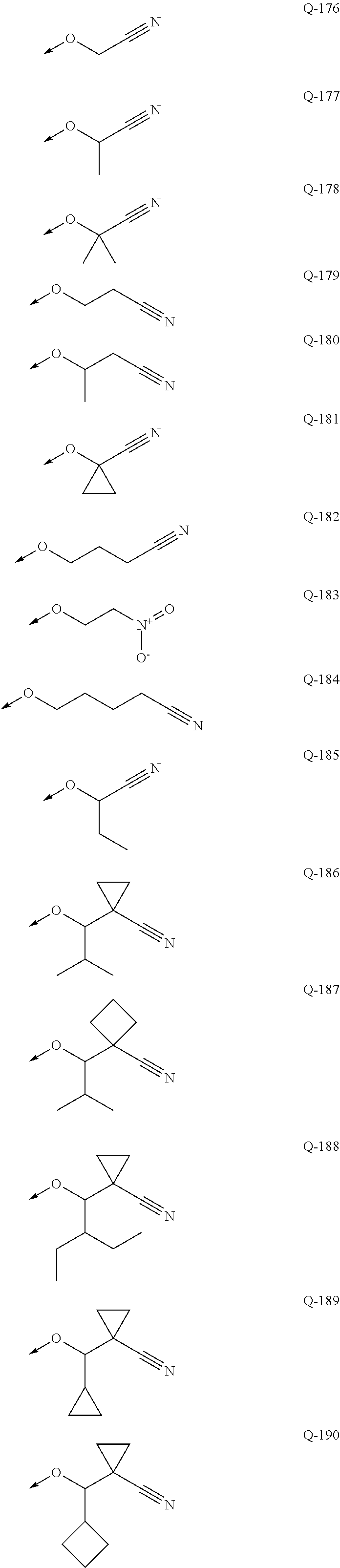

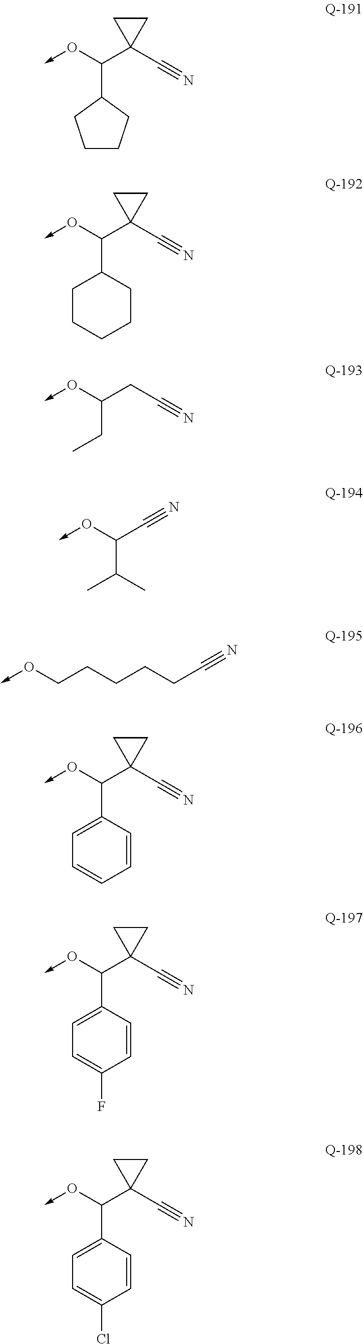

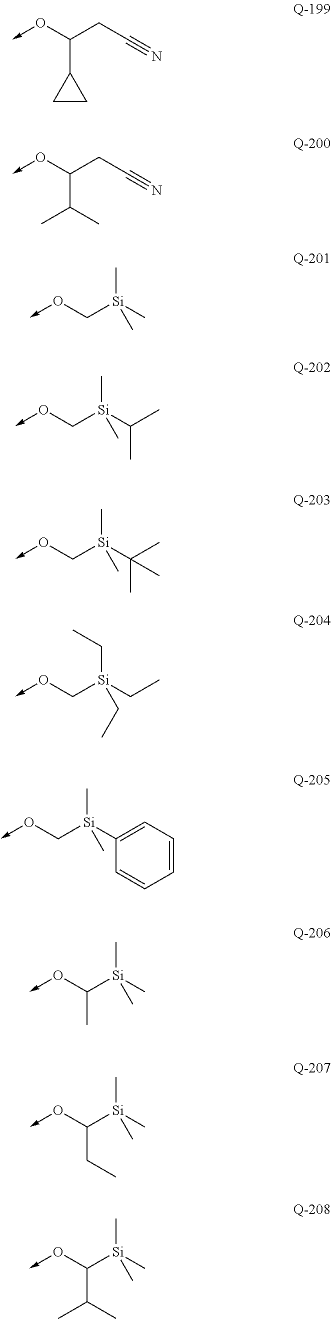

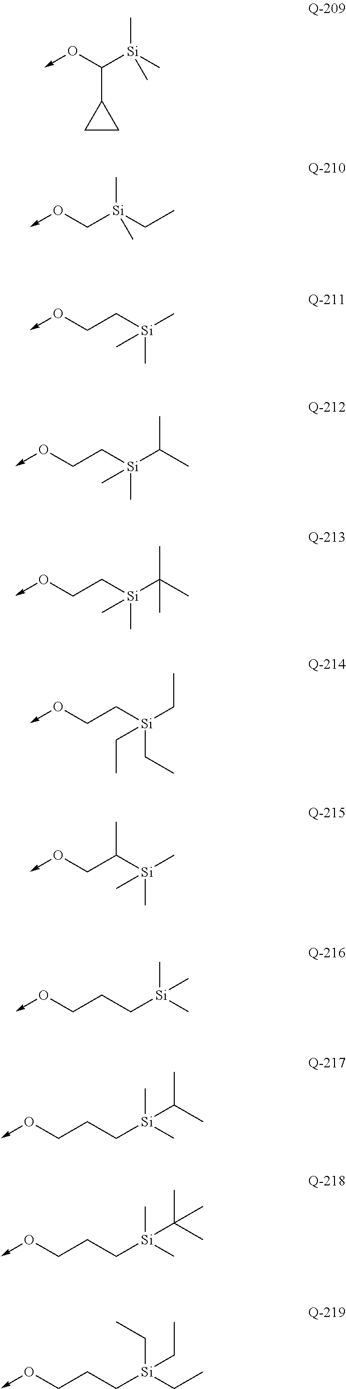

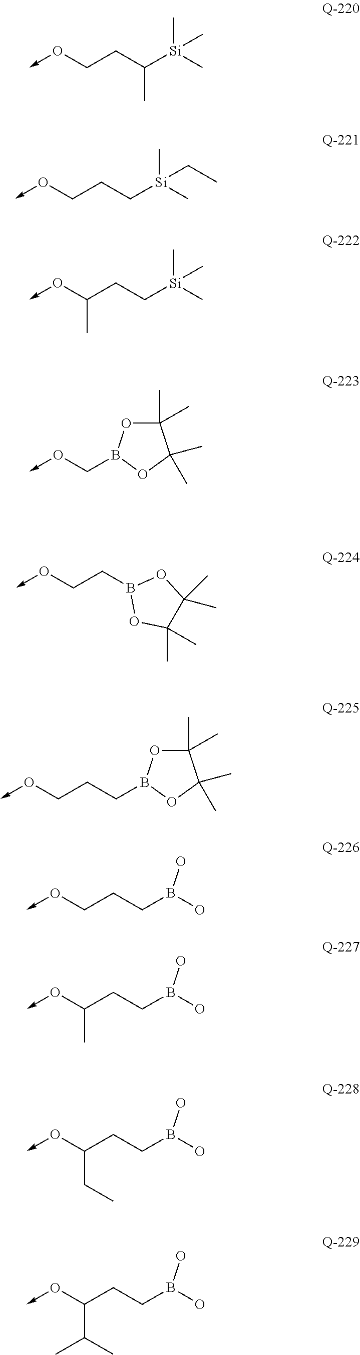

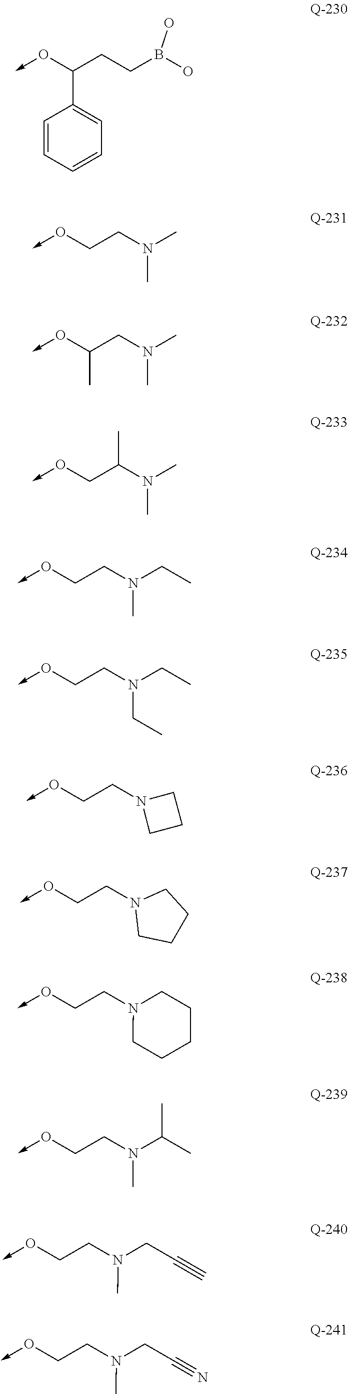

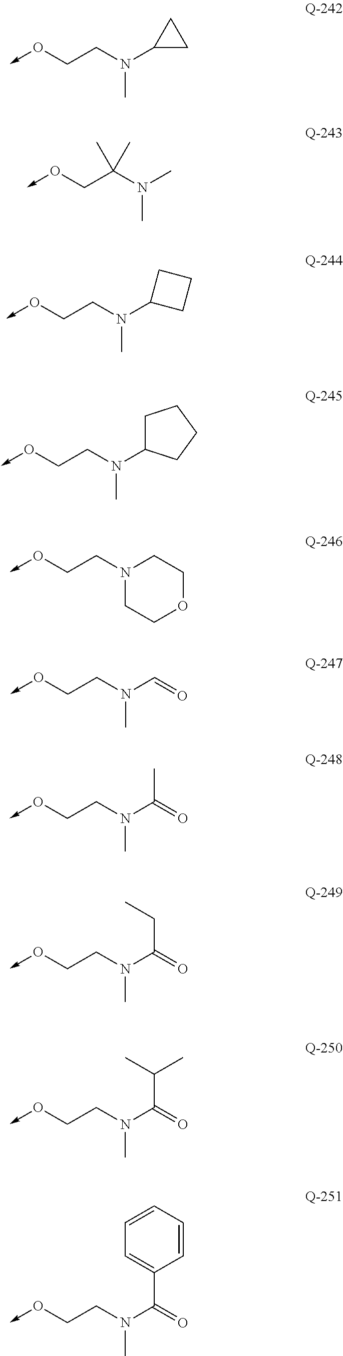

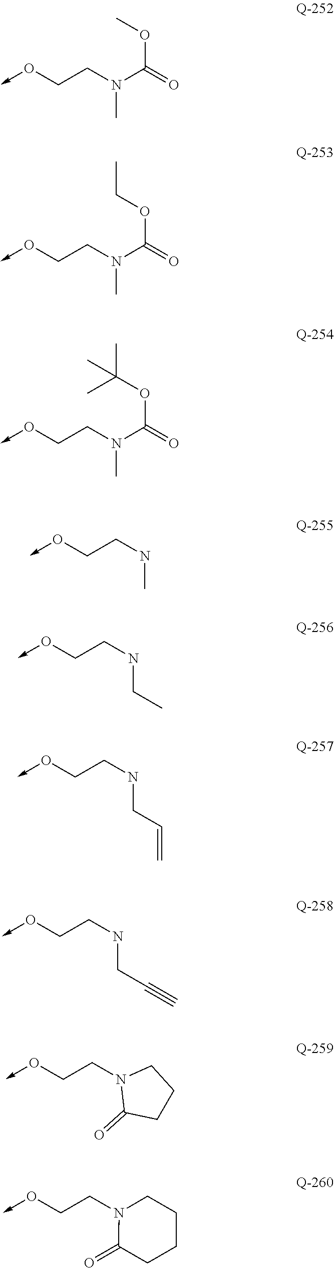

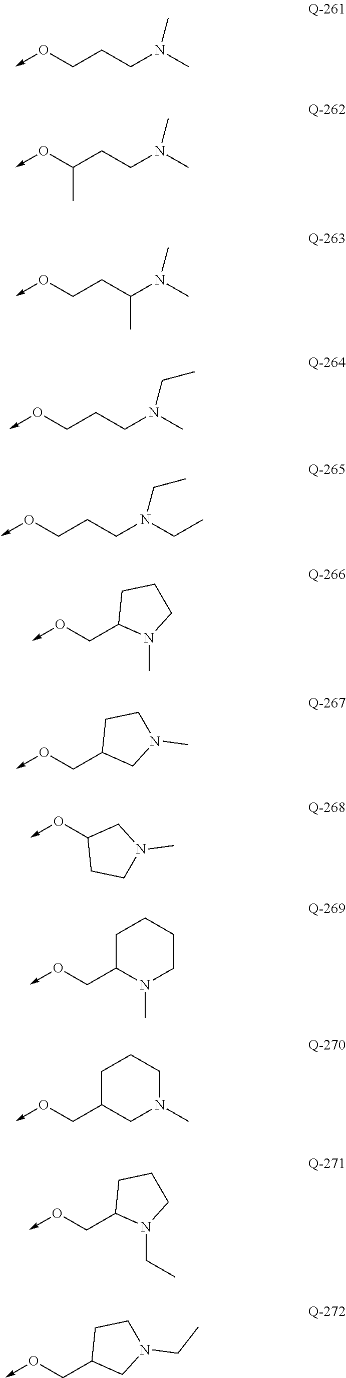

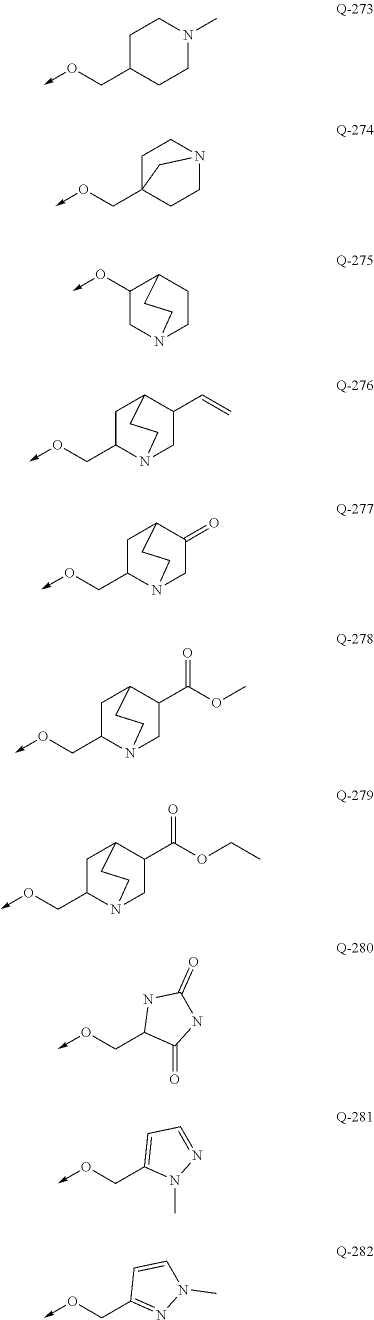

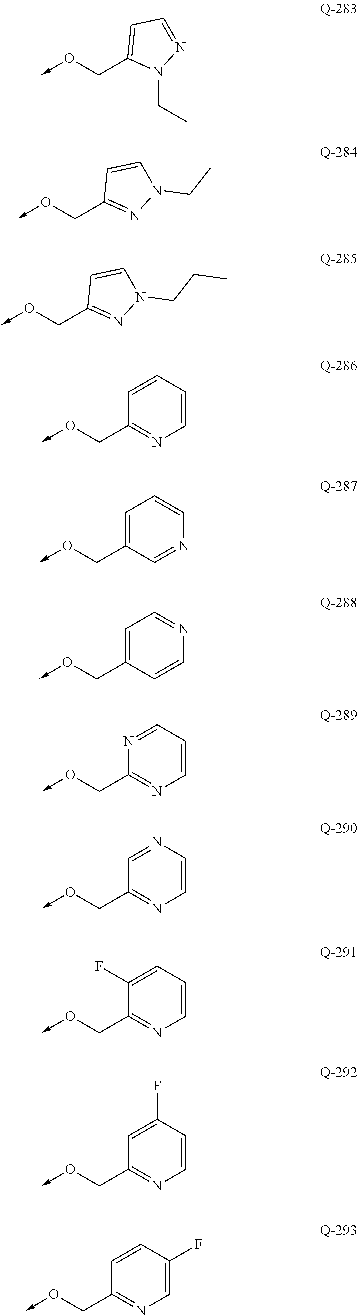

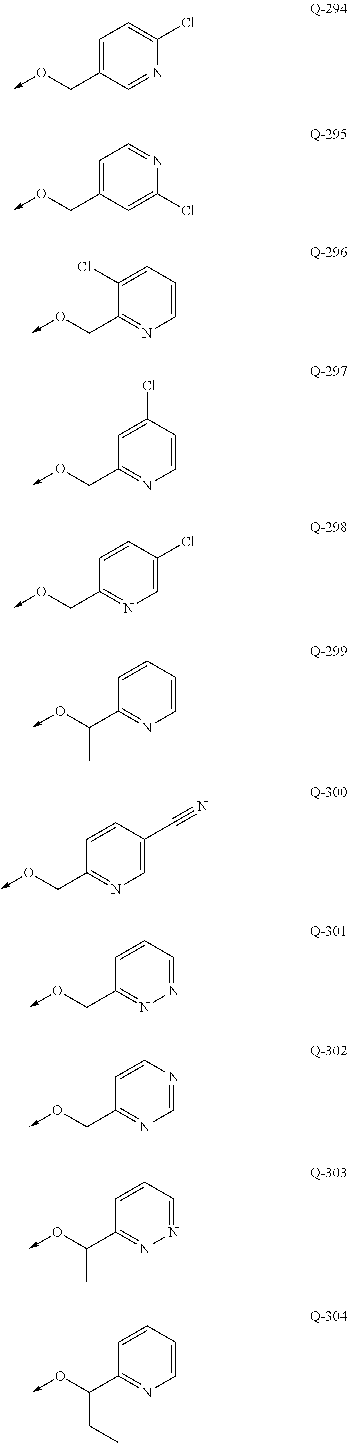

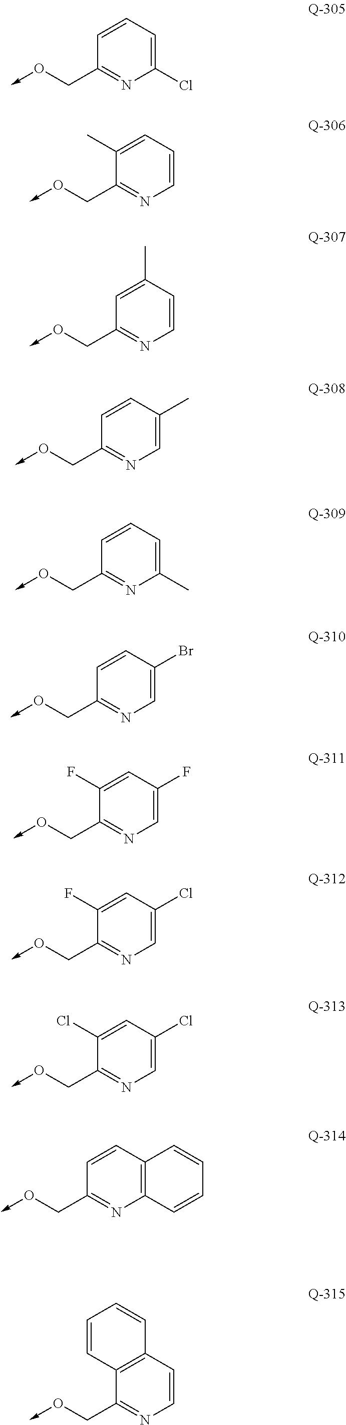

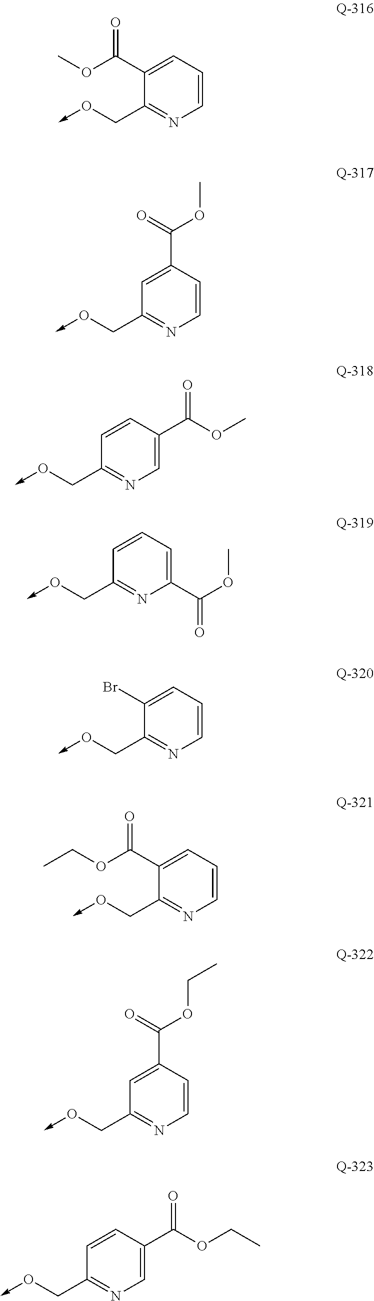

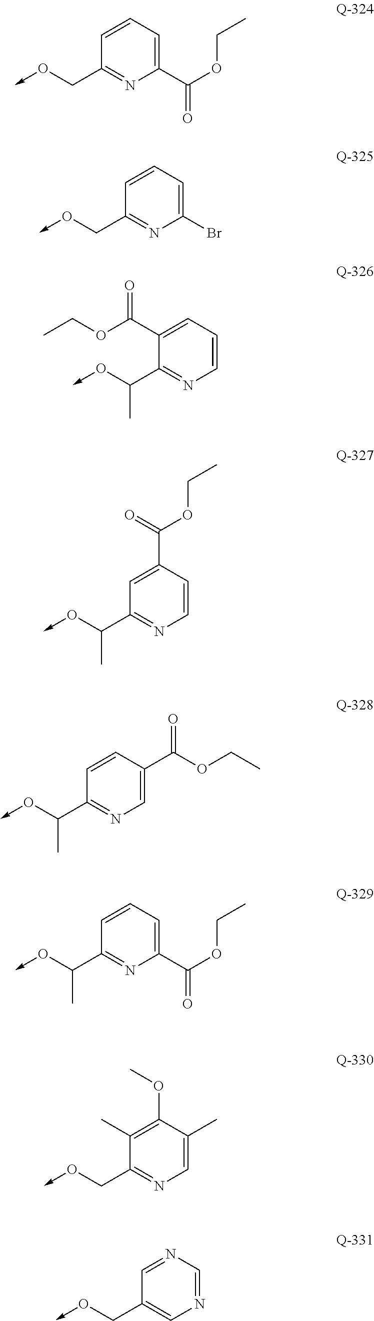

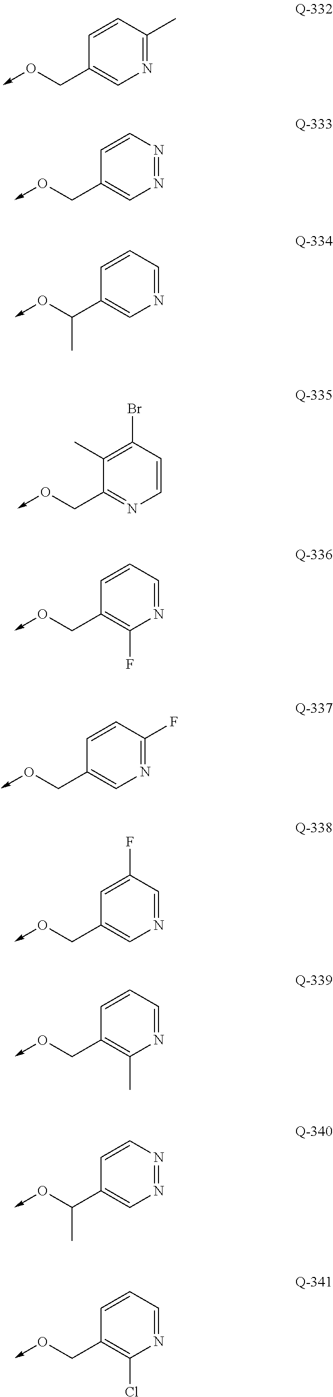

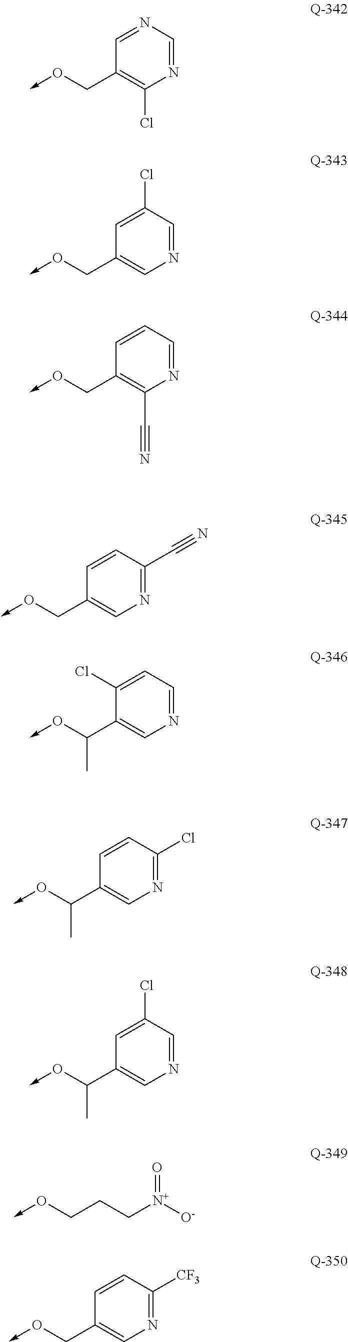

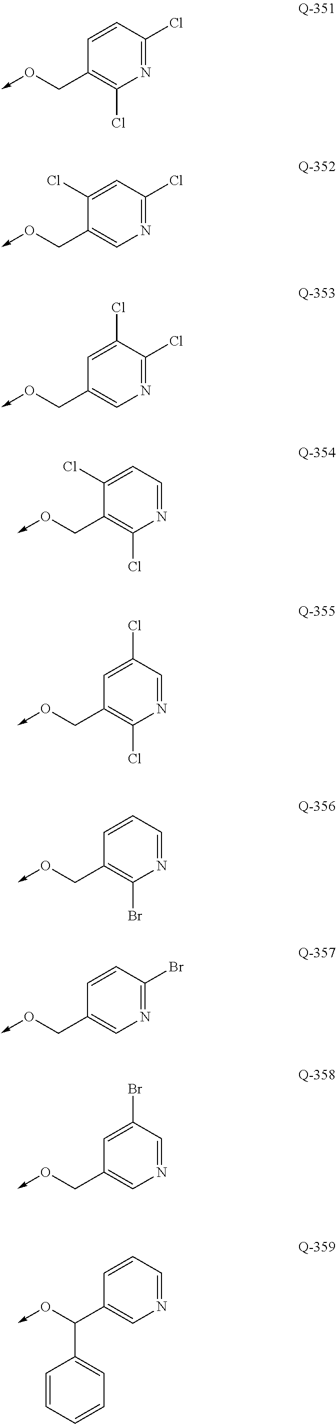

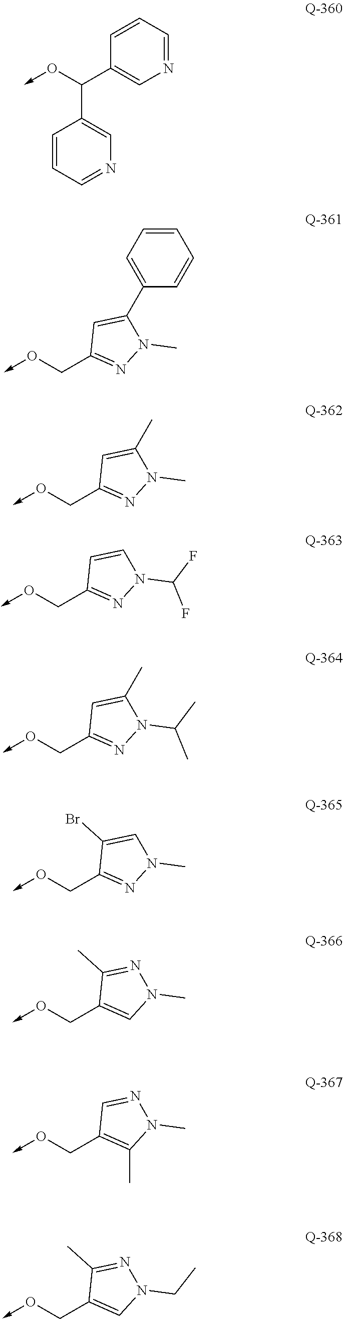

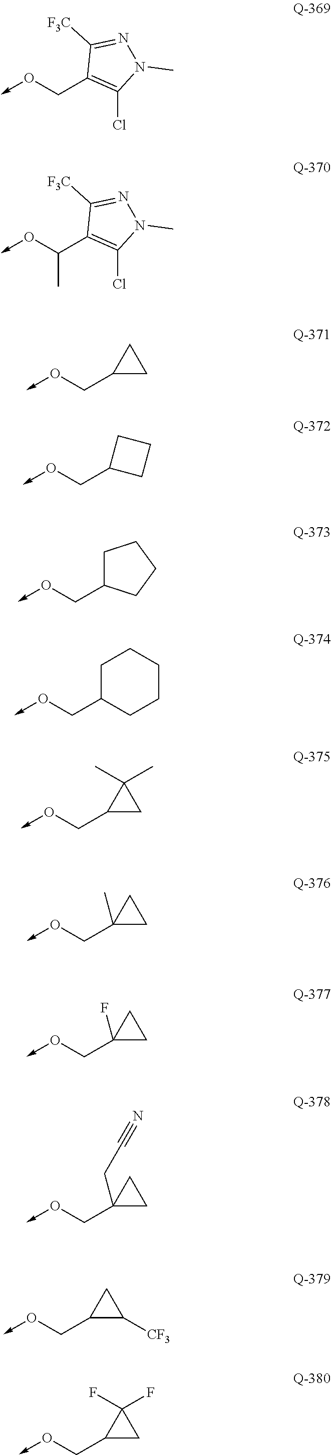

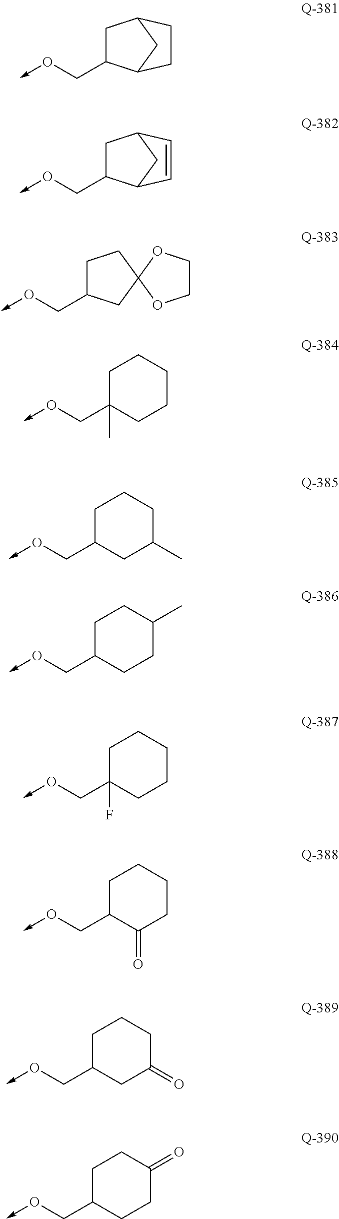

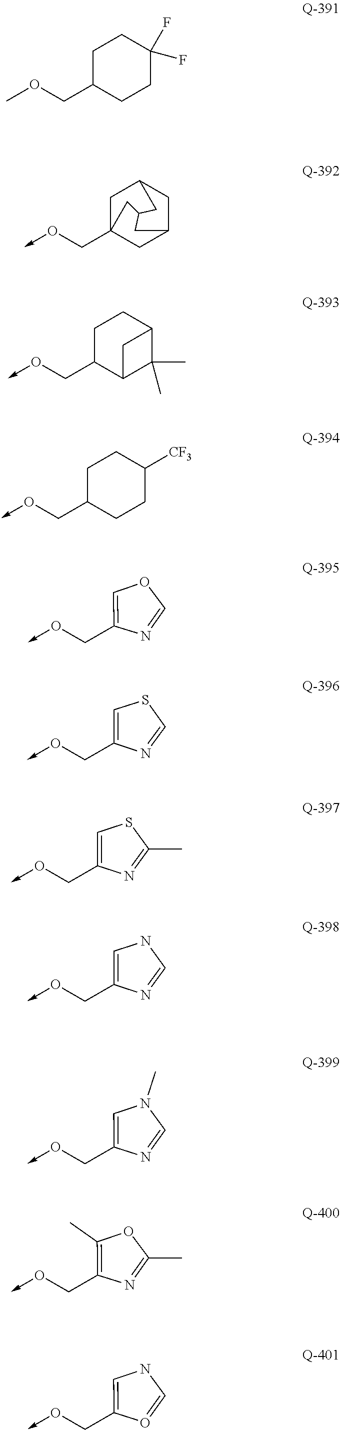

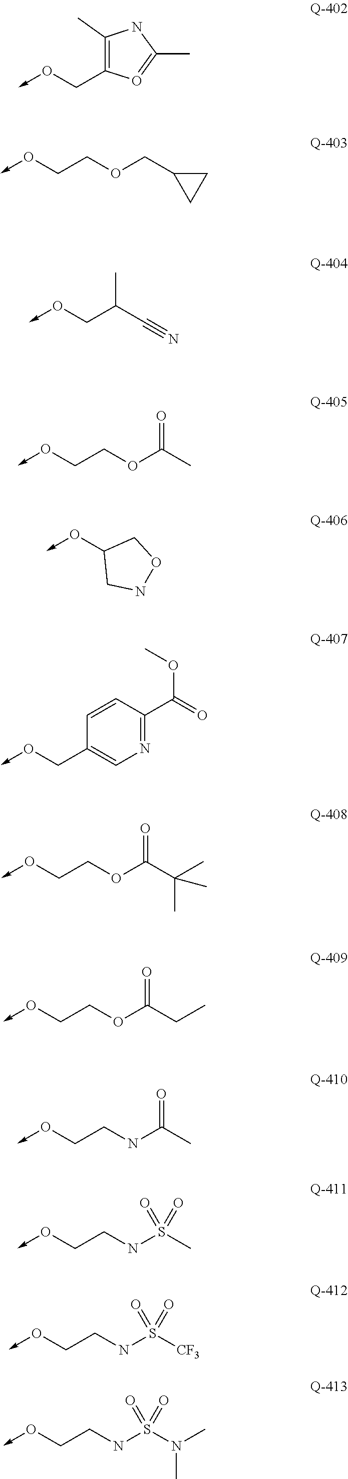

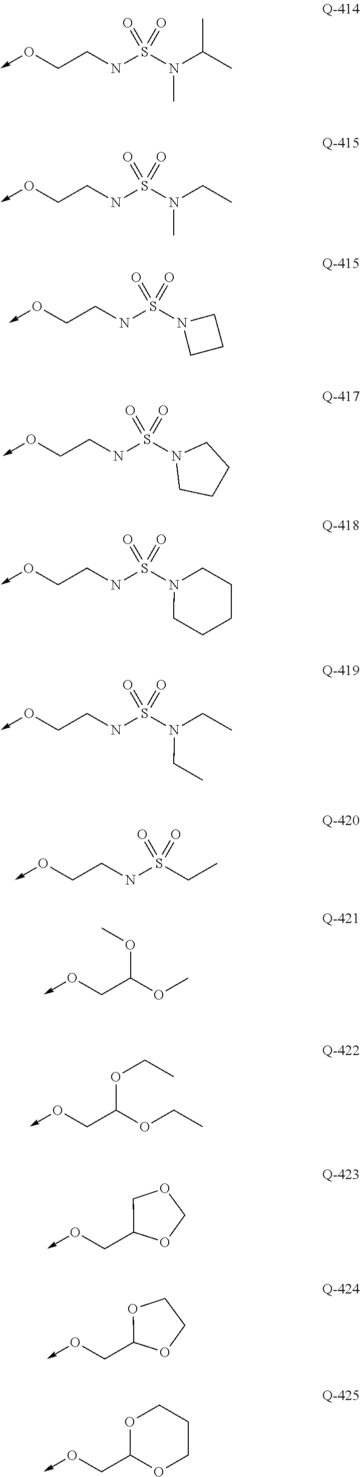

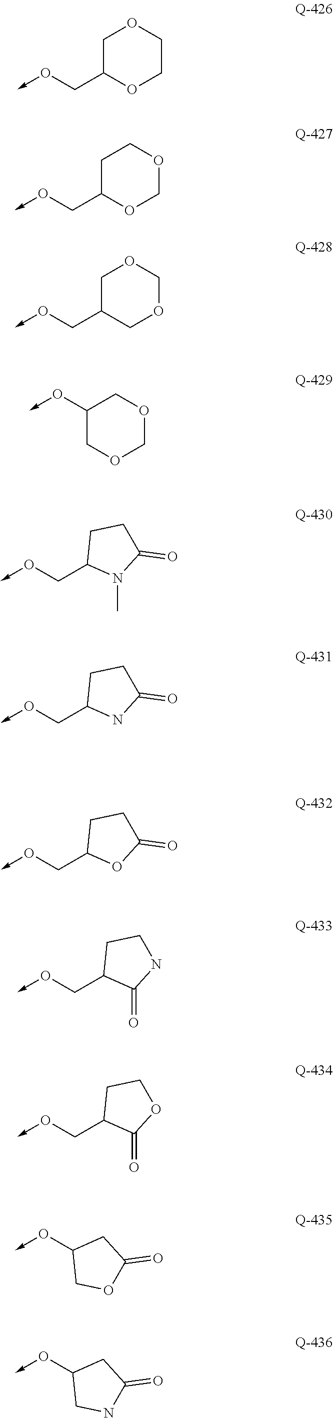

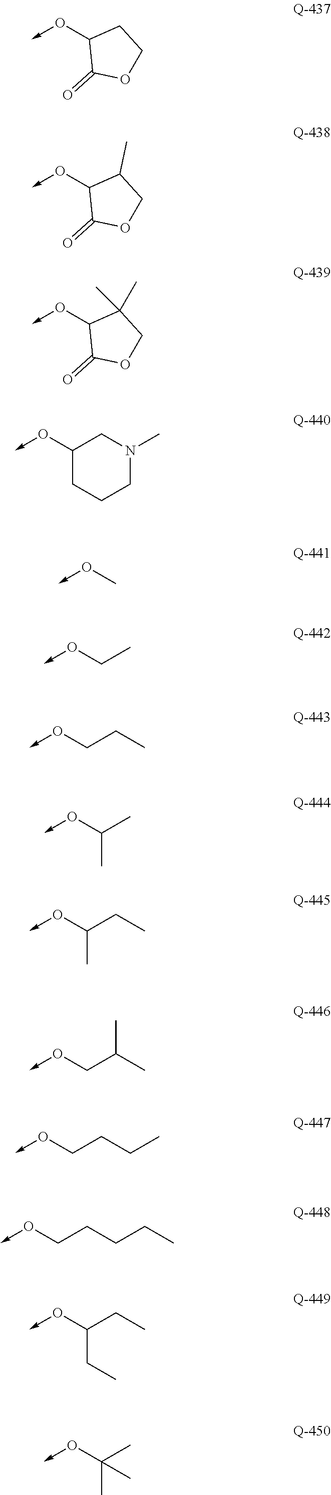

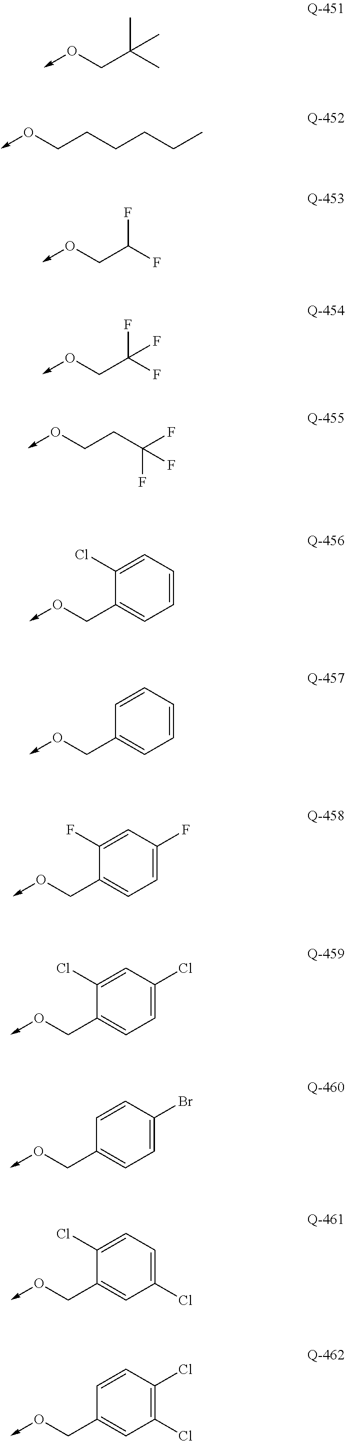

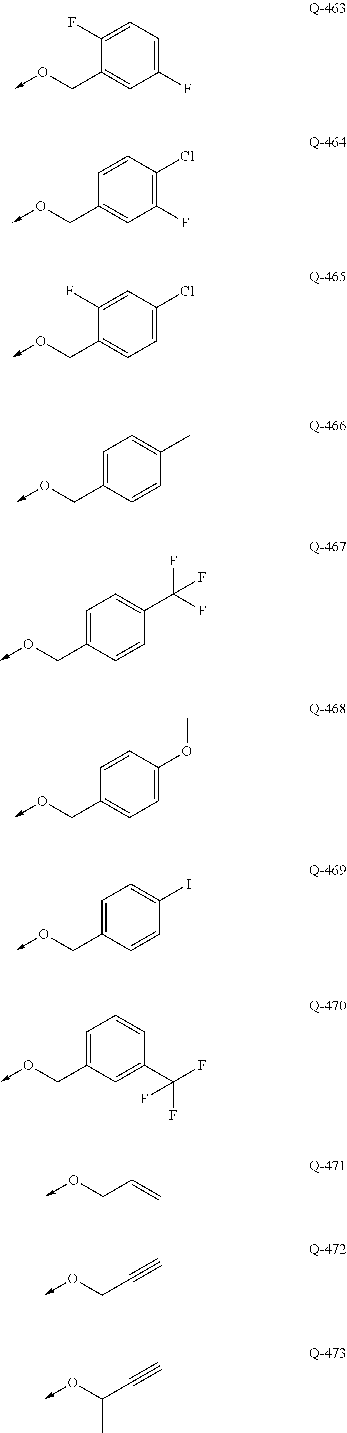

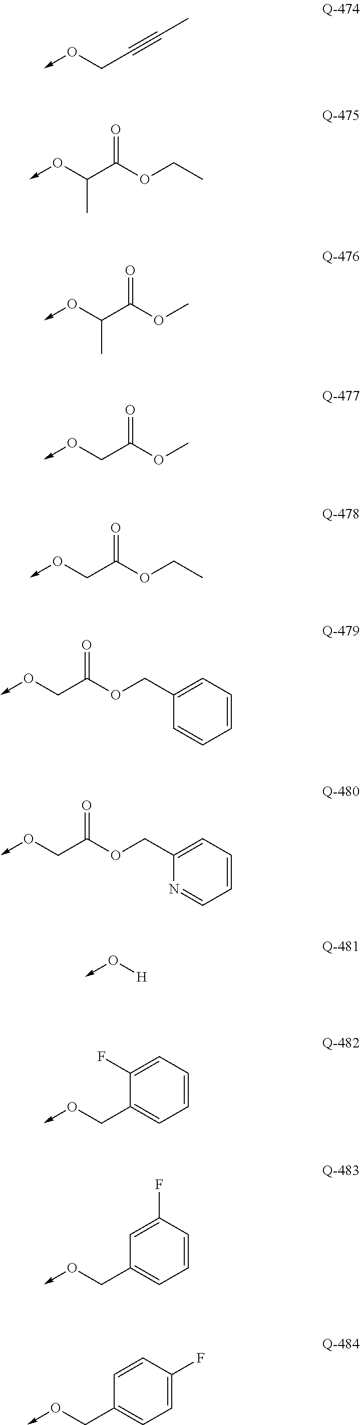

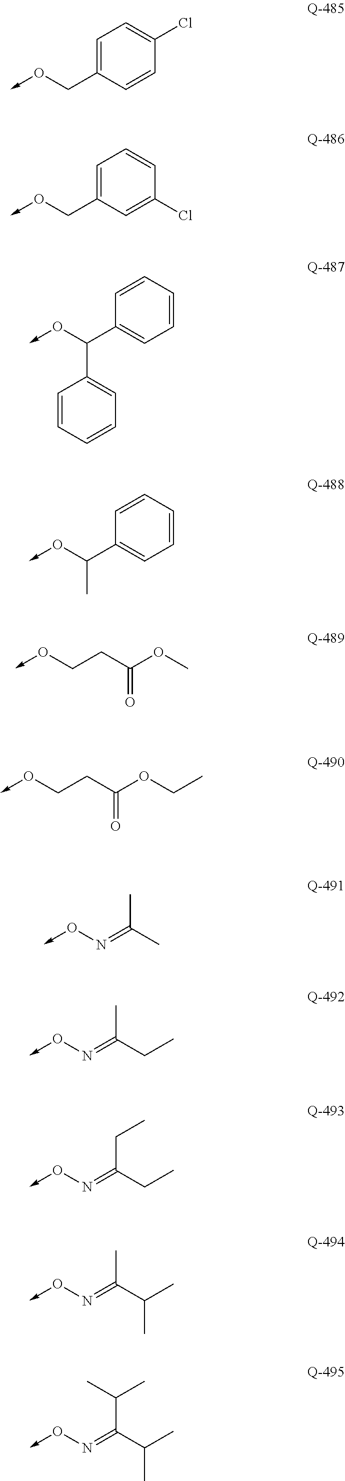

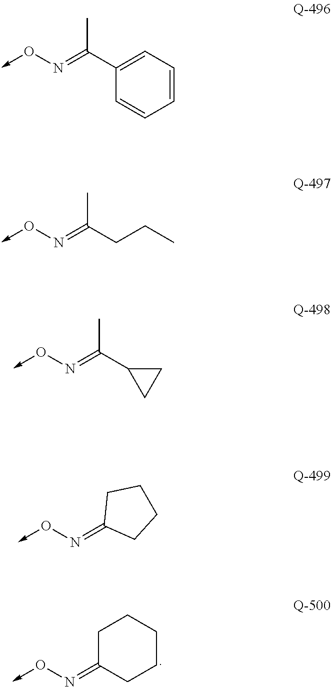

3. The method of claim 1, wherein said compound of the general formula (I) corresponds to any one of the following structures:

| Compound | IUPAC name | Structure |
|---|---|---|
| (a) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 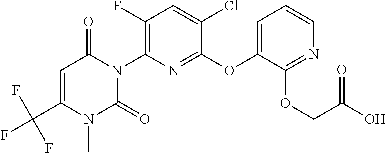 [SMILES: Cn1c(C(F)(F)F)cc(=O)n(-c2nc(Oc3cccnc3OCC(=O)O)c(Cl)cc2F)c1=O] |
| (b) | ethyl {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetate | 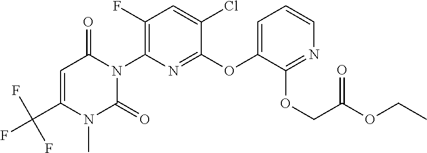 [SMILES: CCOC(=O)COc1ncccc1Oc1nc(-n2c(=O)cc(C(F)(F)F)n(C)c2=O)c(F)cc1Cl] |
| (c) | methyl {[3-({3-chloro-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 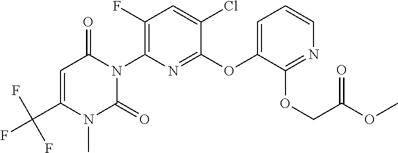 [SMILES: COC(=O)COc1ncccc1Oc1nc(-n2c(=O)cc(C(F)(F)F)n(C)c2=O)c(F)cc1Cl] |
| (d) | pyridazin-3-ylmethyl{[3-({3-chloro-5-fluoro- 6-[3-methyl-2,6-dioxo-4-(trifluoromethyl)- 3,6-dihydropyrimidin-1(2H)-yl]pyridin-2- yl}oxy)pyridin-2-yl]oxy}acetate | 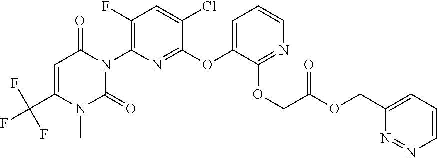 [SMILES: Cn1c(C(F)(F)F)cc(=O)n(-c2nc(Oc3cccnc3OCC(=O)OCc3cccnn3)c(Cl)cc2F)c1=O] |
| (e) | pyridin-2-ylmethyl{[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 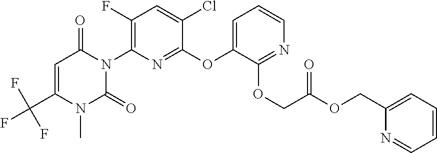 [SMILES: Cn1c(C(F)(F)F)cc(=O)n(-c2nc(Oc3cccnc3OCC(=O)OCc3ccccn3)c(Cl)cc2F)c1=O] |
| (f) | pyridin-4-ylmethyl{[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 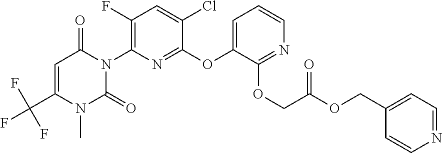 [SMILES: Cn1c(C(F)(F)F)cc(=O)n(-c2nc(Oc3cccnc3OCC(=O)OCc3ccncc3)c(Cl)cc2F)c1=O] |
| (g) | (4-trifluoromethylpyridin-3-yl)methyl{ [3-({3- chloro-5-fluoro-6-[3-methyl-2,6-dioxo-4- (trifluoromethyl)-3,6-dihydropyrimidin- 1(2H)-yl]pyridin-2-yl}oxy)pyridin-2- ylJoxy}acetate | 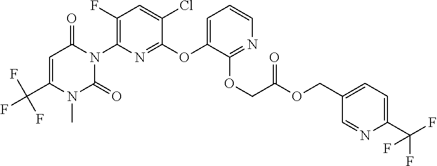 [SMILES: Cn1c(C(F)(F)F)cc(=O)n(-c2nc(Oc3cccnc3OCC(=O)OCc3ccc(C(F)(F)F)nc3)c(Cl)cc2F)c1=O] |
| (h) | ethyl [2-({3-chloro-5-fluoro-6-[3-methyl-2,6- dioxo-4-(trifluoromethyl)-3,6- dihydropyrimidin-1(2H)-yl]pyridin-2- yl}oxy)phenoxy]acetate | 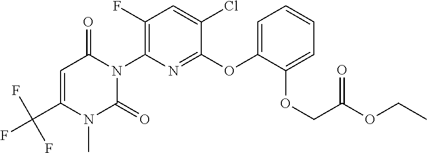 [SMILES: CCOC(=O)COc1ccccc1Oc1nc(-n2c(=O)cc(C(F)(F)F)n(C)c2=O)c(F)cc1Cl] |
| (i) | methyl [2-({3-chloro-5-fluoro-6-[3-methyl- 2,6-dioxo-4-(trifluoromethyl)-3,6- dihydropyrimidin-1(2H)-yl]pyridin-2- yl}oxy)phenoxy]acetate | 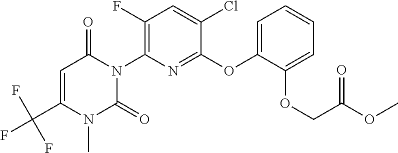 [SMILES: COC(=O)COc1ccccc1Oc1nc(-n2c(=O)cc(C(F)(F)F)n(C)c2=O)c(F)cc1Cl] |
| (j) | [2-({3-chloro-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 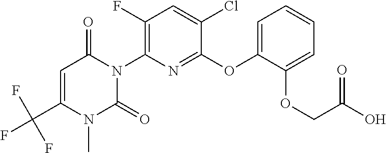 [SMILES: Cn1c(C(F)(F)F)cc(=O)n(-c2nc(Oc3ccccc3OCC(=O)O)c(Cl)cc2F)c1=O] |
| (k) | ethyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 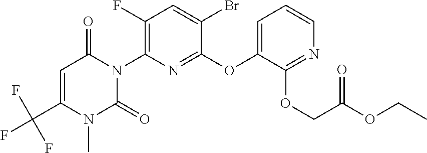 [SMILES: CCOC(=O)COc1ncccc1Oc1nc(-n2c(=O)cc(C(F)(F)F)n(C)c2=O)c(F)cc1Br] |
| (l) | methyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 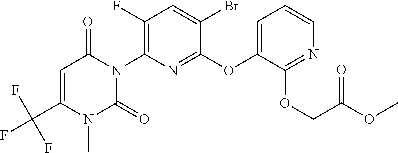 [SMILES: COC(=O)COc1ncccc1Oc1nc(-n2c(=O)cc(C(F)(F)F)n(C)c2=O)c(F)cc1Br] |
| (m) | {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetic acid | 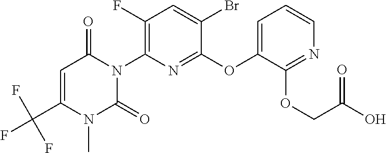 [SMILES: Cn1c(C(F)(F)F)cc(=O)n(-c2nc(Oc3cccnc3OCC(=O)O)c(Br)cc2F)c1=O] |
| (n) | ethyl [2-({3-bromo-5-fluoro-6-[3-methyl-2,6- dioxo-4-(trifluoromethyl)-3,6- dihydropyrimidin-1(2H)-yl]pyridin-2- yl}oxy)phenoxy]acetate | 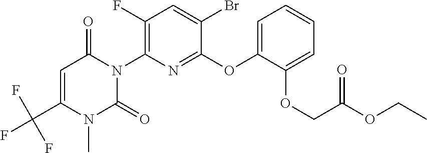 [SMILES: CCOC(=O)COc1ccccc1Oc1nc(-n2c(=O)cc(C(F)(F)F)n(C)c2=O)c(F)cc1Br] |
| (o) | methyl [2-({3-bromo-5-fluoro-6-[3-methyl- 2,6-dioxo-4-(trifluoromethyl)-3,6- dihydropyrimidin-1(2H)-yl]pyridin-2- yl}oxy)phenoxy]acetate | 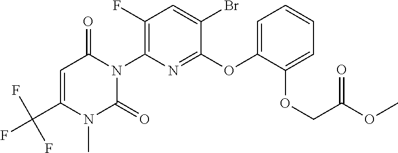 [SMILES: COC(=O)COc1ccccc1Oc1nc(-n2c(=O)cc(C(F)(F)F)n(C)c2=O)c(F)cc1Br] |
| (p) | [2-({3-bromo-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 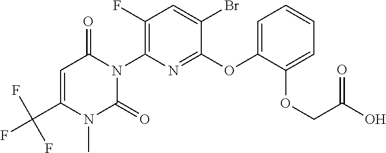 [SMILES: Cn1c(C(F)(F)F)cc(=O)n(-c2nc(Oc3ccccc3OCC(=O)O)c(Br)cc2F)c1=O] |
| (q) | ethyl {[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetate | 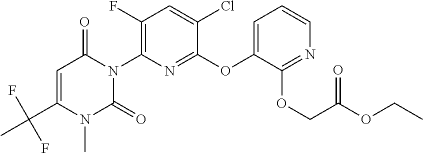 [SMILES: CCOC(=O)COc1ncccc1Oc1nc(-n2c(=O)cc(C(C)(F)F)n(C)c2=O)c(F)cc1Cl] |
| (r) | methyl {[3-({3-chloro-5-fluoro-6-[3-methyl- 2,6-dioxo-4-(1,1-difluoroethyl)-3,6- dihydropyrimidin-1(2H)-yl]pyridin-2- yl}oxy)pyridin-2-yl]oxy}acetate | 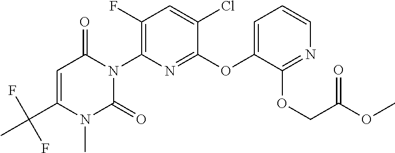 [SMILES: COC(=O)COc1ncccc1Oc1nc(-n2c(=O)cc(C(C)(F)F)n(C)c2=O)c(F)cc1Cl] |
| (s) | {[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetic acid | 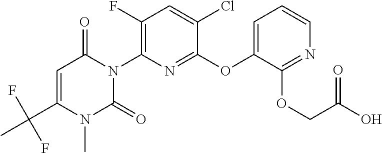 [SMILES: Cn1c(C(C)(F)F)cc(=O)n(-c2nc(Oc3cccnc3OCC(=O)O)c(Cl)cc2F)c1=O] |
| (t) | ethyl [2-({3-chloro-5-fluoro-6-[3-methyl-2,6- dioxo-4-(1,1-difluoroethyl)-3,6- dihydropyrimidin-1(2H)-yl]pyridin-2- yl}oxy)phenoxy]acetate | 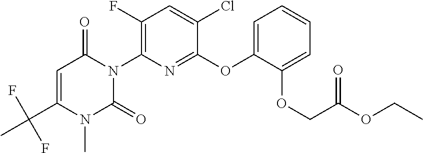 [SMILES: CCOC(=O)COc1ccccc1Oc1nc(-n2c(=O)cc(C(C)(F)F)n(C)c2=O)c(F)cc1Cl] |
| (u) | methyl [2-({3-chloro-5-fluoro-6-[3-methyl- 2,6-dioxo-4-(1,1-difluoroethyl)-3,6- dihydropyrimidin-1(2H)-yl]pyridin-2- yl}oxy)phenoxy]acetate | 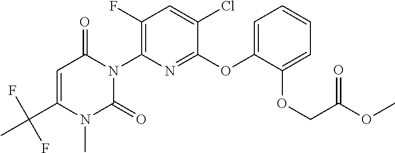 [SMILES: COC(=O)COc1ccccc1Oc1nc(-n2c(=O)cc(C(C)(F)F)n(C)c2=O)c(F)cc1Cl] |
| (v) | [2-({3-chloro-5-fluoro-6-[3-methyl-2,6-dioxo- 4-(1,1-difluoroethyl)-3,6-dihydropyrimidin- 1(2H)-yl]pyridin-2-yl}oxy)phenoxy]acetic acid | 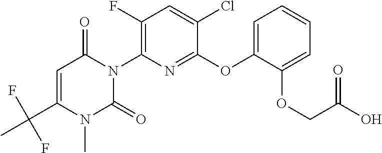 [SMILES: Cn1c(C(C)(F)F)cc(=O)n(-c2nc(Oc3ccccc3OCC(=O)O)c(Cl)cc2F)c1=O] |
| (x) | ethyl {[3-({3-chloro-5-fluoro-6-[3-methyl-2,6- dioxo-4-(trifluoromethyl)-3,6- dihydropyrimidin-1(2H)-yl]pyridin-2- yl}sulfanyl)pyridin-2-yl]oxy}acetate | 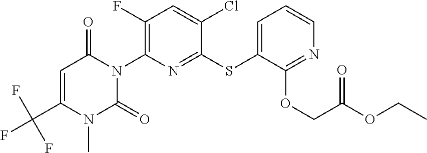 [SMILES: CCOC(=O)COc1ncccc1Sc1nc(-n2c(=O)cc(C(F)(F)F)n(C)c2=O)c(F)cc1Cl] |
| (y) | methyl {[3-({3-chloro-5-fluoro-6-[3-methyl- 2,6-dioxo-4-(trifluoromethyl)-3,6- dihydropyrimidin-1(2H)-yl]pyridin-2- yl}sulfanyl)pyridin-2-yl]oxy}acetate | 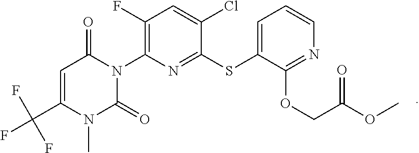 [SMILES: COC(=O)COc1ncccc1Sc1nc(-n2c(=O)cc(C(F)(F)F)n(C)c2=O)c(F)cc1Cl] |

4. The method of claim 3, wherein said compound corresponds to:
(a) ethyl {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetate;
(b) methyl {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetate;
(c) {[3-({3-chloro-5-fluoro-6-[3-methyl-2,6-dioxo-4-(trifluoromethyl)-3,6-dihydropyrimidin-1(2H)-yl]pyridin-2-yl}oxy)pyridin-2-yl]oxy}acetic acid;
(h) ethyl [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetate;
(i) methyl [2-({3-chloro-5-fluoro-6-[3-methyl-2,6-dioxo-4-(trifluoromethyl)-3,6-dihydropyrimidin-1(2H)-yl}pyridin-2-yl]oxy)phenoxy]acetate;
(j) [2-({3-chloro-5-fluoro-6-[3-methyl-2,6-dioxo-4-(trifluoromethyl)-3,6-dihydropyrimidin-1(2H)-yl]pyridin-2-yl}oxy)phenoxy]acetic acid;
(x) ethyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate;
(y) methyl {[3-({3-chloro-5-fluoro-6-[3-methyl-2,6-dioxo-4-(trifluoromethyl)-3,6-dihydropyrimidin-1(2H)-yl]pyridin-2-yl}sulfanyl)pyridin-2-yl]oxy}acetate.

5. The method of any of claims 1-4, wherein the heterologous HemG protein has herbicide-insensitive protoporphyrinogen oxidase activity.

6. The method of any of claims 1-5, wherein the heterologous HemG protein has at least 85% sequence identity to a polypeptide sequence selected from the group consisting of SEQ ID NOs:1-20 and 65-193.

7. The method of any of claims 1-6, wherein the DNA sequence encoding the heterologous HemG protein is selected from the group consisting of SEQ ID NOs:22-64 and 194-322.

8. The method of any of claims 1-7, wherein the heterologous HemG protein comprises an amino acid sequence selected from the group consisting of SEQ ID NOs:1-20 and 65-193.

9. The method of any of claims 1-8, wherein the DNA sequence encoding a heterologous HemG protein is operably linked to a DNA sequence encoding a chloroplast transit peptide (CTP).

10. The method of claim 9, wherein the CTP comprises an amino acid sequence with at least 97% sequence identity to a sequence selected from the group consisting of SEQ ID NOs:323-328, 340, and 342-407.

11. The method of claim 9, wherein the DNA sequence encoding the CTP comprises at least 97% identity to a sequence selected from the group consisting of SEQ ID NOs:329-339, 341, and 408-483.

12. The method of any of claims 1-11, wherein said recombinant DNA molecule further comprises a heterologous promoter operably linked to the DNA sequence encoding said HemG protein.

13. The method of any of claims 1-12, wherein said herbicidally active compound is applied to the area at a rate of about 0.02 g a.i./ha to about 750 g a.i./ha, about 0.05 g a.i./ha to about 400 g a.i./ha, or about 0.25 g a.i./ha to about 300 g a.i./ha.

14. The method of any of claims 1-13 further defined as comprising applying said compound to said area at least twice.

15. The method of any of claims 1-14, wherein the herbicidally active compound is applied in an amount that does not damage said plant comprising the recombinant DNA molecule.

16. The method of any of claims 1-15, wherein the plant comprising the recombinant DNA molecule is a monocotyledonous plant or wherein the plant comprising the recombinant DNA molecule is a dicotyledonous plant.

17. The method of any of claims 1-16, wherein the method further comprises applying to said area an effective amount of at least a second herbicide.

18. The method of claim 17, wherein the second herbicide is selected from the group consisting of: an ACCase inhibitor, an ALS inhibitor, an EPSPS inhibitor, a synthetic auxin, a photosynthesis inhibitor, a glutamine synthesis inhibitor, a HPPD inhibitor, a PPO inhibitor, and a long-chain fatty acid inhibitor.

19. The method of claim 18, wherein the ACCase inhibitor is an aryloxyphenoxy propionate or a cyclohexanedione; the ALS inhibitor is a sulfonylurea, imidazolinone, triazoloyrimidine, or a triazolinone; the EPSPS inhibitor is glyphosate; the synthetic auxin is a phenoxy herbicide, a benzoic acid, a carboxylic acid, or a semicarbazone; the photosynthesis inhibitor is a triazine, a triazinone, a nitrile, a benzothiadiazole, or a urea; the glutamine synthesis inhibitor is glufosinate; the HPPD inhibitor is an isoxazole, a pyrazolone, or a triketone; the PPO inhibitor is a diphenylether, a N-phenylphthalimide, an aryl triazinone, or a pyrimidinedione; or the long-chain fatty acid inhibitor is a chloroacetamide, an oxyacetamide, or a pyrazole.

20. The method of any of claims 1-19, wherein said applying of the compound is carried out pre-emergence.

21. The method of any of claims 1-19, wherein said applying of the compound is carried out post-emergence.

22. The method of any of claims 1-21, wherein said applying of the compound comprises contacting said plant with the compound.

23. The method of any of claims 1-22, wherein said applying of the compound comprises an over the top application of said compound.

24. The method of any of claims 1-23, wherein said applying of the compound results in an increase in the growth or yield of said plant relative to a plant of the same genotype cultivated in a growth area in which said compound has not been applied.


SEQUENCE LISTING (483 sequences)

SEQ ID NO: 1 - protein, 179 aa, Enterobacter cloacae [sequence omitted]

SEQ ID NO: 2 - protein, 178 aa, Pantoea ananatis
MKALILFSTRDGQTQKIASAIADEIKGQQSCDVINIQDAKTLDWQQYDRVLIGASIRYGHFQPVVNEFVKHNLLALQQRVSGFFSVNLTARKPEKRSPETNAYTVKFLAQSPWQPDCCAVFAGALYYPRYRWFDRVMIQFIMRMTGGETDASKEVEYTDWQQVQRFARDFAQLPGKSY

SEQ ID NO: 3 - protein, 177 aa, Pantoea stewartii
MKALILYSTRDGQTRKIASSIADVIRQQQQCDVLNIKDASLPDWAQYDRVLIGASIRYGHFQPVVDKFVKQHLHELQQRTSGFFSVNLTARKPEKRSPETNAYTQKFLAHSPWQPDCCAVFAGALYYPRYRWFDRVMIQLIMRMTGGETDSTKEVEYTDWQQVSTFANDFAQLPGKS

SEQ ID NO: 4 - protein, 181 aa, Escherichia coli
MKTLILFSTRDGQTREIASYLASELKELGIQADVANVHRIEEPQWENYDRVVIGASIRYGHYHSAFQEFVKKHATRLNSMPSAFYSVNLVARKPEKRTPQTNSYARKFLMNSQWRPDRCAVIAGALRYPRYRWYDRFMIKLIMKMSGGETDTRKEVVYTDWEQVANFAREIAHLTDKPTLK

SEQ ID NO: 5 - protein, 178 aa, Erwinia toletana
MKALILFSSREGQTREIASYIANSIKEEMECDVFNILRVEQIDWSQYDRVLIGGSIHYGHFHPAVAKFVKRHLHELQQRSSGFFCVNLTARKADKRTPQTNAYMRKFLLQSPWQPDCCAVFAGALRYTRYRWFDRVMIQLIMRMTGGETDTSKEVEYTDWTQVARFAQEFAHLPGKTQ

SEQ ID NO: 6 - protein, 179 aa, Pectobacterium carotovorum
MKALIVFSSRDGQTRAIASYIANTLKGTLECDVVNVLNANDIDLSQYDRVAIGASIRYGRFHPAVNQFIRKHLTSLQQLPSAFFSVNLTARKPEKRTIQTNAYTRKFLLNSPWQPDLCCVFAGALRYPRYRWFDRVMIQLIMRITGGETDSTKEIEYTDWQQVARFAQDFAQLAAKNPA

SEQ ID NO: 7 - protein, 179 aa, Shimwellia blattae
MKTLILFSTRDGQTHKIARHIAGVLEEQGKACELVDLLQPGEPDWSTVECVVLGASIRYGHFHKSFIRFVNTHAQRLNNMPGALFTVNLVARKPEKQSPQTNSYTRKFLAASPWQPQRCQVFAGALRYPRYSWYDRMMIRLIMKMAGGETDTRKEVEYTDWQSVTRFAREIAQLPGETR

SEQ ID NO: 8 - protein, 178 aa, Pantoea stewartii
MKALILFSSRDGQTQLIASSIAKELEGKQACDVLNILDTTNVEWTQYDRVLIGASIRYGHFHPAVAEFVKRHQRELQQRSSGFFSVNLTARKPEKRSPETNAYTAKFLNQSPWQPDCCAVFAGALRYPRYRWFDRIMIQLIMRMTGGETDSSKEVEYTDWQQVTRFAQEFARLPGKTS

SEQ ID NO: 9 - protein, 180 aa, Enterobacter cloacae
MKTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQVASFAREIVQLTRSSRL

SEQ ID NO: 10 - protein, 177 aa, Enterobacter mori
MKILILFSTRDGQTREIAASLASELKEQAFDVDVVNLHRAENIAWEEYDGVVIGASIRYGHFHSTLNSFVKKHQQALKKLPGAFYSVNLVARKPEKRTPQTNSYTRKFLLDSPWQPDLSAVFAGALRYPRYNWYDRIMIRLIMKITGGETDTRKEVVYTDWQQVTHFAHEIVQLVRK

SEQ ID NO: 11 - protein, 178 aa, Enterobacter cloacae
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 12 - protein, 177 aa, Pantoea ananatis
KALILFSTRDGQTQKIASAIADEIKGQQSCDVINIQDAKTLDWQQYDRVLIGASIRYGHFQPVVNEFVKHNLLALQQRVSGFFSVNLTARKPEKRSPETNAYTVKFLAQSPWQPDCCAVFAGALYYPRYRWFDRVMIQFIMRMTGGETDASKEVEYTDWQQVQRFARDFAQLPGKSY

SEQ ID NO: 13 - protein, 180 aa, Escherichia coli
KTLILFSTRDGQTREIASYLASELKELGIQADVANVHRIEEPQWENYDRVVIGASIRYGHYHSAFQEFVKKHATRLNSMPSAFYSVNLVARKPEKRTPQTNSYARKFLMNSQWRPDRCAVIAGALRYPRYRWYDRFMIKLIMKMSGGETDTRKEVVYTDWEQVANFAREIAHLTDKPTLK

SEQ ID NO: 14 - protein, 177 aa, Erwinia toletana
KALILFSSREGQTREIASYIANSIKEEMECDVFNILRVEQIDWSQYDRVLIGGSIHYGHFHPAVAKFVKRHLHELQQRSSGFFCVNLTARKADKRTPQTNAYMRKFLLQSPWQPDCCAVFAGALRYTRYRWFDRVMIQLIMRMTGGETDTSKEVEYTDWTQVARFAQEFAHLPGKTQ

SEQ ID NO: 15 - protein, 178 aa, Pectobacterium carotovorum
KALIVFSSRDGQTRAIASYIANTLKGTLECDVVNVLNANDIDLSQYDRVAIGASIRYGRFHPAVNQFIRKHLTSLQQLPSAFFSVNLTARKPEKRTIQTNAYTRKFLLNSPWQPDLCCVFAGALRYPRYRWFDRVMIQLIMRITGGETDSTKEIEYTDWQQVARFAQDFAQLAAKNPA

SEQ ID NO: 16 - protein, 178 aa, Shimwellia blattae
KTLILFSTRDGQTHKIARHIAGVLEEQGKACELVDLLQPGEPDWSTVECVVLGASIRYGHFHKSFIRFVNTHAQRLNNMPGALFTVNLVARKPEKQSPQTNSYTRKFLAASPWQPQRCQVFAGALRYPRYSWYDRMMIRLIMKMAGGETDTRKEVEYTDWQSVTRFAREIAQLPGETR

SEQ ID NO: 17 - protein, 179 aa, Enterobacter cloacae
KTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQVASFAREIVQLTRSSRL

SEQ ID NO: 18 - protein, 178 aa, synthetic construct
  REGION 1..178: note = Protein variant
KALVLYSTRDGQTHAIASYIASCMKEKAECDVIDLTHGEHVNLTQYDQVLIGANIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 19 - protein, 172 aa, synthetic construct
  REGION 1..172: note = Protein variant
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKL

SEQ ID NO: 20 - protein, 179 aa, synthetic construct
  REGION 1..179: note = Protein variant
KTLILFSTRDGQTREIAAFLASELKEQGIYADVINLNRTEEIAWQEYDRVVIGASIRYGHFHPAVDRFVKKHTETLNSLPGAFFSVNLVARKAEKRTPQTNSYTRKFLLNSPWKPAACAVFAGALRYPRYRWYDRFMIRLIMKMTGGETDTRKEVVYTDWSQIASFAREIVQLTRSSRL

SEQ ID NO: 21 - protein, 504 aa, Amaranthus tuberculatus
MGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 22 - DNA, 537 bp, synthetic construct
  misc_feature 1..537: note = Codon optimized for E. Coli expression
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 23 - DNA, 534 bp, synthetic construct
  misc_feature 1..534: note = Codon optimized for E. Coli expression
  source 1..534: mol_type = other DNA
atgaaagcgctgattctgtttagcacccgcgatggccagacccagaaaattgcgagcgcgattgcggatgaaattaaaggccagcagagctgcgatgtgattaacattcaggatgcgaaaaccctggattggcagcagtatgatcgcgtgctgattggcgcgagcattcgctatggccattttcagccggtggtgaacgaatttgtgaaacataacctgctggcgctgcagcagcgcgtgagcggcttttttagcgtgaacctgaccgcgcgcaaaccggaaaaacgcagcccggaaaccaacgcgtataccgtgaaatttctggcgcagagcccgtggcagccggattgctgcgcggtgtttgcgggcgcgctgtattatccgcgctatcgctggtttgatcgcgtgatgattcagtttattatgcgcatgaccggcggcgaaaccgatgcgagcaaagaagtggaatataccgattggcagcaggtgcagcgctttgcgcgcgattttgcgcagctgccgggcaaaagctat

SEQ ID NO: 24 - DNA, 531 bp, synthetic construct
  misc_feature 1..531: note = Codon optimized for E. Coli expression
  source 1..531: mol_type = other DNA
atgaaagcgctgattctgtatagcacccgcgatggccagacccgcaaaattgcgagcagcattgcggatgtgattcgccagcagcagcagtgcgatgtgctgaacattaaagatgcgagcctgccggattgggcgcagtatgatcgcgtgctgattggcgcgagcattcgctatggccattttcagccggtggtggataaatttgtgaaacagcatctgcatgaactgcagcagcgcaccagcggcttttttagcgtgaacctgaccgcgcgcaaaccggaaaaacgcagcccggaaaccaacgcgtatacccagaaatttctggcgcatagcccgtggcagccggattgctgcgcggtgtttgcgggcgcgctgtattatccgcgctatcgctggtttgatcgcgtgatgattcagctgattatgcgcatgaccggcggcgaaaccgatagcaccaaagaagtggaatataccgattggcagcaggtgagcacctttgcgaacgattttgcgcagctgccgggcaaaagc

SEQ ID NO: 25 - DNA, 546 bp, Escherichia coli
  source 1..546: mol_type = genomic DNA
gtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataa

SEQ ID NO: 26 - DNA, 534 bp, synthetic construct
  misc_feature 1..534: note = Codon optimized for E. Coli expression
  source 1..534: mol_type = other DNA
atgaaagcgctgattctgtttagcagccgcgaaggccagacccgcgaaattgcgagctatattgcgaacagcattaaagaagaaatggaatgcgatgtgtttaacattctgcgcgtggaacagattgattggagccagtatgatcgcgtgctgattggcggcagcattcattatggccattttcatccggcggtggcgaaatttgtgaaacgccatctgcatgaactgcagcagcgcagcagcggctttttttgcgtgaacctgaccgcgcgcaaagcggataaacgcaccccgcagaccaacgcgtatatgcgcaaatttctgctgcagagcccgtggcagccggattgctgcgcggtgtttgcgggcgcgctgcgctatacccgctatcgctggtttgatcgcgtgatgattcagctgattatgcgcatgaccggcggcgaaaccgataccagcaaagaagtggaatataccgattggacccaggtggcgcgctttgcgcaggaatttgcgcatctgccgggcaaaacccag

SEQ ID NO: 27 - DNA, 537 bp, synthetic construct
  misc_feature 1..537: note = Codon optimized for E. Coli expression
  source 1..537: mol_type = other DNA
atgaaagcgctgattgtgtttagcagccgcgatggccagacccgcgcgattgcgagctatattgcgaacaccctgaaaggcaccctggaatgcgatgtggtgaacgtgctgaacgcgaacgatattgatctgagccagtatgatcgcgtggcgattggcgcgagcattcgctatggccgctttcatccggcggtgaaccagtttattcgcaaacatctgaccagcctgcagcagctgccgagcgcgttttttagcgtgaacctgaccgcgcgcaaaccggaaaaacgcaccattcagaccaacgcgtatacccgcaaatttctgctgaacagcccgtggcagccggatctgtgctgcgtgtttgcgggcgcgctgcgctatccgcgctatcgctggtttgatcgcgtgatgattcagctgattatgcgcattaccggcggcgaaaccgatagcaccaaagaaattgaatataccgattggcagcaggtggcgcgctttgcgcaggattttgcgcagctggcggcgaaaaacccggcg

SEQ ID NO: 28 - DNA, 537 bp, synthetic construct
  misc_feature 1..537: note = Codon optimized for E. Coli expression
  source 1..537: mol_type = other DNA
atgaagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcgg

SEQ ID NO: 29 - DNA, 534 bp, synthetic construct
  misc_feature 1..534: note = Codon optimized for E. Coli expression
  source 1..534: mol_type = other DNA
atgaaagcgctgattctgtttagcagccgcgatggccagacccagctgattgcgagcagcattgcgaaagaactggaaggcaaacaggcgtgcgatgtgctgaacattctggataccaccaacgtggaatggacccagtatgatcgcgtgctgattggcgcgagcattcgctatggccattttcatccggcggtggcggaatttgtgaaacgccatcagcgcgaactgcagcagcgcagcagcggcttttttagcgtgaacctgaccgcgcgcaaaccggaaaaacgcagcccggaaaccaacgcgtataccgcgaaatttctgaaccagagcccgtggcagccggattgctgcgcggtgtttgcgggcgcgctgcgctatccgcgctatcgctggtttgatcgcattatgattcagctgattatgcgcatgaccggcggcgaaaccgatagcagcaaagaagtggaatataccgattggcagcaggtgacccgctttgcgcaggaatttgcgcgcctgccgggcaaaaccagc

SEQ ID NO: 30 - DNA, 540 bp, synthetic construct
  misc_feature 1..540: note = Codon optimized for E. Coli expression
  source 1..540: mol_type = other DNA
atgaaaaccctgattctgtttagcacccgcgatggccagacccgcgaaattgcggcgtttctggcgagcgaactgaaagaacagggcatttatgcggatgtgattaacctgaaccgcaccgaagaaattgcgtggcaggaatatgatcgcgtggtgattggcgcgagcattcgctatggccattttcatccggcggtggatcgctttgtgaaaaaacataccgaaaccctgaacagcctgccgggcgcgttttttagcgtgaacctggtggcgcgcaaagcggaaaaacgcaccccgcagaccaacagctatacccgcaaatttctgctgaacagcccgtggaaaccggcggcgtgcgcggtgtttgcgggcgcgctgcgctatccgcgctatcgctggtatgatcgctttatgattcgcctgattatgaaaatgaccggcggcgaaaccgatacccgcaaagaagtggtgtataccgattggagccaggtggcgagctttgcgcgcgaaattgtgcagctgacccgcagcagccgcctg

SEQ ID NO: 31 - DNA, 531 bp, synthetic construct
  misc_feature 1..531: note = Codon optimized for E. Coli expression
  source 1..531: mol_type = other DNA
atgaaaattctgattctgtttagcacccgcgatggccagacccgcgaaattgcggcgagcctggcgagcgaactgaaagaacaggcgtttgatgtggatgtggtgaacctgcatcgcgcggaaaacattgcgtgggaagaatatgatggcgtggtgattggcgcgagcattcgctatggccattttcatagcaccctgaacagctttgtgaaaaaacatcagcaggcgctgaaaaaactgccgggcgcgttttatagcgtgaacctggtggcgcgcaaaccggaaaaacgcaccccgcagaccaacagctatacccgcaaatttctgctggatagcccgtggcagccggatctgagcgcggtgtttgcgggcgcgctgcgctatccgcgctataactggtatgatcgcattatgattcgcctgattatgaaaattaccggcggcgaaaccgatacccgcaaagaagtggtgtataccgattggcagcaggtgacccattttgcgcatgaaattgtgcagctggtgcgcaaa

SEQ ID NO: 32 - DNA, 540 bp, synthetic construct
  misc_feature 1..540: note = Codon optimized for dicot expression
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 33 - DNA, 537 bp, synthetic construct
  misc_feature 1..537: note = Codon optimized for dicot expression
  source 1..537: mol_type = other DNA
atgaaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggagagaccgacgcatcgaaagaggtggagtacactgactggcagcaggtgcagcggttcgcgcgagacttcgcgcagttaccgggtaagtcctactga

SEQ ID NO: 34 - DNA, 534 bp, synthetic construct
  misc_feature 1..534: note = Codon optimized for dicot expression
  source 1..534: mol_type = other DNA
atgaaggcgctgatcttgtactcaaccagggacggtcagactcgcaagattgcaagtagcattgcggacgtcatcaggcagcagcagcagtgcgacgtcttaaacattaaagacgcatcacttcctgactgggcccaatatgaccgagtgctcatcggagctagcatccgttacgggcatttccagcccgttgtagacaagttcgtgaagcagcacttgcacgagcttcagcagcggacctccggcttcttctccgtgaacctgacggcgaggaagcctgaaaaaaggagccctgagaccaatgcctacacccagaaattcttggcgcactccccttggcagcccgattgctgtgccgttttcgcgggggccctttactaccccaggtaccgttggttcgaccgggtgatgatccagttgattatgcgcatgactggtggagagaccgactctaccaaggaagtggagtacactgactggcagcaggtgagtaccttcgccaacgattttgcccagcttccaggcaagagctaa

SEQ ID NO: 35 - DNA, 546 bp, synthetic construct
  misc_feature 1..546: note = Codon optimized for dicot expression
  source 1..546: mol_type = other DNA
atgaagaccttgattctattctccacaagggacggccagactagggagatcgcttcctacctggccagcgagctaaaggagcttggcattcaggcagacgtggctaacgtgcaccgaattgaggagccgcagtgggagaactacgatcgggtcgtgatcggcgccagcatccggtatggacactaccacagcgcgttccaggagttcgtgaaaaagcacgcgacccgtctgaatagcatgccatcagcgttctactcggtcaacctcgtggctcgtaagcccgagaagcggacaccccagaccaactcgtatgccaggaagttccttatgaactcgcagtggcgaccggaccgctgcgcggtgatcgccggtgcgctcaggtaccctcgttataggtggtacgacaggtttatgattaaacttataatgaaaatgagcggcggagagaccgacaccagaaaagaggtggtttacacagactgggagcaggtagcaaacttcgctagggagattgctcacctcaccgacaagccgaccttgaagtaa

SEQ ID NO: 36 - DNA, 537 bp, synthetic construct
  misc_feature 1..537: note = Codon optimized for dicot expression
  source 1..537: mol_type = other DNA
atgaaggcccttatactgttcagttccagagaaggccagacgagggagatagcgagttacattgccaactcgataaaggaggaaatggaatgcgacgtgttcaacatccttcgtgtggagcagatcgactggtctcaatacgaccgcgtcctgatcgggggctcgatacactacggccatttccacccagcggtggcaaaatttgtcaagaggcacctccatgagttgcaacagaggtcttccggctttttctgcgtcaacctgacggccaggaaggccgacaagcggactccccagaccaatgcctacatgagaaagttcttgttgcagtccccatggcaacccgattgctgcgccgtgtttgcgggggcccttaggtacacccgttacaggtggttcgacagggtaatgattcagctgatcatgaggatgacgggcggagagactgacacatcgaaggaggtggagtacacagactggacgcaggtcgcccgcttcgcgcaggagttcgcccatttgcccggcaaaactcagtga

SEQ ID NO: 37 - DNA, 540 bp, synthetic construct
  misc_feature 1..540: note = Codon optimized for dicot expression
  source 1..540: mol_type = other DNA
atgaaggctcttatcgtattctcttcgagggatggccaaacccgagcgatcgcgtcttatattgctaataccctcaaagggaccctagagtgcgacgtcgtcaacgtcctcaatgctaacgacattgatttgagccagtacgaccgtgtggccattggcgcctccattcgctacgggaggttccacccagctgttaaccagtttatccggaagcaccttacgagcctccagcagctaccatctgcgttcttctccgtgaacctcacagctcggaagcccgagaagaggactatacaaaccaacgcgtacactaggaagtttctactgaactcgccgtggcagccggacctgtgctgcgtgttcgcgggagcccttcgctatccccgttacaggtggtttgaccgagtgatgattcaactcataatgcgcataacggggggcgagacagactccaccaaggagatcgagtacaccgactggcagcaggtcgcgcgattcgcccaggattttgcacagcttgccgcaaagaacccggcatga

SEQ ID NO: 38 - DNA, 540 bp, synthetic construct
  misc_feature 1..540: note = Codon optimized for dicot expression
  source 1..540: mol_type = other DNA
atgaagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcggtag

SEQ ID NO: 39 - DNA, 537 bp, synthetic construct
  misc_feature 1..537: note = Codon optimized for dicot expression
  source 1..537: mol_type = other DNA
atgaaggccctaattttattcagtagtagggacggccagacccagcttatagcatcgtctatcgccaaggagctcgaagggaagcaggcgtgcgacgtgttgaatatcctcgacacgactaatgtggagtggacccagtacgaccgcgtgctgattggagcatccatccggtacgggcactttcaccctgcggtcgccgagttcgtaaagcgtcaccagcgagagctacagcagagaagtagtggctttttctctgtgaacttgacggcccgtaagccggaaaagaggtcccccgagactaacgcctataccgccaagttccttaaccaaagtccatggcagcctgactgttgcgctgtgttcgctggggctttgcgataccctcggtaccgctggttcgacagaattatgatccagctaatcatgcggatgactgggggtgagacagattcttcaaaggaggtcgagtacaccgactggcagcaggtgacccgcttcgcgcaagagttcgccaggcttccgggaaagaccagttga

SEQ ID NO: 40 - DNA, 543 bp, synthetic construct
  misc_feature 1..543: note = Codon optimized for dicot expression
  source 1..543: mol_type = other DNA
atgaagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcaggtcgcgtcgtttgccagagagatagtccagttgaccaggtcatcgcgcttgtga

SEQ ID NO: 41 - DNA, 534 bp, synthetic construct
  misc_feature 1..534: note = Codon optimized for dicot expression
  source 1..534: mol_type = other DNA
atgaagatattaatccttttctccacccgtgacggccaaacccgtgagattgcggcgtccttggcgtccgaactcaaggagcaggcattcgacgtggacgtcgtcaaccttcaccgggccgagaacatcgcatgggaggagtacgacggtgttgtcatcggagcgtccatcaggtacggccactttcatagtaccctgaactcatttgtcaagaagcatcagcaggctcttaagaagcttcccggggctttctacagcgtgaacctcgtcgcccggaagcctgagaagcgcacaccgcagaccaatagctacacccgcaagttcctcttggattccccgtggcagcccgacctttcagccgtgttcgccggggcactcaggtaccctcggtacaattggtacgaccgtatcatgattagacttatcatgaagattacaggcggcgagactgataccaggaaggaagtagtctacacagactggcagcaggtcactcactttgctcacgagatcgtccagctcgtgcggaagtag

SEQ ID NO: 42 - DNA, 537 bp, synthetic construct
  misc_feature 1..537: note = Codon optimized for dicot expression
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 43 - DNA, 534 bp, synthetic construct
  misc_feature 1..534: note = Codon optimized for dicot expression
  source 1..534: mol_type = other DNA
aaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggagagaccgacgcatcgaaagaggtggagtacactgactggcagcaggtgcagcggttcgcgcgagacttcgcgcagttaccgggtaagtcctactga

SEQ ID NO: 44 - DNA, 543 bp, synthetic construct
  misc_feature 1..543: note = Codon optimized for dicot expression
  source 1..543: mol_type = other DNA
aagaccttgattctattctccacaagggacggccagactagggagatcgcttcctacctggccagcgagctaaaggagcttggcattcaggcagacgtggctaacgtgcaccgaattgaggagccgcagtgggagaactacgatcgggtcgtgatcggcgccagcatccggtatggacactaccacagcgcgttccaggagttcgtgaaaaagcacgcgacccgtctgaatagcatgccatcagcgttctactcggtcaacctcgtggctcgtaagcccgagaagcggacaccccagaccaactcgtatgccaggaagttccttatgaactcgcagtggcgaccggaccgctgcgcggtgatcgccggtgcgctcaggtaccctcgttataggtggtacgacaggtttatgattaaacttataatgaaaatgagcggcggagagaccgacaccagaaaagaggtggtttacacagactgggagcaggtagcaaacttcgctagggagattgctcacctcaccgacaagccgaccttgaagtaa

SEQ ID NO: 45 - DNA, 534 bp, synthetic construct
  misc_feature 1..534: note = Codon optimized for dicot expression
  source 1..534: mol_type = other DNA
aaggcccttatactgttcagttccagagaaggccagacgagggagatagcgagttacattgccaactcgataaaggaggaaatggaatgcgacgtgttcaacatccttcgtgtggagcagatcgactggtctcaatacgaccgcgtcctgatcgggggctcgatacactacggccatttccacccagcggtggcaaaatttgtcaagaggcacctccatgagttgcaacagaggtcttccggctttttctgcgtcaacctgacggccaggaaggccgacaagcggactccccagaccaatgcctacatgagaaagttcttgttgcagtccccatggcaacccgattgctgcgccgtgtttgcgggggcccttaggtacacccgttacaggtggttcgacagggtaatgattcagctgatcatgaggatgacgggcggagagactgacacatcgaaggaggtggagtacacagactggacgcaggtcgcccgcttcgcgcaggagttcgcccatttgcccggcaaaactcagtga

SEQ ID NO: 46 - DNA, 537 bp, synthetic construct
  misc_feature 1..537: note = Codon optimized for dicot expression
  source 1..537: mol_type = other DNA
aaggctcttatcgtattctcttcgagggatggccaaacccgagcgatcgcgtcttatattgctaataccctcaaagggaccctagagtgcgacgtcgtcaacgtcctcaatgctaacgacattgatttgagccagtacgaccgtgtggccattggcgcctccattcgctacgggaggttccacccagctgttaaccagtttatccggaagcaccttacgagcctccagcagctaccatctgcgttcttctccgtgaacctcacagctcggaagcccgagaagaggactatacaaaccaacgcgtacactaggaagtttctactgaactcgccgtggcagccggacctgtgctgcgtgttcgcgggagcccttcgctatccccgttacaggtggtttgaccgagtgatgattcaactcataatgcgcataacggggggcgagacagactccaccaaggagatcgagtacaccgactggcagcaggtcgcgcgattcgcccaggattttgcacagcttgccgcaaagaacccggcatga

SEQ ID NO: 47 - DNA, 537 bp, synthetic construct
  misc_feature 1..537: note = Codon optimized for dicot expression
  source 1..537: mol_type = other DNA
aagaccttgatcctattctccaccagggacggccaaacacacaagatcgcaaggcacatcgcaggagtcctcgaagagcaggggaaggcctgcgagttggtcgatctgttacagcccggcgaaccagactggagtaccgttgaatgcgtcgttctaggggccagcattagatatggtcacttccataagtctttcatcaggttcgtaaacactcacgcgcagcgcttgaataatatgccaggcgcccttttcacagttaacttagtcgcccgaaagcccgagaagcagagtccacagacgaactcttacacccgcaagtttctcgccgcctccccttggcagccacagcgatgccaagttttcgcgggcgctttgaggtaccctaggtactcgtggtacgacagaatgatgatacgtttgataatgaagatggccgggggcgagactgacacaaggaaggaggttgagtacactgactggcagtcggtgactcggttcgcgagggagatcgctcagctgccgggagagacgcggtag

SEQ ID NO: 48 - DNA, 540 bp, synthetic construct
  misc_feature 1..540: note = Codon optimized for dicot expression
  source 1..540: mol_type = other DNA
aagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcaggtcgcgtcgtttgccagagagatagtccagttgaccaggtcatcgcgcttgtga

SEQ ID NO: 49 - DNA, 537 bp, synthetic construct
  misc_feature 1..537: note = Recombinant
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacggcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 50 - DNA, 537 bp, synthetic construct
  misc_feature 1..537: note = Recombinant
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcaactgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 51 - DNA, 534 bp, synthetic construct
  misc_feature 1..534: note = Recombinant
  source 1..534: mol_type = other DNA
aaggccttgatcctgttctctacacgcgacggacagacacagaagatcgcatctgccatcgctgatgagataaaggggcagcaatcgtgcgacgtgattaacatacaggatgccaaaaccctcgactggcagcagtacgaccgggtactcatcggcgcctccattcgttacgggcatttccagcccgttgtgaatgagtttgtcaagcacaacctcttggccctacagcagagagtttccggattcttctccgtgaacttgacagcccgaaagccagagaagcggagccccgagactaacgcttatacagtcaaattcttggcgcagtcaccctggcaaccggactgctgcgctgtttttgcgggggccctgtactacccacggtaccggtggttcgatagggtgatgatacagttcataatgcgaatgacggggggggagaccgacgcatcgaaagaggtggagtacactgactggcagcaggtgcagcggttcgcgcgagacttcgcgcagttaccgggtaagtcctactga

SEQ ID NO: 52 - DNA, 537 bp, synthetic construct
  misc_feature 1..537: note = Recombinant
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgaatattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 53 - DNA, 537 bp, synthetic construct
  misc_feature 1..537: note = Recombinant
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatagtacaagaaggccctctag

SEQ ID NO: 54 - DNA, 540 bp, synthetic construct
  misc_feature 1..540: note = Recombinant
  source 1..540: mol_type = other DNA
aagaccctaatactgttctctacccgcgacgggcagacaagggagatcgccgcgttccttgcctcggagctgaaggagcaggggatttacgctgacgtcataaaccttaaccggacggaggagatagcttggcaggagtatgatagagtcgtaatcggggcgtcgatccgatacgggcatttccaccctgctgtcgaccgcttcgtgaagaagcacacagagacactcaactcactgcccggcgcctttttctctgtaaaccttgttgcccggaaagccgagaagagaacgccgcagacgaactcatacaccaggaagttcctattaaacagcccgtggaagccagcggcctgcgcggtctttgctggggccctccgctaccctagataccgctggtacgacaggttcatgatacgactgattatgaaaatgacaggcggggagacggatacccgaaaggaggtagtctacactgactggtcgcagatcgcgtcgtttgccagagagatagtccagttgaccaggtcatcgcgcttgtga

SEQ ID NO: 55 - DNA, 540 bp, synthetic construct
  misc_feature 1..540: note = Codon optimized for monocot expression
  source 1..540: mol_type = other DNA
atgaaggcgctcgtgctctacagcacacgcgacggccagactcatgcgatcgcctcttacatcgcgtcctgtatgaaggagaaggccgagtgcgacgtcatcgatctcacgcacggggagcacgtgaatcttacgcagtacgaccaagtgctgataggcgcctctatccgttacggccattttaacgccgtcctcgacaaattcatcaagcgcaatgtagaccagctgaacaacatgccctccgcgttcttttgcgtgaacctgacggctcggaagcctgagaagcgaacacctcagaccaacccatacgtgcggaaattcctactcgcaacgccatggcagcccgccctgtgcggggttttcgcaggggcgctacgctatccgcgttaccgctggatcgataaggtgatgatccagctaataatgcgcatgaccggcggcgagacagacacatcgaaggaagtcgaatacacagactgggaacaggtgaagaagtttgcagaggatttcgccaagctctcatacaaaaaggcattgtga

SEQ ID NO: 56 - DNA, 537 bp, synthetic construct
  misc_feature 1..537: note = Codon optimized for monocot expression
  source 1..537: mol_type = other DNA
atgaaggcgcttatactgttctcgacacgcgacggtcagacgcagaaaatcgcctcagccatcgccgacgagatcaagggccagcagagctgcgatgtgatcaatattcaggacgccaaaactctcgactggcagcagtatgaccgcgtgctcattggcgcatcaatccgctacgggcatttccagccagtcgtcaatgagtttgtgaaacataacctcttggcattgcagcagcgggtgtctggcttcttctccgtgaaccttacagctagaaaaccagagaagcggtcgcccgagactaacgcctacaccgttaagttccttgcgcagtcaccgtggcagcctgattgctgcgcggtcttcgccggggcactgtactaccctcgataccggtggtttgatagggtaatgatccagttcataatgcgcatgaccggtggggagaccgacgcaagtaaagaagttgagtacacggattggcagcaggtgcaaaggttcgcacgcgacttcgcgcagctcccgggcaagtcttactga

SEQ ID NO: 57 - DNA, 534 bp, synthetic construct
  misc_feature 1..534: note = Codon optimized for monocot expression
  source 1..534: mol_type = other DNA
atgaaagccctgatcctctattccaccagggacggccagacccgcaagatagcctcctccatcgctgatgtcatccgccagcagcagcagtgcgacgttttaaacattaaggacgcttcactgcctgattgggcccagtatgaccgcgtcctgatcggcgcgtcgattcggtacggccacttccagcctgtggttgacaagttcgtcaagcagcacctgcatgagctgcagcagcgaactagcgggttcttcagtgtgaacctgacagctagaaagcccgaaaagagatccccagaaaccaacgcctatacgcagaaattccttgctcactcaccctggcagcctgactgttgtgccgtcttcgcgggcgccttgtactatccccgctaccgctggttcgatagggtgatgatccagctgattatgagaatgacgggaggggagaccgattcgaccaaggaggtagagtacactgactggcaacaggtgtcaactttcgcaaacgacttcgcacaactacccggtaagtcttga

SEQ ID NO: 58 - DNA, 546 bp, synthetic construct
  misc_feature 1..546: note = Codon optimized for monocot expression
  source 1..546: mol_type = other DNA
atgaaaaccctaatactgttctcgacccgcgacggccagacgcgtgagattgcgagctacctggcctccgagctcaaggagctggggatccaagccgatgtcgcgaacgtgcaccgcattgaggagccgcagtgggagaattacgatcgcgttgtgataggggccagcatccgctatggccactaccactcggcctttcaggagtttgtaaagaaacacgccacaagattaaactccatgcctagcgccttctactccgtcaaccttgtcgcgcgcaagccggagaagcggacacctcagacgaactcctacgcgcggaagttcctgatgaacagccagtggcggccggacagatgtgctgttattgcgggagccctgagatacccgaggtaccggtggtacgataggtttatgattaaacttattatgaagatgtctggtggggagactgacaccaggaaggaggtggtatatacagactgggagcaggtcgccaatttcgctcgggaaatcgcgcatctgacagacaagcctacactgaagtag

SEQ ID NO: 59 - DNA, 537 bp, synthetic construct
  misc_feature 1..537: note = Codon optimized for monocot expression
  source 1..537: mol_type = other DNA
atgaaggccctgatcctctttagctctagggagggccagacccgcgagatcgcgtcatatatcgcgaattccataaaggaggagatggagtgcgatgtgtttaacatccttagggtggagcaaatagactggtctcagtatgaccgtgtgctcataggggggagcatccactacggccactttcacccggccgtggcgaaattcgtcaagcgacacctccacgagcttcagcagcgctcctcagggttcttctgcgtcaacctgacagcaagaaaggcagataaacgcaccccgcagacgaacgcctacatgaggaagttccttctgcagtctccttggcagcccgattgctgcgcggtgttcgccggtgcactgcgctatacgcgctatagatggtttgatagagtcatgattcagctcatcatgcggatgaccggcggggaaacggatactagtaaggaggtggagtacacggactggacccaggtggcacgtttcgcccaggagtttgcacatcttcctgggaagacccaatga

SEQ ID NO: 60 - DNA, 540 bp, synthetic construct
  misc_feature 1..540: note = Codon optimized for monocot expression
  source 1..540: mol_type = other DNA
atgaaggcgctaattgtgttcagctccagggatggccagacgagggctatagcatcctatatcgccaataccttgaaaggaacgctcgagtgtgacgtggtcaacgtcttgaacgccaatgacattgacctttcccagtacgaccgagttgccataggcgcgtcgatccgctacgggcgatttcaccctgcagtcaaccagtttatacggaagcatttgacctcgctgcagcagctcccgtcagccttcttctctgtgaatttaaccgcgcggaagcctgagaaacggacgatccaaacaaacgcctatacccgaaagttcctcctgaacagcccatggcagccagacctgtgctgtgtcttcgccggcgcgttgcggtatccccgctacaggtggttcgatagagtgatgatccagctcatcatgaggatcaccgggggagagaccgatagtaccaaggagatcgagtacacggactggcagcaggtggctcgcttcgcccaggacttcgctcagttggccgcaaagaatccagcataa

SEQ ID NO: 61 - DNA, 540 bp, synthetic construct
  misc_feature 1..540: note = Codon optimized for monocot expression
  source 1..540: mol_type = other DNA
atgaagacactgatcctgttctcgactcgagatggccagactcataaaattgcgcgccacattgcgggggtcctggaggagcagggcaaagcgtgcgagctcgtggacttactccagcccggggagccggactggagcacggtggagtgcgtcgttctgggcgcttccatacgttacgggcatttccacaaaagtttcatccggttcgtcaacacccacgctcaacggctgaacaacatgcctggcgcgctattcactgttaacttagtggctcgtaagcccgagaagcagtctccgcagactaactcctacacaaggaaatttctagcagcaagcccatggcaaccgcagcggtgccaggtgttcgctggagctctgcgctatcctaggtacagttggtacgacagaatgatgatacggttgattatgaagatggcaggcggggagacggacaccaggaaagaggtcgaatacactgactggcaatcagtcactcggtttgctagagagatcgcgcaattaccaggtgagacgcggtaa

SEQ ID NO: 62 - DNA, 537 bp, synthetic construct
  misc_feature 1..537: note = Codon optimized for monocot expression
  source 1..537: mol_type = other DNA
atgaaggctctcatactgttcagctcgagagacgggcagacccagctgatcgcctcctccatagcaaaggagctagagggcaagcaagcctgcgacgtgctcaatattctcgacacaaccaacgtggagtggactcagtacgacagagtcctaatcggcgcgtccatcagatacggccacttccatcccgccgtcgctgaattcgtgaaacgccaccagcgtgagctccagcagcgcagcagcggcttcttcagcgtgaatcttactgcgagaaagccggaaaagcggagtcccgagactaacgcttatacggcaaagttcctcaaccaatctccctggcaaccagactgctgtgccgtgttcgctggggcactgaggtatccgcgctatcggtggttcgatagaatcatgatacagctgataatgcgtatgactggtggggagacggattccagtaaagaggtagagtatactgattggcagcaggtcactaggttcgcgcaggagtttgctaggctgccgggcaagacatcctga

SEQ ID NO: 63 - DNA, 543 bp, synthetic construct
  misc_feature 1..543: note = Codon optimized for monocot expression
  source 1..543: mol_type = other DNA
atgaaaaccttaatcttgttcagcacccgcgacggccagacgcgtgaaatcgcagcgttcctcgcttcggagctcaaggaacagggaatttacgccgacgtcattaacctaaaccgtaccgaagagattgcgtggcaggagtatgaccgcgtggtgattggcgcttctatccgctatggccacttccacccggctgttgaccggttcgtgaagaagcacacggagaccttgaactcactgccgggggcattctttagcgtaaatctggtggcgcgcaaggccgagaagcgcaccccccagacgaacagctacacccgcaaatttttacttaactccccatggaaacctgcggcctgcgcagtgttcgcaggagctctccgctatcctcgctatcgatggtacgatcggttcatgattcggctgattatgaaaatgacgggcggcgagacggatacgcgaaaggaagttgtctacactgactggtcccaggtggcctcgtttgcaagggagatcgtacagctcactcgatctagtaggctctga

SEQ ID NO: 64 - DNA, 534 bp, synthetic construct
  misc_feature 1..534: note = Codon optimized for monocot expression
  source 1..534: mol_type = other DNA
atgaagattctcatcttattttccacccgagacggccaaacccgcgagattgcggcgtccctcgcctccgagttgaaggagcaggcgtttgatgtggatgtggtcaacctccaccgcgcagaaaacatagcgtgggaggagtacgatggggtcgtcatcggagcgtcaatccgctacggacatttccactcaacgctgaattcatttgtgaagaagcaccaacaagcgctcaagaagctgcccggagcattctacagcgtcaacctcgtggctcggaagccggaaaagcgcaccccgcaaacaaacagctacacacgcaagtttctgctcgactcgccctggcaacccgacctgagtgccgttttcgccggggcactgcgctatccccgttacaactggtacgatcgcataatgattcgactgatcatgaagattacaggcggggaaaccgatactcggaaggaggtggtgtatacagactggcagcaggttacccacttcgcccacgagatcgtccagctcgttcgtaagtga

SEQ ID NO: 65 - protein, 179 aa, Rahnella aquatilis
MKALILYSSRDGQTHAIASYIANELKEKCSCDVVDLAHAERVDLKSYDQVMIGASIRYGHFNPVLDKFVKKHAGILNHMPSAFFGVNLTARKPEKRTPQTNAYVRKFLLASPWEPAMCGVFAGALRYPRYRWFDKVMIQLIMRMTGGETDTRKEVEYTDWQQVAKFAEDFGQISYKKSH

SEQ ID NO: 66 - protein, 181 aa, unidentified
  source 1..181: note = Salmonella enterica subsp enterica serovar
MKTLILFSTRDGQTREIASYLASELKEMGIWADVVNLHRAEEPDWDSYDRVVIGASIRYGHYHSAFQEFVKKYATRLNGMPSAFYSVNLVARKAEKRTPQTNSYARKFLMSSPWRPDYCAVIAGALRYPRYRWYDRLMIKLIMKMSGGETDTSKEVVYTDWEQVAHFAREIAHLTNKSSAK

SEQ ID NO: 67 - protein, 179 aa, Rahnella aquatilis
MKALILYSSRDGQTHAIASYIANELKEKCSCDVVDLAHAERVDLKSYDQVMIGASIRYGHFNPVLDKFVKKHAETLNRMPSAFFGVNLTARKPEKRTPQTNAYVRKFLLASPWEPAMCGVFAGALRYPRYRWFDKVMIQLIMRMTGGETDTRKEVEYTDWQQVAKFAEDFGQISYKKSH

SEQ ID NO: 68 - protein, 175 aa, Vibrio pacinii
MAKALFLYSTREGQTKKIFQHISEQMKEFDCEMIDLHSVESVDFSQYQRVLIGASIRYGHLNKKLYQFIEHNLSQLQTAKSAFFCVNLTARKEDQAKDTPEGSAYIKTFLSKSPWQPELIGVFAGALYYPRYNWFDKTMIKFIMSMTGGETDTSKEVEYTNWGKVTLFADKFQNL

SEQ ID NO: 69 - protein, 174 aa, Xenorhabdus nematophila
MSYLLLYSTQDGQTKKIIMRIAENLRRSGVSCDLRDLAAVKQVNLASYQKVMVGASIRYGHFNSVLHKFVTHHQKQLNQKPTAFFGVNLTARKPEKRTPETNAYVRKFLMKSPWQPDLCEVFAGALLYPRYKWLDRVMIQIIMRMTGGETDTTKEIEYTDWAQVDRFSEMFLQI

SEQ ID NO: 70 - protein, 174 aa, Vibrio diazotrophicus
MKALLLYSTREGQTKKIMHHIAQQLQGYECQFVDLHECHTMDLTQYDKVLIGASIRYGKLNAKLYQFIDAHIKQLEQVKAAFYCVNLTARKVEQGKDTPEGSVYIKTFLKKSPWQPSLIGVFAGALYYPRYRPIDRMMIRFIMKLTGGETDTTKEVEYTDWEKVSLFAKKFEQL

SEQ ID NO: 71 - protein, 174 aa, Grimontia hollisae
MKKALLLHSSREGQTLKILRAIESELSESYQCELVDLHEMPEVNWEDYDKVLIGASIRYGHLNKKLYRFIETNLSSLKNKKAAFFCVNLTARKPGKDTPEGSVYMKKFLKRSPWQPQLLDVFAGALYYPRYRFFDRVMIQFIMKMTGGETDPTKEIEYTNWDRVKVFSGQFRSL

SEQ ID NO: 72 - protein, 177 aa, unidentified
  source 1..177: note = Gilliamella apicola
MKVLLLYTTHEKQTFKIMQRIENQLAGKCDCDVIELLPSTNIDLTKYQAVLLGCSIRYGFYSKVMKKFIDNNYQQLNKMRSGFFGVNVVARKPHKNTPETNSYTRKFLAKIAWQPTIKAVFAGALYYPKYNWFDRNMVRFIMWLGKGDTDVTKPIIEYTDWAKVDQFAELFYTQTYS

SEQ ID NO: 73 - protein, 174 aa, Shewanella oneidensis
MQTLIIYSTIDGQTLEICRKIKAFAERAGEKVSLFSLEQAEAINLADVDKVLIGASIRYGKHRPELYQFVNRNHAVLSAKVNGFFTVNVVARKPLKNTPETNPYMQKFLKLSLWQPQHLAVFAGKIDYPKYGLFDRTMICFIMWMTKGPTDLKGTFEFTDWAKVEAFGTHFSKL

SEQ ID NO: 74 - protein, 175 aa, Shewanella benthica
MNKILIIYSSVHGQTRKICEYIASQLRASDYGCEVRLASLEEQFDLDSFDKIIIGASIRHGKHNPEVYHFIDTHLVALETKSSSFFSVSLVARKASRNTPESNPYMQAFLSKTLWRPNLVKVFAGKLDYQGYNWLDRSIIRFIMWITKGPTAVNTKIEYTDWQLVDVFVKELELL

SEQ ID NO: 75 - protein, 176 aa, Marinomonas ushuaiensis
MSTTLLIYSTTDGHTKKISQKIQEIIEAKGQKVTLLPIEEITAAALDSHDKIVIGASIRYGKHQKVVADFIEQNKTTLESKPSAFYTVNLVARKPEKCQPDTNPYIIKFLSQLDWQPSLQGVFAGKLDYQKYGFIDRNMIRFIMWMTKGPTDPKTNIEFTNWEAVDQFANGVVELS

SEQ ID NO: 76 - protein, 173 aa, Shewanella woodyi
MSNILIIYSSVHGQTRKICAYLEDKFVALGDKVTMSSLDQVPDLNDFDKVVLGASIRHGKHNPNVYDFISQNRGILEKKTSSFFSVNLVARKPAKNTPQTNPYMLAFIEKSEWKPNLLQVFAGSLNYQGYGIVDRNIIRFIMWMTKGPTDAQTNIEYTDWAKVDLFSSEFHAL

SEQ ID NO: 77 - protein, 176 aa, Agarivorans albus
MKLLVLYSSCEGQTLKIAKHIVSQQAGEVAADYIQIDSNQQSLDLTAYDKVLIGASIRYGKFRPQLYSLLAEYQNQLANVPVAFFGVCLTARKPEKNTPETSVYMKKLNLNAAWMPKLQAVFAGALLYSKYTWWQALLIQFIMKMTGGSTDRSQDLELTDWAKVDEFATQFAKLDK

SEQ ID NO: 78 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAAALRYPRYRWIDKVMIQLIMRMTGGETDTSKEVEYTDWEQVKKFAEDFAKLSYKKAL

SEQ ID NO: 79 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGAARYPRYRWIDKVMIQLIMRMTGGETDTSKEVEYTDWEQVKKFAEDFAKLSYKKAL

SEQ ID NO: 80 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGAFRYPRYRWIDKVMIQLIMRMTGGETDTSKEVEYTDWEQVKKFAEDFAKLSYKKAL

SEQ ID NO: 81 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGAIRYPRYRWIDKVMIQLIMRMTGGETDTSKEVEYTDWEQVKKFAEDFAKLSYKKAL

SEQ ID NO: 82 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGAVRYPRYRWIDKVMIQLIMRMTGGETDTSKEVEYTDWEQVKKFAEDFAKLSYKKAL

SEQ ID NO: 83 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALAYPRYRWIDKVMIQLIMRMTGGETDTSKEVEYTDWEQVKKFAEDFAKLSYKKAL

SEQ ID NO: 84 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRAPRYRWIDKVMIQLIMRMTGGETDTSKEVEYTDWEQVKKFAEDFAKLSYKKAL

SEQ ID NO: 85 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRHPRYRWIDKVMIQLIMRMTGGETDTSKEVEYTDWEQVKKFAEDFAKLSYKKAL

SEQ ID NO: 86 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRMPRYRWIDKVMIQLIMRMTGGETDTSKEVEYTDWEQVKKFAEDFAKLSYKKAL

SEQ ID NO: 87 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRWPRYRWIDKVMIQLIMRMTGGETDTSKEVEYTDWEQVKKFAEDFAKLSYKKAL

SEQ ID NO: 88 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYARYRWIDKVMIQLIMRMTGGETDTSKEVEYTDWEQVKKFAEDFAKLSYKKAL

SEQ ID NO: 89 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYDRYRWIDKVMIQLIMRMTGGETDTSKEVEYTDWEQVKKFAEDFAKLSYKKAL

SEQ ID NO: 90 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYERYRWIDKVMIQLIMRMTGGETDTSKEVEYTDWEQVKKFAEDFAKLSYKKAL

SEQ ID NO: 91 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYKRYRWIDKVMIQLIMRMTGGETDTSKEVEYTDWEQVKKFAEDFAKLSYKKAL

SEQ ID NO: 92 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYLRYRWIDKVMIQLIMRMTGGETDTSKEVEYTDWEQVKKFAEDFAKLSYKKAL

SEQ ID NO: 93 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYQRYRWIDKVMIQLIMRMTGGETDTSKEVEYTDWEQVKKFAEDFAKLSYKKAL

SEQ ID NO: 94 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYRRYRWIDKVMIQLIMRMTGGETDTSKEVEYTDWEQVKKFAEDFAKLSYKKAL

SEQ ID NO: 95 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYSRYRWIDKVMIQLIMRMTGGETDTSKEVEYTDWEQVKKFAEDFAKLSYKKAL

SEQ ID NO: 96 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYTRYRWIDKVMIQLIMRMTGGETDTSKEVEYTDWEQVKKFAEDFAKLSYKKAL

SEQ ID NO: 97 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPAYRWIDKVMIQLIMRMTGGETDTSKEVEYTDWEQVKKFAEDFAKLSYKKAL

SEQ ID NO: 98 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPEYRWIDKVMIQLIMRMTGGETDTSKEVEYTDWEQVKKFAEDFAKLSYKKAL

SEQ ID NO: 99 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPGYRWIDKVMIQLIMRMTGGETDTSKEVEYTDWEQVKKFAEDFAKLSYKKAL

SEQ ID NO: 100 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPHYRWIDKVMIQLIMRMTGGETDTSKEVEYTDWEQVKKFAEDFAKLSYKKAL

SEQ ID NO: 101 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPIYRWIDKVMIQLIMRMTGGETDTSKEVEYTDWEQVKKFAEDFAKLSYKKAL

SEQ ID NO: 102 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPKYRWIDKVMIQLIMRMTGGETDTSKEVEYTDWEQVKKFAEDFAKLSYKKAL

SEQ ID NO: 103 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPLYRWIDKVMIQLIMRMTGGETDTSKEVEYTDWEQVKKFAEDFAKLSYKKAL

SEQ ID NO: 104 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPNYRWIDKVMIQLIMRMTGGETDTSKEVEYTDWEQVKKFAEDFAKLSYKKAL

SEQ ID NO: 105 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPQYRWIDKVMIQLIMRMTGGETDTSKEVEYTDWEQVKKFAEDFAKLSYKKAL

SEQ ID NO: 106 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPSYRWIDKVMIQLIMRMTGGETDTSKEVEYTDWEQVKKFAEDFAKLSYKKAL

SEQ ID NO: 107 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRARWIDKVMIQLIMRMTGGETDTSKEVEYTDWEQVKKFAEDFAKLSYKKAL

SEQ ID NO: 108 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRCRWIDKVMIQLIMRMTGGETDTSKEVEYTDWEQVKKFAEDFAKLSYKKAL

SEQ ID NO: 109 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRLRWIDKVMIQLIMRMTGGETDTSKEVEYTDWEQVKKFAEDFAKLSYKKAL

SEQ ID NO: 110 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRWRWIDKVMIQLIMRMTGGETDTSKEVEYTDWEQVKKFAEDFAKLSYKKAL

SEQ ID NO: 111 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYAWIDKVMIQLIMRMTGGETDTSKEVEYTDWEQVKKFAEDFAKLSYKKAL

SEQ ID NO: 112 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRAIDKVMIQLIMRMTGGETDTSKEVEYTDWEQVKKFAEDFAKLSYKKAL

SEQ ID NO: 113 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRFIDKVMIQLIMRMTGGETDTSKEVEYTDWEQVKKFAEDFAKLSYKKAL

SEQ ID NO: 114 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRIIDKVMIQLIMRMTGGETDTSKEVEYTDWEQVKKFAEDFAKLSYKKAL

SEQ ID NO: 115 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRKIDKVMIQLIMRMTGGETDTSKEVEYTDWEQVKKFAEDFAKLSYKKAL

SEQ ID NO: 116 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRLIDKVMIQLIMRMTGGETDTSKEVEYTDWEQVKKFAEDFAKLSYKKAL

SEQ ID NO: 117 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRPIDKVMIQLIMRMTGGETDTSKEVEYTDWEQVKKFAEDFAKLSYKKAL

SEQ ID NO: 118 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRRIDKVMIQLIMRMTGGETDTSKEVEYTDWEQVKKFAEDFAKLSYKKAL

SEQ ID NO: 119 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRSIDKVMIQLIMRMTGGETDTSKEVEYTDWEQVKKFAEDFAKLSYKKAL

SEQ ID NO: 120 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRTIDKVMIQLIMRMTGGETDTSKEVEYTDWEQVKKFAEDFAKLSYKKAL

SEQ ID NO: 121 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRVIDKVMIQLIMRMTGGETDTSKEVEYTDWEQVKKFAEDFAKLSYKKAL

SEQ ID NO: 122 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRYIDKVMIQLIMRMTGGETDTSKEVEYTDWEQVKKFAEDFAKLSYKKAL

SEQ ID NO: 123 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWADKVMIQLIMRMTGGETDTSKEVEYTDWEQVKKFAEDFAKLSYKKAL

SEQ ID NO: 124 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIAKVMIQLIMRMTGGETDTSKEVEYTDWEQVKKFAEDFAKLSYKKAL

SEQ ID NO: 125 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIKKVMIQLIMRMTGGETDTSKEVEYTDWEQVKKFAEDFAKLSYKKAL

SEQ ID NO: 126 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWINKVMIQLIMRMTGGETDTSKEVEYTDWEQVKKFAEDFAKLSYKKAL

SEQ ID NO: 127 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIQKVMIQLIMRMTGGETDTSKEVEYTDWEQVKKFAEDFAKLSYKKAL

SEQ ID NO: 128 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWITKVMIQLIMRMTGGETDTSKEVEYTDWEQVKKFAEDFAKLSYKKAL

SEQ ID NO: 129 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDAVMIQLIMRMTGGETDTSKEVEYTDWEQVKKFAEDFAKLSYKKAL

SEQ ID NO: 130 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDQVMIQLIMRMTGGETDTSKEVEYTDWEQVKKFAEDFAKLSYKKAL

SEQ ID NO: 131 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDRVMIQLIMRMTGGETDTSKEVEYTDWEQVKKFAEDFAKLSYKKAL

SEQ ID NO: 132 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDSVMIQLIMRMTGGETDTSKEVEYTDWEQVKKFAEDFAKLSYKKAL

SEQ ID NO: 133 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDTVMIQLIMRMTGGETDTSKEVEYTDWEQVKKFAEDFAKLSYKKAL

SEQ ID NO: 134 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDVVMIQLIMRMTGGETDTSKEVEYTDWEQVKKFAEDFAKLSYKKAL

SEQ ID NO: 135 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKAMIQLIMRMTGGETDTSKEVEYTDWEQVKKFAEDFAKLSYKKAL

SEQ ID NO: 136 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVAIQLIMRMTGGETDTSKEVEYTDWEQVKKFAEDFAKLSYKKAL

SEQ ID NO: 137 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVCIQLIMRMTGGETDTSKEVEYTDWEQVKKFAEDFAKLSYKKAL

SEQ ID NO: 138 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVIIQLIMRMTGGETDTSKEVEYTDWEQVKKFAEDFAKLSYKKAL

SEQ ID NO: 139 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVLIQLIMRMTGGETDTSKEVEYTDWEQVKKFAEDFAKLSYKKAL

SEQ ID NO: 140 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVSIQLIMRMTGGETDTSKEVEYTDWEQVKKFAEDFAKLSYKKAL

SEQ ID NO: 141 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVVIQLIMRMTGGETDTSKEVEYTDWEQVKKFAEDFAKLSYKKAL

SEQ ID NO: 142 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMAQLIMRMTGGETDTSKEVEYTDWEQVKKFAEDFAKLSYKKAL

SEQ ID NO: 143 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMLQLIMRMTGGETDTSKEVEYTDWEQVKKFAEDFAKLSYKKAL

SEQ ID NO: 144 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMMQLIMRMTGGETDTSKEVEYTDWEQVKKFAEDFAKLSYKKAL

SEQ ID NO: 145 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMVQLIMRMTGGETDTSKEVEYTDWEQVKKFAEDFAKLSYKKAL

SEQ ID NO: 146 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQAIMRMTGGETDTSKEVEYTDWEQVKKFAEDFAKLSYKKAL

SEQ ID NO: 147 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQCIMRMTGGETDTSKEVEYTDWEQVKKFAEDFAKLSYKKAL

SEQ ID NO: 148 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQFIMRMTGGETDTSKEVEYTDWEQVKKFAEDFAKLSYKKAL

SEQ ID NO: 149 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQGIMRMTGGETDTSKEVEYTDWEQVKKFAEDFAKLSYKKAL

SEQ ID NO: 150 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQHIMRMTGGETDTSKEVEYTDWEQVKKFAEDFAKLSYKKAL

SEQ ID NO: 151 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQIIMRMTGGETDTSKEVEYTDWEQVKKFAEDFAKLSYKKAL

SEQ ID NO: 152 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQKIMRMTGGETDTSKEVEYTDWEQVKKFAEDFAKLSYKKAL

SEQ ID NO: 153 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQMIMRMTGGETDTSKEVEYTDWEQVKKFAEDFAKLSYKKAL

SEQ ID NO: 154 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQRIMRMTGGETDTSKEVEYTDWEQVKKFAEDFAKLSYKKAL

SEQ ID NO: 155 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQTIMRMTGGETDTSKEVEYTDWEQVKKFAEDFAKLSYKKAL

SEQ ID NO: 156 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLAMRMTGGETDTSKEVEYTDWEQVKKFAEDFAKLSYKKAL

SEQ ID NO: 157 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLLMRMTGGETDTSKEVEYTDWEQVKKFAEDFAKLSYKKAL

SEQ ID NO: 158 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLMMRMTGGETDTSKEVEYTDWEQVKKFAEDFAKLSYKKAL

SEQ ID NO: 159 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLVMRMTGGETDTSKEVEYTDWEQVKKFAEDFAKLSYKKAL

SEQ ID NO: 160 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIARMTGGETDTSKEVEYTDWEQVKKFAEDFAKLSYKKAL

SEQ ID NO: 161 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIDRMTGGETDTSKEVEYTDWEQVKKFAEDFAKLSYKKAL

SEQ ID NO: 162 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLILRMTGGETDTSKEVEYTDWEQVKKFAEDFAKLSYKKAL

SEQ ID NO: 163 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLISRMTGGETDTSKEVEYTDWEQVKKFAEDFAKLSYKKAL

SEQ ID NO: 164 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIVRMTGGETDTSKEVEYTDWEQVKKFAEDFAKLSYKKAL

SEQ ID NO: 165 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMAMTGGETDTSKEVEYTDWEQVKKFAEDFAKLSYKKAL

SEQ ID NO: 166 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRATGGETDTSKEVEYTDWEQVKKFAEDFAKLSYKKAL

SEQ ID NO: 167 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMAGGETDTSKEVEYTDWEQVKKFAEDFAKLSYKKAL

SEQ ID NO: 168 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTAGETDTSKEVEYTDWEQVKKFAEDFAKLSYKKAL

SEQ ID NO: 169 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTDGETDTSKEVEYTDWEQVKKFAEDFAKLSYKKAL

SEQ ID NO: 170 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTHGETDTSKEVEYTDWEQVKKFAEDFAKLSYKKAL

SEQ ID NO: 171 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTKGETDTSKEVEYTDWEQVKKFAEDFAKLSYKKAL

SEQ ID NO: 172 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTNGETDTSKEVEYTDWEQVKKFAEDFAKLSYKKAL

SEQ ID NO: 173 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGAETDTSKEVEYTDWEQVKKFAEDFAKLSYKKAL

SEQ ID NO: 174 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGKETDTSKEVEYTDWEQVKKFAEDFAKLSYKKAL

SEQ ID NO: 175 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGMETDTSKEVEYTDWEQVKKFAEDFAKLSYKKAL

SEQ ID NO: 176 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGRETDTSKEVEYTDWEQVKKFAEDFAKLSYKKAL

SEQ ID NO: 177 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGSETDTSKEVEYTDWEQVKKFAEDFAKLSYKKAL

SEQ ID NO: 178 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIALIMRMTGGETDTSKEVEYTDWEQVKKFAEDFAKLSYKKAL

SEQ ID NO: 179 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMICLIMRMTGGETDTSKEVEYTDWEQVKKFAEDFAKLSYKKAL

SEQ ID NO: 180 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIELIMRMTGGETDTSKEVEYTDWEQVKKFAEDFAKLSYKKAL

SEQ ID NO: 181 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIGLIMRMTGGETDTSKEVEYTDWEQVKKFAEDFAKLSYKKAL

SEQ ID NO: 182 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIHLIMRMTGGETDTSKEVEYTDWEQVKKFAEDFAKLSYKKAL

SEQ ID NO: 183 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIKLIMRMTGGETDTSKEVEYTDWEQVKKFAEDFAKLSYKKAL

SEQ ID NO: 184 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMILLIMRMTGGETDTSKEVEYTDWEQVKKFAEDFAKLSYKKAL

SEQ ID NO: 185 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIMLIMRMTGGETDTSKEVEYTDWEQVKKFAEDFAKLSYKKAL

SEQ ID NO: 186 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIRLIMRMTGGETDTSKEVEYTDWEQVKKFAEDFAKLSYKKAL

SEQ ID NO: 187 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMISLIMRMTGGETDTSKEVEYTDWEQVKKFAEDFAKLSYKKAL

SEQ ID NO: 188 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQNIMRMTGGETDTSKEVEYTDWEQVKKFAEDFAKLSYKKAL

SEQ ID NO: 189 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQQIMRMTGGETDTSKEVEYTDWEQVKKFAEDFAKLSYKKAL

SEQ ID NO: 190 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQSIMRMTGGETDTSKEVEYTDWEQVKKFAEDFAKLSYKKAL

SEQ ID NO: 191 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQVIMRMTGGETDTSKEVEYTDWEQVKKFAEDFAKLSYKKAL

SEQ ID NO: 192 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQWIMRMTGGETDTSKEVEYTDWEQVKKFAEDFAKLSYKKAL

SEQ ID NO: 193 - protein, 179 aa, synthetic construct
  REGION 1..179: note = PPO variant
MKALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQYIMRMTGGETDTSKEVEYTDWEQVKKFAEDFAKLSYKKAL

SEQ ID NO: 194 - DNA, 540 bp, unidentified
  source 1..540: mol_type = genomic DNA; note = Ahnella aquatilis
atgaaggctcttatcctctactctagcagagacggacagacacacgccattgcctcctacatcgcaaatgaactcaaggagaagtgctcctgcgatgtcgtggacctggcgcacgctgagcgggttgatctgaagtcatacgaccaggtcatgattggcgcttccatacgctacggacacttcaatcccgttctcgacaagtttgtgaagaagcacgccgggatcttaaaccacatgccatctgccttcttcggcgtgaacttaacagcccggaagcccgagaagcgtacaccacagacgaacgcctacgtccgcaagttcttgctcgcatcaccctgggagcccgcaatgtgcggcgtgttcgctggtgccctgcgatacccgcgctaccgctggttcgacaaggtcatgattcagctcatcatgcggatgacaggaggcgaaacggacactaggaaggaagtagagtacaccgactggcaacaagtggccaaattcgcagaggacttcgggcagattagctacaagaagtctcattag

SEQ ID NO: 195 - DNA, 546 bp, unidentified
  source 1..546: mol_type = genomic DNA; note = Salmonella enterica subsp enterica serovar
atgaagacgctcattctcttctcgacgcgggacgggcaaactcgggaaatcgcgtcatacctggcgtccgagctcaaggagatgggcatctgggccgatgtcgtgaacctccacagagcggaggagcccgattgggactcctacgacagggttgtcataggtgcctcgatccgctacggacattatcactccgctttccaggagttcgttaagaagtacgccacgcgattgaacggtatgccaagtgccttctactcggtgaatcttgttgcacggaaggccgagaagcgaactccacagaccaacagctacgcacggaaattcctcatgtcaagtccttggagacctgattattgcgccgtgattgcgggtgccttgcgttaccctcgctatcgctggtacgaccgactcatgatcaaacttatcatgaagatgtctggcggcgagaccgacacgagcaaggaggtggtgtacactgattgggaacaagtggcgcacttcgcacgagagattgcacaccttacgaacaagtcctcggccaaatag

SEQ ID NO: 196 - DNA, 540 bp, Rahnella aquatilis
  source 1..540: mol_type = genomic DNA
atgaaggccctcatcttgtactccagccgcgacgggcaaacccatgctatcgcctcttacattgcgaacgagcttaaggagaagtgttcctgcgatgtcgtggacctcgcccacgcggaaagagtggacctcaagtcgtatgaccaagtgatgatcggcgcttccatccgatacggccacttcaatccagtgctcgacaagttcgttaagaagcacgcggagactcttaaccgtatgccctcagccttcttcggagtcaacttgaccgctaggaaacctgagaagcgcacgccgcagaccaatgcttacgtgaggaagttcctccttgcaagcccttgggagccagccatgtgcggtgtgttcgctggtgccctccgctacccacgctaccgttggtttgacaaggtcatgattcagttgattatgagaatgacaggcggtgagacagacacacgcaaggaagtcgagtacaccgattggcagcaagtggctaagttcgcagaggacttcggtcagatcagctacaagaaatctcattga

SEQ ID NO: 197 - DNA, 528 bp, Vibrio pacinii
  source 1..528: mol_type = genomic DNA
atggcaaaggcactcttcctctactctacccgcgagggccagaccaagaagatcttccagcacatctccgagcagatgaaggagttcgattgcgagatgatcgacctgcactccgtcgagtccgtggacttctcccagtaccaacgggtactcatcggcgcatccatccgatacggccaccttaacaagaaactctatcaattcatcgagcacaatctgagccagttgcagaccgctaagtcggcgttcttctgcgtgaacctcactgctcgaaaggaggaccaggcgaaggatactcctgagggctcggcctacatcaagacgttcttatccaagtcgccttggcagccggaactaataggcgtgttcgctggtgccctgtactaccctcgctacaactggttcgacaagacgatgattaagttcatcatgtccatgacgggcggcgagaccgacacctccaaggaggtggagtacactaactggggcaaggtgaccttgttcgcagacaagttccagaacctctag

SEQ ID NO: 198 - DNA, 525 bp, Xenorhabdus nematophila
  source 1..525: mol_type = genomic DNA
atgtcctacctgctcttgtacagcacccaggatggtcagaccaagaagatcatcatgcgtattgccgagaacctccgccgctctggtgtttcgtgcgacctgagagacttggccgcagtgaagcaagtgaacttagcgagctaccagaaggtgatggtcggtgcctcgatccggtacggccacttcaactccgttctgcacaagttcgtgacccaccaccagaagcagctgaaccagaagccgacggccttcttcggagtcaacctcacagcaaggaagccggagaagcgcactccggagactaacgcctacgttcgcaagttcctcatgaagtcgccctggcagcctgacctttgtgaggtcttcgctggcgctctcctgtacccgcgttacaagtggctcgaccgtgtgatgatccagattattatgcggatgactggtggcgagaccgataccaccaaggagatcgagtacaccgattgggcacaagtcgatcggttcagtgagatgttccttcagatttga

SEQ ID NO: 199 - DNA, 525 bp, Vibrio diazotrophicus
  source 1..525: mol_type = genomic DNA
atgaaggcactcctcttgtactccacccgcgagggccagaccaagaagattatgcaccacatcgcccagcagctccagggctatgaatgccagttcgtggacctccacgagtgccacactatggacttgactcaatacgataaggtcttaattggtgcctccattcgttacgggaagctcaacgcaaagctgtaccaattcatcgacgcccacatcaagcagctcgaacaagttaaggccgccttctattgcgtcaacctcaccgcacgcaaggtggaacagggcaaggatactcccgaagggtcagtgtacatcaagacgttcctcaagaagtcgccctggcagccgagcctgatcggcgtgttcgccggtgcattgtactatccgcgctaccgtccaatcgaccgcatgatgataaggttcatcatgaagctcacaggcggcgaaactgataccaccaaggaggtcgagtacaccgactgggagaaggtctcactgttcgcaaagaagttcgagcagttgtga

SEQ ID NO: 200 - DNA, 525 bp, Grimontia hollisae
  source 1..525: mol_type = genomic DNA
atgaagaaggccctcctcctgcattcttcgcgcgagggccagacacttaagatcctcagggccatcgagtcagaactaagcgagtcataccagtgcgagcttgtggacctgcacgagatgccagaggtgaactgggaagattacgataaggtgcttatcggtgcctccatcagatacggccacttgaacaagaagctgtaccgtttcatcgagactaacctctcgtccctcaagaacaagaaggccgcgttcttctgcgtgaacttgaccgcgaggaagcccggcaaggacacgccggagggctcggtgtacatgaagaaattcctcaagcgttcgccgtggcagccgcagctccttgatgtgttcgctggcgctctgtactacccacgctaccgattcttcgaccgtgtgatgatccagttcattatgaagatgactggtggagagaccgacccaactaaggaaatcgagtacaccaactgggacagggtgaaggtcttctcaggccaatttcggtcgttgtga

SEQ ID NO: 201 - DNA, 534 bp, unidentified
  source 1..534: mol_type = genomic DNA; note = Gilliamella apicola
atgaaggtgctcttgctttacaccactcacgagaagcagacctttaagatcatgcagcgcatcgagaaccagctcgccgggaagtgcgactgcgacgtcattgaactccttccctctacgaacatcgaccttacgaagtaccaggccgtcctcctcgggtgctccattcgctacggcttctactctaaggtgatgaagaagttcatcgacaacaactaccagcagttgaacaagatgaggtccggcttcttcggcgtaaacgtggtggcacgcaagccgcacaagaacacgccggagaccaacagttacacgcgcaagttcctggccaagatcgcgtggcagcctaccatcaaggcggtgttcgcgggcgctctttactacccaaagtacaactggttcgaccggaacatggtgaggttcatcatgtggcttgggaagggcgacaccgacgtcaccaagccaattattgagtacacggactgggccaaggtggaccagttcgcagaactgttctacacgcagacgtactcgtga

SEQ ID NO: 202 - DNA, 525 bp, Shewanella oneidensis
  source 1..525: mol_type = genomic DNA
atgcagaccctcatcatctactccacaatagacggtcagacacttgaaatctgccgtaagatcaaagcgttcgccgagcgcgctggcgagaaggtgtccctgttctcactggaacaggctgaggccatcaacctggcggacgttgataaggttctcatcggcgcatccatccgctatggcaagcaccgtcccgagctgtaccaattcgtcaaccgcaaccacgccgtactatcagccaaagtcaacggcttcttcaccgtgaatgtggtggcgaggaagccattgaagaacactccggagaccaacccttacatgcagaagttcctcaagttgtcgctctggcagcctcaacaccttgcagtgttcgccgggaagatagactacccgaagtacgggctattcgaccgaaccatgatctgcttcatcatgtggatgaccaaagggccgacggacctgaaaggcaccttcgagttcacagactgggccaaggtcgaggctttcgggacacacttctccaagttgtga

SEQ ID NO: 203 - DNA, 528 bp, Shewanella benthica
  source 1..528: mol_type = genomic DNA
atgaacaagattctcatcatctactctagcgtccacgggcagaccaggaagatttgcgagtacatcgccagtcagctcagggcgtccgattatgggtgcgaggtccgactggcctcgctggaggaacagttcgatctggactcattcgacaagatcatcatcggtgcctccatccgccacggcaagcacaacccggaggtgtaccacttcatcgacactcaccttgtggcacttgagacgaagtccagctcgttcttctccgtgtccctagtcgcccgcaaggcaagtcgcaacacgccggagtcaaacccgtacatgcaagcgtttctgtccaagactctgtggagaccaaacctggtcaaggtgttcgccggaaagctcgactaccagggctacaactggcttgatcgcagcatcataaggtttatcatgtggatcacgaagggccctacggccgtgaacactaagatcgagtacaccgactggcaacttgtggatgtgttcgtgaaggaattggagctgctttag

SEQ ID NO: 204 - DNA, 531 bp, Marinomonas ushuaiensis
  source 1..531: mol_type = genomic DNA
atgtccaccaccctcctgatctactcgactacagacgggcatacgaagaagatttcccagaagatccaggagatcatcgaggcgaagggccagaaggtcacccttcttcccatcgaggagattacggcggcggccctagactcccacgataagatcgtgatcggcgcgtccatccgctacggcaagcaccagaaggtcgtggcggacttcattgagcagaacaagactactctggagtcaaagccgtccgccttctacaccgtcaacctggtggcccgcaagcctgagaagtgccagcccgacacgaacccttacatcatcaaattcttgtcacagctcgactggcagccctcccttcaaggagtcttcgctggcaaactcgactaccagaagtacggcttcattgacagaaacatgatacggttcatcatgtggatgaccaaaggccctaccgacccaaagacgaacatcgagttcactaactgggaggcggttgaccagttcgccaacggcgttgtggagcttagctga

SEQ ID NO: 205 - DNA, 522 bp, Shewanella woodyi
  source 1..522: mol_type = genomic DNA
atgagcaacatccttattatctactccagtgtgcacggccagacgcggaagatttgcgcttacttggaggacaagttcgttgcccttggcgacaaggtgaccatgtcgtccctcgaccaagtgcccgacctcaacgacttcgacaaggtggtactcggtgccagcatccgacacgggaagcacaacccgaacgtttacgacttcatcagtcagaatcgtggcatcctcgagaagaagaccagctcattcttcagcgtcaacctcgtggcaaggaagcctgcaaagaacactccacagacgaatccctacatgcttgccttcatcgagaagtccgagtggaagcccaacttgcttcaagtgttcgcgggctcgctaaactaccagggttacggcatagtggaccgtaacattatccggttcatcatgtggatgactaagggaccgacggacgcccagaccaacattgagtacactgattgggctaaagttgatctgtttagttccgagttccacgcgctgtag

SEQ ID NO: 206 - DNA, 531 bp, Agarivorans albus
  source 1..531: mol_type = genomic DNA
atgaagttgctcgtgctttactcatcctgtgagggacagactctcaagatcgctaagcacatcgtctctcaacaggccggagaagtggcggccgactacattcagatcgactccaaccagcagtcactggacctcaccgcttacgacaaggtcttaatcggcgcgtccatcagatacgggaagttccgaccgcagctctactccctactggctgagtaccagaaccagctcgcaaacgtccctgtggccttcttcggcgtttgcctcacagcgcgaaagcccgagaagaatacgccggagactagcgtgtacatgaagaagctgaacttgaacgccgcttggatgcctaagctccaggctgtgttcgctggagccctcttgtacagtaagtacacctggtggcaagccctgctcatacagttcatcatgaagatgacgggcgggagcactgaccgctcacaagacctggaactcacagattgggccaaggtggatgagttcgccactcagttcgccaagttggacaagtag

SEQ ID NO: 207 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcagctgcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 208 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggccgctcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 209 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggccttccggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 210 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggccatacggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 211 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggccgtccggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 212 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttgcttacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 213 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcgggctccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 214 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggcatccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 215 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggatgccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 216 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtggccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 217 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacgctcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 218 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacgatcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 219 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacgaacgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 220 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacaaacgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 221 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacctacgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 222 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtaccaacgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 223 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtaccggcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 224 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtactctcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 225 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacactcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 226 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccggcttaccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 227 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccggaataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 228 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgggctaccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 229 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcattaccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 230 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgatataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 231 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgaaataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 232 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgctataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 233 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgaattaccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 234 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcaataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 235 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgtcttaccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 236 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgagctcggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 237 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgatgccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 238 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgactacggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 239 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgatggcggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 240 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgatacgcttggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 241 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccgggctatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 242 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggttcatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 243 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggataatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 244 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggaaaatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 245 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggctaatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 246 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggcctatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 247 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggcggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 248 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtctatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 249 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggactatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 250 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccgggtcatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 251 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtatatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 252 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtgggctgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 253 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgctaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 254 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcaaaaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 255 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcaataaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 256 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatccaaaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 257 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcactaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 258 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacgctgtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 259 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgaccaagtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 260 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgaccgggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 261 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgactctgtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 262 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacactgtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 263 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacgtcgtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 264 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggctatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 265 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtggctatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 266 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgtgcatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 267 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgataatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 268 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgctaatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 269 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgtctatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 270 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtggtcatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 271 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatggctcagctaataatgcggatgactgggggagagacagacacgagcaaggaggtcgagtacacggattgggagcaggttaagaagttcgcggaggattttgcaaagctatcgtacaagaaggccctctag

SEQ ID NO: 272 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgctacagctaataatgcggatgactgggggagagacagacacgagcaaggaggtcgagtacacggattgggagcaggttaagaagttcgcggaggattttgcaaagctatcgtacaagaaggccctctag

SEQ ID NO: 273 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatgcagctaataatgcggatgactgggggagagacagacacgagcaaggaggtcgagtacacggattgggagcaggttaagaagttcgcggaggattttgcaaagctatcgtacaagaaggccctctag

SEQ ID NO: 274 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatggtccagctaataatgcggatgactgggggagagacagacacgagcaaggaggtcgagtacacggattgggagcaggttaagaagttcgcggaggattttgcaaagctatcgtacaagaaggccctctag

SEQ ID NO: 275 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccaggctataatgcggatgactgggggagagacagacacgagcaaggaggtcgagtacacggattgggagcaggttaagaagttcgcggaggattttgcaaagctatcgtacaagaaggccctctag

SEQ ID NO: 276 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagtgcataatgcggatgactgggggagagacagacacgagcaaggaggtcgagtacacggattgggagcaggttaagaagttcgcggaggattttgcaaagctatcgtacaagaaggccctctag

SEQ ID NO: 277 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagttcataatgcggatgactgggggagagacagacacgagcaaggaggtcgagtacacggattgggagcaggttaagaagttcgcggaggattttgcaaagctatcgtacaagaaggccctctag

SEQ ID NO: 278 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagggcataatgcggatgactgggggagagacagacacgagcaaggaggtcgagtacacggattgggagcaggttaagaagttcgcggaggattttgcaaagctatcgtacaagaaggccctctag

SEQ ID NO: 279 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagcatataatgcggatgactgggggagagacagacacgagcaaggaggtcgagtacacggattgggagcaggttaagaagttcgcggaggattttgcaaagctatcgtacaagaaggccctctag

SEQ ID NO: 280 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagataataatgcggatgactgggggagagacagacacgagcaaggaggtcgagtacacggattgggagcaggttaagaagttcgcggaggattttgcaaagctatcgtacaagaaggccctctag

SEQ ID NO: 281 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagaaaataatgcggatgactgggggagagacagacacgagcaaggaggtcgagtacacggattgggagcaggttaagaagttcgcggaggattttgcaaagctatcgtacaagaaggccctctag

SEQ ID NO: 282 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagatgataatgcggatgactgggggagagacagacacgagcaaggaggtcgagtacacggattgggagcaggttaagaagttcgcggaggattttgcaaagctatcgtacaagaaggccctctag

SEQ ID NO: 283 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagcggataatgcggatgactgggggagagacagacacgagcaaggaggtcgagtacacggattgggagcaggttaagaagttcgcggaggattttgcaaagctatcgtacaagaaggccctctag

SEQ ID NO: 284 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagactataatgcggatgactgggggagagacagacacgagcaaggaggtcgagtacacggattgggagcaggttaagaagttcgcggaggattttgcaaagctatcgtacaagaaggccctctag

SEQ ID NO: 285 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctagctatgcggatgactgggggagagacagacacgagcaaggaggtcgagtacacggattgggagcaggttaagaagttcgcggaggattttgcaaagctatcgtacaagaaggccctctag

SEQ ID NO: 286 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctactaatgcggatgactgggggagagacagacacgagcaaggaggtcgagtacacggattgggagcaggttaagaagttcgcggaggattttgcaaagctatcgtacaagaaggccctctag

SEQ ID NO: 287 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatgatgcggatgactgggggagagacagacacgagcaaggaggtcgagtacacggattgggagcaggttaagaagttcgcggaggattttgcaaagctatcgtacaagaaggccctctag

SEQ ID NO: 288 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctagtcatgcggatgactgggggagagacagacacgagcaaggaggtcgagtacacggattgggagcaggttaagaagttcgcggaggattttgcaaagctatcgtacaagaaggccctctag

SEQ ID NO: 289 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatagctcggatgactgggggagagacagacacgagcaaggaggtcgagtacacggattgggagcaggttaagaagttcgcggaggattttgcaaagctatcgtacaagaaggccctctag

SEQ ID NO: 290 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatagatcggatgactgggggagagacagacacgagcaaggaggtcgagtacacggattgggagcaggttaagaagttcgcggaggattttgcaaagctatcgtacaagaaggccctctag

SEQ ID NO: 291 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatactacggatgactgggggagagacagacacgagcaaggaggtcgagtacacggattgggagcaggttaagaagttcgcggaggattttgcaaagctatcgtacaagaaggccctctag

SEQ ID NO: 292 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatatctcggatgactgggggagagacagacacgagcaaggaggtcgagtacacggattgggagcaggttaagaagttcgcggaggattttgcaaagctatcgtacaagaaggccctctag

SEQ ID NO: 293 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaatagtccggatgactgggggagagacagacacgagcaaggaggtcgagtacacggattgggagcaggttaagaagttcgcggaggattttgcaaagctatcgtacaagaaggccctctag

SEQ ID NO: 294 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatggctatgactgggggagagacagacacgagcaaggaggtcgagtacacggattgggagcaggttaagaagttcgcggaggattttgcaaagctatcgtacaagaaggccctctag

SEQ ID NO: 295 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcgggctactgggggagagacagacacgagcaaggaggtcgagtacacggattgggagcaggttaagaagttcgcggaggattttgcaaagctatcgtacaagaaggccctctag

SEQ ID NO: 296 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatggctgggggagagacagacacgagcaaggaggtcgagtacacggattgggagcaggttaagaagttcgcggaggattttgcaaagctatcgtacaagaaggccctctag

SEQ ID NO: 297 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgctggagagacagacacgagcaaggaggtcgagtacacggattgggagcaggttaagaagttcgcggaggattttgcaaagctatcgtacaagaaggccctctag

SEQ ID NO: 298 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgatggagagacagacacgagcaaggaggtcgagtacacggattgggagcaggttaagaagttcgcggaggattttgcaaagctatcgtacaagaaggccctctag

SEQ ID NO: 299 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactcatggagagacagacacgagcaaggaggtcgagtacacggattgggagcaggttaagaagttcgcggaggattttgcaaagctatcgtacaagaaggccctctag

SEQ ID NO: 300 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactaaaggagagacagacacgagcaaggaggtcgagtacacggattgggagcaggttaagaagttcgcggaggattttgcaaagctatcgtacaagaaggccctctag

SEQ ID NO: 301 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactaatggagagacagacacgagcaaggaggtcgagtacacggattgggagcaggttaagaagttcgcggaggattttgcaaagctatcgtacaagaaggccctctag

SEQ ID NO: 302 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactggggctgagacagacacgagcaaggaggtcgagtacacggattgggagcaggttaagaagttcgcggaggattttgcaaagctatcgtacaagaaggccctctag

SEQ ID NO: 303 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggaaagagacagacacgagcaaggaggtcgagtacacggattgggagcaggttaagaagttcgcggaggattttgcaaagctatcgtacaagaaggccctctag

SEQ ID NO: 304 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggatggagacagacacgagcaaggaggtcgagtacacggattgggagcaggttaagaagttcgcggaggattttgcaaagctatcgtacaagaaggccctctag

SEQ ID NO: 305 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggcgggagacagacacgagcaaggaggtcgagtacacggattgggagcaggttaagaagttcgcggaggattttgcaaagctatcgtacaagaaggccctctag

SEQ ID NO: 306 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggtctgagacagacacgagcaaggaggtcgagtacacggattgggagcaggttaagaagttcgcggaggattttgcaaagctatcgtacaagaaggccctctag

SEQ ID NO: 307 - DNA, 540 bp, synthetic construct
  misc_feature 1..540: note = PPO variant
  source 1..540: mol_type = other DNA
atgaaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatcgctctaataatgcggatgactgggggagagacagacacgagcaaggaggtcgagtacacggattgggagcaggttaagaagttcgcggaggattttgcaaagctatcgtacaagaaggccctctag

SEQ ID NO: 308 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgatttgcctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 309 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattgaactgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 310 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattggcctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 311 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcatctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 312 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattaaactgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 313 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattctgctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 314 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattatgctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 315 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcgcctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 316 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattagcctgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 317 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagaacattatgcgcatgaccggcggcgaaaccgataccagcaaagaagtggaatataccgattgggaacaggtgaaaaaatttgcggaagattttgcgaaactgagctataaaaaagcgctg

SEQ ID NO: 318 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagcagattatgcgcatgaccggcggcgaaaccgataccagcaaagaagtggaatataccgattgggaacaggtgaaaaaatttgcggaagattttgcgaaactgagctataaaaaagcgctg

SEQ ID NO: 319 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagagcattatgcgcatgaccggcggcgaaaccgataccagcaaagaagtggaatataccgattgggaacaggtgaaaaaatttgcggaagattttgcgaaactgagctataaaaaagcgctg

SEQ ID NO: 320 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcaggtgattatgcgcatgaccggcggcgaaaccgataccagcaaagaagtggaatataccgattgggaacaggtgaaaaaatttgcggaagattttgcgaaactgagctataaaaaagcgctg

SEQ ID NO: 321 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagtggattatgcgcatgaccggcggcgaaaccgataccagcaaagaagtggaatataccgattgggaacaggtgaaaaaatttgcggaagattttgcgaaactgagctataaaaaagcgctg

SEQ ID NO: 322 - DNA, 537 bp, synthetic construct
  misc_feature 1..537: note = PPO variant
  source 1..537: mol_type = other DNA
atgaaagcgctggtgctgtatagcacccgcgatggccagacccatgcgattgcgagctatattgcgagctgcatgaaagaaaaagcggaatgcgatgtgattgatctgacccatggcgaacatgtgaacctgacccagtatgatcaggtgctgattggcgcgagcattcgctatggccattttaacgcggtgctggataaatttattaaacgcaacgtggatcagctgaacaacatgccgagcgcgtttttttgcgtgaacctgaccgcgcgcaaaccggaaaaacgcaccccgcagaccaacccgtatgtgcgcaaatttctgctggcgaccccgtggcagccggcgctgtgcggcgtgtttgcgggcgcgctgcgctatccgcgctatcgctggattgataaagtgatgattcagtatattatgcgcatgaccggcggcgaaaccgataccagcaaagaagtggaatataccgattgggaacaggtgaaaaaatttgcggaagattttgcgaaactgagctataaaaaagcgctg

SEQ ID NO: 323 - protein, 68 aa, Arabidopsis thaliana
MATATTTATAAFSGVVSVGTETRRIYSFSHLQPSAAFPAKPSSFKSLKLKQSARLTRRLDHRPFVVRC

SEQ ID NO: 324 - protein, 68 aa, synthetic construct
  REGION 1..68: note = APG6 CTP variant
MASSTTTATAAFSGVVSVGTETRRIYSFSHLQPSAAFPAKPSSFKSLKLKQSARLTRRLDHRPFVVRC

SEQ ID NO: 325 - protein, 61 aa, Arabidopsis thaliana
MAPALSRSLYTSPLTSVPITPVSSRLSHLRSSFLPHGGALRTGVSCSWNLEKRCNRFAVKC

SEQ ID NO: 326 - protein, 72 aa, Petunia hybrida
MAQINNMAQGIQTLNPNSNFHKPQVPKSSSFLVFGSKKLKNSANSMLVLKKDSIFMQKFCSFRISASVATAC

SEQ ID NO: 327 - protein, 57 aa, Pisum sativum
MASMISSSAVTTVSRASRGQSAAMAPFGGLKSMTGFPVRKVNTDITSITSNGGRVKC

SEQ ID NO: 328 - protein, 77 aa, Oryza sativa
MAALTTSQLATSATGFGIADRSAPSSLLRHGFQGLKPRSPAGGDATSLSVTTSARATPKQQRSVQRGSRRFPSVVVC

SEQ ID NO: 329 - DNA, 204 bp, synthetic construct
  misc_feature 1..204: note = Codon optimized
  source 1..204: mol_type = other DNA
atggccaccgccaccactaccgccaccgctgcgttctccggcgtggtgagcgtcggcactgagacgcgcaggatctactccttcagccacctccagccttctgctgcgttccccgctaagccgtcttcgttcaagagcctgaagctgaaacagtccgcacgccttacccggcgcctggaccataggccattcgttgtcaggtgc

SEQ ID NO: 330 - DNA, 204 bp, synthetic construct
  misc_feature 1..204: note = Codon optimized
  source 1..204: mol_type = other DNA
atggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 331 - DNA, 204 bp, synthetic construct
  misc_feature 1..204: note = Codon optimized
  source 1..204: mol_type = other DNA
atggctactgctactaccacagctaccgctgcattctctggtgttgtgagtgttggaaccgagacacgtagaatttactctttctcacacttgcaacctagcgcagccttccctgccaagccatcatcctttaagtccttgaagctgaaacagtcggcgaggcttacgaggcgcctcgatcatagaccctttgtggtccgatgc

SEQ ID NO: 332 - DNA, 204 bp, synthetic construct
  misc_feature 1..204: note = Codon optimized
  source 1..204: mol_type = other DNA
atggcaaccgcgactaccaccgcaacggcagctttctccggggtagtttcagtcgggacggaaacccgcaggatctattcgttcagccatttgcaaccttccgcggcctttcccgccaaaccctcgtcttttaaatcgctgaaactcaaacagtcagcacggttgacccgaagattggaccaccgcccatttgtagtgaggtgc

SEQ ID NO: 333 - DNA, 204 bp, synthetic construct
  misc_feature 1..204: note = Codon optimized
  source 1..204: mol_type = other DNA
atggccacggccacgaccacggcaacagcggccttttcgggcgttgtatccgtcgggacagaaaccagacgcatatatagcttctcacacctgcaacctagtgccgcttttccggccaaacctagctcgtttaaatcgctgaagctcaaacagagcgctcggttaactagacgactggaccataggccatttgtcgtccgctgc

SEQ ID NO: 334 - DNA, 204 bp, synthetic construct
  misc_feature 1..204: note = Recombinant
  source 1..204: mol_type = other DNA
atggcttcctccacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgt

SEQ ID NO: 335 - DNA, 183 bp, synthetic construct
  misc_feature 1..183: note = Codon optimized for dicot expression
  source 1..183: mol_type = other DNA
atggctcctgctttgagtagaagtctctacacatctcctttgacttcagttccaatcactcctgtctcttctcgtctctctcatctgagaagctcgtttctcccacacggcggcgctttaagaaccggcgtttcgtgtagctggaatctcgaaaagagatgtaaccgattcgccgtgaagtgt

SEQ ID NO: 336 - DNA, 183 bp, synthetic construct
  misc_feature 1..183: note = Codon optimized for monocot expression
  source 1..183: mol_type = other DNA
atggctccggctctcagccgctccctctacaccagccctctcacctccgtgcccatcaccccggtgtcctcccgcctctcccacctccgctcctccttcctccctcacggcggcgcgctccgcaccggcgtgtcctgctcctggaacctggagaagcgctgcaaccgcttcgccgtgaagtgc

SEQ ID NO: 337 - DNA, 216 bp, synthetic construct
  misc_feature 1..216: note = Recombinant
  source 1..216: mol_type = other DNA
atggcccagatcaacaacatggcccagggcatccagaccctgaaccctaactctaacttccacaagccgcaagtgcccaagtctagctccttcctcgtgttcggctccaagaagctcaagaatagcgccaattccatgctggtcctgaagaaagactcgatcttcatgcagaagttctgctcctttcgcatcagtgcttcggttgcgactgcctgc

SEQ ID NO: 338 - DNA, 171 bp, synthetic construct
  misc_feature 1..171: note = Recombinant
  source 1..171: mol_type = other DNA
atggcttctatgatatcctcttccgctgtgacaacagtcagccgtgcctctagggggcaatccgccgcaatggctccattcggcggcctcaaatccatgactggattcccagtgaggaaggtcaacactgacattacttccattacaagcaatggtggaagagtaaagtgc

SEQ ID NO: 339 - DNA, 231 bp, synthetic construct
  misc_feature 1..231: note = Recombinant
  source 1..231: mol_type = other DNA
atggcggctctcaccacgtcccagctcgccacctcggccaccggcttcggcatcgccgacaggtcggcgccgtcgtcgctgctccgccacgggttccagggcctcaagccccgcagccccgccggcggcgacgcgacgtcgctcagcgtgacgaccagcgcgcgcgcgacgcccaagcagcagcggtcggtgcagcgtggcagccggaggttcccctccgtcgtcgtgtgc

SEQ ID NO: 340 - protein, 35 aa, unidentified
  source 1..35: note = Gossypium raimondii
MLNIAPSCVLASGISKPVTKMASTENKDDHSSAKR

SEQ ID NO: 341 - DNA, 105 bp, synthetic construct
  misc_feature 1..105: note = Codon optimized for dicot expression
  source 1..105: mol_type = other DNA
atgcttaacattgcgccgagttgtgttttggccagcgggatctctaagcccgtgaccaagatggctagcacggagaacaaggacgaccacagcagcgccaagagg

SEQ ID NO: 342 - protein, 45 aa, Canavalia rosea
MVAVFNDVVFPPSQTLLRPSFHSPTFFFSSPTPKFTRTRPNRILR

SEQ ID NO: 343 - protein, 60 aa, Senna obtusifolia
MPAIAMASLTDLPSLSPTQTLVHSNTSFISSRTCFVCPIIPFPSRSQLNRRIACIRSNVR

SEQ ID NO: 344 - protein, 55 aa, unidentified
  source 1..55: note = Nicotiana benthamiana
MTTTPVANHPNIFTHRSPPSSSSSSPSAFLTRTSFLPFSSICKRNSVNCNGWRTR

SEQ ID NO: 345 - protein, 34 aa, Brassica napus
MDFSLLRPASTQPFLSPFSNPFPRSRPYKPLNLR

SEQ ID NO: 346 - protein, 37 aa, Adansonia digitata
MAILIDLSLLRSSPSVFSFSKPNHRIPPRIYKPFKLR

SEQ ID NO: 347 - protein, 47 aa, unidentified
  source 1..47: note = Rosa hybrida osiana
MTTLSRLADLPSFAAPPPLLTHRPPPSVFLTPKPTKPSPPHHFFKLR

SEQ ID NO: 348 - protein, 46 aa, Xanthium strumarium
MSSLTDLPSLNHYRTCSPRPFPISRQTSSSINPNNLTTSNRWRRFR

SEQ ID NO: 349 - protein, 35 aa, Conyza canadensis
MTSLTNFTPLKLTNPNYLNTTTTYNHRKLSNFRFR

SEQ ID NO: 350 - protein, 45 aa, Amaranthus palmeri
MSAMALSSSILQCPPHSDISFRFSAYTATRSPFFFGRPRKLSYIH

SEQ ID NO: 351 - protein, 47 aa, Spinacia oleracea
MSAMALSSTMALSLPQSSMSLSHCRHNRITILIPSSSLRRRGGSSIR

SEQ ID NO: 352 - protein, 61 aa, Kochia scoparia
MSAMASPSIIPQSFLQRSPTSLQSRSNYSKNHIIISISTPCSHGKNQRRFLRKTTHFRSIH

SEQ ID NO: 353 - protein, 34 aa, Setaria italica
MVAAAMATAPSAGVPPLRGTRGPARFRIRGVSVR

SEQ ID NO: 354 - protein, 27 aa, Allium cepa
MATTTAAAAVTISIPKKPVFIRRPRLR

SEQ ID NO: 355 - protein, 33 aa, Digitaria sanguinalis
MLSSTATASSASSHHPYRSASARASSTRLRPVL

SEQ ID NO: 356 - protein, 49 aa, Amaranthus palmeri
MVIQSITHLSPKLALPSPLSISAKNYPVAVMGNISEREEPTSAKRVAVV

SEQ ID NO: 357 - protein, 48 aa, Spinacia oleracea
MVILPVSQLSTNLGLSLVSPTKNNPVMGNVSERNQVNQPISAKRVAVV

SEQ ID NO: 358 - protein, 51 aa, Sedum album
MLSLSSSHSSATTYSLRQRYSTTTKGSLNQPEMASAENPSSKGSGKRGAVV

SEQ ID NO: 359 - protein, 38 aa, Camelina sativa
MELSLLRPSTQSLLPSFSKPNLRLHVYKPLKLRCSVAG

SEQ ID NO: 360 - protein, 43 aa, Cucumis melo
MATGATLLTDLPFRRPHPLTLLRPSDIPSFYPLHISLQNNRLR

SEQ ID NO: 361 - protein, 30 aa, Eragrostis tef
MVAAAATMATAAPPLRAPQTLARPRRGSVR

SEQ ID NO: 362 - protein, 32 aa, Canavalia rosea
MYVSPASNNPRACLKLSQEMASSAADGNPRSV

SEQ ID NO: 363 - protein, 42 aa, Eragrostis tef
MLSSAATASSASAHPYRPASARASRSVLAMAGSDDTRAAPAR

SEQ ID NO: 364 - protein, 41 aa, Andropogon gerardii
MVAATAMATAASAAAPLLNGTRRPARLRHRGLRVRCAAVAG

SEQ ID NO: 365 - protein, 24 aa, Digitaria sanguinalis
MLSSTATASSASSHHPYRSASARA

SEQ ID NO: 366 - protein, 23 aa, Eragrostis tef
MLSSAATASSASAHPYRPASARA

SEQ ID NO: 367 - protein, 28 aa, Amaranthus graecizans
MSAMALSSSILQCPPHSDISFRFFAHTR

SEQ ID NO: 368 - protein, 13 aa, Ambrosia trifida
MASPTIVDNQKPA

SEQ ID NO: 369 - protein, 14 aa, Brassica napus
MASNAAADHDKLSG

SEQ ID NO: 370 - protein, 13 aa, unidentified
  source 1..13: note = Rosa hybrida osiana
MASPSPGDKHSSV

SEQ ID NO: 371 - protein, 55 aa, Amaranthus cruentus
MKGRKRRITRESAREMSAMALSSSILQCPPHSDISFRFSAHSPTHSPIFFGRPRK

SEQ ID NO: 372 - protein, 45 aa, Taraxacum officinale
MTYLTDVGSLNCYRSWPSLPAPGTVGALTSKNPRYLITYGPAHRK

SEQ ID NO: 373 - protein, 67 aa, Canavalia rosea
MVAVFNDVVFPPSQTLLRPSFHSPTFFFSSPTPKFTRTRPNRILRCSIAQESTTSPSQSRESAPLDC

SEQ ID NO: 374 - protein, 52 aa, Eragrostis tef
MVAAAATMATAAPPLRAPQTLARPRRGSVRCAVVSDAAEAPAAPGARLSADC

SEQ ID NO: 375 - protein, 51 aa, Allium cepa
MATTTAAAAVTISIPKKPVFIRRPRLRCSAVASDAIISNEAPTGTTISADC

SEQ ID NO: 376 - protein, 77 aa, Taraxacum officinale
MTYLTDVGSLNCYRSWPSLPAPGTVGALTSKNPRYLITYGPAHRKCNSWRFRCSIAKDSPITPPISNESNSQPLLDC

SEQ ID NO: 377 - protein, 71 aa, Cucumis melo
MATGATLLTDLPFRRPHPLTLLRPSDIPSFYPLHISLQNNRLRSHFRCSIAEGSTALSPSNASSQSSILDC

SEQ ID NO: 378 - protein, 71 aa, Xanthium strumarium
MSSLTDLPSLNHYRTCSPRPFPISRQTSSSINPNNLTTSNRWRRFRCSIANDTPISPPISSDSTSHPFLDC

SEQ ID NO: 379 - protein, 65 aa, Taraxacum officinale
MTYLTDVGSLNCYRSWPSLPAPGTVGALTSKNPRYLITYGPAHRKDSPITPPISNESNSQPLLDC

SEQ ID NO: 380 - protein, 60 aa, Senna obtusifolia
MPAIAIASLTDLPSLSPTQTLVHSNTSFISSRTCFVCPIIPFPSRSQLNRRIACIRSNVR

SEQ ID NO: 381 - protein, 19 aa, Eragrostis tef
MVAAAEAPAAPGARLSADC

SEQ ID NO: 382 - protein, 25 aa, Allium cepa
MATTTASDAIISNEAPTGTTISADC

SEQ ID NO: 383 - protein, 12 aa, Allium cepa
MATTGTTISADC

SEQ ID NO: 384 - protein, 19 aa, Cucumis melo
MATALSPSNASSQSSILDC

SEQ ID NO: 385 - protein, 32 aa, Xanthium strumarium
MSSLTDLPSLNHYRTCSPPISSDSTSHPFLDC

SEQ ID NO: 386 - protein, 56 aa, synthetic construct
  REGION 1..56: note = Recombinant
MATTTAAAAVTISIPKKPVFIRRPRLRGPVDCRGLHASDAIISNEAPTGTTISADC

SEQ ID NO: 387 - protein, 76 aa, Eragrostis tef
MAAAPPLAADMVLPSPCPAAVAPTPVVAAAWGAARAGSVRCKATQLRMMRTGGPVAPVAGRRRRAPLSVRCDASSR

SEQ ID NO: 388 - protein, 80 aa, Setaria italica
MAAAPPLSADALSFLPSAAAPAAAAPTPVVAAAWGAARAAGSVRGKAALRMARRGSGLAPVVGRRPRRPPLSVRCDATSR

SEQ ID NO: 389 - protein, 84 aa, Acalypha ostryifolia
MATTTATTSFSGVSICPPHQTNRTSLFPPQSLSFPSSKHGSLVNSVQFNRSRRARRNHFSLTSITNAPRRKRLLSVRCDASATS

SEQ ID NO: 390 - protein, 78 aa, Adansonia digitata
MAASSSSVVSFSGISLCSTHSISNKTYLFSAHPRISVSFPSKPNSLKSFKQLQLKKNGLFEKFSRTSSRSFVVRCDAS

SEQ ID NO: 391 - protein, 82 aa, Taraxacum officinale
MATTASFSGVRIHAPSSTCIDRTTLFAQPSVSFSSFSKPRRTTLRSLKLRSRSNDVLLRTRTGDRFGGKSSRSFVVRCDASS

SEQ ID NO: 392 - protein, 77 aa, Amaranthus cruentus
MATATTSFPGAYLRVPPKNGVRNALFSQSIVSIARNSRKPKSLKSLKLSTNSFNFGLHKSCRKGSKSGSFVVRCDAA

SEQ ID NO: 393 - protein, 80 aa, Amaranthus cruentus
MAIATTSFPGTYLRVPPKNGVRNALFSRSVVSNGVNSRKPNSLESLKSSRNSSNVCLSTSFGHYRKSSKSGSFFVRCNAA

SEQ ID NO: 394 - protein, 77 aa, Amaranthus palmeri
MATATTSFPGAYLRVPPKNGVRNALFSQSIVSIALNSRKPKSFKSLKSSANSCNFGLHKSYRKGSKSGSFVVRCDAA

SEQ ID NO: 395 - protein, 80 aa, Amaranthus palmeri
MATATTSFPGTYLRVPPKNGVRNALFSRSVVSNGVNSRKPNSLKSLKLSRNSSNVCLYTSFGHYRKSSKSGSFIIRCNAA

SEQ ID NO: 396 - protein, 80 aa, Amaranthus palmeri
MATATTSFPGTYLRVPPKNGVRNALFSRSVVSNGVNSRKPKSLKSLKSSRNSSNVCLYTSFGHYRKSSKSGSFIIRCNAA

SEQ ID NO: 397 - protein, 80 aa, unidentified
  source 1..80: note = Amaranthus rudis
MATATTSFPGTYLRVPPKNGVRNALFSRSVVSNGVDSRKPNSLKSMKLSRNSSNVCLYTSFGHYRKSSKSGSFIVRCNAA

SEQ ID NO: 398 - protein, 80 aa, Amaranthus viridis
MAIATTSFPGTYLRVPPKNGVRNALFSRSVVSNGVNSRKPNSLESLKSSRNSSNVCLSTSFGHYRKGSKSGSFVVRCDAA

SEQ ID NO: 399 - protein, 71 aa, Ambrosia trifida
MSTMSTLFHLPSSLCTDRTITSSFAQPSVSVNSFSKPRRVALRSLKLKTRSNDVLLRKSSRSLVVRCDASS

SEQ ID NO: 400 - protein, 87 aa, Conyza canadensis
MATAAFSGVPCIDRTSLLSAQPSSSSSSSVVVCYSSFSKPGTTLLPSLKLKSSRNNNNSNVFLFGNTRKTSRLSFLVRCDSSSSSSS

SEQ ID NO: 401 - protein, 71 aa, Cucumis melo
MAFSTAPFYAIGIRFPSHSSSISSTTNALILKSPLALALTAKPKSPLLLKRNVGCQRFGRNSRFVVRCDAS

SEQ ID NO: 402 - protein, 78 aa, Kochia scoparia
MATATTSFPGAYLHLPPKNGVRNALFSQPICSSNLNLKKPNSLKSVKLSRSSGNALFYKNAKKNSKFGSLVVRCDAAG

SEQ ID NO: 403 - protein, 84 aa, unidentified
  source 1..84: note = Rosa hybrida
MASSTTSFAASGVGLRLPQSVSTKCCSKASLFPHPTLSLTFHARPQFFRGLASRQFNPNGAFGTGSGRLGRTPNPFVVRSEASS

SEQ ID NO: 404 - protein, 80 aa, Sedum album
MAASAATITSSISAITPKPSSFSSSPSVTVPRFSVSCSAIPRPHKNPCSLKFRVKDSRFNGIVKKRSNSNSFVVRCDTSS

SEQ ID NO: 405 - protein, 79 aa, Sedum album
MAADAATITAGITLTTARRSSSSIAPQFSVCCSAITNTQKNLSFLKLRVKDATLTTRIEGIQKKRYNSASFVVRCDASS

SEQ ID NO: 406 - protein, 81 aa, Spinacia oleracea
MATATTSFLGAYLRVPPNNGVRNALFSQPFLSLRIKSKRTKSLNSLKFTGDSSKILLFKCSRPFEKGLKSGSFVVRCDAAG

SEQ ID NO: 407 - protein, 79 aa, Allium cepa
MAATSSATTHLPFFSPHTKHAKTNSFFASLPVSAYSTKNSISFKALKAVRWSETFGQSKKANGFAKRKQFAVVRCDASS

SEQ ID NO: 408 - DNA, 135 bp, synthetic construct
  misc_feature 1..135: note = Recombinant
  source 1..135: mol_type = other DNA
atggtggctgtgttcaacgacgtagtgttccctccttcgcagacccttcttcgcccctccttccacagcccgacgttcttttttagcagccccacaccaaagttcacgcgtacgaggccgaatagaatactgcgg

SEQ ID NO: 409 - DNA, 180 bp, synthetic construct
  misc_feature 1..180: note = Recombinant
  source 1..180: mol_type = other DNA
atgccggcgatagcaatggcttctttaactgatctgccgtcgttgagccccacacagaccctcgttcactcgaacacgagcttcatttcatcgagaacctgcttcgtctgtccgatcatccccttcccatcgaggtcgcaactgaaccgccgcatcgcctgcatcaggtccaacgtaagg

SEQ ID NO: 410 - DNA, 165 bp, synthetic construct
  misc_feature 1..165: note = Recombinant
  source 1..165: mol_type = other DNA
atgaccacaaccccggtagcaaaccaccccaatatcttcactcaccgaagccctccgtcatcttcctcgtcctcacccagcgcgtttctgacccgcacctcctttctgcccttctctagcatctgcaaaaggaactctgtgaactgcaatgggtggcgaacccgg

SEQ ID NO: 411 - DNA, 102 bp, synthetic construct
  misc_feature 1..102: note = Recombinant
  source 1..102: mol_type = other DNA
atggacttcagtctccttaggcccgcttcgacgcagccgttcctctcacccttctccaatcccttcccacggagtaggccatacaagccacttaatctgagg

SEQ ID NO: 412 - DNA, 111 bp, synthetic construct
  misc_feature 1..111: note = Recombinant
  source 1..111: mol_type = other DNA
atggccatcttgattgacctctccctcctgaggtcctctccgtcggtcttctccttctccaagccgaaccacaggataccaccgcggatctacaagccgttcaagttgagg

SEQ ID NO: 413 - DNA, 141 bp, synthetic construct
  misc_feature 1..141: note = Recombinant
  source 1..141: mol_type = other DNA
atgaccacgctttccaggctcgctgaccttccttcttttgctgcccctcctcctctcttgacccaccggccccctccttcagttttcctgactccgaagccgacaaagccgtcacctccacatcacttctttaaactgcgc

SEQ ID NO: 414 - DNA, 138 bp, synthetic construct
  misc_feature 1..138: note = Recombinant
  source 1..138: mol_type = other DNA
atgtcgtccctaacggacctcccctccctgaatcactataggacgtgcagcccgcgcccattccccatctccaggcagaccagttcatcaattaacccaaacaacttgacgaccagtaaccgttggcgcaggttcagg

SEQ ID NO: 415 - DNA, 105 bp, synthetic construct
  misc_feature 1..105: note = Recombinant
  source 1..105: mol_type = other DNA
atgacgagtctcaccaacttcaccccgctcaagctgacgaaccccaactacctcaacacgaccaccacctacaaccaccgtaagctctccaacttccggttccgc

SEQ ID NO: 416 - DNA, 135 bp, synthetic construct
  misc_feature 1..135: note = Recombinant
  source 1..135: mol_type = other DNA
atgtcggccatggcgctgtccagcagcattctacagtgcccgcctcactcagacatatccttccgcttctcggcatacactgccacccgctcacctttcttcttcgggaggccaaggaaactatcttacatccac

SEQ ID NO: 417 - DNA, 141 bp, synthetic construct
  misc_feature 1..141: note = Recombinant
  source 1..141: mol_type = other DNA
atgtcggccatggcattgagctccaccatggccctcagcctgccacaatctagcatgtccttgagccactgcagacacaatagaataactattctgatcccctcgagctcgttacggcgacggggaggttcctcgatccgc

SEQ ID NO: 418 - DNA, 183 bp, synthetic construct
  misc_feature 1..183: note = Recombinant
  source 1..183: mol_type = other DNA
atgtctgctatggcgagcccctccatcatcccgcagtcgttcctccagcgaagcccgacctccttgcaatctcgatccaactactcgaagaaccacatcatcatctccatcagcaccccgtgctctcatgggaagaaccagcgacgtttcttgcgaaagaccacccacttccgatccatccac

SEQ ID NO: 419 - DNA, 102 bp, synthetic construct
  misc_feature 1..102: note = Recombinant
  source 1..102: mol_type = other DNA
atggtcgccgctgcaatggctacagccccttccgctggagtccctcctcttagagggacaaggggtccagcaaggtttagaatccggggagtgtcagtgcgt

SEQ ID NO: 420 - DNA, 81 bp, synthetic construct
  misc_feature 1..81: note = Recombinant
  source 1..81: mol_type = other DNA
atggccactaccacagcagccgcggcggtcaccatcagcattcctaaaaagcctgtttttatccgccgcccacgacttcgt

SEQ ID NO: 421 - DNA, 99 bp, synthetic construct
  misc_feature 1..99: note = Recombinant
  source 1..99: mol_type = other DNA
atgttgtctagcactgctactgcaagttctgcatcctcacaccacccctaccgttcagcttctgcaagggcttcgtcgacacgtctccgcccggtcctt

SEQ ID NO: 422 - DNA, 147 bp, synthetic construct
  misc_feature 1..147: note = Recombinant
  source 1..147: mol_type = other DNA
atggtcattcagtcaattacgcatctttctcccaagctcgcactgccctctccgctgtcgatctcggctaagaactacccggtggccgtgatggggaatatcagcgagagggaggagccaacttctgctaaaagggtggccgtggtg

SEQ ID NO: 423 - DNA, 144 bp, synthetic construct
  misc_feature 1..144: note = Recombinant
  source 1..144: mol_type = other DNA
atggtcattctacccgtgtcccagctctcgactaatttggggctttcccttgttagtccaacgaagaacaacccggtgatgggcaacgtgtccgagaggaaccaggtgaaccagccaatctccgccaagcgcgttgctgtcgtg

SEQ ID NO: 424 - DNA, 153 bp, synthetic construct
  misc_feature 1..153: note = Recombinant
  source 1..153: mol_type = other DNA
atgctctcactgagcagctcccactcatccgcgacaacgtattctctccggcaacggtactctacaacgaccaaaggttcgttgaaccagcctgagatggccagcgccgaaaacccttccagcaagggatcaggtaagagaggagcagtggtg

SEQ ID NO: 425 - DNA, 114 bp, synthetic construct
  misc_feature 1..114: note = Recombinant
  source 1..114: mol_type = other DNA
atggagctgagcctcctaagaccgtctactcagtcattgctcccctcgttcagcaagcctaatttgcggctccacgtgtacaagccccttaagctccgatgcagcgtagccggt

SEQ ID NO: 426 - DNA, 114 bp, synthetic construct
  misc_feature 1..114: note = Recombinant
  source 1..114: mol_type = other DNA
atggagctaagcctcctaagaccgtctactcagtcattgctcccctcgttcagcaagcctaatttgcggctccacgtgtacaagccccttaagctccgatgcagcgtagccggt

SEQ ID NO: 427 - DNA, 129 bp, synthetic construct
  misc_feature 1..129: note = Recombinant
  source 1..129: mol_type = other DNA
atggcgacaggagccaccctgctaacagacctgccgttccgtaggccgcacccgcttacgctcttacgtccgagcgatatcccgtccttttacccactacacataagcctacagaacaatcgtttgagg

SEQ ID NO: 428 - DNA, 90 bp, synthetic construct
  misc_feature 1..90: note = Recombinant
  source 1..90: mol_type = other DNA
atggtggctgctgcggcaacgatggctaccgccgcaccaccattaagagcgcctcaaactcttgcacgaccgcgaagaggtagtgtgaga

SEQ ID NO: 429 - DNA, 96 bp, synthetic construct
  misc_feature 1..96: note = Recombinant
  source 1..96: mol_type = other DNA
atgtatgtgtcgcccgcctcgaacaacccacgagcatgcctcaagctgtcacaggaaatggcgtcttcagcagcagacggcaacccaagatccgtt

SEQ ID NO: 430 - DNA, 126 bp, synthetic construct
  misc_feature 1..126: note = Recombinant
  source 1..126: mol_type = other DNA
atgttgtctagcgcagcgacagctagcagcgcaagtgctcatccttatcgacctgcttctgcccgggcgagtaggagcgtgttggctatggctggatcagacgatactagggcagctcctgcccgg

SEQ ID NO: 431 - DNA, 123 bp, synthetic construct
  misc_feature 1..123: note = Recombinant
  source 1..123: mol_type = other DNA
atggtggctgcgaccgcaatggccaccgctgcttcggctgctgcgcctctcctaaacggaacgagacgaccggcacgattgagacatagaggtttacgtgttaggtgtgctgcagtagcagga

SEQ ID NO: 432 - DNA, 72 bp, synthetic construct
  misc_feature 1..72: note = Recombinant
  source 1..72: mol_type = other DNA
atgctttctagcactgccacagcttcctcagcttctagccaccacccgtatcgttcagcttcggcacgtgcc

SEQ ID NO: 433 - DNA, 69 bp, synthetic construct
  misc_feature 1..69: note = Recombinant
  source 1..69: mol_type = other DNA
atgcttagctcagcagctacggcctctagtgcttctgcccatccataccgtcccgcatctgctcgagca

SEQ ID NO: 434 - DNA, 84 bp, synthetic construct
  misc_feature 1..84: note = Recombinant
  source 1..84: mol_type = other DNA
atgagcgcgatggcgctttcttctagcatcttgcaatgccccccccactctgacatttctttccgcttcttcgcccacactcgc

SEQ ID NO: 435 - DNA, 39 bp, synthetic construct
  misc_feature 1..39: note = Recombinant
  source 1..39: mol_type = other DNA
atggcgagtcccacgatcgttgacaaccagaagccagcg

SEQ ID NO: 436 - DNA, 42 bp, synthetic construct
  misc_feature 1..42: note = Recombinant
  source 1..42: mol_type = other DNA
atggctagtaacgccgctgctgaccacgataagctctcgggt

SEQ ID NO: 437 - DNA, 39 bp, synthetic construct
  misc_feature 1..39: note = Recombinant
  source 1..39: mol_type = other DNA
atggcgtcgccgtccccaggcgacaaacattcgtctgta

SEQ ID NO: 438 - DNA, 165 bp, synthetic construct
  misc_feature 1..165: note = Recombinant
  source 1..165: mol_type = other DNA
atgaaggggcggaagagacggatcacgcgggagtctgcaagggagatgtcagcgatggcattgtcttcgagcatactccagtgccctcctcactccgacatctctttccgttttagcgctcactcaccgacacacagccctatcttctttgggcgtcccaggaaa

SEQ ID NO: 439 - DNA, 135 bp, synthetic construct
  misc_feature 1..135: note = Recombinant
  source 1..135: mol_type = other DNA
atgacctacctcactgatgtgggtagtctcaattgctacaggtcctggcctagcctaccggcccctgggacggtcggagcattgacttctaagaacccccgctacttgatcacatacggtccggctcaccgaaag

SEQ ID NO: 440 - DNA, 135 bp, synthetic construct
  misc_feature 1..135: note = Recombinant
  source 1..135: mol_type = other DNA
atgacctacctcactgatgtgggtagtctcaattgctacaggtcttggcctagcctaccggcccctgggacggtcggagcattgacttctaagaacccccgctacttgatcacatacggtccggctcaccgaaag

SEQ ID NO: 441 - DNA, 201 bp, synthetic construct
  misc_feature 1..201: note = Recombinant
  source 1..201: mol_type = other DNA
atggtggctgtgttcaacgacgtagtgttccctccttcgcagacccttcttcgcccctccttccacagcccgacgttcttttttagcagccccacaccaaagttcacgcgtacgaggccgaatagaatactgcggtgctcgattgcgcaggagtctacaacatcgccgtcgcagtcgcgagagtcagctccactcgattgt

SEQ ID NO: 442 - DNA, 156 bp, synthetic construct
  misc_feature 1..156: note = Recombinant
  source 1..156: mol_type = other DNA
atggtggctgctgcggcaacgatggctaccgccgcaccaccattaagagcgcctcaaactcttgcacgaccgcgaagaggtagtgtgagatgtgccgtcgttagcgatgctgcagaagctccggctgctcctggcgctagactctctgcagattgc

SEQ ID NO: 443 - DNA, 153 bp, synthetic construct
  misc_feature 1..153: note = Recombinant
  source 1..153: mol_type = other DNA
atggccactaccacagcagccgcggcggtcaccatcagcattcctaaaaagcctgtttttatccgccgcccacgacttcgttgctcggcagttgcatccgacgcaatcatctccaacgaggcccctacagggacgacaatctcggctgactgt

SEQ ID NO: 444 - DNA, 231 bp, synthetic construct
  misc_feature 1..231: note = Recombinant
  source 1..231: mol_type = other DNA
atgacctacctcactgatgtgggtagtctcaattgctacaggtcctggcctagcctaccggcccctgggacggtcggagcattgacttctaagaacccccgctacttgatcacatacggtccggctcaccgaaagtgcaacagctggcgcttccggtgctctattgcaaaggactcccccatcacgcccccaatttcgaacgagagcaattcacagcccctgctagactgc

SEQ ID NO: 445 - DNA, 213 bp, synthetic construct
  misc_feature 1..213: note = Recombinant
  source 1..213: mol_type = other DNA
atggcgacaggagccaccctgctaacagacctgccgttccgtaggccgcacccgcttacgctcttacgtccgagcgatatcccgtccttttacccactacacataagcctacagaacaatcgtttgaggagtcatttcaggtgctcaatcgccgagggctcgacggcactgagcccatctaacgcatcgtcgcaatcgagtatcttggactgc

SEQ ID NO: 446 - DNA, 213 bp, synthetic construct
  misc_feature 1..213: note = Recombinant
  source 1..213: mol_type = other DNA
atgtcgtccctaacggacctcccctccctgaatcactataggacgtgcagcccgcgcccattccccatctccaggcagaccagttcatcaattaacccaaacaacttgacgaccagtaaccgttggcgcaggttcaggtgctctattgcgaacgacaccccgatcagcccgccgatttccagcgactctacttcccaccctttcttggactgt

SEQ ID NO: 447 - DNA, 195 bp, synthetic construct
  misc_feature 1..195: note = Recombinant
  source 1..195: mol_type = other DNA
atgacctacctcactgatgtgggtagtctcaattgctacaggtcctggcctagcctaccggcccctgggacggtcggagcattgacttctaagaacccccgctacttgatcacatacggtccggctcaccgaaaggactcccccatcacgcccccaatttcgaacgagagcaattcacagcccctgctagactgc

SEQ ID NO: 448 - DNA, 180 bp, synthetic construct
  misc_feature 1..180: note = Recombinant
  source 1..180: mol_type = other DNA
atgccggcgatagcaatagcttctttaactgatctgccgtcgttgagccccacacagaccctcgttcactcgaacacgagcttcatttcatcgagaacctgcttcgtctgtccgatcatccccttcccatcgaggtcgcaactgaaccgccgcatcgcctgcatcaggtccaacgtaagg

SEQ ID NO: 449 - DNA, 57 bp, synthetic construct
  misc_feature 1..57: note = Recombinant
  source 1..57: mol_type = other DNA
atggtggctgctgcggaagctccggctgctcctggcgctagactctctgcagattgc

SEQ ID NO: 450 - DNA, 75 bp, synthetic construct
  misc_feature 1..75: note = Recombinant
  source 1..75: mol_type = other DNA
atggccactaccacagcatccgacgcaatcatctccaacgaggcccctacagggacgacaatctcggctgactgt

SEQ ID NO: 451 - DNA, 36 bp, synthetic construct
  misc_feature 1..36: note = Recombinant
  source 1..36: mol_type = other DNA
atggccactacagggacgacaatctcggctgactgt

SEQ ID NO: 452 - DNA, 57 bp, synthetic construct
  misc_feature 1..57: note = Recombinant
  source 1..57: mol_type = other DNA
atggcgacggcactgagcccatctaacgcatcgtcgcaatcgagtatcttggactgc

SEQ ID NO: 453 - DNA, 96 bp, synthetic construct
  misc_feature 1..96: note = Recombinant
  source 1..96: mol_type = other DNA
atgtcgtccctaacggacctcccctccctgaatcactataggacgtgcagcccgccgatttccagcgactctacttcccaccctttcttggactgt

SEQ ID NO: 454 - DNA, 168 bp, synthetic construct
  misc_feature 1..168: note = Recombinant
  source 1..168: mol_type = other DNA
atggccactaccacagcagccgcggcggtcaccatcagcattcctaaaaagcctgtttttatccgccgcccacgacttcgtgggcccgtcgactgcagaggcctgcatgcatccgacgcaatcatctccaacgaggcccctacagggacgacaatctcggctgactgt

SEQ ID NO: 455 - DNA, 228 bp, Eragrostis tef
  source 1..228: mol_type = genomic DNA
atggcagccgcacctcccctagcagccgacatggtgttaccatccccatgccctgccgcggttgcacctaccccagtggttgcagctgcttggggtgcagcccgagctggatctgttagatgtaaagcgacccaacttcgaatgatgagaactgggggccctgttgctccagttgccggtagacgacgacgagctccattgagtgtacgttgtgatgcttcctccaga

SEQ ID NO: 456 - DNA, 240 bp, Setaria italica
  source 1..240: mol_type = genomic DNA
atggctgccgctcctcccctctctgcagatgcactatcattcctaccatccgccgccgctccggcagccgctgcaccaacacctgttgtagctgcggcatggggagccgcacgagctgcagggtcagttagaggtaaagctgctttgcgtatggctcgaaggggtagtggactggctccagtggttggaagaagacctcgacgacctcctctttcagttagatgtgacgcaacatctcgt

SEQ ID NO: 457 - DNA, 252 bp, Acalypha ostryifolia
  source 1..252: mol_type = genomic DNA
atggctacaaccaccgcgacgacgtctttctcgggcgtctcgatctgcccacctcaccagacgaatcgcacctctttgtttccgccccagtccttgtctttcccctccagtaagcatggcagtcttgtgaactctgtgcaattcaaccgttcgcgacgcgctagacgtaatcacttcagcctcacttccattaccaatgcaccgaggcgcaaaaggttactatctgtccggtgcgacgcgagtgccacatct

SEQ ID NO: 458 - DNA, 234 bp, Adansonia digitata
  source 1..234: mol_type = genomic DNA
atggcggcgtcatcttcgtccgtcgtgagcttctcgggcatctcgttgtgcagtactcactcgatctccaacaagacctatctattctccgcccacccgcgcatttcggtgtcgttccccagtaagcccaatagtttgaagtccttcaagcagctccagctgaagaagaacggactctttgagaagttctctcgtacctccagtcggagcttcgtggtgaggtgcgacgcgtcg

SEQ ID NO: 459 - DNA, 246 bp, Taraxacum officinale
  source 1..246: mol_type = genomic DNA
atggctacaaccgcgagcttctcgggtgttcgtattcacgcgccttcctccacatgtatcgaccggaccactttattcgcccagccttcggtgagcttttcttccttttccaagccgaggcgaacgaccttgaggtcgctgaagctaaggtcgaggtccaacgatgtgttgcttcgcacccgcacaggtgacagattcggcggaaagagctcacgttcatttgttgtgcgctgcgacgcatcttct

SEQ ID NO: 460 - DNA, 231 bp, Amaranthus cruentus
  source 1..231: mol_type = genomic DNA
atggcgaccgcgacgacctcgtttcccggcgcgtacctgcgcgtgccgcccaagaacggggttcgtaacgccctctttagccagtctatcgtgtcaatagcgcgcaactctcggaaacccaaatcgctcaaatcccttaaactatctaccaactcctttaacttcggtctgcacaagtcttgtcgaaagggaagcaaatccgggtcgttcgtagtgcgttgtgacgcggcc

SEQ ID NO: 461 - DNA, 231 bp, Amaranthus viridis
  source 1..231: mol_type = genomic DNA
atggccaccgccacgacgtcctttcccggtgcgtatctgcgagtgcctcccaagaacggcgtccggaacgcgctgttcagccagtccatcgtgagcatcgcgcggaatagtcggaaacctaagtcgctcaaatccttgaaactgtcaacgaactctttcaatttcgggttgcataagtcctgccgaaagggtagcaaatccgggtctttcgttgtgcggtgcgacgcggcc

SEQ ID NO: 462 - DNA, 240 bp, Amaranthus cruentus
  source 1..240: mol_type = genomic DNA
atggccatcgccaccacgagctttccgggaacgtacctccgggtgccgcccaagaacggcgtccgaaacgccctattcagtcgctccgtcgtgtctaatggggtgaactcaaggaagccgaactcgctggagtcgcttaaatcgtcgaggaatagctcgaacgtctgcttgagtacgtcgttcgggcattaccggaaatcgagtaagtcgggctcgttcttcgttcggtgtaacgccgcc

SEQ ID NO: 463 - DNA, 240 bp, Amaranthus hypochondriacus
  source 1..240: mol_type = genomic DNA
atggccatcgccacgacctcgttccccggcacgtacctgcgagtgccgcccaagaacggggtccggaacgcgctgttctctcggtccgtggtcagcaatggtgttaattcacgaaagccgaacagtctggaatctctcaagagcagtcgaaactcctccaacgtctgcctttcgaccagcttcggtcactaccggaagtctagtaagagcgggtcgttctttgtccggtgtaatgctgcc

SEQ ID NO: 464 - DNA, 231 bp, Amaranthus palmeri
  source 1..231: mol_type = genomic DNA
atggcgacggccaccacctcgttccccggcgcatacctccgcgtgccgcccaagaacggcgtccgcaacgcactctttagccagagcatcgtcagtatcgcccttaacagtcgcaagcctaaatcgttcaagtcactaaagtcaagcgctaattcgtgcaactttggacttcacaagtcctaccggaaaggcagcaagtctggcagcttcgtcgttcgttgtgatgccgcc

SEQ ID NO: 465 - DNA, 231 bp, Amaranthus palmeri
  source 1..231: mol_type = genomic DNA
atggccaccgcaacaaccagcttccctggcgcgtaccttcgtgtgccgcccaagaacggcgtccgcaatgcgctgtttagtcagtccatcgtgagtatcgctctcaattcccggaaacctaagagctttaagagcctaaagtcgagcgctaattcttgcaacttcgggcttcacaagagctatcggaaagggtctaagagcgggtcattcgtcgtgcggtgcgacgcggcc

SEQ ID NO: 466 - DNA, 231 bp, Amaranthus palmeri
  source 1..231: mol_type = genomic DNA
atggccacggccacgacctcgtttccgggtgcgtacctgcgagttccgcccaagaacggtgtacggaacgccttgttctcccaatccatcgtgagcatcgccctcaacagtcgcaaaccgaagtcattcaaatccctgaaaagttcggccaatagctgtaacttcgggctgcataagagttaccgcaaggggtcgaaatccgggtcgttcgtcgtccggtgcgacgctgcg

SEQ ID NO: 467 - DNA, 240 bp, Amaranthus palmeri
  source 1..240: mol_type = genomic DNA
atggccaccgccaccacctcgttccccggcacgtacctgcgcgtgccgcccaagaacggcgtcaggaacgcgctgtttagtcgctcagtcgtgtccaacggggtgaactcacgaaagcccaacagtttaaagagcttaaaactgtcgaggaactctagtaatgtctgcctctacacctccttcggacactatcgaaagtccagtaagtcgggctcattcatcatccggtgcaatgcggcc

SEQ ID NO: 468 - DNA, 240 bp, Amaranthus palmeri
  source 1..240: mol_type = genomic DNA
atggcaactgccacgacgtcctttccgggaacgtatctccgcgtgccgcccaagaacggcgtccgcaacgccctgttctcacgatccgtcgttagcaatggcgtcaatagccgcaagcctaagtccctgaaatcgctcaagtcgtcgcgcaactctagtaatgtctgtctctacacatcgttcggacattaccgcaaatcatccaaatccggctcgttcataatccggtgcaatgcggct

SEQ ID NO: 469 - DNA, 240 bp, unidentified
  source 1..240: mol_type = genomic DNA; note = Amaranthus rudis
atggcgacagcgaccacatccttccctggcacttacctgagagtgccgcccaagaatggggtgagaaacgccttgttcagccgcagcgtagtctctaatggggtggatagtcgcaaaccgaatagcctcaagagtatgaagctcagccgcaacagctcaaatgtctgcctctacacgagctttggccactaccgaaagtcctccaagtctgggtcgttcatcgtgcgctgtaacgccgcg

SEQ ID NO: 470 - DNA, 240 bp, unidentified
  source 1..240: mol_type = genomic DNA; note = Amaranthus rudis
atggccacggccaccacctcctttcctggcacatacctccgcgtccctcccaagaatggggtgcgaaacgcactctttagtagatcggtcgtttccaatggtgtcgattcccgcaagccgaactccctcaagtcgatgaagctgtcccgcaactcatcgaacgtttgcctctatacctcgtttgggcactaccgcaagtcgagcaaatcgggctcgttcattgtccggtgtaatgcagcc

SEQ ID NO: 471 - DNA, 240 bp, unidentified
  source 1..240: mol_type = genomic DNA; note = Amaranthus rudis
atggcgacggcgacaacctcgtttccgggaacgtacctgcgcgtgccgcccaagaacggggtgcggaacgccctgttcagccgctccgtcgtgtccaatggcgtcgattcgaggaagcctaactcattgaaatctatgaagttgtctcgtaattccagcaacgtttgcctctacacctcgttcgggcattaccgcaagtcaagcaagtccggatcgtttatcgtgcggtgcaacgctgcg

SEQ ID NO: 472 - DNA, 240 bp, Amaranthus viridis
  source 1..240: mol_type = genomic DNA
atggccattgccaccacgtcgtttcccggcacgtacctgagggttccgcccaagaacggagtccgcaacgcactgtttagtcgctccgtggtgagtaacggggtcaactccagaaaacctaattcgctggagtcccttaaatcgagccggaacagctcgaacgtctgcttgtcaacctcctttggccactaccggaagggctccaagtcgggctcattcgtcgtgcggtgcgatgcggcg

SEQ ID NO: 473 - DNA, 213 bp, Ambrosia trifida
  source 1..213: mol_type = genomic DNA
atgagtacgatgtcaaccctatttcacctcccgtctagcctgtgtaccgacaggacgatcaccagcagcttcgcacaaccgagcgtttcggtcaactcgttctcgaagccgcgccgcgtcgcgctccggtccttaaagctcaaaacgcgaagtaatgacgtcctgctgcggaaatcttcacgttcgctagtcgtgcgttgcgacgccagcagc

SEQ ID NO: 474 - DNA, 261 bp, Conyza canadensis
  source 1..261: mol_type = genomic DNA
atggcgacggccgccttctcgggcgttccgtgcattgaccggacatcactcctctccgcccagccatcgtcctcctcttccagtagcgtcgtggtctgctactcctcctttagcaagccgggcacgaccctattgccgtcgttgaagctcaaaagcagccgcaacaacaacaattcaaacgtattcctcttcgggaacaccaggaaaacatcccgtctgtcattcctagtgcgctgcgattcctcatcttcaagctctagc

SEQ ID NO: 475 - DNA, 213 bp, Cucumis melo
  source 1..213: mol_type = genomic DNA
atggcgtttagcaccgcacccttctacgcaattggtatcagatttcccagccatagctcatcaatctcaagcaccactaacgccctcatccttaaaagtccactggcgttagccctaaccgctaagccgaagtctcccctactcctcaagcgcaacgttggctgccagcgattcgggcgaaactcccgcttcgtcgtgcgctgcgatgcgtcc

SEQ ID NO: 476 - DNA, 234 bp, Kochia scoparia
  source 1..234: mol_type = genomic DNA
atggcgaccgccacgacctccttccccggcgcgtacctccatctcccgcccaagaacggggtccgcaacgccttgttctctcaacccatctgttcatccaacctcaacctcaagaaacctaactctctcaaatcggtgaagctgtcccgcagttccggcaatgccctattctacaagaacgccaagaagaatagtaagttcggcagtctggtcgtgcggtgcgacgcggcggga

SEQ ID NO: 477 - DNA, 234 bp, Kochia scoparia
  source 1..234: mol_type = genomic DNA
atggccaccgcgacgaccagctttcccggcgcgtatctgcacctcccgcccaagaacggcgtgagaaacgcgctgttcagtcaaccgatatgctcgtccaatctcaacctcaagaaacccaattctctgaaaagcgtcaaactgtcgcgtagtagcggcaatgcgctgttctacaagaacgccaagaagaatagcaagttcgggtcgctcgtggtgcgctgcgacgcggcgggc

SEQ ID NO: 478 - DNA, 234 bp, Kochia scoparia
  source 1..234: mol_type = genomic DNA
atggccacagccaccacgtccttccctggggcctacctacatctcccgcccaagaatggcgtgcgaaacgcgctgttcagtcagcctatatgcagcagtaatcttaacctcaagaagcctaattccctcaagtcagtgaaactgagccggtctagcgggaacgcgctgttctacaagaacgccaaaaagaatagcaagttcggctcgctcgtggtccggtgcgacgcggcgggc

SEQ ID NO: 479 - DNA, 252 bp, unidentified
  source 1..252: mol_type = genomic DNA; note = Rosa hybrida
atggcgtcatcgaccacttcgttcgccgccagtggagttggattgcggctccctcagtccgtgagcacgaagtgctgctctaaagcgtcattgttcccacaccccacactatcgttgaccttccacgctaggccacagttctttagaggcttggcgtctcgccagttcaatccaaacggagcgtttgggacgggctccggacggctgggccggacaccaaatccgtttgtcgtcagaagcgaagcgagttct

SEQ ID NO: 480 - DNA, 240 bp, Sedum album
  source 1..240: mol_type = genomic DNA
atggcggcgagcgcggctacgatcacctccagcatatcggcgattaccccgaagccgtcgtccttctcaagcagcccttcggtcaccgtgccccgattctctgtgtcgtgcagcgcgataccgcgtccacacaagaatccctgctcgttgaagttccgggtgaaggactcacggtttaacggaattgtcaagaagcgcagtaacagcaactcattcgtagtacgttgtgacacttcctcg

SEQ ID NO: 481 - DNA, 237 bp, Sedum album
  source 1..237: mol_type = genomic DNA
atggccgccgacgcagctaccattacggcgggtatcactctcacgacggcccgccgctcctcctccagtattgcgccgcagttctcggtgtgttgctcagcgattaccaacacgcaaaaaaatctgagcttcctcaagttgcgcgtgaaagacgccaccttgactacacggattgagggtattcagaagaagcggtacaactccgcgtccttcgtcgtcagatgcgacgcgagcagc

SEQ ID NO: 482 - DNA, 243 bp, Spinacia oleracea
  source 1..243: mol_type = genomic DNA
atggccactgccacgacgtcctttctcggcgcttacttgcgggtgccgcccaacaatggcgtgaggaatgcgctgttcagtcaaccgttcctgtcgcttcgcattaagtccaaacgcactaagagcctcaactcgttgaaattcacaggagactcaagtaagattctgctgtttaagtgctcccggccgtttgagaaggggcttaaatccggctcgttcgtggtgcgctgcgacgcggccggt

SEQ ID NO: 483 - DNA, 237 bp, Allium cepa
  source 1..237: mol_type = genomic DNA
atggcggcaacgagctccgcgaccactcacctcccttttttcagcccgcacaccaaacacgcaaagacaaactctttcttcgcgtcccttccggtcagcgcctactccacgaaaaactctatcagtttcaaggcgctcaaggccgtgcgatggagcgagaccttcgggcaatcgaagaaggccaatggttttgccaaaaggaagcaatttgccgtcgtgcggtgcgatgcgagttca